(19) United States
(12) Patent Application Publication
(10) Pub. No.: US 2023/0338528 A1
(43) Pub. Date: Oct. 26, 2023

(54) SYSTEMS AND METHODS FOR ENHANCED IMMUNOTHERAPIES

(71) Applicant: Hangzhou Qihan Biotech Co., Ltd., Hangzhou City (CN)

(72) Inventors: Yangbin Gao, Hangzhou City (CN); Xiangjun He, Hangzhou City (CN); Yixuan Zhou, Hangzhou City (CN); Chenyang Liao, Hangzhou City (CN); Jiabiao Hu, Hangzhou City (CN); Jing Xu, Hangzhou City (CN); Yanan Yue, Hangzhou City (CN); Luhan Yang, Hangzhou City (CN)

(21) Appl. No.: 18/311,108

(22) Filed: May 2, 2023

Related U.S. Application Data
(63) Continuation of application No. PCT/CN2021/128458, filed on Nov. 3, 2021.

(30) Foreign Application Priority Data
Nov. 3, 2020 (WO) PCT/CN2020/126179
Nov. 3, 2020 (WO) PCT/CN2020/126186
Feb. 24, 2021 (WO) PCT/CN2021/077658

(51) Int. Cl. A61K 39/00; C07K 14/54; C07K 16/42; C07K 14/47; C07K 14/705; C07K 14/74; C07K 14/715; C07K 14/735; C07K 14/495; C12N 9/12; C12N 9/16; C12N 9/48

(52) U.S. Cl. CPC A61K 39/4613; A61K 39/464402; A61K 39/4622; A61K 39/464411; C07K 14/5443; A61K 39/4644; A61K 39/464462; A61K 39/464419; C07K 16/4291; A61K 39/46444; A61K 39/464463; C07K 14/47; A61K 39/464454; C07K 14/70575; A61K 39/464412; A61K 39/464429; C07K 14/70539; C07K 14/70571; A61K 39/464434; C07K 14/715; C07K 14/70596; C07K 14/70525; C07K 14/70532; C07K 14/70578; A61K 39/4621; A61K 39/4631; A61K 39/4637; A61K 39/4635; C07K 14/70535; C07K 14/495; C07K 14/7155; C07K 14/54; C07K 14/70503; C07K 14/4748; C07K 14/5428; C12N 9/12; C12Y 207/11022; C12N 9/16; C12Y 301/03036; C12Y 301/03048; C12N 9/485; C12Y 304/11015; A61K 2239/23

(57) ABSTRACT

The present disclosure describes systems and methods for immunotherapies Immune cells can be engineered to exhibit enhanced half-life as compared to control cell (e.g., a non-engineered immune cell). Immune cells can be engineered to exhibit enhanced proliferation as compared to a control cell. Immune cells can be engineered to effectively and specifically target diseased cells (e.g., cancer cells) that a control cell otherwise is insufficient or unable to target. The engineered Immune cells disclosed herein can be engineered ex vivo, in vitro, and in some cases, in vivo. The engineered Immune cells that are prepared ex vivo or in vitro can be administered to a subject in need thereof to treat a disease (e.g., myeloma or solid tumors). The engineered Immune cells can be autologous to the subject. Alternatively, the engineered immune cells can be allogeneic to the subject.

### CROSS-REFERENCE

This application is a continuation of International Application No. PCT/CN2021/128458, filed on Nov. 3, 2021, which is a continuation-in-part of International Application No. PCT/CN2020/126186, filed Nov. 3, 2020, International Application No. PCT/CN2020/126179, filed Nov. 3, 2020, and International Application No. PCT/CN2021/077658, filed Feb. 24, 2021, each of which is entirely incorporated herein by reference.

### SEQUENCE LISTING

The instant application contains a Sequence Listing which has been submitted electronically in XML format and is hereby incorporated by references in its entirety. Said XML copy, created on May 2, 2023, is named 54809-712_301_SL.xml and is 270,452 bytes in size.

### BACKGROUND

Cancer (e.g., neoplasm, tumor) is a leading cause of death worldwide, accounting for about 10 million deaths annually Cancer continues to bring increasing health, economic, and emotional burden on individuals, families, communities, and countries. Increase understanding of cancer biology (e.g., specifically cancer immune biology) and genetic engineering has encouraged development of adoptive cell therapies (e.g., cellular immunotherapy), with a goal to treat or control a number of different cancers.

### SUMMARY

The present disclosure provides methods and systems for treating cancer. Some aspects of the present disclosure provides engineered immune cells (e.g., engineered natural killer (NK) cells) and methods of use thereof for treatment of cancer, such as, e.g., as hematologic malignancies or solid tumors.

In an aspect, the present disclosure provides a population of engineered NK cells, wherein: an engineered NK cell of the population of engineered NK cells comprises a heterologous polypeptide, wherein the heterologous polypeptide comprises a heterologous IL-15; and a persistence level of the population of engineered NK cells in an environment that is substantially free of an exogenous interleukin-2 (IL-2) is at least about 5% greater than a control persistence level of a comparable population of NK cells in a control environment comprising the exogenous IL-2.

In another aspect, the present disclosure provides a population of engineered NK cells, wherein: an engineered NK cell of the population comprises a heterologous secretory IL-15; and the population of engineered NK cells exhibits a signaling level of an endogenous downstream signaling protein of IL-15 that is at least about 0.1-fold greater than a control signaling level of the endogenous downstream signaling protein of a control population of NK cells lacking the heterologous secretory IL-15.

In another aspect, the present disclosure provides a population of engineered NK cells, wherein: an engineered NK cell of the population of engineered NK cells comprises at least one heterologous hypo-immunity regulator polypeptide comprising one or more members selected from the group consisting of PD-L2, TGF-beta, CD46, CD55, and CD59; and the population of engineered NK cells exhibits enhanced resistance against immune rejection, as compared to that of a control population of NK cells lacking the at least one heterologous hypo-immunity regulator polypeptide.

In another aspect, the present disclosure provides a population of engineered NK cells, wherein: an engineered NK cell of the population of engineered NK cells comprises a plurality of heterologous hypo-immunity regulator polypeptides; and the population of engineered NK cells exhibits enhanced resistance against immune rejection by at least about 5%, as compared to that of a control population of NK cells lacking one or more of the plurality of heterologous hypo-immunity regulator polypeptides.

In another aspect, the present disclosure provides a population of engineered NK cells, wherein: an engineered NK cell of the population of engineered NK cells comprises reduced expression of at least one endogenous immune regulating polypeptide that is not PTPN2; and a persistence level of the population of engineered NK cells that is greater than a control persistence level of a control population of NK cells lacking (i) and/or (ii).

In another aspect, the present disclosure provides a composition comprising the population of engineered NK cells as disclosed herein.

In another aspect, the present disclosure provides a method of generating the population of engineered NK cells as disclosed herein. In some embodiments, the population pf engineered NK cells are generated from induced pluripotent stem cells (iPSCs).

In another aspect, the present disclosure provides a method of treating a subject in need thereof, the method comprising administering to the subject the population of engineered NK cells as disclosed herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1G illustrate engineered NK cells comprising a CD16 variant for enhanced CD16 signaling;

FIGS. 2A-2G illustrate engineered NK cells comprising a chimeric antigen receptor against CD19; and

FIGS. 3A and 3B illustrate engineered T cells comprising heterologous human IL-15.

FIGS. 4A-4E illustrate expression of CD56⁺, NKG2A⁺, NKp30⁺, NKp44⁺, and NKp46⁺ among WT iNK and iNK differentiated from different hnCD16-iPSC clones. (FIG. 4A) the percentage of CD56⁺ cells in the total differentiated cells. (FIG. 4B) the percentage of NKG2A⁺ in the CD56⁺ population. (FIG. 4C) the percentage of NKp30⁺ in the CD56⁺ population. (FIG. 4D) the percentage of NKp44⁺ in the CD56⁺ population. (FIG. 4E) the percentage of NKp46⁺ cells in the CD56⁺ population.

FIG. 5 illustrates an overexpression of CD16 in hnCD16 (a variant with enhanced CD16 signaling) NK-92 cells detected by FACS (fluorescence-activated cell sorting) with PE (phycoerythrin) conjugated anti-CD16 antibody.

FIG. 6 illustrates the surface expression of IL-15 in iNK cells differentiated from hIL15-IL15Ra fused-1 iPSC clones detected by FACS with APC (allophycocyanin) conjugated anti-IL-15 antibody. PW15, PW18, and PW23 are clones expressing membrane-bound IL-15.

FIG. 7 illustrates an in-vitro growth curve of eNK cells differentiated from mbIL-15-expressing iPSC clones (KB-15) cultured with or without IL-2.

FIGS. 8A-8E illustrate expression of CD56⁺, NKG2A⁺, NKp30⁺, NKp44⁺, and NKp46⁺ among WT iNK and iNK differentiated from different mbIL-15-iPSC clones. (FIG. 8A) the percentage of CD56⁺ cells in the total differentiated cells. (FIG. 8B) the percentage of NKG2A⁺ in the CD56⁺ population. (FIG. 8C) the percentage of NKp30⁺ in the CD56⁺ population. (FIG. 8D) the percentage of NKp44⁺ in the CD56⁺ population. (FIG. 8E) the percentage of NKp46⁺ cells in the CD56⁺ population.

FIGS. 9A-9B illustrate NK cell concentrations in the peripheral blood of NCG mice having a Nalm-6 xenograft model at 8, 15 and 22 days post-infusion with engineered mbIL-15 eNK cells. (FIG. 9A) NK cells detected by FACS with CD56-APC antibody. (FIG. 9B) quantification of FIG. 9A. QN-019 denotes eNK expressing aCD19 CAR+hnCD16+membrane-bound IL-15. QN-001 denotes WT eNK.

FIGS. 10A-10C illustrate properties of NK cells differentiated from iPSC clones expressing secreted IL-15. (FIG. 10A) the percentage of CD56⁺ cells in the total differentiated cells among WT iNK cells and iNK differentiated from different sIL15-iPSC clones. (FIG. 10B) the concentration of IL-15 in culture medium from WT iNK cells and iNK cells differentiated from iPSC clones expressing secreted IL-15 (KA08). (FIG. 10C) an in-vitro growth curve of WT eNK cells and eNK cells differentiated from secretory IL-15-expressing iPSC clones (OQ-20), in absence of an exogenous cytokine.

FIG. 11 illustrates a testing scheme for hypoimmunity via editing and differentiating iPSC.

FIGS. 12A-12Q illustrate confirmed establishment of edit-1 clones to edit-9 clones. FIG. 12A illustrates FACS analysis of edit-1 clones (hiPSC electroporated with pre-mixed ribonucleoprotein [RNP] targeting B2M). FIG. 12B illustrates sanger sequencing of edit-1 clones. FIG. 12C illustrates sanger sequencing of edit-2 clones (hiPSC electroporated with RNP targeting CIITA). FIG. 12D illustrates FACS analysis of edit-3 clones (hiPSC electroporated with two RNPs targeting B2M and CIITA). FIG. 12E illustrates sanger sequencing of edit-3 clones. FIG. 12F illustrates FACS analysis of edit-4 clones (hiPSC electroporated with a construct overexpressing PD-L1, PD-L2, TGF-β, HLA-E, HLA-G, CD47, IL-10, CCL-21, CD46, CD55, CD59 and two RNPs, targeting B2M and CIITA). FIG. 12G illustrates sanger sequencing of edit-4 clones. FIG. 12H illustrates FACS analysis of edit-5 clones (hiPSC electroporated with a construct overexpressing of PD-L1, HLA-E, CD47, IL-10, CCL-21 and two RNPs, targeting B2M and CIITA). FIG. 12I illustrates sanger sequencing of edit-5 clones. FIG. 12J illustrates FACS analysis of edit-6 clones (hiPSC electroporated with a construct overexpressing PD-L1, HLA-E, CD47, CD46, CD55, CD59 and two RNPs, targeting B2M and CIITA). FIG. 12K illustrates sanger sequencing of edit-6 clones. FIG. 12L illustrates FACS analysis of edit-7 clones (hiPSC electroporated with a construct overexpressing PD-L1, HLA-E, CD47, CCL-21, CD55 and two RNPs, targeting B2M and CIITA). FIG. 12M illustrates sanger sequencing of edit-7 clones. FIG. 12N illustrates FACS analysis of edit-8 clones (hiPSC electroporated with a construct overexpressing of CD47 and two RNPs, targeting B2M and CIITA). FIG. 12O illustrates sanger sequencing of edit-8 clones. FIG. 12P illustrates FACS analysis of edit-9 clones (hiPSC electroporated with a construct overexpressing PD-L1, PD-L2, TGF-β, HLA-E, HLA-G, CD47, IL-10, CCL-21, CD46, CD55, CD59, two RNPs, targeting B2M and CIITA). FIG. 12Q illustrates sanger sequencing of edit-9 clones. The gene edits from edit-1 through edit-9 are summarized in FIG. 18.

FIGS. 13A-13S illustrate functional properties of edit-1 clones to edit-9 clones. (FIG. 13A) cell lysis when different edited iPSC clones co-incubating with human complement. (FIG. 13B) cell lysis when different edited iPSC clones co-incubating with cord blood-derived natural killer (CBNK). (FIG. 13C) cell counts of CD56⁺ cells among different iNK differentiated from corresponding edited iPSC. The iPSC clone number was shown in the parenthesis. (FIG. 13D) CD56⁺ percentage among different iNK differentiated from corresponding edited iPSC. The iPSC clone number was shown in the parenthesis. ((FIG. 13E) NKG2A⁺ percentage among different iNK differentiated from corresponding edited iPSC. The iPSC clone number was shown in the parenthesis. ((FIG. 13F) NK cell-mediated lysis of K562 cells when co-cultured with corresponding eNKs at a single time point. (FIG. 13G) NK cell-mediated lysis of K562 cells when co-cultured with corresponding eNKs over 6 days. (FIG. 13H) the percentage of proliferating CD8⁺ T cells when iNK with different edits are co-cultured with peripheral blood mononuclear cell (PBMC) from donor 1. (FIG. 13I) the percentage of proliferating CD8⁺ T cells when iNK with different edits are co-cultured with PBMC from donor 2. (FIG. 13J) the percentage of proliferating CD8⁺ T cells when iNK with different edits are co-cultured with PBMC from donor 3. (FIG. 13K) the percentage of proliferating CD8⁺ T cells when iNK with different edits are co-cultured with PBMC from donor 4. (FIG. 13L) the percentage of proliferating CD8⁺ T cells when iNK with different edits are co-cultured with PBMC from donor 5. (FIG. 13M) the percentage of proliferating CD8⁺ T cells when iNK with different edits are co-cultured with PBMC from donor 6. (FIG. 13N) the percentage of proliferating CD4⁺ T cells when iNK with different edits are co-cultured with PBMC from donor 1. (FIG. 13O) the percentage of proliferating CD4⁺ T cells when iNK with different edits are co-cultured with PBMC from donor 2. (FIG. 13P) the percentage of proliferating CD4⁺ T cells when iNK with different edits are co-cultured with PBMC from donor 3. (FIG. 13Q) the percentage of proliferating CD4⁺ T cells when iNK with different edits are co-cultured with PBMC from donor 4. (FIG. 13R) the percentage of proliferating CD4⁺ T cells when iNK with different edits are co-cultured with PBMC from donor 5. (FIG. 13S) the percentage of proliferating CD4⁺ T cells when iNK with different edits are co-cultured with PBMC from donor 6.

FIGS. 14A-14C illustrate a screen of engineered NK cells for persistency. (FIG. 14A) method design for in-vitro screening of the NK persistency related genes comparing culturing with low or high cytokine. (FIG. 14B) the percentage of indel in FCER1G deficient editing when cultured with low or high cytokine. (FIG. 14C) the percentage of indel in PTPN2 deficient editing when cultured with low or high cytokine.

FIGS. 15A-15G illustrate a screen of engineered NK cells for persistency. (FIG. 15A) the method design for in-vivo screening of the NK persistency related genes comparing in vitro culture and in-vivo growth. (FIG. 15B) the percentage of indel in STAT3 deficient editing in mouse liver versus being cultured with high cytokine. (FIG. 15C) the percentage of indel in STAT3 deficient editing in mouse spleen versus being cultured with high cytokine. (FIG. 15D) the percentage of indel in STAT3 deficient editing in mouse bone marrow (BM) versus being cultured with high cytokine. (FIG. 15E) the percentage of indel in PTPN2 deficient editing in mouse liver versus being cultured with high cytokine. (FIG. 15F) the percentage of indel in PTPN2 deficient editing in mouse spleen versus being cultured with high cytokine. (FIG. 15G) the percentage of indel in PTPN2 deficient editing in mouse bone marrow (BM) versus being cultured with high cytokine.

FIGS. 16A-16B illustrate properties of NK92 cells with CD33-CAR integration. (FIG. 16A) schematic of CD33 CAR structure design. TM stands for transmembrane domain; SCFV stands for single chain variable fragment. (FIG. 16B) targeted cytotoxicity of CD33-CAR NK92 cells on KG1 cells.

FIGS. 17A-17D illustrate properties of NK92 cells with BCMA-CAR integration. (FIG. 17A) schematic of BCMA CAR structure design. TM stands for transmembrane domain; SCFV stands for single chain variable fragment. (FIG. 17B) targeted cytotoxicity of BCMA-CAR NK92 cells on RPMI8826 cells. (FIG. 17C) percentage of expression of CD107a in BCMA-CAR NK92 cells versus WT NK92 cells. (FIG. 17D) percentage of expression of IFN-γ in BCMA-CAR NK92 cells versus WT NK92 cells.

FIG. 18 is a summary of the gene edits from edit-1 through edit-9.

FIG. 19A shows different chimeric receptor polypeptide constructs (e.g., different chimeric antigen receptor constructs) comprising an antigen binding moiety capable of specific binding to CD19. FIGS. 19B and 19C show targeted cytotoxicity of NK cells comprising one of the different chimeric receptor polypeptides shown in FIG. 19A, against CD19-presenting target cells.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a,” “an,” and “the” include plural references unless the context clearly dictates otherwise. For example, the term “a chimeric transmembrane receptor” includes a plurality of chimeric transmembrane receptors.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e g fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The term “reprogramming,” “dedifferentiation,” “increasing cell potency,” or “increasing developmental potency,” as used interchangeable herein, generally refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

The term “differentiation” generally refers to a process by which an unspecialized (“uncommitted”) or less specialized cell acquires the features of a specialized cell such as, e.g., an immune cell. A differentiated or differentiation-induced cell is one that has taken on a more specialized (“committed”) position within the lineage of a cell. The term “committed” generally refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

The term “pluripotent” generally refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency can be a continuum of developmental potencies ranging from the incompletely or partially pluripotent cell (e.g., an epiblast stem cell), which is unable to give rise to a complete organism to the more primitive, more pluripotent cell, which is able to give rise to a complete organism (e.g., an embryonic stem cell).

The term “induced pluripotent stem cells” (iPSCs) generally refers to stem cells that are derived from differentiated cells (e.g., differentiated adult, neonatal, or fetal cells) that have been induced or changed (i.e., reprogrammed) into cells capable of differentiating into tissues of all three germ or dermal layers: mesoderm, endoderm, and ectoderm. The iPSCs produced do not refer to cells as they are found in nature. In some cases, iPSCs can be engineered to differentiation directly into committed cells (e.g., natural killer (NK) cells. In some cases, iPSCs can be engineered to differentiate first into tissue-specific stem cells (e.g., hematopoietic stem cells (HSCs)), which can be further induced to differentiate into committed cells (e.g., NK cells).

The term “embryonic stem cell” (ESCs) generally refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. In some cases, ESCs can be engineered to differentiation directly into committed cells (e.g., NK cells). In some cases, ESCs can be engineered to differentiate first into tissue-specific stem cells (e.g., HSCs), which can be further induced to differentiate into committed cells (e.g., NK cells).

The term “isolated stem cells” generally refers to any type of stem cells disclosed herein (e.g., ESCs, HSCs, mesenchymal stem cells (MSCs), etc.) that are isolated from a multicellular organism. For example, HSCs can be isolated from a mammal's body, such as a human body. In another example, an embryonic stem cells can be isolated from an embryo.

The term “isolated” generally refers to a cell or a population of cells, which has been separated from its original environment. For example, a new environment of the isolated cells is substantially free of at least one component as found in the environment in which the “un-isolated” reference cells exist. An isolated cell can be a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an isolated cell is partly or completely separated from at least one component, including other substances, cells or cell populations, as it is found in nature or as it is grown, stored or subsisted in non-naturally occurring environments.

The term “hematopoietic stem and progenitor cells,” “hematopoietic stem cells,”, “hematopoietic progenitor cells,” or “hematopoietic precursor cells,” as used interchangeably herein, generally refers to cells which are committed to a hematopoietic lineage but are capable of further hematopoietic differentiation (e.g., into NK cells) and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). In some cases, HSCs can be CD34+ hematopoietic cells capable of giving rise to both mature myeloid and lymphoid cell types including T cells, NK cells and B cells.

The term “immune cell” generally refers to a differentiated hematopoietic cell. Non-limiting examples of an immune cell can include an NK cell, a T cell, a monocyte, an innate lymphocyte, a tumor-infiltrating lymphocyte, a macrophage, a granulocyte, etc.

The term “NK cell” or “Natural Killer cell” generally refers to a subset of peripheral blood lymphocytes defined by the expression of CD56 or CD16 and the absence of the T cell receptor (CD3). In some cases, NK cells that are phenotypically CD3− and CD56+, expressing at least one of NKG2C and CD57 (e.g., NKG2C, CD57, or both in same or different degrees), and optionally, CD16, but lack expression of one or more of the following: PLZF, SYK, FceRγ, and EAT-2. In some cases, isolated subpopulations of CD56⁺ NK cells can exhibit expression of CD16, NKG2C, CD57, NKG2D, NCR ligands, NKp30, NKp40, NKp46, activating and inhibitory KIRs, NKG2A and/or DNAM-1.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G] dCTP, [TAMRA] dCTP, [JOE] ddATP, [R6G] ddATP, [FAM] ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or flurescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid Amino acid analogues can refer to amino acid derivatives. The term “amino acid” includes both D-amino acids and L-amino acids.

The term “derivative,” “variant,” or “fragment,” as used herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In some cases, an engineered or recombinant polynucleotide (e.g., a genomic DNA sequence) can be modified or altered by a gene editing moiety.

The term “gene editing moiety” generally refers to a moiety which can edit a nucleic acid sequence, whether exogenous or endogenous to a cell comprising the nucleic acid sequence. In some embodiments, a gene editing moiety regulates expression of a gene by editing a nucleic acid sequence. In some cases, a gene editing moiety can regulate expression of a gene by editing genomic DNA sequence. In some cases, a gene editing moiety can regulate expression of a gene by editing an mRNA template. Editing a nucleic acid sequence can, in some cases, alter the underlying template for gene expression.

Alternatively or in addition to, a gene editing moiety can be capable of regulating expression or activity of a gene by specifically binding to a target sequence operatively coupled to the gene (or a target sequence within the gene), and regulating the production of mRNA from DNA, such as chromosomal DNA or cDNA. In some cases, a gene editing moiety can recruit or comprise at least one transcription factor that binds to a specific DNA sequence, thereby controlling the rate of transcription of genetic information from DNA to mRNA. A gene editing moiety can itself bind to DNA and regulate transcription by physical obstruction, for example preventing proteins such as RNA polymerase and other associated proteins from assembling on a DNA template. A gene editing moiety can regulate expression of a gene at the translation level, for example, by regulating the production of protein from mRNA template. In some cases, a gene editing moiety can regulate gene expression by affecting the stability of an mRNA transcript.

The term “antibody” generally refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e. IgA, IgG, IgM, IgD and IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)2, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The term “chimeric polypeptide receptor” generally refers to a non-natural polypeptide receptor comprising one or more antigen binding moieties, each antigen binding moiety capable of binding to a specific antigen. A chimeric polypeptide receptor can be monospecific (i.e., capable of binding to one type of specific antigen). Alternatively, a chimeric polypeptide receptor can be multi-specific (i.e., capable of binding to two or more different types of specific antigens). A chimeric polypeptide receptor can be monovalent (i.e., comprising a single antigen binding moiety). Alternatively, a chimeric polypeptide receptor can be multivalent (i.e., comprising a plurality of antigen binding moieties). In some cases, a chimeric polypeptide receptor can comprise a T-cell receptor (TCR) fusion protein (TFP) or a chimeric antigen receptor (CAR).

The term “antigen binding domain” generally refers to a construct exhibiting preferential binding to a specific target antigen. An antigen binding domain can be a polypeptide construct, such as an antibody, a functional variant thereof (e.g., a designed ankyrin repeat protein (DARPin)), modification thereof, fragment thereof, or a combination thereof. The antigen binding domain can be any antibody as disclosed herein, or a functional variant thereof. Non-limiting examples of an antigen binding domain can include a murine antibody, a human antibody, a humanized antibody, a camel Ig, a shark heavy-chain-only antibody (VNAR), Ig NAR, a chimeric antibody, a recombinant antibody, or antibody fragment thereof. Non-limiting examples of antibody fragment include Fab, Fab′, F(ab)′2, F(ab)′3, Fv, single chain antigen binding fragment (scFv), (scFv)2, disulfide stabilized Fv (dsFv), minibody, diabody, triabody, tetrabody, single-domain antigen binding fragments (sdAb, Nanobody), recombinant heavy-chain-only antibody (VHH), and other antibody fragments that maintain the binding specificity of the whole antibody.

The term “designed ankyrin repeat protein” or “DARPin” generally refers to a synthetic polypeptide comprising one or more ankyrin repeat domains, wherein the one or more ankyrin repeat domains are capable of binding to one or more antigens. The ankyrin repeat domains described herein generally comprise at least one ankyrin repeat motif. In some examples, the ankyrin repeat motif comprises of two anti-parallel α-helices followed by a beta-bulge and beta-hairpin containing loop connecting it to the next repeat, each of which has about 33 residues. Recombinant proteins, or binding domains thereof, comprising designed ankyrin repeat motifs may be referred to as DARPin proteins or DARPin polypeptides.

In some cases, the ankyrin repeat domains described herein may comprise (i) a core scaffold that provides structure and (ii) target binding residues that bind to a target (e.g., a target antigen). The structural core may comprise conserved amino acid residues, and the target binding surface may comprise amino acid residues that differ depending on the target. In another example, an ankyrin repeat motif can comprise the following sequence: DxxGxTPLHLAxxxGxxxVVxLLLxxGADVNAx (SEQ ID NO: 260), wherein “x” denotes any amino acid. In another example, an ankyrin repeat motif can comprise the following sequence: DxxGxTPLHLAxxxGxxxLVxVLLxxGADVNAx (SEQ ID NO: 261), wherein “x” denotes any amino acid.

In some cases, multiple ankyrin repeat domains can be linked (either through a covalent bond or non-covalent association) to form bispecific or multi-specific molecules (e.g., bispecific or multi-specific chimeric polypeptide receptors).

Examples and details of DARPin constructs are provided in, for example, International Patent Application No. PCT/EP2001/010454, which is entirely incorporated herein by reference.

The term “safety switch” generally refers to an engineered polypeptide construct designed to prevent potential toxicity or otherwise adverse effects of a cell therapy. When expressed in a cell, the safety switch can induce death of the host cell, thereby inactivating activity of the cell in a host (e.g., in a subject's body). Thus, the safety switch can be a suicide moiety. In some cases, the cell can be programmed to express the suicide moiety at certain stage of its life-cycle (e.g., time-programmed). In some cases, expression of the suicide moiety in a cell can be conditional or inducible. In some examples, conditional regulation (e.g., expression) of a suicide moiety can include control through a small molecule-mediated post-translational activation and tissue-specific and/or temporal transcriptional regulation. Thus, the safety switch can be an inducible suicide moiety. A safety switch can mediate induction of apoptosis, inhibition of protein synthesis, DNA replication, growth arrest, transcriptional and post-transcriptional genetic regulation, and/or antibody-mediated depletion. In some cases, a safety switch can be activated by an exogenous molecule (e.g., a drug or a prodrug) that, when activated, triggers apoptosis and/or cell death of a cell (e.g., engineered NK cell as disclosed herein).

The term “immune regulator polypeptide” generally refers to a polypeptide construct (e.g., protein, antibody, membrane-bound polypeptide, secretory polypeptide, cleavable polypeptide, non-cleavable polypeptide, etc.) capable of regulating or controlling one or more attributes of an immune cell, such as a NK cell. One or more attributes of an immune cell can comprise differentiation of the immune cell, immune cell morphology, expression of a polynucleotide or polypeptide construct within the immune cell, or activity of the immune cell (e.g., cytotoxic activity of an engineered NK cell against a diseased cell, such as a cancer cell). An immune regulator polypeptide can be endogenous to a host cell. Alternatively or in addition to, an immune regulator polypeptide can be heterologous to a hots cell. In some cases, controlling the one or more attributes of the immune cell can be mediated by downregulating expression of the immune regulator polypeptide (e.g., suppression, knock-down or knock-out). Alternatively or in addition to, controlling the one or more attributes of the immune cell can be mediated by upregulating expression of the immune regulator polypeptide (e.g., upregulation of an endogenous gene or knock-in of a heterologous gene encoding the immune regulator polypeptide). Yet in another alternative or additionally, controlling the one or more attributes of the immune cell can be mediated by maintaining expression of the immune regulator polypeptide for time period that is longer than a natural or normal expression profile of the immune regulator polypeptide in a host cell. In some cases, an immune regulator polypeptide can comprise a hypo-immunity regulator. In some case, an immune regulator polypeptide can comprise an immune checkpoint inhibitor.

The term “hypo-immunity regulator” generally refers to a polypeptide construct in a cell, wherein either enhanced expression (e.g., via knock-in of a heterologous gene) or reduced expression (e.g., via knock-out or knock-down of an endogenous gene) of the hypo-immunity regulator in the cell can help the cell to reduce or avoid immune response (e.g., immune attack, such as adaptive immune rejection) from a host's body upon administration to the host's body. In some cases, cells (e.g., engineered NK cells as disclosed herein) can be modified to exhibit either enhanced expression or reduced expression of the hypo-immunity regulator, such that the cells can evade the host immune attack upon second or further infusion of the cells into the host (i.e., recipient). As such, the cells (i) would not be rejected by the host's immune system (e.g., antibody-mediated complement cytotoxicity, or antibody-dependent cellular cytotoxicity (ADCC)) and/or (ii) would be rejected at a slower rate by the host's immune system as compared with a control cell without the enhanced expression or reduced expression of the hypo-immunity regulator. A cell exhibiting the enhanced expression or reduced expression of the hypo-immunity regulator can be referred to as exhibiting “hypo-immunity” or being “immune-privileged.” For example, enhanced hypo-immunity (e.g., enhanced resistance against ADCC) of a population of engineered immune cells (e.g., a population of engineered NK cells) as disclosed herein can be ascertained in vitro (e.g., in the presence of human serum or human complement and an antibody (e.g., SSEA-4 antibody)) or in vivo (e.g., upon administration to a subject's bloodstream).

In some cases, engineering of an immune cell (e.g., NK cells) as disclosed herein can enhance the immune cell's resistance against immune rejection (e.g., ADCC) by at least or up to about 5%, at least or up to about 10%, at least or up to about 15%, at least or up to about 20%, at least or up to about 25%, at least or up to about 30%, at least or up to about 40%, at least or up to about 50%, at least or up to about 60%, at least or up to about 70%, at least or up to about 80%, at least or up to about 85%, at least or up to about 90%, at least or up to about 95%, at least or up to about 100%, at least or up to about 150%, at least or up to about 200%, at least or up to about 300%, at least or up to about 400%, or at least or up to about 500%.

In some cases, the enhanced resistance against immune rejection (e.g., ADCC) can be ascertained in vitro in a medium comprising at least or up to about 5%, at least or up to about 10%, at least or up to about 15%, at least or up to about 20%, at least or up to about 25%, at least or up to about 30%, at least or up to about 40%, at least or up to about 50%, at least or up to about 60%, at least or up to about 70%, or at least or up to about 80%, human complement.

The term “immune checkpoint inhibitor” generally refers to a group of molecules presented on a cell surface of an immune cell (e.g., T cells, myeloid cells, NK cells, B cells, etc.) that can modulate immune response of the cell by down-regulating or inhibiting the immune response of the immune cell against a target cell, such as a cancer cell (i.e., anti-cancer or anti-tumor immune response). The target cell can express a receptor or a ligand of the immune checkpoint inhibitor presented on the surface of the immune cell, to engage with the immune checkpoint inhibitor and down-regulate or inhibit the immune response of the immune cells against the target cell. As such, down-regulating or inhibiting expression of the immune checkpoint inhibitor in the immune cell can, in some cases, enhance or prolong the immune response of the immune cell against a target cell.

The term “immune response” generally refers to T cell mediated and/or B cell mediated immune responses from a host's immune system to an object (e.g., a foreign object). An example of an immune response include T cell responses, e.g., cytokine production and cellular cytotoxicity. IN some cases, an immune response can be indirectly effected by T cell activation, e.g., antibody production (humoral responses) and activation of cytokine responsive cells, such as macrophages.

The term “enhanced expression,” “increased expression,” or “upregulated expression” generally refers to production of a moiety of interest (e.g., a polynucleotide or a polypeptide) to a level that is above a normal level of expression of the moiety of interest in a host strain (e.g., a host cell). The normal level of expression can be substantially zero (or null) or higher than zero. The moiety of interest can comprise an endogenous gene or polypeptide construct of the host strain. The moiety of interest can comprise a heterologous gene or polypeptide construct that is introduced to or into the host strain. For example, a heterologous gene encoding a polypeptide of interest can be knocked-in (KI) to a genome of the host strain for enhanced expression of the polypeptide of interest in the host strain.

The term “enhanced activity,” “increased activity,” or “upregulated activity” generally refers to activity of a moiety of interest (e.g., a polynucleotide or a polypeptide) that is modified to a level that is above a normal level of activity of the moiety of interest in a host strain (e.g., a host cell). The normal level of activity can be substantially zero (or null) or higher than zero. The moiety of interest can comprise a polypeptide construct of the host strain. The moiety of interest can comprise a heterologous polypeptide construct that is introduced to or into the host strain. For example, a heterologous gene encoding a polypeptide of interest can be knocked-in (KI) to a genome of the host strain for enhanced activity of the polypeptide of interest in the host strain.

The term “reduced expression,” “decreased expression,” or “downregulated expression” generally refers to a production of a moiety of interest (e.g., a polynucleotide or a polypeptide) to a level that is below a normal level of expression of the moiety of interest in a host strain (e.g., a host cell). The normal level of expression is higher than zero. The moiety of interest can comprise an endogenous gene or polypeptide construct of the host strain. In some cases, the moiety of interest can be knocked-out or knocked-down in the host strain. In some examples, reduced expression of the moiety of interest can include a complete inhibition of such expression in the host strain.

The term “reduced activity,” “decreased activity,” or “downregulated activity” generally refers to activity of a moiety of interest (e.g., a polynucleotide or a polypeptide) that is modified to a level that is below a normal level of activity of the moiety of interest in a host strain (e.g., a host cell). The normal level of activity is higher than zero. The moiety of interest can comprise an endogenous gene or polypeptide construct of the host strain. In some cases, the moiety of interest can be knocked-out or knocked-down in the host strain. In some examples, reduced activity of the moiety of interest can include a complete inhibition of such activity in the host strain.

The term “subject,” “individual,” or “patient,” as used interchangeably herein, generally refers to a vertebrate, preferably a mammal such as a human Mammals include, but are not limited to, murines, simians, humans, farm animals, sport animals, and pets. Tissues, cells and their progeny of a biological entity obtained in vivo or cultured in vitro are also encompassed.

The term “treatment” or “treating” generally refers to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” generally refers to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” generally refers to that quantity of a composition that is sufficient to delay the manifestation, arrest the progression, relieve or alleviate at least one symptom of a disorder treated by the methods of the present disclosure.

A. Overview

T cells are part of the adaptive immune system and can be primed to recognize a specific threat by recognizing immune proteins (i.e., antigens) on a foreign cell surface. In contrast, NK cells are part of the innate immune response and can respond to a broad range of objects that consider to be “non-self” Generally, unlike T cells, NK cells can attack their target cells without sensitization (i.e., antigen-specific priming) to eliminate foreign substances.

Unmodified NK cells derived from a subject (e.g., derived from HSCs of the subject) can be cultured and expanded ex vivo, then administered to the subject as a treatment to attack their target cells, e.g., cancer cells. However, NK cell-based therapies can be limited due to short half-life and/or poor proliferation of NK cells ex vivo or in vivo. In addition, unmodified NK cells can be ineffective in targeting harder-to-treat cancers, such as myeloma or solid tumors. Furthermore, ex vivo production of NK cells based on blood-derived stem cells (e.g., HSCs) can yield a limited supply of NK cells for effective adoptive immunotherapy.

Thus, there remains a significant unmet need for NK cells sourced and engineered to exhibit, for example, enhanced proliferation, half-life, and cytotoxic activity against target cells.

B. Engineered Immune Cells

The present disclosure describes systems and methods for immunotherapies Immune cells can be engineered to exhibit enhanced half-life as compared to control cell (e.g., a non-engineered immune cell). Immune cells can be engineered to exhibit enhanced proliferation as compared to a control cell. Immune cells can be engineered to effectively and specifically target diseased cells (e.g., cancer cells) that a control cell otherwise is insufficient or unable to target. The engineered Immune cells disclosed herein can be engineered ex vivo, in vitro, and in some cases, in vivo. The engineered Immune cells that are prepared ex vivo or in vitro can be administered to a subject in need thereof to treat a disease (e.g., myeloma or solid tumors). The engineered Immune cells can be autologous to the subject. Alternatively, the engineered immune cells can be allogeneic to the subject.

In some cases, engineered immune cells (e.g., engineered NK cells) disclosed herein can be derived from an isolated stem cell (e.g., isolated ESCs). In some cases, engineered immune cells disclosed herein can be derived from induced stem cells (e.g., iPSCs).

In some cases, the stem cell disclosed herein (e.g., isolated stem cell, induced stem cell) can be an autologous cell or derived from the autologous cell. The autologous cell can be obtained from a subject having a condition or is suspected of having the condition. Alternatively, the autologous cell can be obtained from the subject before the subject is found to have the condition. In some cases, the autologous cell can be an allogeneic cell, e.g., a universal stem cell with reduced immunogenicity and with reduced amount or no need for immunosuppressive drugs. The autologous cell can be obtained from a healthy donor.

In some cases, the engineered immune cell (e.g., engineered NK cell) can be an autologous cell. The engineered immune cell can be obtained from a subject having a condition or is suspected of having the condition. Alternatively, the engineered immune cell can be obtained from the subject before the subject is found to have the condition. In some cases, the engineered immune cell can be an allogeneic cell, e.g., for a universal allogenic immunotherapy with reduced immunogenicity and with reduced amount or no need for immunosuppressive drugs. The engineered immune cell can be obtained from a healthy donor.

In some aspects, T cells can be engineered to exhibit enhanced half-life as compared to control cell (e.g., a non-engineered T cell). T cells can be engineered to exhibit enhanced proliferation as compared to a control cell. T cells can be engineered to effectively and specifically target diseased cells (e.g., cancer cells) that a control cell otherwise is insufficient or unable to target. The engineered T cells disclosed herein can be engineered ex vivo, in vitro, and in some cases, in vivo. The engineered T cells that are prepared ex vivo or in vitro can be administered to a subject in need thereof to treat a disease (e.g., myeloma or solid tumors). The engineered T cells can be autologous to the subject. Alternatively, the engineered T cells can be allogeneic to the subject.

In some aspects, NK cells can be engineered to exhibit enhanced half-life as compared to control cell (e.g., a non-engineered NK cell). NK cells can be engineered to exhibit enhanced proliferation as compared to a control cell. NK cells can be engineered to effectively and specifically target diseased cells (e.g., cancer cells) that a control cell otherwise is insufficient or unable to target. The engineered NK cells disclosed herein can be engineered ex vivo, in vitro, and in some cases, in vivo. The engineered NK cells that are prepared ex vivo or in vitro can be administered to a subject in need thereof to treat a disease (e.g., myeloma or solid tumors). The engineered NK cells can be autologous to the subject. Alternatively, the engineered NK cells can be allogeneic to the subject.

In one aspect, the present disclosure provides an engineered immune cell (e.g., an engineered NK cell). The engineered immune cell can comprise a cytokine (e.g., a secretory cytokine) that is heterologous to the immune cell. In some cases, the heterologous cytokine can comprise a heterologous interleukin (IL) (e.g., a heterologous secretory IL-15). The engineered immune cell can further comprise one or both of: (i) a CD16 variant for enhanced CD16 signaling as compared to a control cell and (ii) a chimeric polypeptide receptor comprising an antigen binding moiety capable of binding to an antigen. In some examples, the antigen is not CD19. Thus, the antigen binding moiety may not and need not exhibit any specific binding to CD19, but rather a specific binding to an antigen (e.g., one or more antigens) that is not CD19.

The engineered immune cell (e.g., an engineered NK cell) as disclosed herein can comprise a heterologous receptor that is a respective receptor of the heterologous cytokine as disclosed herein (e.g., heterologous IL-15 receptor (IL-15R, such as IL-15α or IL-15β) for heterologous IL-15). Alternatively, the engineered immune cell may not and need not comprise any heterologous receptor that is a respective receptor of the heterologous cytokine. For example, the engineered immune cell comprising a heterologous IL (e.g., IL-15) lacks a heterologous receptor of the heterologous IL (e.g., IL-15R).

In one aspect, the present disclosure provides an engineered immune cell (e.g., an engineered NK cell). The engineered immune cell can comprise a cytokine (e.g., a secretory cytokine) that is heterologous to the immune cell. The heterologous cytokine can further comprise a heterologous interleukin (IL) (e.g., a heterologous secretory IL-15). The engineered immune cell may and need not comprise a heterologous receptor that is a respective receptor of the heterologous cytokine (e.g., a heterologous IL-15R).

The heterologous cytokine (e.g., the heterologous IL) as disclosed herein can be of the same species as that of the engineered immune cell (e.g., the engineered NK cell). For example, both the heterologous cytokine and the engineered immune cell can be of human origin. Alternatively, the heterologous cytokine can be of a different species than that of the engineered immune cell.

A heterologous cytokine (e.g., the heterologous IL) as disclosed herein can be introduced to the engineered immune cell (e.g., engineered NK cell) by contacting a heterologous polynucleotide encoding the heterologous cytokine to the engineered immune cell. The heterologous polynucleotide can be integrated into the engineered immune cell's chromosome (e.g., nuclear chromosome). Alternatively, the heterologous polynucleotide may not and need not be integrated into the chromosome of the engineered immune cell. In an example, a mRNA encoding a heterologous cytokine can be introduced (or inserted into) the engineered immune cell.

In some cases, the heterologous cytokine as disclosed herein can be a heterologous IL. A heterologous IL as disclosed herein can comprise at least 1, 2, 3, 4, 5, or more different types of heterologous ILs. A heterologous IL as disclosed herein can comprise at most 5, 4, 3, or 2 different type of heterologous ILs. Alternatively, the heterologous IL can be a single type of heterologous IL. Non-limiting examples of the heterologous IL can include, but are not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36. In some examples, the heterologous IL can comprise one or more members selected from the group consisting of IL2, IL4, IL6, IL7, IL9, IL10, IL11, IL12, IL15, IL18, IL21, and functional modifications thereof. For example, the engineered immune cell (e.g., an engineered NK cell) as disclosed herein can comprise at least a portion of heterologous human IL-15 (or a gene encoding thereof).

The heterologous cytokine (e.g., the heterologous IL) as disclosed herein can be a secretory cytokine. Alternatively, the heterologous cytokine may not and need not be secreted by the engineered immune cell. In such a case, for example, the heterologous cytokine can be bound to a cell surface of the engineered immune cell.

In some cases, the heterologous cytokine (e.g., the heterologous IL) as disclosed herein can be a secretory cytokine. An expression cassette encoding the heterologous cytokine can be introduced to the engineered immune cell. The expression cassette can further encode an additional heterologous polypeptide, e.g., a heterologous receptor. Within the expression cassette, a first polynucleotide sequence encoding the heterologous cytokine and a second polynucleotide sequence encoding the additional heterologous polypeptide (e.g., the heterologous receptor) can be coupled to each other via a polynucleotide linker encoding a cleavage linker. In some examples, the heterologous receptor can be a respective receptor of the heterologous cytokine (e.g., heterologous IL-15a or IL-15β for heterologous IL-15). Alternatively, the expression cassette may not and need not encode any additional heterologous polypeptide other than the heterologous cytokine.

A cleavable linker as disclosed herein can comprise a self-cleaving peptide, such as a self-cleaving 2A peptide. Self-cleaving peptides can be found in members of the Picornaviridae virus family, including aphthoviruses such as foot-and-mouth disease virus (FMDV), equine rhinitis A virus (ERAV), Thosea asigna virus (TaV) and porcine tescho virus-1 (PTV-I), and cardioviruses such as Theilovirus (e.g., Theiler's murine encephalomyelitis) and encephalomyocarditis viruses. Non-limiting examples of the self-cleaving 2A peptide can include “F2A”, “E2A”, “P2A”, “T2A”, and functional variants thereof.

In some cases, the heterologous cytokine (e.g., the heterologous IL) as disclosed herein can be bound to a cell surface the engineered immune cell (e.g., the engineered NK cell). In some examples, the engineered immune cell can be genetically modified such that a heterologous polynucleotide sequence encoding the heterologous cytokine is coupled to a gene encoding an endogenous transmembrane protein of the engineered immune cell. In an example, the endogenous transmembrane protein can be a respective receptor of the heterologous cytokine (e.g., heterologous IL-15a or IL-15β for heterologous IL-15). In some examples, an expression cassette encoding a heterologous fusion polypeptide comprising (i) the heterologous cytokine that is coupled to (ii) a heterologous receptor can be introduced to the engineered immune cell. Here, the heterologous cytokine may not and need not be cleavable from the heterologous receptor. Non-limiting examples of the heterologous receptor can include a respective receptor of the heterologous cytokine (e.g., heterologous IL-15a or IL-15β for heterologous IL-15), or a different receptor such as a common gamma chain (γ_{C}) receptor or a modification thereof.

An expression cassette as disclosed herein can be integrated into the genome of the engineered cell (e.g., the engineered NK cell) via action of a gene editing moiety as disclosed herein. Alternatively, the expression cassette may not and need not be integrated into the genome of the engineered cell.

The engineered immune cell (e.g., the engineered NK cell) as disclosed herein can exhibit enhanced signaling of an endogenous signaling pathway that involves the heterologous cytokine (e.g., the heterologous IL, such as the heterologous IL-15) and/or the heterologous receptor (e.g., the heterologous IL receptor, such as the heterologous IL-15R) as disclosed herein. The enhanced signaling of the endogenous signaling pathway as disclosed herein can be ascertained by a number of methods, including, but are not limited to, (i) phosphorylation of a downstream signaling protein (e.g., JAK3, STAT3, STATS, etc. for IL-15/IL-15R) or (ii) expression of a downstream gene (e.g., Mcl1, Cdk4/6, Mki67, Tnf, Gzmb, Gzmc, Ifng, etc. for IL-15/IL-15R) via Western blotting or polymerase chain reaction (PCR) techniques.

In some cases, enhanced signaling of the endogenous signaling pathway that is induced by the heterologous cytokine and/or the heterologous receptor (e.g., induced by the heterologous cytokine and/or heterologous receptor, such as IL-15/IL-15R as disclosed herein) in the engineered immune cell of the present disclosure can be characterized by an increase in phosphorylation of a downstream signaling protein by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell.

In some cases, enhanced signaling of the endogenous signaling pathway that is induced by the heterologous cytokine and/or the heterologous receptor (e.g., induced by the heterologous cytokine and/or heterologous receptor, such as IL-15/IL-15R as disclosed herein) in the engineered immune cell of the present disclosure can be characterized by an increased expression of a downstream gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell.

Enhanced CD16 signaling (e.g., constitutively activated signaling of CD16) of the engineered immune cell (e.g., engineered NK cell) as disclosed herein can be achieved by having non-cleavable CD16 variant in the subject cell. CD16 (e.g., CD16a) is a transmembrane protein expressed by immune cells (e.g., NK cells), which binds monomeric IgG attached to target cells to activate the immune cells and facilitate antibody-dependent cell-mediated cytotoxicity (ADCC). In a non-engineered immune cell, the binding between CD16 and the monomeric IgG can induce cleavage of the CD16 protein at a cleavage site near the transmembrane domain, to regulates the cell surface density of CD16 upon immune cell activation. Thus, the endogenous CD16 of the engineered immune cell can be modified to enhance its signaling. Alternatively, an enhanced signaling variant of CD16 can be artificially introduced to the engineered immune cell.

In some cases, the engineered immune cell's endogenous gene encoding CD16 can be genetically modified in its ectodomain (e.g., F176V) via action of a gene editing moiety as disclosed herein, such that the modified CD16 exhibits higher binding affinity to its target (e.g., monomeric IgG) as compared to a natural CD16. In some cases, a heterologous gene encoding such modified CD16 can be introduced to the cell.

In some cases, the engineered immune cell's endogenous gene encoding CD16 can be genetically modified via action of a gene editing moiety as disclosed herein, such that the modified CD16 is non-cleavable and can induce enhanced CD16 signaling. In some examples, the cleavage site (e.g., position 195-198) in the membrane-proximal region (position 189-212) of CD16 can be modified or eliminated (e.g., CD16 S197P variant as a non-cleavable CD16 variant). In some cases, a heterologous gene encoding such modified CD16 can be introduced to the cell.

In some cases, a heterologous gene encoding a heterologous CD16 variant that (i) exhibits higher binding affinity to its target (e.g., monomeric IgG) and (ii) is non-cleavable can be introduced to the cell (i.e., hnCD16). In some examples, the heterologous CD16 variant can be a modified CD16 comprising, for example, F176V and S197P, as disclosed herein. In some examples, the heterologous CD variant can be a fusion receptor protein comprising (i) at least a portion of CD16 with an inactivated cleavage site and (ii) an ectodomain of a different cell surface protein, such as a glycoprotein (e.g., CD64), that exhibits enhanced binding to the target (e.g., monomeric IgG) as compared to an unmodified CD16.

A heterologous gene as disclosed herein can be integrated into the genome of the engineered cell (e.g., the engineered NK cell) via action of a gene editing moiety as disclosed herein. Alternatively, the heterologous gene may not and need not be integrated into the genome of the engineered cell.

The enhanced CD16 signaling of the engineered immune cell (e.g., the engineered NK cell) as disclosed herein can be ascertained by a number of methods, including, but are not limited to, (i) phosphorylation of a downstream signaling protein (e.g., SHP-1) via Western blotting or (ii) expression of a downstream gene (e.g., CD25, IFN-gamma, TNF, etc.) via Western blotting or PCR techniques.

In some cases, the CD16 signaling of the engineered immune cell (e.g., the engineered NK cell comprising hnCD16) of the present disclosure can be greater than CD16 signaling of a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, enhanced CD16 signaling of the engineered immune cell (e.g., the engineered NK cell comprising hnCD16) of the present disclosure can be characterized by an increase in phosphorylation of a downstream signaling protein by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell.

In some cases, enhanced CD16 signaling of the engineered immune cell (e.g., the engineered NK cell comprising hnCD16) of the present disclosure can be characterized by an increased expression of a downstream gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell.

In some cases, the CD16 signaling of the engineered immune cell (e.g., the engineered NK cell comprising hnCD16) of the present disclosure can be more prolonged (e.g., a longer duration of time of activated CD16 signaling) than CD16 signaling of a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In one aspect, the present disclosure provides an engineered immune cell (e.g., an engineered NK cell). The engineered immune cell can comprise a heterologous cytokine as disclosed herein, wherein the heterologous cytokine is bound to a membrane (e.g., plasma membrane) of the engineered immune cell. In some cases, the heterologous cytokine can comprise a heterologous IL as disclosed herein (e.g., a heterologous IL-15). The engineered immune cell can further comprise one, two, or all of: (a) a different heterologous cytokine (e.g., a heterologous cytokine as disclosed herein, other than the one that is bound to the membrane of the subject cell), (b) reduced expression or activity of an endogenous immune regulator polypeptide, and (c) a safety switch. In some examples, the endogenous immune regulator polypeptide is not B2M. Thus, the endogenous immune regulator can be, for example, a polypeptide other than B2M.

The engineered immune cell (e.g., an engineered NK cell) can comprise the different heterologous cytokine and one or both of (b) the reduced expression or activity of an endogenous immune regulator polypeptide (e.g., a non-B2M polypeptide) and (c) the safety switch. The engineered immune cell comprise the reduced expression or activity of an endogenous immune regulator polypeptide (e.g., a non-B2M polypeptide) and one or both of (a) the different heterologous cytokine and (c) the safety switch. The engineered immune cell comprise the safety switch and one or both of (a) the different heterologous cytokine and (b) the reduced expression or activity of an endogenous immune regulator polypeptide (e.g., a non-B2M polypeptide). The engineered immune cell comprise all of (a), (b), and (c).

The engineered immune cell (e.g., the engineered NK cell) can exhibit enhanced signaling of an endogenous signaling pathway that involves the heterologous cytokine as compared to a control cell, as disclosed herein.

The expression or activity of the endogenous immune regulator polypeptide can be reduced in the engineered immune cell (e.g., the engineered NK cell), for example, via action of a gene editing moiety as disclosed herein.

The reduced expression or activity of the endogenous immune regulator polypeptide in the engineered immune cell (e.g., the engineered NK cell) as disclosed herein can be ascertained by a number of methods, including, but are not limited to, (i) phosphorylation or dephosphorylation of a downstream signaling protein (e.g., SHP2, Igα/β, Syk, etc. for PD1/PDL1 signaling) or (ii) expression of the endogenous immune regulator polypeptide (e.g., PD1) via Western blotting or PCR techniques.

In some cases, reduced expression of the endogenous immune regulator polypeptide in the engineered immune cell (e.g., the engineered NK cell) as disclosed herein can be characterized by a decrease in the expression of the endogenous immune regulator polypeptide (e.g., PD1) by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell.

In some cases, reduced activity of the endogenous immune regulator polypeptide in the engineered immune cell (e.g., the engineered NK cell) as disclosed herein can be characterized by a decrease in phosphorylation of a downstream signaling protein (e.g., SHP2 for PD1/PDL1 signaling) by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell.

In some cases, reduced activity of the endogenous immune regulator polypeptide in the engineered immune cell (e.g., the engineered NK cell) as disclosed herein can be characterized by an increase in phosphorylation of a downstream target signaling protein (e.g., Igα/β or Syk for PD1/PDL1 signaling) by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell. The downstream target signaling protein may be a protein that is normally inhibited by action of a functional signaling pathway of the endogenous immune regulator polypeptide.

In one aspect, the present disclosure provides an engineered immune cell (e.g., an engineered NK cell). The engineered immune cell can comprise a CD16 variant as disclosed herein for enhanced CD16 signaling in the engineered NK cell. In some cases, the CD16 variant (e.g., a heterologous CD16 variant) can comprise at least a portion of CD16 and at least a portion of CD64 (e.g., hnCD16 as disclosed herein). The engineered immune cell can further comprise reduced expression or activity of an endogenous immune regulator polypeptide as compared to a control cell, as disclosed herein.

The engineered immune cell (e.g., the engineered NK cell) can exhibit enhanced CD16 signaling as compared to a control cell, as disclosed herein.

In one aspect, the present disclosure provides an engineered immune cell (e.g., an engineered NK cell). The engineered immune cell can comprise reduced activity of endogenous cytokine signaling (e.g., endogenous IL signaling, such as endogenous IL-17 signaling) The engineered immune cell can be derived from an isolated stem cell (e.g., an isolated ESC). Alternatively, the engineered immune cell can be derived from an induced stem cell (e.g., an iPSC).

In some cases, the engineered NK cell can be treated with inhibitors (e.g., small molecule inhibitors) of the endogenous cytokine signaling. In some cases, the engineered NK cell can comprise reduced expression of endogenous IL-17 or endogenous receptor thereof (e.g., via indel or transgene mutation, via transient or permanent suppression, etc.). In some cases, the engineered NK cell can comprise reduced expression of endogenous IL-17. In some cases, the engineered NK cell can comprise reduced expression of endogenous IL-17R. In some cases, the engineered NK cell can comprise reduced expression of endogenous IL-17 and endogenous IL-17R.

In some cases, the endogenous cytokine as disclosed herein can be an endogenous IL. An endogenous IL as disclosed herein can comprise at least 1, 2, 3, 4, 5, or more different types of endogenous ILs. An endogenous IL as disclosed herein can comprise at most 5, 4, 3, or 2 different type of endogenous ILs. Alternatively, the endogenous IL can be a single type of endogenous IL. Non-limiting examples of the endogenous IL can include, but are not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36. In some examples, the endogenous IL can be IL-17. Non-limiting examples of endogenous 11-17 can include IL-17A, IL-17F, and natural mutations thereof. For example, the engineered immune cell (e.g., an engineered NK cell) as disclosed herein can exhibit reduced expression or activity of IL-17A or IL-17F.

In some cases, an endogenous gene encoding the endogenous cytokine (e.g., an endogenous IL, such as IL-17) as disclosed herein can be modified via action of a gene editing moiety as disclosed herein.

The endogenous receptor can be a respective receptor of any cytokine as disclosed herein (e.g., a respective receptor of any IL as disclosed herein). In some cases, the endogenous receptor can be a respective receptor of IL (e.g., IL-17R for IL-7 signaling) Non-limiting examples of IL-17R can include IL-17RA, IL-17RB, IL-17RC, IL-17RD, IL-17RE, and variants thereof. In an example, the endogenous IL-17R comprises IL-17RA.

In some cases, the reduced expression or activity of the endogenous cytokine (e.g., an endogenous IL, such as IL-17) or endogenous receptor thereof as disclosed herein can be ascertained by a number of methods, including, but are not limited to, (i) phosphorylation of a downstream signaling protein (e.g., PI3K, Act1, MAP3K, MEK1/2, MKK3/6, MKK4/7, MKK3/6, ERK, p38, JNK, etc. for IL-17) or (ii) expression of a downstream gene via Western blotting or PCT techniques. In some examples, a downstream gene of IL cytokine, such as IL-17, can include a chemokine (e.g., CXCL1, CXCL2, CXCL8, CXCL9, CXCL10, CCL2, CCL20, etc.), a cytokine (e.g., IL-6, TNFa, G-CSF, GM-CSF, etc.), an acute phase response molecule (e.g., SAA, CRP, lipocalin 2/24p3, etc.), and/or an enzyme (e.g., a metalloproteinase, such as MMP1, MMP3, MMP9, MMP13).

In some cases, reduced expression or activity of the endogenous cytokine (e.g., the endogenous IL, such as IL-17) or endogenous receptor thereof in the engineered immune cell (e.g., engineered NK cell) as disclosed herein can be characterized by a decrease in phosphorylation of a downstream signaling protein of the endogenous cytokine by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell.

In some cases, reduced expression or activity of the endogenous cytokine (e.g., the endogenous IL, such as IL-17) or endogenous receptor thereof in the engineered immune cell (e.g., engineered NK cell) as disclosed herein can be characterized by a decrease in the expression of a downstream gene of the endogenous cytokine by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell.

In some cases, the engineered immune cell (e.g., the engineered NK cell) as disclosed herein can exhibit enhanced expression profile of a specific cell marker for a committed immune cell (e.g., a NK cell marker) as compared to a control cell that does not exhibit the reduced activity of the endogenous cytokine signaling (e.g., endogenous IL signaling, such as endogenous IL-17 signaling) as disclosed herein. For example, non-limiting examples of a specific cell marker for committed NK cells can include CD57 or killer immunoglobulin-like receptors (KIR). KIR can comprise KIR2D and/or KIR3D. KIR2D can comprise KIR2DL1, KIR2DL2, KIR2DL3, KIR2DL4, KIR2DL5A, KIR2DL5B, KIR2DS1, KIR2DS2, KIR2DS3, KIR2D S4, and/or KIR2DS5. KIR3D can comprise KIR3DL1, KIR3DL2, KIR3DL3, and/or KIR3DS1.

The enhanced expression profile of the specific cell marker for the committed immune cell (e.g., CD57 or KIR for NK cells) as disclosed herein can be ascertained by a number of methods, including, but are not limited to, Western blotting or PCR techniques.

In some cases, the expression of the specific cell marker for a committed immune cell (e.g., CD57 or KIR or NK cells) in the engineered immune cell of the present disclosure can be greater than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In one aspect, the present disclosure provides an engineered immune cell (e.g., an engineered NK cell). The engineered immune cell can comprise one or both of: (i) a heterologous transcription factor (e.g., a heterologous STAT), (ii) reduced activity of endogenous cytokine signaling (e.g., endogenous IL signaling as disclosed herein), and (iii) reduced expression or activity of endogenous enzyme (e.g., a ligase, such as CBL-B). The engineered immune cell can be derived from an isolated stem cell (e.g., an isolated ESC). Alternatively, the engineered immune cell can be derived from an induced stem cell (e.g., an iPSC).

The heterologous transcription factor can comprise at least 1, 2, 3, 4, 5, or more different types of heterologous transcription factor. The heterologous transcription factor can comprise at most 5, 4, 3, or 2 different types of transcription factor. Alternatively, the heterologous transcription factor can have a single type of transcription factor. The transcription factor can be involved in the engineered immune cell's immune activity, proliferation, apoptosis, and/or differentiation. In some cases, the heterologous transcription factor for the engineered immune cell (e.g., the engineered NK cell) can be STAT. Non-limiting examples of STAT can include STAT1, STAT2, STAT3, STAT4, STAT3, STAT4, STAT5A, STAT5B, STAT6, and modifications thereof. In an example, STAT can comprise STAT3. In another example, STAT can comprise STAT5B.

In some cases, the engineered immune cell (e.g., the engineered NK cell) as disclosed herein can exhibit enhanced survival in the presence of tumor cells as compared to a control cell without (i) the heterologous transcription factor (e.g., the heterologous STAT) or (ii) the reduced activity of endogenous cytokine signaling (e.g., endogenous IL-17 signaling). In some case, the engineered immune cell can, in the presence of tumor cells, survive longer than the control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In one aspect, the present disclosure provides an engineered immune cell (e.g., an engineered NK cell). The engineered immune cell can exhibit reduced expression or activity of a specific endogenous cell marker for a committed immune cell as disclosed herein (e.g., a NK cell marker, such as KIR) as compared to a control cell. In some examples, the specific endogenous cell marker is KIR. The engineered immune cell can further comprise one or more of: (a) a chimeric polypeptide receptor comprising an antigen binding moiety capable of binding to an antigen, as disclosed herein, (b) a heterologous cytokine (e.g., a heterologous IL, such as IL-15), as disclosed herein, (c) a CD16 variant for enhanced CD16 signaling as compared to a control cell, wherein the CD16 variant is heterologous to the engineered NK cell, as disclosed herein, (d) an immune regulator polypeptide as disclosed herein, wherein the immune regulator polypeptide is heterologous to the engineered immune cell, and (e) reduced expression or activity of an endogenous immune regulator polypeptide, as disclosed herein.

The engineered immune cell can comprise the chimeric polypeptide receptor and one or more of (e.g., 1, 2, 3, or 4 of): (b) the heterologous cytokine, (c) the CD16 variant for enhanced CD16 signaling, (d) the heterologous immune regulator, and (e) the reduced expression or activity of an endogenous immune regulator polypeptide.

The engineered immune cell can comprise the heterologous cytokine and one or more of (e.g., 1, 2, 3, or 4 of): (a) the chimeric polypeptide receptor, (c) the CD16 variant for enhanced CD16 signaling, (d) the heterologous immune regulator, and (e) the reduced expression or activity of an endogenous immune regulator polypeptide.

The engineered immune cell can comprise the CD16 variant for enhanced CD16 signaling and one or more of (e.g., 1, 2, 3, or 4 of): (a) the chimeric polypeptide receptor, (b) the heterologous cytokine, (d) the heterologous immune regulator, and (e) the reduced expression or activity of an endogenous immune regulator polypeptide.

The engineered immune cell can comprise the heterologous immune regulator and one or more of (e.g., 1, 2, 3, or 4 of): (a) the chimeric polypeptide receptor, (b) the heterologous cytokine, (c) the CD16 variant for enhanced CD16 signaling, and (e) the reduced expression or activity of an endogenous immune regulator polypeptide.

The engineered immune cell can comprise the reduced expression or activity of an endogenous immune regulator polypeptide and one or more of (e.g., 1, 2, 3, or 4 of): (a) the chimeric polypeptide receptor, (b) the heterologous cytokine, (c) the CD16 variant for enhanced CD16 signaling, and (d) the heterologous immune regulator.

The reduced expression or activity of the specific endogenous cell marker for the committed immune cell (e.g., KIR for NK cells) as disclosed herein can be ascertained by a number of methods, including, but are not limited to, Western blotting or PCR techniques.

In some cases, the expression of the specific endogenous cell marker for a committed immune cell (e.g., KIR or NK cells) in the engineered immune cell of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In one aspect, the present disclosure provides an engineered immune cell (e.g., an engineered NK cell). The engineered immune cell can exhibit reduced expression or activity of one or more (e.g., 1, 2, 3, 4, 5, or more) endogenous immune checkpoint inhibitors (e.g., CD94, CD96, TGF beta receptor, SHIP2, etc.). In some cases, the engineered immune cell can exhibit reduced expression or activity of one or more (e.g., 1, 2, 3, 4, 5, or more) of: (i) endogenous CD94, (ii) endogenous CD96, (iii) endogenous TGF beta receptor, and (iv) endogenous SHIP (e.g., SHIP2).

In some cases, the engineered immune cell can exhibit reduced expression or activity of endogenous CD94 and also reduced expression or activity of one or more of (e.g., 1, 2, or all of): (ii) endogenous CD96, (iii) endogenous TGF beta receptor, and (iv) endogenous SHIP (e.g., SHIP2).

In some cases, the engineered immune cell can exhibit reduced expression or activity of endogenous CD96 and also reduced expression or activity of one or more of (e.g., 1, 2, or all of): (i) endogenous CD94, (iii) endogenous TGF beta receptor, and (iv) endogenous SHIP (e.g., SHIP2).

In some cases, the engineered immune cell can exhibit reduced expression or activity of endogenous TGF beta receptor and also reduced expression or activity of one or more of (e.g., 1, 2, or all of): (i) endogenous CD94, (ii) endogenous CD96, and (iv) endogenous SHIP (e.g., SHIP2).

In some cases, the engineered immune cell can exhibit reduced expression or activity of endogenous SHIP (e.g., SHIP2) and also reduced expression or activity of one or more of (e.g., 1, 2, or all of): (i) endogenous CD94, (ii) endogenous CD96, and (iii) endogenous TGF beta receptor.

In some cases, the reduced expression or activity of the immune checkpoint inhibitor (e.g., CD94, CD96, TGF beta receptor, SHIP2, etc.) in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, the reduced expression or activity of the endogenous CD94 in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, the reduced expression or activity of the endogenous CD96 in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, the reduced expression or activity of the endogenous TGF beta receptor in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, the reduced expression or activity of the endogenous SHIP (e.g., SHIP2) in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In one aspect, the present disclosure provides an engineered immune cell (e.g., an engineered NK cell). The engineered immune cell can exhibit reduced expression or activity of an endogenous immune regulator polypeptide, as disclosed herein. In some cases, the endogenous immune regulator polypeptide comprise one or more (e.g., 1, 2, 3, 4, 5, or more) hypo-immunity regulators. In some cases, the engineered immune cell exhibits reduced expression or activity of one or more (e.g., 1, 2, 3, 4, 5, or more) hypo-immunity regulators from: (i) endogenous CD80, (ii) endogenous CD86, (iii) endogenous ICOSL, (iv) endogenous CD40L, (v) endogenous MICA or MICB, or (vi) endogenous NKG2DL. The engineered immune cell can be derived from an isolated stem cell (e.g., an isolated ESC). Alternatively, the engineered immune cell can be derived from an induced stem cell (e.g., an iPSC).

In some cases, the reduced expression or activity of the endogenous hypo-immunity regulator (e.g., CD80, CD86, ICOSL, CD40L, MICA, MICB, NKG2DL, etc.) in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as ascertained by Western blotting or PCT techniques, as disclosed herein.

In some cases, the reduced expression or activity of the endogenous CD80 in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, the reduced expression or activity of the endogenous CD86 in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, the reduced expression or activity of the endogenous ICOSL in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, the reduced expression or activity of the endogenous hypo-immunity regulator CD40L in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, the reduced expression or activity of the endogenous MICA or MICB in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In some cases, the reduced expression or activity of the endogenous NKG2DL in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold.

In one aspect, the present disclosure provides an engineered immune cell (e.g., an engineered NK cell). The engineered immune cell can exhibit reduced expression or activity of an endogenous immune regulator polypeptide, as disclosed herein. In some cases, the endogenous immune regulator polypeptide comprise a hypo-immunity regulator (e.g., ICAM1). The engineered immune cell further comprises one or more of: (a) a chimeric polypeptide receptor comprising an antigen binding moiety capable of binding to an antigen, as disclosed herein, (b) a heterologous cytokine (e.g., a heterologous IL, such as IL-15), as disclosed herein, and (c) a CD16 variant for enhanced CD16 signaling as compared to a control cell.

In some cases, the engineered immune cell (e.g., the engineered NK cell) comprises a chimeric polypeptide receptor as disclosed herein and one or both of: (b) the heterologous cytokine (e.g., a heterologous IL, such as IL-15), as disclosed herein and (c) the CD16 variant for enhanced CD16 signaling.

In some cases, the engineered immune cell (e.g., the engineered NK cell) comprise the heterologous cytokine (e.g., a heterologous IL, such as IL-15), as disclosed herein and one or both of: (a) the chimeric polypeptide receptor as disclosed herein and (c) the CD16 variant for enhanced CD16 signaling.

In some cases, the engineered immune cell (e.g., the engineered NK cell) comprises the CD16 variant for enhanced CD16 signaling and one or both of: (a) the chimeric polypeptide receptor as disclosed herein and (b) the heterologous cytokine (e.g., a heterologous IL, such as IL-15), as disclosed herein.

In some cases, the reduced expression or activity of the endogenous ICAM1 in the engineered immune cell (e.g., the engineered NK cell) of the present disclosure can be less than expression of the same by a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 4-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as ascertained by Western blotting or PCT techniques, as disclosed herein.

In one aspect, the present disclosure provides an engineered immune cell (e.g., an engineered NK cell). The engineered immune cell can exhibit reduced expression or activity of an endogenous immune regulator polypeptide, as disclosed herein. In some cases, the endogenous immune regulator polypeptide comprise a hypo-immunity regulator (e.g., ICAM1). The engineered immune cell can be derived from an induced stem cell (e.g., an iPSC).

In some cases, the reduced expression or activity of the endogenous ICAM1 in the engineered immune cell (e.g., the engineered NK cell) of the present disclosure can be less than expression of the same by a control cell, as disclosed herein.

In one aspect, the present disclosure provides an engineered immune cell (e.g., an engineered NK cell). The engineered immune cell can comprise an immune regulator polypeptide as disclosed herein, wherein the immune regulator polypeptide is heterologous to the engineered immune cell. In some cases, the immune regulator polypeptide comprises a hypo-immunity regulator. The hypo-immunity regulator can be PDL2. Alternatively or in addition to, the hypo-immunity regulator can be TGF-beta.

In one aspect, the present disclosure provides an engineered immune cell (e.g., an engineered NK cell). The engineered immune cell can comprise an immune regulator polypeptide as disclosed herein, wherein the immune regulator polypeptide is heterologous to the engineered immune cell. In some cases, the immune regulator polypeptide comprises a hypo-immunity regulator. The hypo-immunity regulator can comprise one or more (e.g., 1, 2, 3, 4, or more) members from: (i) a heterologous CCL21, (ii) a heterologous IL-10, (iii) a heterologous CD46, (iv) a heterologous CD55, and (v) a heterologous CD59. The engineered immune cell can be derived from an isolated stem cell (e.g., an isolated ESC). Alternatively, the engineered immune cell can be derived from an induced stem cell (e.g., an iPSC).

In some cases, the engineered immune cell (e.g., the engineered NK cell) as disclosed herein can comprise the heterologous CCL21 and one or more of (e.g., 1, 2, 3, or all of): (ii) a heterologous IL-10, (iii) a heterologous CD46, (iv) a heterologous CD55, and (v) a heterologous CD59.

In some cases, the engineered immune cell (e.g., the engineered NK cell) as disclosed herein can comprise the heterologous IL-10 and one or more of (e.g., 1, 2, 3, or all of): (i) a heterologous CCL21, (iii) a heterologous CD46, (iv) a heterologous CD55, and (v) a heterologous CD59.

In some cases, the engineered immune cell (e.g., the engineered NK cell) as disclosed herein can comprise the heterologous CD46 and one or more of (e.g., 1, 2, 3, or all of): (i) a heterologous CCL21, (ii) a heterologous IL-10, (iv) a heterologous CD55, and (v) a heterologous CD59.

In some cases, the engineered immune cell (e.g., the engineered NK cell) as disclosed herein can comprise the heterologous CD55 and one or more of (e.g., 1, 2, 3, or all of): (i) a heterologous CCL21, (ii) a heterologous IL-10, (iii) a heterologous CD46, and (v) a heterologous CD59.

In some cases, the engineered immune cell (e.g., the engineered NK cell) as disclosed herein can comprise the heterologous CD59 and one or more of (e.g., 1, 2, 3, or all of): (i) a heterologous CCL21, (ii) a heterologous IL-10, (iii) a heterologous CD46, and (iv) a heterologous CD55.

In one aspect, the present disclosure provides an engineered immune cell (e.g., an engineered NK cell). The engineered immune cell can comprise a heterologous cytokine (e.g., a heterologous IL), as disclosed herein that is not IL-15. In some examples, the heterologous cytokine comprises IL-21 or variants thereof. The engineered immune cell can be derived from an induced stem cell (e.g., iPSC).

In some cases, a control cell can be a cell can be an immune cell, such as a NK cell, used for comparison purposes. In some cases, a control cell can be a cell that does not comprise a heterologous cytokine (e.g., IL-15). In some cases, a control cell can be a cell that does not comprise a CD16 variant for enhanced CD16 signaling. In some cases, a control cell can be a cell that a chimeric polypeptide receptor comprising an antigen binding moiety capable of binding to an antigen. In some cases, a control cell can be a cell that comprises a heterologous IL-15R. In some cases, a control cell can be a cell that does not comprise a membrane bound heterologous cytokine (e.g., IL-15). In some cases, a control cell can be a cell that does not exhibit reduced expression or activity of an endogenous immune regulator polypeptide. In some cases, a control cell can be a cell that does not exhibit reduced expression or activity of an endogenous cytokine (e.g., IL-17) or a receptor thereof (e.g., IL-17R). In some cases, a control cell can be a cell that does not comprise a heterologous transcription factor (e.g., STAT). In some cases, a control cell can be a cell that does not exhibit reduced expression or activity of a specific endogenous cell marker for a committed immune cell (e.g., a NK cell marker, such as KIR). In some cases, a control cell can be a cell that does not comprise a heterologous immune regulator polypeptide. In some cases, a control cell can be a cell that does not exhibit reduced expression or activity of one or more (e.g., 1, 2, 3, or 4) of: endogenous CD94, endogenous CD96, endogenous TGF beta receptor, or endogenous SHIP2. In some cases, a control cell can be a cell that does not exhibit reduced expression or activity of one or more (e.g., 1, 2, 3, 4, or 5) of: endogenous CD80, endogenous CD86, endogenous ICOSL, endogenous CD40L, endogenous MICA or MICB, or endogenous NKG2DL. In some cases, a control cell can be a cell that does not exhibit reduced expression or activity of ICAM1. In some cases, a control cell can be a cell that does not comprise a heterologous PDL2 or heterologous TGF beta. In some cases, a control cell can be a cell that does not comprise one or more (e.g., 1, 2, 3, 4, or 5) of: heterologous CCL21, heterologous IL-10, heterologous CD46, heterologous CD55, or heterologous CD59. In some cases, a control cell can be a cell that does not comprise heterologous IL-21. In some cases, a control cell can be a cell that is not derived from a cell line. In some cases, a control cell can be a cell that is not derived from an isolated ESC. In some cases, a control cell can be a cell that is not derived from an iPSC.

In some embodiments, the present disclosure provides a population of engineered immune cells comprising any one of the engineered immune cells as disclosed herein (e.g., a population of engineered NK cells comprising any one of the engineered NK cells as disclosed herein). An engineered immune cell (e.g., an engineered NK cell) of the population of immune cells (e.g., the population of NK cells) can comprise a heterologous polypeptide, wherein the heterologous polypeptide comprises a heterologous IL-15 (e.g., heterologous secretory IL-15, and/or membrane-bound Il-15, such as IL15-IL15 receptor fusion). An activity level (e.g., persistence level) of the population of engineered immune cells in an environment that is substantially free of an exogenous interleukin (e.g., IL-2, IL-15, etc.) can be at least about 5%, at least about 10%, at least about 15%, at least about 20%, at least about 30%, at least about 40%, at least about 50%, at least about 60%, at least about 70%, at least about 80%, at least about 85%, at least about 90%, at least about 95%, at least about 99%, at least about or more greater than a control persistence level of a comparable population of immune cells (e.g., engineered to comprise a comparable heterologous IL-15 as disclosed herein) in a control environment comprising the exogenous interleukin. Such persistence level may ascertained after the population of immune cells are in the environment for at least or up to about 1 day, at least or up to about 2 days, at least or up to about 3 days, at least or up to about 4 days, at least or up to about 5 days, at least or up to about 6 days, at least or up to about 7 days, at least or up to about 8 days, at least or up to about 9 days, at least or up to about 10 days, at least or up to about 11 days, at least or up to about 12 days, at least or up to about 13 days, at least or up to about 14 days, at least or up to about 15 days, at least or up to about 16 days, at least or up to about 17 days, at least or up to about 18 days, at least or up to about 19 days, at least or up to about 20 days, at least or up to about 21 days, at least or up to about 22 days, at least or up to about 23 days, at least or up to about 24 days, at least or up to about 25 days, at least or up to about 26 days, at least or up to about 27 days, at least or up to about 28 days, at least or up to about 5 weeks, at least or up to about 6 weeks, or at least or up to about 8 weeks. The amount of the exogenous interleukin (e.g., IL-2, IL-15, etc.) in the environment can be at least or up to about 1 unit per milliliter (U/ml), at least or up to about 5 U/mL, at least or up to about 10 U/mL, at least or up to about 15 U/mL, at least or up to about 20 U/mL, at least or up to about 30 U/mL, at least or up to about 40 U/mL, at least or up to about 50 U/mL, at least or up to about 60 U/mL, at least or up to about 80 U/mL, at least or up to about 100 U/mL, at least or up to about 150 U/mL, at least or up to about 200 U/mL, at least or up to about 300 U/mL, at least or up to about 400 U/mL, or at least or up to about 500 U/mL. The environment can be in vitro, ex vivo, or in vivo.

The population of engineered NK cells as disclosed herein can exhibit at least or up to about 10%, at least or up to about 20%, at least or up to about 30%, at least or up to about 40%, at least or up to about 50%, at least or up to about 60%, at least or up to about 70%, at least or up to about 80%, at least or up to about 90%, at least or up to about 95%, or more persistence (or survival rate) after being in the environment that is substantially free of the exogenous interleukin (e.g., IL-2) for at least or up to about 1 day, at least or up to about 2 days, at least or up to about 3 days, at least or up to about 4 days, at least or up to about 5 days, at least or up to about 6 days, at least or up to about 7 days, at least or up to about 8 days, at least or up to about 9 days, at least or up to about 10 days, at least or up to about 11 days, at least or up to about 12 days, at least or up to about 13 days, at least or up to about 14 days, at least or up to about 3 weeks, or at least or up to about 4 weeks.

For example, the population of engineered NK cells as disclosed herein can exhibit at least about 50% survival after at least about 5 days (50%, after 9 days, 25% after 15 days) in the environment that is substantially free of the exogenous interleukin (e.g., IL-2). For example, the population of engineered NK cells as disclosed herein can exhibit at least about 50% survival after at least about 9 days in the environment that is substantially free of the exogenous interleukin (e.g., IL-2). For example, the population of engineered NK cells as disclosed herein can exhibit at least about 25% survival after at least about 15 days in the environment that is substantially free of the exogenous interleukin (e.g., IL-2).

The population of engineered NK cells as disclosed herein can exhibit enhanced persistence by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 200-fold, at least or up to about 300-fold, at least or up to about 400-fold, or at least or up to about 500-fold, after being in the environment substantially free of an exogenous interleukin (e.g., IL-2, IL-15, etc.) for at least or up to about 1 day, at least or up to about 2 days, at least or up to about 3 days, at least or up to about 4 days, at least or up to about 5 days, at least or up to about 6 days, at least or up to about 7 days, at least or up to about 8 days, at least or up to about 9 days, at least or up to about 10 days, at least or up to about 11 days, at least or up to about 12 days, at least or up to about 13 days, at least or up to about 14 days, at least or up to about 2 weeks, at least or up to about 3 weeks, at least or up to about 4 weeks, at least or up to about 6 weeks, or at least or up to about 8 weeks, as compared to that of a comparable population of NK cells lacking the heterologous polypeptide comprising the heterologous IL-15 (e.g., lacking a heterologous membrane-bound IL-15). For example, the population of engineered NK cells as disclosed herein can exhibit enhanced persistence by at least about 7-fold after at least about 5 days in the environment, as compared to that of a comparable population of NK cells lacking the heterologous IL-15.

Without wishing to be bound by theory, having such enhanced persistence level by having the heterologous polypeptide comprising the enhanced IL-15 signaling (e.g., via comprising a heterologous polypeptide that comprises heterologous IL-15) can reduce the amount of exogenous proteins (e.g., IL-2) required for the production of the engineered immune cells, enhance the efficiency of producing the engineered immune cells, and/or reduce overall cost of the immune cell therapy. Any enhanced level of persistence ascertained in vitro can be translated to an event in vivo (e.g., in the blood stream of a subject in need thereof).

An exogenous interleukin, as disclosed herein, can comprise an interleukin that is not secreted by the cell or the population of cells (e.g., the engineered immune cell(s), such as the engineered NK cells as disclosed herein), and can be artificially added to the environment. In an in vitro environment, an exogenous interleukin may comprise a recombinant interleukin protein that is added to a medium. In an in vivo environment (e.g., in plasma), an exogenous interleukin may comprise a recombinant interleukin protein that is administered to a subject in need thereof.

The term “persistence” as used herein may generally refer to a presence of at least a portion of a population of cells (e.g., a population of engineered immune cells, such as a population of engineered NK cells as disclosed herein) remaining in an environment after introducing the population of cells to the environment (e.g., in an in vitro medium, in the serum after intravenous (IV) administration, etc.). A persistence may be ascertained by a duration of time that at least a portion of the population of cells remain in the environment at a detectable level. In some examples, persistence of a population of cells may correlate to the half-life of the population of cells in the environment (e.g., medium, blood stream, etc.). In some cases, a population of cells of interest (e.g., a population of engineered immune cells, such as a population of engineered NK cells) may not grow (e.g., proliferate) over time, and a persistence level of the population of cells as disclosed herein can be ascertained by measuring a decrease (or a rate thereof) in a size of the population of cells over time. For example, a population of cells having a greater persistence level (e.g., at least 5% greater) in an environment after a period of time (e.g., after at least about 5 days) than a control population of cells in a comparable environment after a comparable period of time may indicate that a greater proportion (e.g., at least 5% greater) of the size of the population of cells have survived as compared to the control population of cells.

C. Additional Aspects of the Engineered Immune Cells

In some embodiments of any one of the engineered immune cell (e.g., the engineered NK cell) disclosed herein, the engineered immune cell can comprise a heterologous cytokine (e.g., a heterologous IL, such as IL-15) as disclosed herein. The heterologous cytokine can be a heterologous secretory cytokine. The heterologous cytokine can be a membrane-bound cytokine. In some cases, the engineered immune cell (e.g., the engineered NK cell) comprises a heterologous receptor that is a respective receptor of the heterologous cytokine (e.g., a heterologous IL-15R), as disclosed herein. In some cases, the heterologous cytokine and the respective heterologous receptor can be provided as a fusion protein (e.g., IL15-IL15R fusion). As such, the engineered immune cell (e.g., the engineered NK cell) can exhibit enhanced signaling of the endogenous signaling pathway induced by the heterologous cytokine and/or the heterologous receptor (e.g., induced by the heterologous cytokine and/or heterologous receptor, such as IL-15/IL-15R) as disclosed herein.

In some embodiments of any one of the engineered immune cell (e.g., the engineered NK cell) disclosed herein, the engineered immune cell can exhibit reduced expression or activity of endogenous CD38 as compared to a control cell. Such engineered immune cell may be used to treat a subject who has or is suspected of having white blood cell cancer, such as multiple myeloma (MM).

In some embodiments of any one of the engineered immune cell (e.g., the engineered NK cell) disclosed herein, expression or activity of endogenous CD38 of the engineered immune cell may not and need not be modified. Such engineered immune cell may be used to treat a subject who has or is suspected of having a disease (e.g., cancer, tumor) that is not multiple myeloma.

In some embodiments of any one of the engineered immune cell (e.g., the engineered NK cell) disclosed herein, the engineered immune cell can comprise a heterologous IL-15 or a fragment thereof, and the heterologous IL-15 or the fragment thereof can be secreted by the engineered immune cell.

In some embodiments of any one of the engineered immune cell (e.g., the engineered NK cell) disclosed herein, the engineered immune cell can comprise a heterologous IL-15 or a fragment thereof, and the heterologous IL-15 or the fragment thereof can be bound to a cell surface membrane of the engineered immune cell.

In some embodiments of any one of the engineered immune cell (e.g., the engineered NK cell) disclosed herein, the engineered immune cell can comprise at least one chimeric polypeptide receptor comprising an antigen binding moiety capable of binding to an antigen, as provided in the present disclosure. In some examples, the engineered immune cell can comprise a plurality of different chimeric polypeptide receptors to specifically bind a plurality of different antigens. In some examples, the engineered immune cell can comprise at least one chimeric polypeptide receptor that comprises a plurality of antigen binding moieties to specifically bind a plurality of different antigens.

In some embodiments of any one of the engineered immune cell (e.g., the engineered NK cell) disclosed herein, the engineered immune cell can comprise a safety switch capable of effecting death of the engineered immune cell. The engineered immune cell can comprise a gene encoding the safety switch (e.g., integrated into the genome of the immune cell), via action of the gene editing moiety, as disclosed herein. In some cases, a prodrug can be introduced to the engineered immune cell (e.g., administered to a subject comprising the engineered immune cell) in the event of an adverse event or when the adaptive immunotherapy is no longer necessary, and the prodrug can be activated by the safety switch molecule to kill the subject immune cell. In some cases, the safety switch can comprise one or more members selected from the group consisting of caspase (e.g., caspase 3, 7, or 9), thymidine kinase, cytosine deaminase, modified EGFR, B-cell CD20, and functional variants thereof. In some cases, the safety switch can be activated via an activator (e.g., a small molecule or a protein, such as an antibody) for post-translational, temporal, and/or site-specific regulation of death (or depletion) of the subject engineered immune cell. Non-limiting examples of a safety switch and its activator can include Caspase 9 (or caspase 3 or 7) and AP1903; thymidine kinase (TK) and ganciclovir (GCV); and cytosine deaminase (CD) and 5-fluorocytosine (5-FC). Alternatively or in addition to, modified epidermal growth factor receptor (EGFR) containing epitope recognized by an antibody (e.g., anti-EGFR Ab, such as cetuximab) can be used to deplete the engineered immune cells when the subject cells are exposed to the antibody. In some cases, the engineered immune cells (e.g., the engineered NK cells) as disclosed herein can comprise a safety switch protein selected from the group consisting of caspase 9 (caspase 3 or 7), thymidine kinase, cytosine deaminase, modified EGFR, and B-cell CD20.3

In some embodiments of any one of the engineered immune cell (e.g., the engineered NK cell) disclosed herein, the engineered immune cell can comprise heterologous immune receptor polypeptide. The immune regulator polypeptide can comprise one or more (e.g., 1, 2, 3, 4, or more) members selected from the group consisting of HLA-E, CD47, CD113, PDL1, PDL2, A2AR, HLA-G, TGF-beta, CCL21, IL10, CD46, CD55, and CD59.

In some embodiments of any one of the engineered immune cell (e.g., the engineered NK cell) disclosed herein, the engineered immune cell can exhibits reduced expression or activity of an endogenous immune regulator polypeptide, as disclosed herein. The endogenous immune regulator polypeptide comprises an immune checkpoint inhibitor or a hypo-immunity regulator (or both).

In some cases, the immune checkpoint inhibitor as disclosed herein can comprise one or more (e.g., 1, 2, 3, 4, or more) members selected from the group consisting of PD1, CTLA-4, TIM-3, KIR2D, CD94, NKG2A, NKG2D, TIGIT, CD96, LAG3, TIGIT, TGF beta receptor, and 2B4. In some cases, the immune checkpoint inhibitor can comprise SHIP2.

In some cases, the hypo-immunity regulator as disclosed herein can comprise one or more (e.g., 1, 2, 3, 4, or more) members selected from the group consisting of B2M, CIITA, TAP1, TAP2, tapasin, NLRC5, RFXANK, RFX5, RFXAP, CD80, CD86, ICOSL, CD40L, ICAM1, MICA, MICB, ULBP1, HLA-E, CD47, CD113, PDL1, PDL2, A2AR, HLA-G, TGF-beta, CCL21, IL10, CD46, CD55, and CD59.

Having a reduced expression or activity level of any one of the endogenous immune regulator polypeptides (e.g., immune checkpoint inhibitor, hypo-immunity regulator) as disclosed herein can enhance one or more functions of the engineered immune cell (e.g., engineered NK cell) as disclosed herein, e.g., enhanced persistence or survival, enhanced resistance against immune rejection (e.g., ADCC cytotoxicity), enhanced cytotoxicity against a target cell (e.g., cancer cell), etc.

In some embodiments of any one of the engineered immune cell (e.g., the engineered NK cell) disclosed herein, the engineered immune cell can comprise a CD16 variant for enhanced CD16 signaling as compared to a control cell, wherein the CD16 variant is heterologous to the engineered immune cell.

In some embodiments of any one of the engineered immune cell (e.g., the engineered NK cell) disclosed herein, the engineered immune cell can exhibit enhanced cytotoxicity against a target cell as compared to a control cell. In some cases, the engineered immune cell as disclosed herein can exhibit cytotoxicity (e.g., in vitro, ex vivo, or in vivo) against a target cell or a target population of cells that is greater than that of a control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as ascertained by, e.g., tracking a change in the number of the target population of cells.

In some embodiments of any one of the engineered immune cell (e.g., the engineered NK cell) disclosed herein, the engineered immune cell can induce reduced immune response from separate immune cells (e.g., separate T cells and/or B-cells in vitro, or a host's immune cells upon administration of the engineered immune cell to the host) as compared to a control cell. In some cases, the engineered immune cell as disclosed herein can reduce the immune response from the separate immune cells by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to an immune response from the separate immune cells when exposed to a control cell, as ascertained by, e.g., measuring (i) a change in the number of the initial population of the engineered immune cells upon exposure to the separate immune cells or (ii) a change in cytokine release of the separate immune cells upon exposure to the initial population of the engineered immune cells.

In some embodiments of any one of the engineered immune cell (e.g., the engineered NK cell) disclosed herein, the engineered immune cell can exhibit enhanced half-life upon exposure to separate immune cells (e.g., separate T cells and/or B-cells in vitro, or a host's immune cells upon administration of the engineered immune cell to the host) as compared to a control cell. In some cases, upon exposure to the separate immune cells (e.g., in vitro or in vivo), the half-life of the engineered immune cells can be greater than that of the control cell by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as ascertained by monitoring the number of the engineered immune cells over time (e.g., via FACS).

In some embodiments of any one of the engineered immune cell (e.g., the engineered NK cell) disclosed herein, the engineered immune cell can effect enhanced function or pathological condition of a bodily tissue of a subject as compared to a control cell. In some cases, treatment with the engineered immune cell can effect enhanced function or pathological condition of a bodily tissue of a subject by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell or vehicle (i.e., no cells).

In some embodiments of any one of the engineered immune cell (e.g., the engineered NK cell) disclosed herein, the engineered immune cell can effect delayed degeneration of function or pathological condition of a bodily tissue of a subject as compared to a control cell. In some cases, treatment with the engineered immune cell can effect delayed degeneration of function or pathological condition of a bodily tissue of a subject by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control cell or vehicle (i.e., no cells).

In some embodiments of any one of the engineered immune cell (e.g., the engineered NK cell) disclosed herein, the bodily tissue can comprise one or more (e.g., 1, 2, 3, 4, or more) members selected from the group consisting of blood, plasma, serum, urine, perilymph fluid, feces, saliva, semen, amniotic fluid, cerebrospinal fluid, bile, sweat, tears, sputum, synovial fluid, vomit, bone, heart, thymus, artery, blood vessel, lung, muscle, stomach, intestine, liver, pancreas, spleen, kidney, gall bladder, thyroid gland, adrenal gland, mammary gland, ovary, prostate gland, testicle, skin, adipose, eye, brain, infected tissue, diseased tissue, malignant tissue, calcified tissue, and healthy tissue.

In some embodiments of any one of the engineered immune cell (e.g., the engineered NK cell) disclosed herein, the engineered immune cell can induce immune response towards a target cell. The target can be, for example, a diseased cell, a cancer cell, a tumor cell, etc.

In some embodiments of any one of the engineered immune cell (e.g., the engineered NK cell) disclosed herein, a heterologous gene can be operatively coupled to (e.g., for knock-in) a constitutive, inducible, temporal, tissue-specific, and/or cell type-specific promoter. Non-limiting examples of a promoter of interest can include CMV, EF1a, PGK, CAG, and UBC. Non=limiting examples of an insertion site can include AAVS1, CCR5, ROSA26, collagen, HTRP, H11, B2M, GAPDH, TCR, RUNX1, TAP1, TAP2, tapasin, NLRC5, CIITA, RFXANK, CIITA, RFX5, RFXAP, TCR a orb constant region, NKG2A, NKG2D, CD38, CIS, CBL-B, SOCS2, PD1, CTLA4, LAG3, TIM3, and TIGIT.

In some embodiments of any one of the engineered immune cell (e.g., the engineered NK cell) disclosed herein can exhibit reduced expression or activity of one or more (e.g., 1, 2, 3, 4, or more) of the following endogenous genes for enhancing function of the engineered immune cell in a tumor microenvironment (i.e., tumor microenvironment gene or “TME”). In some cases, having reduced expression or activity of a TME can enhance the engineered immune cell's immune activity against a target cell. In some cases, a TME gene may be an immune checkpoint inhibitor. Non-limiting examples of the TME can include: NKG2A, NKG2D, PD1, CTLA4, LAG3, TIM3, TIGIT, KIR2D, CD94, CD96, TGF beta receptor, 2B4, and SHIP2.

In some embodiments of any one of the engineered immune cell (e.g., the engineered NK cell) disclosed herein can exhibit one or more (e.g., 1, 2, 3, 4, or more) heterologous genes (e.g., knocked-in) for, e.g., enhanced function: CD137, CD80, CD86, DAP10 (e.g., with or without point mutation).

In some embodiments of any one of the engineered immune cell (e.g., the engineered NK cell) disclosed herein can exhibit reduced expression or activity of one or more (e.g., 1, 2, 3, 4, or more) of the following endogenous genes for, e.g., hypo-immunity: B2M, CIITA, TAP1, TAP2, tapasin, NLRC5, RFXANK, RFX5, RFXAP, CD80, CD86, ICOSL, CD40L, ICAM1, MICA, MICB, and a NKG2DL (e.g., ULBP1).

In some embodiments of any one of the engineered immune cell (e.g., the engineered NK cell) disclosed herein can exhibit one or more (e.g., 1, 2, 3, 4, or more) heterologous genes (e.g., knocked-in) for, e.g., hypo-immunity: HLA-E, CD47, CD113, PDL1, PDL2, A2AR, HLA-G, TGF-beta, CCL21, IL10, CD46, CD55, and CD59.

In some embodiments of any one of the engineered immune cell (e.g., the engineered NK cell) disclosed herein can exhibit one or more (e.g., 1, 2, 3, 4, or more) heterologous genes (e.g., knocked-in): CD3, CD4, CD80, 41BBL, and CD131.

D. Chimeric Antigen Receptor

The engineered immune cell (e.g., the engineered NK cell) of the present disclosure can comprise a chimeric polypeptide receptor as disclosed herein (e.g., at least 1, 2, 3, 4, 5, or more different types of chimeric polypeptide receptors). The engineered immune cell can be engineered to express a chimeric polypeptide receptor transiently or permanently. In some cases, a recombinant chimeric polypeptide receptor can be delivered to the engineered immune cell via, e.g., a liposome, and be incorporated into the engineered immune cell via membrane fusion. In some cases, a heterologous polynucleotide construct (e.g., DNA or RNA) encoding the chimeric polypeptide receptor can be delivered to the engineered immune cell. The heterologous polynucleotide construct (i.e., a gene) encoding the heterologous polynucleotide construct can be incorporated into the chromosome of the engineered immune cell (i.e., chromosomal gene) or, alternatively, may not or need not be integrated into the chromosome of the engineered immune cell as disclosed herein.

A chimeric polypeptide receptor can comprises a T cell receptor fusion protein (TFP). The term “T cell receptor fusion protein” or “TFP” generally refers to a recombinant polypeptide construct comprising (i) one or more antigen binding moieties (e.g., monospecific or multispecific), (ii) at least a portion of TCR extracellular domain, (iii) at least a portion of TCR transmembrane domain, and (iv) at least a portion of TCR intracellular domain.

In some cases, an endogenous T cell receptor (TCR) of the engineered immune cell (e.g., the engineered NK cell) of the present disclosure can be inactivated. In some examples, a function of the endogenous TCR of the engineered immune cell can be inhibited by an inhibitor. In some examples, a gene encoding a subunit of the endogenous TCR can be inactivated (e.g., edited via action of the gene editing moiety as disclosed herein) such that the endogenous TCR is inactivated. The gene encoding the subunit of endogenous TCR can be one or more of: TCRα, TCRβ, CD3ε, CD3δ, CD3γ, and CD3ζ.

A chimeric polypeptide receptor can comprises a chimeric antigen receptor (CAR). The term “chimeric antigen receptor” or “CAR” generally refers to a recombinant polypeptide construct comprising at least an extracellular antigen binding domain, a transmembrane domain, and a cytoplasmic signaling domain (also referred to herein as “an intracellular or intrinsic signaling domain”) comprising a functional signaling domain derived from a stimulatory molecule. In some cases, the stimulatory molecule may be the zeta chain associated with the T cell receptor complex. In some cases, the intracellular signaling domain further comprises one or more functional signaling domains derived from at least one costimulatory molecule. In some cases, the costimulatory molecule may comprise 4-1BB (i.e., CD137), CD27, and/or CD28. In one aspect, the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv, DARPin, etc.) during cellular processing and localization of the CAR to the cellular membrane.

A CAR may be a first-, second-, third-, or fourth-generation CAR system, a functional variant thereof, or any combination thereof. First-generation CARs (e.g., CD19R or CD19CAR) include an antigen binding domain with specificity for a particular antigen (e.g., an antibody or antigen-binding fragment thereof such as a DARPin, an scFv, a Fab fragment, a VHH domain, or a VH domain of a heavy-chain only antibody), a transmembrane domain derived from an adaptive immune receptor (e.g., the transmembrane domain from the CD28 receptor), and a signaling domain derived from an adaptive immune receptor (e.g., one or more (e.g., three) ITAM domains derived from the intracellular region of the CD3ζ receptor or FcεRIγ). Second-generation CARs modify the first-generation CAR by addition of a co-stimulatory domain to the intracellular signaling domain portion of the CAR (e.g., derived from co-stimulatory receptors that act alongside T-cell receptors such as CD28, CD137/4-1BB, and CD134/OX40), which abrogates the need for administration of a co-factor (e.g., IL-2) alongside a first-generation CAR. Third-generation CARs add multiple co-stimulatory domains to the intracellular signaling domain portion of the CAR (e.g., CD3-CD28-OX40, or CD3-CD28-41BB). Fourth-generation CARs modify second- or third-generation CARs by the addition of an activating cytokine (e.g., IL-12, IL-23, or IL-27) to the intracellular signaling portion of the CAR (e.g., between one or more of the costimulatory domains and the CD3 ITAM domain) or under the control of a CAR-induced promoter (e.g., the NFAT/IL-2 minimal promoter). In some cases, a CAR may be a new generation CAR system that is different than the first-, second-, third-, or fourth-generation CAR system as disclosed herein.

A hinge domain (e.g., the linker between the extracellular antigen binding domain and the transmembrane domain) of a CAR in the engineered immune cell (e.g., engineered NK cell) as disclosed herein can comprise a full length or at least a portion of the native or modified transmembrane region of CD3D, CD3E, CD3G, CD3c CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor polypeptide.

A transmembrane domain of a CAR in the engineered immune cell (e.g., engineered NK cell) as disclosed herein can comprise a full length or at least a portion of the native or modified transmembrane region of CD3D, CD3E, CD3G, CD3c CD4, CD8, CD8a, CD8b, CD27, CD28, CD40, CD84, CD166, 4-1BB, OX40, ICOS, ICAM-1, CTLA-4, PD-1, LAG-3, 2B4, BTLA, CD16, IL7, IL12, IL15, KIR2DL4, KIR2DS1, NKp30, NKp44, NKp46, NKG2C, NKG2D, or T cell receptor polypeptide.

The hinge domain and the transmembrane domain of a CAR as disclosed herein (e.g., for the engineered immune cell, such as the engineered NK cell) can be derived from the same protein (e.g., CD8). Alternatively, the hinge domain and the transmembrane domain of the CAR as disclosed herein can be derived from different proteins.

A signaling domain of a CAR can comprise at least or up to about 1 signaling domain, at least or up to about 2 signaling domains, at least or up to about 3 signaling domains, at least or up to about 4 signaling domains, at least or up to about 5 signaling domains, at least or up to about 6 signaling domains, at least or up to about 7 signaling domains, at least or up to about 8 signaling domains, at least or up to about 9 signaling domains, or at least or up to about 10 signaling domains.

A signaling domain (e.g., a signaling peptide of the intracellular signaling domain) of a CAR in the engineered immune cell (e.g., engineered NK cell) as disclosed herein can comprise a full length or at least a portion of a polypeptide of CD3, 2B4, DAP10, DAP12, DNAM1, CD137 (41BB), IL21, IL7, IL12, IL15, NKp30, NKp44, NKp46, NKG2C, NKG2D, or any combination thereof.

Alternatively or in addition to (i.e., a co-stimulatory domain), the signaling domain CAR in the engineered immune cell (e.g., engineered NK cell) as disclosed herein can comprise a full length or at least a portion of a polypeptide of CD27, CD28, 4-1BB, OX40, ICOS, PD-1, LAG-3, 2B4, BTLA, DAP10, DAP12, CTLA-4, or NKG2D, or any combination thereof.

In some cases, the engineered immune cell (e.g., the engineered NK cell) as disclosed herein comprises the chimeric polypeptide receptor (e.g., CAR) that comprises at least CD8 transmembrane domain and one or more of: (i) 2B4 signaling domain and (ii) DAP10 signaling domain. In some cases, the engineered cell (e.g., the engineered NK cell) as disclosed herein comprises the chimeric polypeptide receptor (e.g., TFP or CAR) that comprises at least (i) CD8 transmembrane domain, (ii) 2B4 signaling domain, and (iii) DAP10 signaling domain. The 2B4 signaling domain can be flanked by the CD8 transmembrane domain and the DAP10 signaling domain. Alternatively, the DAP10 signaling domain can be flanked by the CD8 transmembrane domain and the 2B4 signaling domain. In some cases, the chimeric polypeptide receptor as disclosed herein can further comprise yet an additional signaling domain derived from CD3.

An antigen (i.e., a target antigen) of an antigen binding moiety of a chimeric polypeptide receptor (e.g., TFP or CAR) as disclosed herein can be a cell surface marker, a secreted marker, or an intracellular marker.

Non-limiting examples of an antigen (i.e., a target antigen) of an antigen binding moiety of a chimeric polypeptide receptor (e.g., TFP or CAR) as disclosed herein can include ADGRE2, carbonic anhydrase IX (CA1X), CCRI, CCR4, carcinoembryonic antigen (CEA), CD3, CD5, CD8, CD10, CD19, CD20, CD22, CD30, CD33, CD34, CD38, CD41, CD44, CD44V6, CD49f, CD56, CD70, CD74, CD99, CD123, CD133, CD138, CD269 (BCMA), CD S, CLEC12A, an antigen of a cytomegalovirus (CMV) infected cell (e.g., a cell surface antigen), epithelial glycoprotein2 (EGP 2), epithelial glycoprotein-40 (EGP-40), epithelial cell adhesion molecule (EpCAM), EGFRvIII, receptor tyrosine-protein kinases erb-B2,3,4, EGFIR, EGFR-VIII, ERBB folate-binding protein (FBP), fetal acetylcholine receptor (AChR), folate receptor-a, Ganglioside G2 (GD2), Ganglioside G3 (GD3), gp100, human Epidermal Growth Factor Receptor 2 (HER-2), human telomerase reverse transcriptase (hTERT), ICAM-1, Integrin B7, Interleukin-13 receptor subunit alpha-2 (IL-13Rα2), κ-light chain, kinase insert domain receptor (KDR), Kappa, Lewis A (CA19.9), Lewis Y (LeY), L1 cell adhesion molecule (L1-CAM), LILRB2, MART-1, melanoma antigen family A 1 (MAGE-A1), MICA/B, Mucin 1 (Muc-1), Mucin 16 (Muc-16), Mesothelin (MSLN), NKCSI, NKG2D ligand, c-Met, cancer-testis antigen NY-ESO-1, NY-ESO-2, oncofetal antigen (h5T4), PRAIVIE, prostate stem cell antigen (PSCA), PRAME prostate-specific membrane antigen (PSMA), ROR1, tumor-associated glycoprotein 72 (TAG-72), TIM-3, TRBCI, TRBC2, vascular endothelial growth factor R2 (VEGF-R2), Wilms tumor protein (WT-1), and various pathogen antigen (e.g., pathogen antigens derived from a virus, bacteria, fungi, parasite and protozoa capable of causing diseases). In some examples, a pathogen antigen is derived from HIV, HBV, EBV, HPV, Lasse Virus, Influenza Virus, or Coronavirus.

Additional examples of the antigen of the antigen binding moiety of the chimeric polypeptide receptor as disclosed herein can include 1-40-β-amyloid, 4-1BB, SAC, 5T4, activin receptor-like kinase 1, ACVR2B, adenocarcinoma antigen, AGS-22M6, alpha-fetoprotein, angiopoietin 2, angiopoietin 3, anthrax toxin, AOC3 (VAP-1), B7-H3, Bacillus anthracis anthrax, BAFF, beta-amyloid, B-lymphoma cell, C242 antigen, C5, CA-125, Canis lupus familiaris IL31, carbonic anhydrase 9 (CA-IX), cardiac myosin, CCL11 (eotaxin-1), CCR4, CCR5, CD11, CD18, CD125, CD140a, CD147 (basigin), CD15, CD152, CD154 (CD40L), CD19, CD2, CD20, CD200, CD22, CD221, CD23 (IgE receptor), CD25 (a chain of IL-2receptor), CD27, CD274, CD28, CD3, CD3 epsilon, CD30, CD33, CD37, CD38, CD4, CD40, CD40 ligand, CD41, CD44 v6, CD5, CD51, CD52, CD56, CD6, CD70, CD74, CD79B, CD80, CEA, CEA-related antigen, CFD, ch4D5, CLDN18.2, Clostridium difficile, clumping factor A, CSF1R, CSF2, CTLA-4, C-X-C chemokine receptor type 4, cytomegalovirus, cytomegalovirus glycoprotein B, dabigatran, DLL4, DPP4, DR5, E. coli shiga toxin type-1, E. coli shiga toxin type-2, EGFL7, EGFR, endotoxin, EpCAM, episialin, ERBB3, Escherichia coli, F protein of respiratory syncytial virus, FAP, fibrin II beta chain, fibronectin extra domain-B, folate hydrolase, folate receptor 1, folate receptor alpha, Frizzled receptor, ganglioside GD2, GD2, GD3 ganglioside, glypican 3, GMCSF receptor α-chain, GPNMB, growth differentiation factor 8, GUCY2C, hemagglutinin, hepatitis B surface antigen, hepatitis B virus, HER1, HER2/neu, HER3, HGF, HHGFR, histone complex, HIV-1, HLA-DR, HNGF, Hsp90, human scatter factor receptor kinase, human TNF, human beta-amyloid, ICAM-1 (CD54), IFN-α, IFN-γ, IgE, IgE Fc region, IGF-1 receptor, IGF-1, IGHE, IL17A, IL17F, IL20, IL-12, IL-13, IL-17, IL-1β, IL-22, IL-23, IL-31RA, IL-4, IL-5, IL-6, IL-6 receptor, IL-9, ILGF2, influenza A hemagglutinin, influenza A virus hemagglutinin, insulin-like growth factor I receptor, integrin α4β7, integrin α4, integrin α5β1, integrin α7β7, integrin αIIbβ3, integrin αvβ3, interferon α/β receptor, interferon gamma-induced protein, ITGA2, ITGB2 (CD18), KIR2D, Lewis-Y antigen, LFA-1 (CD11a), LINGO-1, lipoteichoic acid, LOXL2, L-selectin (CD62L), LTA, MCP-1, mesothelin, MIF, MS4A1, MSLN, MUC1, mucin CanAg, myelin-associated glycoprotein, myostatin, NCA-90 (granulocyte antigen), neural apoptosis-regulated proteinase 1, NGF, N-glycolylneuraminic acid, NOGO-A, Notch receptor, NRP1, Oryctolagus cuniculus, OX-40, oxLDL, PCSK9, PD-1, PDCD1, PDGF-R a, phosphate-sodium co-transporter, phosphatidylserine, platelet-derived growth factor receptor beta, prostatic carcinoma cells, Pseudomonas aeruginosa, rabies virus glycoprotein, RANKL, respiratory syncytial virus, RHD, Rhesus factor, RON, RTN4, sclerostin, SDC1, selectin P, SLAMF7, SOST, sphingosine-1-phosphate, Staphylococcus aureus, STEAP1, TAG-72, T-cell receptor, TEM1, tenascin C, TFPI, TGF-β1, TGF-β2, TGF-β, TNF-α, TRAIL-R1, TRAIL-R2, tumor antigen CTAA16.88, tumor specific glycosylation of MUC1, tumor-associated calcium signal transducer 2, TWEAK receptor, TYRP1 (glycoprotein 75), VEGFA, VEGFR1, VEGFR2, vimentin, and VWF.

Additional examples of the antigen of the antigen binding moiety of the chimeric polypeptide receptor as disclosed herein can include 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain.

Additional examples of the antigen of the antigen binding moiety of the chimeric polypeptide receptor as disclosed herein can include an antibody, a fragment thereof, or a variant thereof. Such antibody can be a natural antibody (e.g., naturally secreted by a subject's immune cell, such as B cells), a synthetic antibody, or a modified antibody. In some cases, he antigen of the antigen binding moiety of the chimeric polypeptide receptor as disclosed herein can include an Fc domain of an antibody from the group comprising 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, zlintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizumab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, Betalutin (177Lu-tetraxetan-tetulomab), bevacizumab, BEVZ92 (bevacizumab biosimilar), bezlotoxumab, BGB-A317, BHQ880, BI 836880, BI-505, biciromab, bimagrumab, bimekizumab, bivatuzumab mertansine, BIW-8962, blinatumomab, blosozumab, BMS-936559, BMS-986012, BMS-986016, BMS-986148, BMS-986178, BNC101, bococizumab, brentuximab vedotin, BrevaRex, briakinumab, brodalumab, brolucizumab, brontictuzumab, C2-2b-2b, canakinumab, cantuzumab mertansine, cantuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatumumab, concizumab, Cotara (iodine 1-131 derlotuximab biotin), cR6261, crenezumab, DA-3111 (trastuzumab biosimilar), dacetuzumab, daclizumab, dalotuzumab, dapirolizumab pegol, daratumumab, Daratumumab Enhanze (daratumumab), Darleukin, dectrekumab, demcizumab, denintuzumab mafodotin, denosumab, Depatuxizumab, Depatuxizumab mafodotin, derlotuximab biotin, detumomab, DI-B4, dinutuximab, diridavumab, DKN-01, DMOT4039A, dorlimomab aritox, drozitumab, DS-1123, DS-8895, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, FF-21101, FGFR2 Antibody-Drug Conjugate, Fibromun, ficlatuzumab, figitumumab, firivumab, flanvotumab, fletikumab, fontolizumab, foralumab, foravirumab, FPA144, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hu14.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D11, imciromab, imgatuzumab, IMGN529, IMMU-102 (yttrium Y-90 epratuzumab tetraxetan), IMMU-114, ImmuTune IMP701 Antagonist Antibody, INCAGN1876, inclacumab, INCSHR1210, indatuximab ravtansine, indusatumab vedotin, infliximab, inolimomab, inotuzumab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L19IL2/L19TNF, Labetuzumab, Labetuzumab Govitecan, LAG525, lambrolizumab, lampalizumab, L-DOS47, lebrikizumab, lemalesomab, lenzilumab, lerdelimumab, Leukotuximab, lexatumumab, libivirumab, lifastuzumab vedotin, ligelizumab, lilotomab satetraxetan, lintuzumab, lirilumab, LKZ145, lodelcizumab, lokivetmab, lorvotuzumab mertansine, lucatumumab, lulizumab pegol, lumiliximab, lumretuzumab, LY3164530, mapatumumab, margetuximab, maslimomab, matuzumab, mavrilimumab, MB311, MCS-110, MEDI0562, MEDI-0639, MEDI0680, MEDI-3617, MEDI-551 (inebilizumab), MEDI-565, MEDI6469, mepolizumab, metelimumab, MGB453, MGD006/S80880, MGD007, MGD009, MGD011, milatuzumab, Milatuzumab-SN-38, minretumomab, mirvetuximab soravtansine, mitumomab, MK-4166, MM-111, MM-151, MM-302, mogamulizumab, MOR202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratumab, olokizumab, omalizumab, OMP-131R10, OMP-305B83, onartuzumab, ontuxizumab, opicinumab, oportuzumab monatox, oregovomab, orticumab, otelixizumab, otlertuzumab, OX002/MEN1309, oxelumab, ozanezumab, ozoralizumab, pagibaximab, palivizumab, panitumumab, pankomab, PankoMab-GEX, panobacumab, parsatuzumab, pascolizumab, pasotuxizumab, pateclizumab, patritumab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, pritumumab, PRO 140, Proxinium, PSMA ADC, quilizumab, racotumomab, radretumab, rafivirumab, ralpancizumab, ramucirumab, ranibizumab, raxibacumab, refanezumab, regavirumab, REGN1400, REGN2810/SAR439684, reslizumab, RFM-203, RG7356, RG7386, RG7802, RG7813, RG7841, RG7876, RG7888, RG7986, rilotumumab, rinucumab, rituximab, RM-1929, R07009789, robatumumab, roledumab, romosozumab, rontalizumab, rovelizumab, ruplizumab, sacituzumab govitecan, samalizumab, SAR408701, SAR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIV1A, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, SYD985, SYM004 (futuximab and modotuximab), Sym015, TAB08, tabalumab, tacatuzumab tetraxetan, tadocizumab, talizumab, tanezumab, Tanibirumab, taplitumomab paptox, tarextumab, TB-403, tefibazumab, Teleukin, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tositumomab, tovetumab, tralokinumab, trastuzumab, trastuzumab emtansine, TRBS07, TRC105, tregalizumab, tremelimumab, trevogrumab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorsetuzumab mafodotin, votumumab, YYB-101, zalutumumab, zanolimumab, zatuximab, ziralimumab, and zolimomab aritox.

In some cases, the engineered immune cell (e.g., the engineered NK cell) as disclosed herein can comprise the chimeric polypeptide receptor (e.g., TFP or CAR) that comprises the antigen binding domain, and the antigen binding domain can be capable of binding specifically and preferentially to an antigen comprising one or more (e.g., 1, 2, 3, 4, or more) members selected from the group comprising BCMA, CD20, CD22, CD30, CD33, CD38, CD70, CD123, Kappa, Lewis Y, NKG2D ligand, ROR1, NY-ESO-1, NY-ESO-2, MART-1, and gp100. Non-limiting examples of the NKG2D ligand comprises one or more (e.g., 1, 2, 3, 4, or more) members selected from the group comprising of MICA, MICB, ULBP1, ULBP2, ULBP3, ULBP4, ULBP5, and ULBP6.

In some cases, the engineered immune cell (e.g., the engineered NK cell) as disclosed herein can comprise the chimeric polypeptide receptor (e.g., TFP or CAR) that comprises the antigen binding domain capable of specifically binding an antigen of a target cell, and the engineered immune cell can exhibit reduced expression or activity of an endogenous gene encoding the same antigen of the chimeric polypeptide receptor. As such, a population of the engineered immune cells can avoid targeting and killing each other, e.g., upon administration to a subject in need thereof.

In some cases, the engineered immune cell (e.g., the engineered NK cell) as disclosed herein can comprise the chimeric polypeptide receptor (e.g., TFP or CAR) that comprises the antigen binding domain, and the antigen binding domain can be capable of binding specifically and preferentially to CD23. In some cases, the engineered immune cell's endogenous gene encoding CD23 can be modified to effect reduced expression or activity of the endogenous CD23.

In some cases, the engineered immune cell (e.g., the engineered NK cell) as disclosed herein can comprise the chimeric polypeptide receptor (e.g., TFP or CAR) that comprises the antigen binding domain, and the antigen binding domain can be capable of binding specifically and preferentially to CD123. In some cases, the engineered immune cell's endogenous gene encoding CD123 can be modified to effect reduced expression or activity of the endogenous CD123.

In some cases, the engineered immune cell (e.g., the engineered NK cell) as disclosed herein can comprise the chimeric polypeptide receptor (e.g., TFP or CAR) that comprises the antigen binding domain, and the antigen binding domain can be capable of binding specifically and preferentially to CD38. In some cases, the engineered immune cell's endogenous gene encoding CD38 can be modified to effect reduced expression or activity of the endogenous CD38. In some cases, the subject engineered immune cells comprising the chimeric polypeptide receptor against CD38 can be capable of targeting and effecting death (or degradation) of plasma cells.

In some cases, the engineered immune cell (e.g., the engineered NK cell) as disclosed herein can comprise the chimeric polypeptide receptor (e.g., TFP or CAR) that comprises the antigen binding domain, and the antigen binding domain can be capable of binding specifically and preferentially to CD38. In some examples, the engineered immune cell is an engineered NK cell that is derived from an isolated ESC or an induced stem cell (e.g., iPSC). In some cases, the engineered immune cell's endogenous gene encoding CD38 can be modified to effect reduced expression or activity of the endogenous CD38.

In some cases, the engineered immune cell (e.g., the engineered NK cell) as disclosed herein can comprise the chimeric polypeptide receptor (e.g., TFP or CAR) that comprises the antigen binding domain, and the antigen binding domain can be capable of binding specifically and preferentially to CD7. In some cases, the engineered immune cell's endogenous gene encoding CD7 can be modified to effect reduced expression or activity of the endogenous CD7.

E. Stem Cells

Any one of the engineered immune cell (e.g., the engineered NK cell) disclosed herein can be derived from an isolated stem cell (e.g., an ESC) or an induced stem cell (iPSC). The isolated stem cell or the induced stem cell can be modified (e.g., genetically modified) to generate the engineered immune cell.

In some cases, pluripotency of stem cells (e.g., ESCs or iPSCs) can be determined, in part, by assessing pluripotency characteristics of the cells. Pluripotency characteristics can include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

In some cases, stem cells (e.g., ESCs or iPSCs) can be genetically modified to generate (e.g., induce differentiation into) CD34+ hematopoietic stem cells. The stem cells can be genetically modified to express any one of the heterologous polypeptides (e.g., cytokines, receptors, etc.) as disclosed herein prior to, subsequent to, or during the induced hematopoietic stem cell differentiation. The stem cells can be genetically modified to reduce expression or activity of any one of the endogenous genes or polypeptides (e.g., cytokines, receptors, etc.) as disclosed herein prior to, subsequent to, or during the induced hematopoietic stem cell differentiation. In some cases, such genetically modified CD34+ hematopoietic stem cell is or is a source of any one of the engineered immune cell of the present disclosure.

In some examples, stem cells as disclosed herein can be cultured in APEL media with ROCKi (Y-27632) (e.g., at about 10 micromolar (μM)), SCF (e.g., at about 40 nanograms per milliner (ng/mL) of media), VEGF (e.g., at about 20 ng/mL of media), and BMP-4 (e.g., at about 20 ng/mL of media) to differentiate into CD34+ hematopoietic stem cells.

In some cases, the CD34+ hematopoietic stem cells (e.g., genetically modified with one or more features of any one of the engineered immune cell of the present disclosure) can be induced to differentiate in to a committed immune cell, such as T cells or NK cells. As such, in some cases, the induced differentiation process generates any one of the engineered NK cell of the present disclosure.

In some examples, genetically modified CD34+ hematopoietic stem cells are cultured in the presence of IL-3 (e.g., about 5 ng/mL), IL-7 (e.g., about 20 ng/mL), IL-15 (e.g., about 10 ng/mL), SCF (e.g., about 20 ng/mL), and Flt3L (e.g., about 10 ng/mL) to differentiate into CD45+NK cells.

In some cases, the CD45+NK cells can be expanded in culture, e.g., in a media comprising IL-2, mbIL-21 aAPC using Gas Permeable Rapid Expansion (G-Rex) platform.

In some cases, iPSC-derived NK cells as disclosed herein can be cultured with one or more heterologous cytokines comprising 11-2, IL-15, or IL-21. In some cases, iPSC-derived NK cells as disclosed herein can be cultured with (e.g., for cell expansion) one or more heterologous cytokines selected from the group consisting of 11-2, IL-15, and IL-21. In some cases, iPSC-derived NK cells as disclosed herein can be cultured with two or more heterologous cytokines selected from the group consisting of 11-2, IL-15, and IL-21 (e.g., IL-2 and IL-15, IL-2 and IL-21, or IL-15 and IL-21), either simultaneously or sequentially in any order. In some cases, iPSC-derived NK cells as disclosed herein can be cultured with all of 11-2, IL-15, and IL-21, either simultaneous or sequentially in any order.

F. Gene Editing or Genetic Material Delivery

The gene editing moiety as disclosed herein can comprise a CRISPR-associated polypeptide (Cas), zinc finger nuclease (ZFN), zinc finger associate gene regulation polypeptides, transcription activator-like effector nuclease (TALEN), transcription activator-like effector associated gene regulation polypeptides, meganuclease, natural master transcription factors, epigenetic modifying enzymes, recombinase, flippase, transposase, RNA-binding proteins (RBP), an Argonaute protein, any derivative thereof, any variant thereof, or any fragment thereof. In some embodiments, the actuator moiety comprises a Cas protein, and the system further comprises a guide RNA (gRNA) which complexes with the Cas protein. In some embodiments, the actuator moiety comprises an RBP complexed with a gRNA which is able to form a complex with a Cas protein. In some embodiments, the gRNA comprises a targeting segment which exhibits at least 80% sequence identity to a target polynucleotide. In some embodiments, the Cas protein substantially lacks DNA cleavage activity.

In some cases, a suitable gene editing moiety comprises CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); any derivative thereof, any variant thereof; and any fragment thereof.

Non-limiting examples of Cas proteins include c2c1, C2c2, c2c3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cul966, and homologs or modified versions thereof.

In some cases, the gene editing moiety as disclosed herein can be fused with an additional functional moiety (e.g., to form a fusion moiety), and non-limiting examples of a function of the additional functional moiety can include methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, and demyristoylation activity. For example, a fusion protein can be a fusion in a Cas protein and an effector or repressor functional moiety.

Alternatively or in addition to, gene editing (e.g., knock in) or delivery of heterologous genetic material can be achieved other viral and non-viral based gene transfer methods can be used to introduce nucleic acids in host cells (e.g., stem cells, hematopoietic stem cells, etc. as disclosed herein). Such methods can be used to administer nucleic acids encoding polypeptide molecules of the present disclosure to cells in culture (or in a host organism). Viral vector delivery systems can include DNA and RNA viruses, which can have either episomal or integrated genomes after delivery to the cell. Non-viral vector delivery systems can include DNA plasmids, RNA (e.g. a transcript of a vector described herein), naked nucleic acid, and nucleic acid complexed with a delivery vehicle, such as a liposome.

RNA or DNA viral based systems can be used to target specific cells and traffick the viral payload to the nucleus of the cell. Viral vectors can be used to treat cells in vitro, and the modified cells can optionally be administered (ex vivo). Alternatively, viral vectors can be administered directly (in vivo) to the subject. Viral based systems can include retroviral, lentivirus, adenoviral, adeno-associated and herpes simplex virus vectors for gene transfer. Integration in the host genome can occur with the retrovirus, lentivirus, and adeno-associated virus gene transfer methods, which can result in long term expression of the inserted transgene.

Methods of non-viral delivery of nucleic acids can include lipofection, nucleofection, microinjection, biolistics, virosomes, liposomes, immunoliposomes, polycation or lipid:nucleic acid conjugates, naked DNA, artificial virions, and agent-enhanced uptake of DNA. Cationic and neutral lipids that are suitable for efficient receptor-recognition lipofection of polynucleotides can be used.

Alternatively or in addition to, antisense oligonucleotides can be utilized to suppress or silence a target gene expression. Non-limiting examples of antisense oligonucleotides can include short hairpin RNA (shRNA), microRNA (miRNA), and small interfering RNA (siRNA).

G. Co-Therapy

The engineered immune cell (e.g., the engineered NK cell) of the present disclosure can be combined with a co-therapeutic agent to treat a subject in need thereof. In some cases, the engineered immune cell can be administered to the subject prior to, concurrent with, or subsequent to administration of the co-therapeutic agent to the subject.

In one aspect, the present disclosure provides a composition comprising (a) any one of the engineered immune cell (e.g., the engineered NK cell) disclosed herein and (b) a co-therapeutic agent (i.e., a separate therapeutic agent) (e.g., an antibody, such as anti-CD20 antibody or anti-PD1 antibody). In some cases, the engineered immune cell can comprise one or more of: (i) a heterologous cytokine (e.g., a heterologous IL, such as IL-15) as disclosed herein, (ii) a CD16 variant for enhanced CD16 signaling as disclosed herein, and (iii) a chimeric polypeptide receptor comprising an antigen binding moiety capable of binding to an antigen, as disclose herein. In some examples, the co-therapeutic agent comprises an anti-CD20 antibody.

In some cases, the engineered immune cell can comprise the heterologous cytokine (e.g., IL-15) as disclosed herein and one or both of: (ii) the CD16 variant for enhanced CD16 signaling and (iii) the chimeric polypeptide receptor comprising the antigen binding moiety.

In some cases, the engineered immune cell can comprise the CD16 variant for enhanced CD16 signaling and one or both of: (i) the heterologous cytokine (e.g., IL-15) and (iii) the chimeric polypeptide receptor comprising the antigen binding moiety.

In some cases, the engineered immune cell can comprise the chimeric polypeptide receptor comprising the antigen binding moiety and one or both of: (i) the heterologous cytokine (e.g., IL-15) and (ii) the CD16 variant for enhanced CD16 signaling.

Non-limiting examples of a co-therapeutic agent can include cytotoxic agents, chemotherapeutic agents, growth inhibitory agents, agents used in radiation therapy, anti-angiogenesis agents, apoptotic agents, anti-tubulin agents, and other agents to treat cancer, for example, anti-CD20 antibodies, anti-PD1 antibodies (e.g., Pembrolizumab) platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets PDGFR-β, BlyS, APRIL, BCMA receptor(s), TRAIL/Apo2, other bioactive and organic chemical agents, and the like.

The term “cytotoxic agent” generally refers to a substance that inhibits or prevents the function of cells and/or causes destruction of cells. Non-limiting examples of a cytotoxic agent can include radioactive isotopes (e.g., At211, I131, I125, Y90, Re186, Re188, Sm153, Bi212, P32, and radioactive isotopes of Lu), chemotherapeutic agents, e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin.

Non-limiting examples of a chemotherapeutic agent can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics; dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aidophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, for example taxanes including TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin. Additional chemotherapeutic agents include the cytotoxic agents useful as antibody drug conjugates, such as maytansinoids (DM1, for example) and the auristatins MMAE and MMAF, for example.

Examples of a chemotherapeutic agent can also include “anti-hormonal agents” or “endocrine therapeutics” that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD) leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGFR); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

Examples of a chemotherapeutic agent can also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, feMzumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 antibody genetically modified to recognize interleukin-12 p40 protein.

Examples of a chemotherapeutic agent can also include “tyrosine kinase inhibitors” such as an EGFR-targeting agent (e.g., small molecule, antibody, etc.); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®).

Examples of a chemotherapeutic agent can also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Examples of a chemotherapeutic agent can also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate: immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (ENBREL®), infliximab (REMICADE®), adalimumab (HUMIRA®), certolizumab pegol (CIMZIA®), golimumab (SIMPONI®), Interleukin 1 (IL-1) blockers such as anakinra (KINERET®), T-cell costimulation blockers such as abatacept (ORENCIA®), Interleukin 6 (IL-6) blockers such as tocilizumab (ACTEMERA®); Interleukin 13 (IL-13) blockers such as lebrikizumab; Interferon alpha (IFN) blockers such as rontalizumab; beta 7 integrin blockers such as rhuMAb Beta7; IgE pathway blockers such as Anti-M1 prime; Secreted homotrimeric LTa3 and membrane bound heterotrimer LTa/β2 blockers such as Anti-lymphotoxin alpha (LTa); miscellaneous investigational agents such as thioplatin, PS-341, phenylbutyrate, ET-18-OCH3, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifamib (R11577); orafenib, ABT510; Bcl-2 inhibitor such as oblimersen sodium (GENASENSE R); pixantrone; famesyltransferase inhibitors such as lonafamib (SCH 6636, SARASAR™); and pharmaceutically acceptable salts, acids or derivatives of any of the above; as well as combinations of two or more of the above.

The term “growth inhibitory agent” generally refers to a compound or composition which inhibits growth and/or proliferation of a cell (e.g., a cell whose growth is dependent on PD-L1 expression) either in vitro or in vivo. The growth inhibitory agent may be one which significantly reduces the percentage of cells in S phase. Non-limiting examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as the anthracycline antibiotic doxorubicin ((85-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione), epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

H. Methods of Use

The engineered immune cell (e.g., the engineered NK cell) of the present disclosure can be used (e.g., administered) to treat a subject in need thereof. The subject can have or can be suspected of having a condition, such as a disease (e.g., cancer, tumor, tissue degeneration, fibrosis, etc.). A cell (e.g., a stem cell or a committed adult cell) can be obtained from the subject, and such cell can be cultured ex vivo and genetically modified to generate any subject engineered immune cell (e.g., any engineered NK cell) as disclosed herein. Subsequently, the engineered immune cell can be administered to the subject for adaptive immunotherapy.

The subject can be treated (e.g., administered with) a population of engineered immune cells (e.g., engineered NK cells) of the present disclosure for at least or up to about 1 dose, at least or up to about 2 doses, at least or up to about 3 doses, at least or up to about 4 doses, at least or up to about 5 doses, at least or up to about 6 doses, at least or up to about 7 doses, at least or up to about 8 doses, at least or up to about 9 doses, or at least or up to about 10 doses.

In one aspect, the present disclosure provides a method comprising (a) obtaining a cell from a subject; and (b) generating, from the cell, any one of the engineered immune cell (e.g., the engineered NK cell) disclosed herein. In some cases, the cell obtained from the subject is ESC. In some cases, the cell (e.g., a fibroblast, such as an adult skin fibroblast) obtained from the subject is modified and transformed into an iPSC.

In one aspect, the present disclosure provides a method comprising administering to a subject in need thereof a population of NK cells comprising any one of the engineered immune cell (e.g., the engineered NK cell) disclosed herein. In some cases, the method can further comprise administering to the subject a co-therapeutic agent (e.g., a chemotherapeutic agent, anti-CD20 antibody, etc.).

In one aspect, the present disclosure provides a method comprising administering to a subject in need thereof any one of the composition disclosed herein. In some cases, the composition can comprise (i) any one of the engineered immune cell (e.g., the engineered NK cell) disclosed herein and (ii) a co-therapeutic agent (e.g., a chemotherapeutic agent, anti-CD20 antibody, etc.).

Any one of the methods disclosed herein can be utilized to treat a target cell, a target tissue, a target condition, or a target disease of a subject.

A target disease can be a viral, bacterial, and/or parasitic infection; inflammatory and/or autoimmune disease; or neoplasm such as a cancer and/or tumor.

A target cell can be a diseased cell. A diseased cell can have altered metabolic, gene expression, and/or morphologic features. A diseased cell can be a cancer cell, a diabetic cell, and an apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

A variety of target cells can be killed using any one of the engineered immune cell (e.g., the engineered NK cell) disclosed herein. A target cell can include a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

progenitor cells, including rodent stem cells, rodent progenitor cells, human stem cells, human progenitor cells, etc. Clonal cells can comprise the progeny of a cell. A target cell can comprise a target nucleic acid. A target cell can be in a living organism. A target cell can be a genetically modified cell. A target cell can be a host cell.

A target cell can be a totipotent stem cell, however, in some embodiments of this disclosure, the term “cell” may be used but may not refer to a totipotent stem cell. A target cell can be a plant cell, but in some embodiments of this disclosure, the term “cell” may be used but may not refer to a plant cell. A target cell can be a pluripotent cell. For example, a target cell can be a pluripotent hematopoietic cell that can differentiate into other cells in the hematopoietic cell lineage but may not be able to differentiate into any other non-hematopoietic cell. A target cell may be able to develop into a whole organism. A target cell may or may not be able to develop into a whole organism. A target cell may be a whole organism.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and a apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

Non-limiting examples of cells which can be target cells include, but are not limited to, lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

Of particular interest are cancer cells. In some embodiments, the target cell is a cancer cell. Non-limiting examples of cancer cells include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

In some cases, the target cell (e.g., B cells) as disclosed herein is associated or is suspected of being associated with an autoimmune disease. The subject being treated with any one of the engineered immune cell (e.g., engineered NK cell) of the present disclosure can have or can be suspected of having an autoimmune disease.

Non-limiting examples of an autoimmune disease can include acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, antibody-mediated transplantation rejection, anti-GBM/Anti-TBM nephritis, antiphospholipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, autoimmune urticaria, axonal & neuronal neuropathies, Balo disease, Behcet's disease, bullous pemphigoid, cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, hypergammaglobulinemia, idiopathic thrombocytopenic purpura (ITP), IgA nephropathy, IgG4-related sclerosing disease, immunoregulatory lipoproteins, inclusion body myositis, inflammatory bowel disease, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), lupus (SLE), lyme disease, Meniere's disease, microscopic polyangiitis, mixed connective tissue disease (MCTD), monoclonal gammopathy of undetermined significance (MGUS), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis (Granulomatosis with Polyangiitis (GPA)).

In some cases, the autoimmune disease comprises one or more members selected from the group comprising rheumatoid arthritis, type 1 diabetes, systemic lupus erythematosus (lupus or SLE), myasthenia gravis, multiple sclerosis, scleroderma, Addison's Disease, bullous pemphigoid, pemphigus vulgaris, Guillain-Barré syndrome, Sjogren syndrome, dermatomyositis, thrombotic thrombocytopenic purpura, hypergammaglobulinemia, monoclonal gammopathy of undetermined significance (MGUS), Waldenstrom's macroglobulinemia (WM), chronic inflammatory demyelinating polyradiculoneuropathy (CIDP), Hashimoto's Encephalopathy (HE), Hashimoto's Thyroiditis, Graves' Disease, Wegener's Granulomatosis, and antibody-mediated transplantation rejection (e.g., for tissue transplants such as renal transplant). In examples, the autoimmune disease can be type 1 diabetes, lupus, or rheumatoid arthritis.

In some cases, the target disease is T cell leukemia (TCL), such as T-cell acute lymphoblastic leukemia (T-ALL). For example, any one of the engineered immune cell (e.g., the engineered NK cell) disclosed herein that comprises one or more of: (i) a chimeric polypeptide receptor comprising an antigen binding domain capable of binding to CD7 as disclosed herein, (ii) a heterologous cytokine (e.g., IL-15) as disclosed herein, and (iii) reduced expression or activity of endogenous gene encoding the same cytokine as the heterologous cytokine (e.g., endogenous CD7) can be administered to a subject in need thereof to treat TCL.

In some cases the target disease is acute myeloid leukemia (AML). For example, any one of the engineered immune cell (e.g., the engineered NK cell) disclosed herein that comprises one or more of: (i) a chimeric polypeptide receptor comprising an antigen binding domain capable of binding to an antigen (e.g., CD33 and/or CD123) as disclosed herein, (ii) a heterologous cytokine (e.g., IL-15) as disclosed herein, and (iii) a CD16 variant for enhanced CD16 signaling as disclosed herein can be administered to a subject in need thereof to treat AML.

In some cases, the target disease is non-Hodgkin's lymphoma (NHL).

In some cases, the target disease is chronic lymphocytic leukemia (CLL).

In some cases, the target disease is B-cell leukemia (BCL). For example, any one of the engineered immune cell (e.g., the engineered NK cell) disclosed herein that comprises one or more of: (i) a chimeric polypeptide receptor comprising an antigen binding domain capable of binding to CD19 as disclosed herein, (ii) a heterologous cytokine (e.g., IL-15) as disclosed herein, and (iii) a CD16 variant for enhanced CD16 signaling as disclosed herein can be administered to a subject in need thereof to treat BCL.

In some cases, the target disease is non-small-cell lung carcinoma (NSCLC).

In some cases, the target cells form a tumor (i.e., a solid tumor). A tumor treated with the methods herein can result in stabilized tumor growth (e.g., one or more tumors do not increase more than 1%, 5%, 10%, 15%, or 20% in size, and/or do not metastasize). In some cases, a tumor is stabilized for at least about 1, 2, 3, 4, 5, 6, 7, 8, 9, 10, 11, 12, or more weeks. In some cases, a tumor is stabilized for at least about 1, 2, 3, 4, 5, 6, 7, 8, 9, 10, 11, 12, or more months. In some cases, a tumor is stabilized for at least about 1, 2, 3, 4, 5, 6, 7, 8, 9, 10, or more years. In some cases, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95% or more. In some cases, the tumor is completely eliminated, or reduced below a level of detection. In some cases, a subject remains tumor free (e.g. in remission) for at least about 1, 2, 3, 4, 5, 6, 7, 8, 9, 10, 11, 12, or more weeks following treatment. In some cases, a subject remains tumor free for at least about 1, 2, 3, 4, 5, 6, 7, 8, 9, 10, 11, 12, or more months following treatment. In some cases, a subject remains tumor free for at least about 1, 2, 3, 4, 5, 6, 7, 8, 9, 10, or more years after treatment.

### EXAMPLES

### Example 1: Engineered NK Cells

Table 1 illustrates examples of engineered NK cells with or without genetic modifications, along with possible functions, and therapeutic indications. Examples of therapeutic indications can include acute myeloid leukemia (AML), multiple myeloma (MM), Myelodysplastic syndrome (MDS), B cell leukemia, T cell leukemia, solid tumor, and blood cancer.

The NK cells as disclosed herein can be generated by engineering NK cells derived from a subject. Alternatively, the NK cells as disclosed herein can be derived from stem cells, such as isolated stem cells (e.g., embryonic stem cells) or induced stem cells (e.g., iPSCs). For NK cells derived from stem cells, one or more genetic modifications as disclosed herein can be introduced at (A) the stem cell state, (B) the hematopoietic stem cell state, and/or (C) the NK cell state. Yet in another alternative, the NK cells as disclosed herein can be NK cell lines.

TABLE 1

| No. | Genetic modifications | Functions | Therapeutic indications |
|---|---|---|---|
| 1 | none | Non-engineered NK | AML, MM, MDS |
| 2 | aCD33-CAR or aCD123-CAR + | Target specificity + persistency + | AML |
| | IL15 + hnCD16 | ADCC | |
| 3 | aCD19-CAR + IL15 + hnCD16 | Target specificity + persistency + | B cell leukemia +/− |
| | | ADCC | anti-CD20 |
| 4 | aCD19-CAR + IL15 + hnCD16 + | Target specificity + persistency + | B cell leukemia +/− |
| | iCaspase9 | ADCC + | anti-CD20 |
| | | safety switch | |
| 5 | aCD7-CAR + IL15 + CD7-KO | Target specificity + persistency | T cell leukemia |
| 6 | hnCD16 + IL15 | ADCC + persistency | Solid tumor + |
| | | | anti-PD-1 |
| 7 | hnCD16-CD64 + IL15 | ADCC + persistency | Solid tumor + |
| | | | anti-PD-1 |
| 8 | hnCD16 + IL15 + CD38-KO | ADCC + persistency | Multiple Myeloma −/+ |
| | | | anti-CD38 |
| 9 | aBCMA-CAR + hnCD16 + IL15 + | Target specificity + persistency + | Multiple Myeloma −/+ |
| | CD38-KO | ADCC | anti-CD38 |
| 10 | CAR + IL15 + hnCD16 + TME | Target specificity + persistency + | Blood cancer, |
| | genes | ADCC + | Solid tumor |
| | | enhanced activity | |
| 11 | CAR + IL15 + hnCD16 + TME | Target specificity + persistency + | Blood cancer, |
| | genes + | ADCC + | Solid tumor |
| | hypo-immunity genes (for re- | enhanced activity + hypo- | |
| | dosing) | immunity (for re-dosing) | |
| 12 | CAR + IL15 + hnCD16 + TME | Target specificity + persistency + | Blood cancer, |
| | genes + | ADCC + | Solid tumor |
| | hypo-immunity genes (for re- | enhanced activity + hypo- | |
| | dosing) + iCaspase 9 | immunity (for re-dosing) + Safety | |
| | | switch | |

### Example 2: Enhanced Function in Tumor Microenvironment (TME)

For improved function in tumor microenvironment (e.g., proliferation, persistence, hypo-immunity, anti-tumor activity, etc.), cells of interest can be engineered to carry (i) one or more enhanced or introduced genes (e.g., introduced transgenes) and/or (ii) one or more reduced expression level of endogenous genes (e.g., loss-of-function of genes of interest). The reduced expression level of the endogenous genes can be induced by, e.g., CRISPP/Cas and one or more guide nucleic acid molecules, such as the non-limiting exemplary guide RNA sequences provided in TABLE 3. The cells of interest can be stem cells, such as isolated stem cells (e.g., embryonic stem cells) or induced stem cells (e.g., iPSCs). The cells of interest can be immune cells (e.g., NK cells). Such immune cells can be derived from the stem cells as disclosed herein. Alternatively, such immunes can be immune cell lines (e.g., NK cell lines).

For example, for improved function in tumor microenvironment, NK cells can be engineered to carry certain transgenes and/or loss-of-function of genes of interest, as shown in TABLE 3.

TABLE 3
guide RNA sequences for enhanced function in tumor microenvironment

| SEQ | | | |
|---|---|---|---|
| ID | | | |
| NO | Target name | Guide RNA Sequence | Guide RNA position |
| 44 | TGFBR3 | AGCAGTGCTGCCCACAGCAT | chr1:91683782- |
| | | | 91683804:− |
| 45 | TGFBR3 | GTGATCGGAGCACTCCTGAC | chr1:91695701- |
| | | | 91695723:− |
| 46 | TGFBR3 | TCTGGACACCCTAACCGTGA | chr1:91695744- |
| | | | 91695766:− |
| 47 | FCER1G | GGACAATTCCATACAGAAAC | chr1:161218029- |
| | | | 161218051:− |
| 48 | FCER1G | CTTCAGTCGACAGTAGAGGA | chr1:161218055- |
| | | | 161218077:− |
| 49 | FCER1G | TCAGAGTCTCGTAAGTCTCC | chr1:161218898- |
| | | | 161218920:− |
| 50 | CTLA4 | CCTTGTGCCGCTGAAATCCA | chr2:203867951- |
| | | | 203867973:− |
| 51 | CTLA4 | TGAACCTGGCTACCAGGACC | chr2:203867980- |
| | | | 203868002:+ |
| 52 | CTLA4 | GTACCCACCGCCATACTACC | chr2:203870878- |
| | | | 203870900:+ |
| 53 | TGFBR2 | GGACGATGTGCAGCGGCCAC | chr3:30606907- |
| | | | 30606929:− |
| 54 | TGFBR2 | TGCTGGCGATACGCGTCCAC | chr3:30606928- |
| | | | 30606950:− |
| 55 | TGFBR2 | CCGACTTCTGAACGTGCGGT | chr3:30606955- |
| | | | 30606977:− |
| 56 | CISH | AGTCCACGTCGGCCACCAGA | chr3:50607666- |
| | | | 50607688:+ |
| 57 | CISH | TCGGGCCTCGCTGGCCGTAA | chr3:50608105- |
| | | | 50608127:+ |
| 58 | CISH | GGGTTCCATTACGGCCAGCG | chr3:50608110- |
| | | | 50608132:− |
| 59 | CBLB | AAATTCTCGGAGGATGCTCC | chr3:105659009- |
| | | | 105659031:+ |
| 60 | CBLB | AGAATAATGTCGAAGTTGCC | chr3:105659027- |
| | | | 105659049:− |
| 61 | CBLB | GGGCAACTTCGACATTATTC | chr3:105659029- |
| | | | 105659051:+ |
| 62 | CD96 | GGCACAGTAGAAGCCGTATT | chr3:111545202- |
| | | | 111545224:− |
| 63 | CD96 | AGGCACAGTAGAAGCCGTAT | chr3:111545203- |
| | | | 111545225:− |
| 64 | CD96 | ACGGCTTCTACTGTGCCTAT | chr3:111545208- |
| | | | 111545230:+ |
| 65 | TIGIT | TATCGTTCACGGTCAGCGAC | chr3:114295768- |
| | | | 114295790:− |
| 66 | TIGIT | TCGCTGACCGTGAACGATAC | chr3:114295772- |
| | | | 114295794:+ |
| 67 | TIGIT | CGCTGACCGTGAACGATACA | chr3:114295773- |
| | | | 114295795:+ |
| 68 | TET2 | CAGGACTCACACGACTATTC | chr4:105234149- |
| | | | 105234171:− |
| 69 | TET2 | GTGTAGATGGATTAGGACTC | chr4:105235322- |
| | | | 105235344:− |
| 70 | TET2 | CTTATGGTCAAATAACGACT | chr4:105237052- |
| | | | 105237074:− |
| 71 | HAVCR2 | ATTGCAAAGCGACAACCCAA | chr5:157087109- |
| | | | 157087131:+ |
| 72 | HAVCR2 | GCGACAACCCAAAGGTTGTG | chr5:157087117- |
| | | | 157087139:+ |
| 73 | HAVCR2 | CGACAACCCAAAGGTTGTGA | chr5:157087118- |
| | | | 157087140:+ |
| 74 | IL17A | TATTCCGGTTATGGATGTTC | chr6:52187719- |
| | | | 52187741:− |
| 75 | IL17A | TGGTATTGGTATTCCGGTTA | chr6:52187728- |
| | | | 52187750:− |
| 76 | IL17A | GGATCGGTTGTAGTAATCTG | chr6:52187763- |
| | | | 52187785:− |
| 77 | TGFBR1 | CCTCCTCGTGCTGGCGGCGG | chr9:99105244- |
| | | | 99105266:+ |
| 78 | TGFBR1 | TTGACTTAATTCCTCGAGAT | chr9:99128981- |
| | | | 99129003:+ |
| 79 | TGFBR1 | CATACAAACGGCCTATCTCG | chr9:99128992- |
| | | | 99129014:− |
| 80 | LAG3 | TTCGCAGAAGGCTGAGATCC | chr12:6773283- |
| | | | 6773305:− |
| 81 | LAG3 | CGCTCAGCACCGTGTAGCGG | chr12:6773778- |
| | | | 6773800:− |
| 82 | LAG3 | CGCTACACGGTGCTGAGCGT | chr12:6773782- |
| | | | 6773804:+ |
| 83 | KLRD1 | GATTGGACTCTCTTACAGTG | chr12:10313442- |
| | | | 10313464:+ |
| 84 | KLRD1 | ACAGTGAGGAGCACACCGCC | chr12:10313456- |
| | | | 10313478:+ |
| 85 | KLRD1 | GGAGCACACCGCCTGGTTGT | chr12:10313463- |
| | | | 10313485:+ |
| 86 | KLRC1 | GAAGCTCATTGTTGGGATCC | chr12:10450538- |
| | | | 10450560:− |
| 87 | KLRC1 | CTCCAGAGAAGCTCATTGTT | chr12:10450545- |
| | | | 10450567:− |
| 88 | KLRC1 | GCTCCAGAGAAGCTCATTGT | chr12:10450546- |
| | | | 10450568:− |
| 89 | SOCS2 | CTGCGGTGCCTTGAGCCCTC | chr12:93572904- |
| | | | 93572926:+ |
| 90 | SOCS2 | CTTGAGCCCTCCGGGAATGG | chr12:93572913- |
| | | | 93572935:+ |
| 91 | SOCS2 | TTCGCCAGACGCGCCGCCTG | chr12:93572988- |
| | | | 93573010:− |
| 92 | CDK8 | TTGCTCCAAGAAGTAGTTCA | chr13:26385299- |
| | | | 26385321:− |
| 93 | CDK8 | CTTGCTCCAAGAAGTAGTTC | chr13:26385300- |
| | | | 26385322:− |
| 94 | CDK8 | CTGAACTACTTCTTGGAGCA | chr13:26385301- |
| | | | 26385323:+ |
| 95 | SOCS1 | GCGCGTGATGCGCCGGTAAT | chr16:11255272- |
| | | | 11255294:+ |
| 96 | SOCS1 | GCCGGTAATCGGCGTGCGAA | chr16:11255283- |
| | | | 11255305:+ |
| 97 | SOCS1 | TCCGTTCGCACGCCGATTAC | chr16:11255284- |
| | | | 11255306:− |
| 98 | STAT3 | ATGGTTCCACGGACTGGATC | chr17:42322465- |
| | | | 42322487:+ |
| 99 | STAT3 | TGGTTCCACGGACTGGATCT | chr17:42322466- |
| | | | 42322488:+ |
| 100 | STAT3 | TAAGACCCAGATCCAGTCCG | chr17:42322471- |
| | | | 42322493:− |
| 101 | SOCS3 | GTCGCGGATCAGAAAGGTGC | chr17:78358880- |
| | | | 78358902:+ |
| 102 | SOCS3 | CGGATCAGAAAGGTGCCGGC | chr17:78358884- |
| | | | 78358906:+ |
| 103 | SOCS3 | CAGCAGGTTCGCCTCGCCGC | chr17:78358919- |
| | | | 78358941:+ |
| 104 | TGFB1 | CGGCCCACGTAGTACACGAT | chr19:41331110- |
| | | | 41331132:+ |
| 105 | TGFB1 | GCATAACCCGGGCGCCTCGG | chr19:41331168- |
| | | | 41331190:− |
| 106 | TGFB1 | CCGAGGCGCCCGGGTTATGC | chr19:41331171- |
| | | | 41331193:+ |
| 107 | BCL3 | AGGTCGCAGCCGCCGCGCAG | chr19:44748965- |
| | | | 44748987:− |
| 108 | BCL3 | GTAGTAAAGCGCCTCCGGCC | chr19:44749020- |
| | | | 44749042:− |
| 109 | BCL3 | GGTAGTAAAGCGCCTCCGGC | chr19:44749021- |
| | | | 44749043:− |
| 110 | INPP5D | TGGACTCGCTGGCACGCACG | chr2:233060562- |
| | | | 233060584:− |
| 111 | INPP5D | TGTTGCCATGGTTCCAGCAG | chr2:233060484- |
| | | | 233060506:− |
| 112 | INPP5D | CAACATCACCCGCTCCAAGG | chr2:233060502- |
| | | | 233060524:+ |
| 113 | PDCD1 | CGACTGGCCAGGGCGCCTGT | chr2:241858808- |
| | | | 241858830:+ |
| 114 | PDCD1 | TGCAGATCCCACAGGCGCCC | chr2:241858815- |
| | | | 241858837:− |
| 115 | PDCD1 | ACGACTGGCCAGGGCGCCTG | chr2:241858807- |
| | | | 241858829:+ |
| 116 | KIR3DL3 | AAAGAAGAGGAGGATAGCA | chr19:54735304- |
| | | A | 54735326:− |
| 117 | KIR3DL3 | AAGAAGAGGAGGATAGCAA | chr19:54735303- |
| | | A | 54735325:− |
| 118 | KIR3DL2 | AATGGACCAAGAGCCTGCGG | chr19:54866380- |
| | | | 54866402:+ |
| 119 | KIR3DL2 | ACCACAGTCAGGTCTTGAGG | chr19:54866701- |
| | | | 54866723:+ |
| 120 | KIR3DL3 | AGAAGAGGAGGATAGCAAA | chr19:54735302- |
| | | G | 54735324:− |
| 121 | KIR3DL1, KIR3DL2, KIR3DS1 | AGATTTGGAGCTTGGTTCTG | chr19:54859106- |
| | | | 54859128:− |
| 122 | KIR2DL3, KIR3DL3 | AGGGAACAGAACAGTGAAC | chr19:54752244- |
| | | A | 54752266:+ |
| 123 | KIR2DL4 | AGTACCTAATCACAGCATGC | chr19:54813138- |
| | | | 54813160:− |
| 124 | KIR2DS4 | AGTCATCCTGCAATGTTGGT | chr19:54837637- |
| | | | 54837659:+ |
| 125 | KIR2DS4 | ATAGATGGAGCTGCAGGACA | chr19:54839494- |
| | | | 54839516:− |
| 126 | KIR2DL1, KIR2DL2 | CAATCAGAATGTGCAGGTGT | chr19:54782929- |
| | | | 54782951:− |
| 127 | KIR2DL3, KIR3DL3 | CAGGGAACAGAACAGTGAAC | chr19:54752243- |
| | | | 54752265:+ |
| 128 | KIR2DL1, KIR2DS1, KIR2DS5 | CAGTCGCATGACGCAAGACC | chr19:54773523- |
| | | | 54773545:+ |
| 129 | KIR3DL1, KIR3DS1 | CCAGAGGGCCGGTCCACACA | chr19:54817547- |
| | | | 54817569:+ |
| 130 | KIR3DL1, KIR3DS1 | CCATGATGCTTGCCCTTGCA | chr19:54819908- |
| | | | 54819930:+ |
| 131 | KIR3DL1, KIR3DS1 | CCATGTGTGGACCGGCCCTC | chr19:54817547- |
| | | | 54817569:− |
| 132 | KIR2DL1, KIR2DL2, KIR2DL3, | CCCATGATGCAAGACCTTGC | chr19:54742174- |
| | KIR2DS2, KIR2DS3 | | 54742196:+ |
| 133 | KIR3DL1, KIR3DS1 | CCCTGCAAGGGCAAGCATCA | chr19:54819908- |
| | | | 54819930:− |
| 134 | KIR2DL1, KIR2DS1, KIR2DS5 | CCGTTGACCTTGGGCCCTGC | chr19:54775308- |
| | | | 54775330:− |
| 135 | KIR2DL1, KIR2DL2, KIR2DL3, | CCTGCAAGGTCTTGCATCAT | chr19:54742174- |
| | KIR2DS2, KIR2DS3 | | 54742196:− |
| 136 | KIR2DL4 | CGGGACACAGAACAGTGAAC | chr19:54813717- |
| | | | 54813739:+ |
| 137 | KIR3DL2 | CGGGGGACAGAACAGTGAAT | chr19:54866397- |
| | | | 54866419:+ |
| 138 | KIR3DL1, KIR3DL2, KIR3DS1 | CTCGTTGGACAGATCCATGA | chr19:54853890- |
| | | | 54853912:+ |
| 139 | KIR2DL1, KIR2DL2, KIR2DL3, | GAAAGAGCCGAAGCATCTGT | chr19:54744008- |
| | KIR2DL4, KIR2DS1, KIR2DS2, | | 54744030:− |
| | KIR2DS3, KIR2DS4, KIR2DS5, | | |
| | KIR3DL1, KIR3DL2, KIR3DS1 | | |
| 140 | KIR2DS1, KIR2DS2, KIR2DS3, | GAAAGGGATTTTGACCACTG | chr19:54847217- |
| | KIR2DS4, KIR2DS5, KIR3DS1 | | 54847239:− |
| 141 | KIR3DL1, KIR3DS1 | GAGAGAGAAGGTTTCTCATA | chr19:54821567- |
| | | | 54821589:− |
| 142 | KIR2DL1, KIR2DL2, KIR2DL3, | GCACAGAGAAGGGAAGTTTA | chr19:54742089- |
| | KIR2DS2, KIR2DS3 | | 54742111:+ |
| 143 | KIR2DL1, KIR2DL2, KIR3DL1, | GCAGGAGACAACTTTGGATC | chr19:54866670- |
| | KIR3DL2 | | 54866692:− |
| 144 | KIR3DL1, KIR3DL3, KIR3DS1 | GCATCTGTAGGTCCCTGCAA | chr19:54727818- |
| | | | 54727840:− |
| 145 | KIR2DS4 | GCTGCAGGGGGCCTGGCCAC | chr19:54835089- |
| | | | 54835111:+ |
| 146 | KIR3DL1, KIR3DS1 | GGCAAGCATCATGGGACCGA | chr19:54819899- |
| | | | 54819921:− |
| 147 | KIR2DL4 | GGGACACAGAACAGTGAACA | chr19:54813718- |
| | | | 54813740:+ |
| 148 | KIR3DL1 | GTACAAGATGGTATCTGTAG | chr19:54830202- |
| | | | 54830224:− |
| 149 | KIR2DL1, KIR2DL2, KIR2DL3 | GTACACGATGATATCTGTTG | chr19:54752470- |
| | | | 54752492:− |
| 150 | KIR3DL2 | GTACACGCTGGTATCTGTTA | chr19:54866625- |
| | | | 54866647:− |
| 151 | KIR2DL4 | GTCTCTTGCTCCTCTGAGAA | chr19:54813922- |
| | | | 54813944:− |
| 152 | KIR3DL1 | TACAAGATGGTATCTGTAGG | chr19:54830201- |
| | | | 54830223:− |
| 153 | KIR2DL1, KIR2DL2, KIR2DL3 | TACACGATGATATCTGTTGG | chr19:54783739- |
| | | | 54783761:− |
| 154 | KIR3DL1, KIR3DS1 | TACCATCTATCCAGGGAGGG | chr19:54821666- |
| | | | 54821688:+ |
| 155 | KIR2DS4 | TCCCAGTGACCCTCTGGACA | chr19:54837844- |
| | | | 54837866:+ |
| 156 | KIR2DS4, KIR3DL2 | TCCTGCAAGGACAGGCATCA | chr19:54837771- |
| | | | 54837793:− |
| 157 | KIR2DS4 | TCCTGCAGCTCCATCTATCC | chr19:54839499- |
| | | | 54839521:+ |
| 158 | KIR3DL1, KIR3DL2, KIR3DS1 | TCTGTCCAACGAGGCGTGAG | chr19:54819846- |
| | | | 54819868:− |
| 159 | KIR2DL1, KIR2DL2, KIR2DL3, | TGCAATGTTGGTCAGATGTC | chr19:54742049- |
| | KIR2DS2, KIR2DS3, KIR3DL3 | | 54742071:+ |
| 160 | KIR2DL4 | TGCTGTGATTAGGTACTCAG | chr19:54813144- |
| | | | 54813166:+ |
| 161 | KIR2DL1, KIR2DS1, KIR2DS5 | TGTTACTCACTCCCCCTATC | chr19:54773571- |
| | | | 54773593:+ |
| 162 | KIR2DL4 | TTCACACAGAGAAAAATCAC | chr19:54813899- |
| | | | 54813921:+ |
| 163 | MIR30E | CTACTGTAAACATCCTTGAC | chr1:40754367- |
| | | | 40754389:+ |
| 164 | MIR30E | TGTTCAGAGGAGCTTTCAGT | chr1:40754401- |
| | | | 40754423:+ |
| 165 | MIR29C | ACATCATAACCGATTTCAAA | chr1:207801859- |
| | | | 207801881:+ |
| 166 | MIR29C | ACAGGCTGACCGATTTCTCC | chr1:207801908- |
| | | | 207801930:− |
| 167 | MIR29B2 | CCTAAAACACTGATTTCAAA | chr1:207802445- |
| | | | 207802467:+ |
| 168 | MIR29B2 | TTCTGGAAGCTGGTTTCACA | chr1:207802500- |
| | | | 207802522:− |
| 169 | MIR16 | TTCAGCAGCACAGTTAATAC | chr13:50048984- |
| | | | 50049006:+ |
| 170 | MIR16 | CCTTAGCAGCACGTAAATAT | chr13:50049029- |
| | | | 50049051:− |
| 171 | MIR15A | ATTTTTGAGGCAGCACAATA | chr13:50049125- |
| | | | 50049147:+ |
| 172 | MIR15A | AGTAAAGTAGCAGCACATAA | chr13:50049173- |
| | | | 50049195:− |
| 173 | MIR27A | GGGCTTAGCTGCTTGTGAGC | chr19:13836485- |
| | | | 13836507:− |
| 174 | MIR27A | ACACCAAGTCGTGTTCACAG | chr19:13836458- |
| | | | 13836480:− |
| 175 | MIR15B | AGTACTGTAGCAGCACATCA | chr3:160404600- |
| | | | 160404622:+ |
| 176 | MIR15B | TTTGCTGCTCTAGAAATTTA | chr3:160404655- |
| | | | 160404677:+ |
| 177 | MIR16 | CTCTAGCAGCACGTAAATAT | chr3:160404751- |
| | | | 160404773:+ |
| 178 | MIR16 | CTAAAGCAGCACAGTAATAT | chr3:160404797- |
| | | | 160404819:− |
| 179 | MIR378A | CCTAGAAATAGCACTGGACT | chr5:149732855- |
| | | | 149732877:+ |
| 180 | MIR378A | GCTATTTCTAGGTAACACAC | chr5:149732844- |
| | | | 149732866:− |
| 181 | MIR146A | GCTTTGAGAACTGAATTCCA | chr5:160485368- |
| | | | 160485390:+ |
| 182 | MIR146A | CTTTGAGAACTGAATTCCAT | chr5:160485369- |
| | | | 160485391:+ |
| 183 | MIR146A | GCTGAAGAACTGAATTTCAG | chr5:160485408- |
| | | | 160485430:− |
| 184 | MIR29A | TTTCTAGCACCATCTGAAAT | chr7:130876751- |
| | | | 130876773:− |
| 185 | MIR29A | TCATTATAACCGATTTCAGA | chr7:130876730- |
| | | | 130876827:+ |
| 186 | MIR29B1 | CCAAGAACACTGATTTCAAA | chr7:130877462- |
| | | | 130877484:+ |
| 187 | MIR29B1 | TTCAGGAAGCTGGTTTCATA | chr7:130877516- |
| | | | 130877538:− |
| 188 | MIR223 | GTAAGCATGTGCCGCACTTG | chrX:66018954- |
| | | | 66018976:− |
| 189 | MIR223 | CTTGTCAAATACACGGAGCG | chrX:66018887- |
| | | | 66018909:− |

Optimized transgene sequence for KLRD1
(SEQ ID NO. 15):
ATGGCGGTGTTCAAAACAACGCTGTGGCGGCTCATCTCTG
GCACCTTGGGCATAATATGCTTGTCCCTTATGTCTACTCT
CGGCATTCTGCTCAAAAATAGCTTCACGAAGCTGTCTATA
GAACCTGCGTTTACCCCCGGACCCAACATCGAGTTGCAGA
AAGACTCCGACTGCTGTTCCTGTCAAGAAAAATGGGTCGG
ATACCGATGCAACTGTTACTTTATTTCCTCTGAGCAGAAA
ACCTGGAATGAGAGCCGACATCTGTGTGCAAGTCAGAAGT
CTAGCCTTTTGCAATTGCAAAACACCGACGAGCTTGACTT
CATGTCCTCTTCCCAACAATTCTATTGGATCGGGCTTAGC
TATTCCGAAGAGCACACCGCATGGCTTTGGGAGAATGGGA
GCGCACTGAGCCAATACCTGTTTCCTTCATTCGAAACCTT
TAACACTAAGAATTGTATCGCTTACAATCCTAATGGTAAT
GCTCTGGATGAAAGTTGCGAGGATAAAAATCGCTATATTT
GTAAGCAGCAATTGATTTAA
Optimized transgene sequence for CD96
(SEQ ID NO. 16):
ATGGAAAAAAAGTGGAAATACTGCGCCGTGTACTACATAA
TACAGATTCACTTTGTCAAAGGAGTATGGGAGAAAACAGT
GAACACAGAAGAGAACGTATATGCAACATTGGGATCTGAT
GTGAATCTCACGTGCCAGACGCAGACCGTAGGCTTTTTCG
TTCAAATGCAATGGTCCAAAGTAACGAATAAAATAGACCT
CATCGCCGTATATCATCCGCAATATGGTTTCTATTGCGCG
TACGGCAGACCATGCGAGAGCCTCGTCACCTTCACCGAGA
CTCCGGAGAATGGCAGCAAGTGGACGCTTCACCTGCGGAA
TATGAGCTGTAGCGTGTCCGGAAGATATGAATGCATGTTG
GTGCTGTATCCCGAGGGTATACAGACTAAGATCTACAATC
TGCTCATTCAAACGCACGTAACTGCTGACGAATGGAACTC
AAATCACACCATTGAAATAGAGATCAATCAGACTCTGGAG
ATACCTTGTTTTCAAAATTCCTCCAGCAAAATAAGTTCAG
AGTTTACTTATGCCTGGAGTGTGGAGGACAATGGCACACA
AGAAACCTTGATTTCTCAAAACCACCTCATTTCAAACTCC
ACCTTGTTGAAGGACCGGGTAAAGTTGGGAACGGACTACA
GACTGCATCTGTCCCCCGTGCAAATATTTGACGACGGCAG
GAAGTTCTCCTGTCACATAAGAGTAGGACCTAACAAGATT
CTCAGATCTTCAACTACCGTAAAAGTGTTTGCTAAGCCCG
AGATCCCCGTAATCGTGGAGAACAATTCCACAGACGTACT
CGTCGAGCGCAGATTCACCTGTTTGTTGAAAAACGTTTTT
CCCAAAGCCAATATCACGTGGTTTATCGATGGGTCCTTCC
TTCACGATGAGAAGGAGGGCATCTATATTACAAACGAGGA
AAGGAAGGGGAAAGATGGATTTCTTGAACTCAAATCCGTT
CTGACTCGCGTCCACTCCAATAAGCCAGCACAAAGCGATA
ATTTGACTATCTGGTGCATGGCGTTGTCCCCCGTGCCGGG
CAACAAGGTTTGGAACATCTCTTCCGAAAAAATAACCTTC
CTCTTGGGCTCCGAGATTTCATCTACCGACCCTCCATTGT
CCGTAACCGAGTCTACACTCGACACTCAGCCGTCCCCTGC
CTCTTCCGTGAGTCCTGCACGCTACCCCGCGACTAGCTCT
GTGACACTGGTTGACGTATCAGCATTGAGGCCTAACACTA
CTCCTCAACCATCAAATTCATCTATGACAACCCGGGGTTT
CAATTATCCATGGACTTCCTCCGGCACGGACACAAAAAAA
TCAGTCTCACGAATTCCTTCCGAAACATACTCATCCAGCC
CAAGCGGCGCGGGTTCCACATTGCATGATAACGTATTTAC
CAGTACTGCACGGGCCTTCAGCGAAGTACCTACAACTGCG
AACGGATCTACAAAAACCAATCATGTTCACATAACTGGCA
TCGTTGTGAATAAACCCAAAGACGGCATGTCATGGCCAGT
GATAGTTGCCGCATTGCTCTTCTGCTGCATGATCCTGTTT
GGCCTGGGAGTCCGCAAATGGTGTCAGTATCAGAAAGAGA
TAATGGAGCGACCGCCTCCTTTCAAACCCCCTCCACCTCC
CATAAAGTACACGTGTATTCAGGAACCTAATGAAAGCGAC
CTTCCCTACCATGAGATGGAGACTCTTTAG
Optimized transgene sequence for CD244
(SEQ ID NO. 17):
ATGCTTGGTCAAGTTGTAACTCTTATCCTGTTGTTGCTGC
TCAAAGTGTACCAAGGCAAAGGCTGTCAAGGCTCTGCTGA
CCATGTCGTTTCAATAAGCGGTGTTCCCTTGCAACTCCAA
CCCAATTCCATACAAACCAAGGTCGATAGTATCGCTTGGA
AGAAGCTCCTGCCGTCCCAAAATGGGTTTCATCACATATT
GAAATGGGAAAACGGTTCCCTTCCATCAAACACGTCCAAC
GACAGATTCTCCTTTATCGTAAAAAACCTCAGTCTGCTCA
TCAAAGCTGCTCAACAGCAAGATTCAGGGCTTTATTGCCT
GGAAGTTACTAGCATTTCTGGCAAGGTCCAGACCGCCACC
TTTCAAGTGTTCGTGTTTGATAAGGTCGAGAAGCCGCGGC
TCCAAGGACAGGGAAAAATTCTGGACCGAGGGAGATGCCA
GGTCGCGCTGAGTTGCCTCGTGAGCCGCGATGGAAATGTA
TCTTACGCTTGGTACCGGGGCAGCAAGTTGATCCAGACCG
CGGGCAATCTGACATACCTTGACGAGGAGGTCGACATCAA
CGGCACACATACTTACACTTGCAATGTGAGTAACCCTGTA
TCCTGGGAGTCCCATACGCTGAATCTGACACAAGACTGCC
AAAACGCCCATCAAGAGTTCCGATTTTGGCCCTTTCTGGT
TATCATCGTAATATTGAGTGCTTTGTTCCTTGGTACATTG
GCCTGTTTCTGTGTCTGGCGACGGAAACGCAAGGAAAAGC
AGAGCGAGACAAGCCCTAAAGAGTTTTTGACCATATACGA
GGATGTCAAGGACCTGAAAACAAGAAGGAATCATGAACAA
GAGCAGACTTTCCCCGGCGGAGGTTCTACCATATATTCTA
TGATTCAAAGTCAAAGCAGCGCGCCCACCTCACAAGAGCC
TGCGTATACTTTGTATTCCCTTATTCAACCTTCACGGAAA
AGCGGGTCCAGGAAGAGGAATCATAGCCCGTCCTTTAATT
CAACTATCTATGAAGTCATTGGAAAGAGTCAGCCTAAGGC
ACAAAATCCTGCGAGGCTCTCCCGGAAGGAATTGGAAAAC
TTTGACGTATACTCCTAG
Optimized transgene sequence for CCR4
(SEQ ID NO. 18):
ATGAACCCTACGGACATTGCGGATACTACCCTCGATGAGT
CAATTTATAGCAATTATTACCTTTATGAATCAATCCCCAA
GCCGTGCACCAAGGAAGGCATTAAAGCCTTTGGTGAACTC
TTTCTTCCACCTCTGTATTCACTTGTGTTTGTTTTTGGAC
TGCTCGGCAACTCAGTTGTGGTGCTGGTACTCTTTAAATA
CAAAAGACTCCGCAGCATGACCGATGTGTACCTCTTGAAT
CTGGCAATATCTGACCTCCTGTTTGTATTCTCCCTCCCCT
TTTGGGGATATTACGCTGCTGATCAATGGGTTTTTGGGTT
GGGCCTCTGCAAAATGATCAGCTGGATGTACCTCGTGGGA
TTTTACTCCGGGATTTTCTTTGTCATGCTCATGTCCATCG
ACCGATACCTTGCCATAGTTCATGCGGTCTTTTCTTTGCG
GGCCCGAACCCTGACATACGGCGTTATCACGAGCCTGGCT
ACGTGGAGCGTGGCTGTTTTCGCGAGCCTCCCTGGCTTTC
TCTTTTCAACCTGCTACACGGAGCGCAACCATACCTATTG
CAAAACTAAATACTCACTTAACTCCACTACTTGGAAGGTG
CTCTCCAGTTTGGAAATCAATATTTTGGGCCTGGTTATCC
CTCTGGGCATAATGTTGTTTTGCTATTCCATGATCATAAG
AACATTGCAACACTGCAAGAACGAGAAAAAAAATAAGGCC
GTAAAAATGATATTTGCAGTAGTTGTCCTTTTTCTTGGTT
TTTGGACGCCCTATAACATAGTACTGTTCTTGGAAACTTT
GGTAGAACTGGAGGTGCTCCAAGATTGTACATTCGAAAGA
TATTTGGACTATGCCATTCAAGCTACCGAAACTCTCGCTT
TCGTGCATTGCTGTCTGAACCCCATAATATACTTTTTCCT
CGGCGAAAAATTCCGAAAATACATACTCCAGCTCTTCAAA
ACCTGCCGGGGCCTCTTTGTACTGTGCCAATACTGTGGCT
TGCTCCAAATATATAGCGCGGACACACCATCTTCCTCCTA
CACACAAAGTACTATGGACCATGACCTTCACGATGCGTTG
TAG
Optimized transgene sequence for CCR9
(SEQ ID NO. 19):
ATGACCCCCACGGACTTTACTTCCCCGATTCCAAATATGG
CGGACGACTATGGCAGCGAATCCACGAGCTCCATGGAGGA
CTATGTGAATTTCAATTTCACCGATTTCTATTGCGAGAAG
AACAATGTTCGACAGTTCGCCTCCCACTTCCTGCCACCAC
TGTACTGGCTGGTATTCATCGTGGGGGCGCTGGGAAATTC
CCTGGTCATACTTGTATATTGGTACTGTACCCGCGTAAAA
ACTATGACGGATATGTTTCTCCTCAACCTTGCGATAGCTG
ATCTGCTTTTTTTGGTCACGCTGCCCTTCTGGGCAATCGC
AGCGGCAGATCAGTGGAAATTCCAAACCTTTATGTGCAAA
GTCGTGAATTCCATGTATAAGATGAATTTTTATTCATGCG
TGCTTCTCATAATGTGCATTAGTGTTGATCGGTATATAGC
CATCGCCCAGGCGATGAGGGCTCATACCTGGCGCGAGAAA
CGACTCCTGTACTCCAAAATGGTATGTTTCACCATCTGGG
TACTTGCCGCTGCACTTTGCATACCTGAAATTTTGTACTC
CCAAATTAAAGAGGAGTCCGGGATTGCAATTTGCACAATG
GTCTACCCTAGCGATGAATCAACTAAACTTAAATCTGCGG
TGCTTACTCTCAAAGTTATACTCGGGTTCTTTCTTCCCTT
TGTCGTCATGGCCTGCTGCTATACTATTATAATACATACG
TTGATACAGGCAAAGAAATCCAGCAAACATAAGGCGTTGA
AAGTCACTATCACAGTGTTGACTGTGTTCGTGTTGTCTCA
ATTTCCATACAATTGTATATTGCTGGTTCAAACAATAGAC
GCCTATGCAATGTTCATCTCCAATTGTGCGGTTTCTACTA
ACATCGACATTTGCTTCCAAGTCACCCAAACTATCGCATT
CTTCCATTCATGCTTGAATCCGGTACTTTATGTATTTGTC
GGCGAGAGGTTTCGCAGAGATTTGGTCAAAACCCTCAAGA
ACCTTGGGTGCATCAGCCAAGCGCAATGGGTATCATTTAC
GCGCCGGGAGGGGTCACTGAAACTCAGTAGTATGCTTCTG
GAAACTACTAGTGGGGCGCTCTCCCTGTGA
Optimized transgene sequence for CXCR6
(SEQ ID NO. 20):
ATGGCGGAACACGATTACCACGAGGATTATGGCTTTAGCA
GCTTTAACGATTCCAGCCAGGAGGAACATCAGGACTTTCT
GCAATTCTCCAAAGTATTTTTGCCATGCATGTATCTGGTG
GTGTTTGTGTGTGGCCTCGTTGGAAATAGTCTCGTTCTCG
TAATATCCATATTTTACCACAAACTGCAATCTCTCACCGA
TGTGTTCCTTGTAAACCTGCCCCTTGCCGATCTCGTCTTT
GTATGTACACTGCCGTTTTGGGCATATGCTGGCATACACG
AATGGGTATTTGGGCAGGTTATGTGCAAATCACTTCTTGG
AATCTATACGATAAACTTCTATACAAGCATGCTTATTCTC
ACTTGCATTACCGTGGACAGATTCATAGTGGTGGTGAAGG
CGACCAAAGCGTATAACCAACAAGCTAAACGAATGACTTG
GGGGAAGGTCACCAGCCTTTTGATCTGGGTTATCTCACTT
CTTGTGTCTCTTCCACAAATAATCTATGGGAACGTCTTTA
ATCTTGACAAATTGATTTGCGGATACCATGACGAAGCGAT
CAGTACTGTAGTATTGGCGACACAGATGACGCTTGGATTC
TTTCTCCCGCTTCTGACGATGATCGTTTGTTATTCCGTTA
TCATTAAAACCCTGCTGCATGCTGGCGGGTTTCAAAAACA
CAGATCTCTCAAGATAATTTTCCTTGTGATGGCCGTGTTC
CTCCTTACCCAAATGCCGTTCAATCTTATGAAGTTTATAC
GCTCAACTCATTGGGAGTATTACGCTATGACCTCTTTTCA
TTACACAATCATGGTGACTGAAGCCATTGCGTATCTTCGC
GCTTGTCTCAACCCGGTTCTCTATGCATTCGTGTCCCTGA
AATTTAGAAAGAACTTCTGGAAGTTGGTCAAAGACATTGG
CTGTTTGCCTTATCTGGGAGTCAGCCATCAATGGAAGAGT
AGTGAGGATAACAGCAAAACGTTTTCCGCCTCCCATAACG
TTGAGGCAACATCAATGTTTCAACTCTAG
Optimized transgene sequence for CCR2
(SEQ ID NO. 21):
ATGCTTAGTACCAGCCGAAGTAGATTTATAAGAAACACAA
ACGAGTCTGGGGAGGAGGTGACGACTTTTTTTGATTATGA
TTATGGCGCGCCGTGCCATAAATTTGACGTTAAACAAATA
GGAGCCCAACTGCTCCCTCCATTGTATTCCCTGGTGTTCA
TATTCGGCTTCGTGGGAAACATGCTGGTGGTGCTTATATT
GATCAACTGTAAAAAGCTCAAATGTCTTACCGATATTTAT
CTCCTTAATCTCGCAATAAGCGATTTGCTTTTCTTGATCA
CTCTCCCCCTTTGGGCCCATTCCGCTGCTAACGAATGGGT
TTTCGGCAACGCGATGTGTAAACTTTTCACCGGCTTGTAC
CATATCGGCTACTTCGGCGGCATATTCTTTATAATACTTT
TGACTATCGACCGCTACCTCGCTATCGTCCACGCGGTCTT
TGCTCTTAAGGCAAGGACGGTAACCTTTGGAGTCGTTACC
TCCGTTATCACTTGGCTGGTGGCCGTGTTTGCCTCCGTTC
CCGGCATAATTTTTACAAAATGCCAGAAAGAGGATTCAGT
ATATGTCTGTGGGCCCTATTTCCCCCGGGGATGGAATAAT
TTCCATACAATCATGCGAAATATACTGGGACTCGTACTTC
CACTTCTTATAATGGTTATATGCTACTCCGGAATCTTGAA
AACACTTCTCAGATGTCGGAATGAGAAGAAGCGCCATCGA
GCCGTGCGAGTGATTTTTACCATCATGATCGTATACTTCT
TGTTCTGGACACCGTACAACATCGTCATATTGCTGAATAC
TTTCCAAGAATTTTTTGGCCTTTCCAACTGCGAAAGCACC
TCTCAACTGGATCAAGCGACTCAGGTGACAGAAACCTTGG
GGATGACTCACTGCTGCATTAACCCGATAATTTACGCGTT
CGTGGGCGAGAAGTTTCGACGATATTTGAGCGTGTTCTTC
CGGAAGCACATCACCAAAAGATTTTGCAAGCAATGCCCAG
TCTTTTATCGAGAAACCGTAGATGGAGTTACGAGTACGAA
CACTCCATCCACTGGCGAGCAAGAAGTGTCAGCAGGACTT
TAA
Optimized transgene sequence for CXCR2
(SEQ ID NO. 22):
ATGGAGGATTTTAACATGGAGAGTGACTCCTTTGAAGATT
TCTGGAAAGGGGAGGACCTCAGTAATTACTCATACTCTTC
TACCCTCCCGCCATTCTTGCTCGACGCGGCACCCTGCGAG
CCCGAGAGTCTTGAGATAAACAAATATTTTGTAGTGATAA
TTTACGCCCTTGTGTTCTTGCTTTCCTTGTTGGGCAATAG
CTTGGTGATGCTGGTCATCCTGTACTCACGGGTGGGACGG
TCCGTTACCGACGTGTACCTCCTGAATCTGGCCCTCGCGG
ACTTGCTTTTTGCCCTGACTTTGCCAATATGGGCAGCTAG
CAAAGTCAATGGCTGGATATTTGGAACCTTTCTGTGTAAG
GTCGTGTCACTGTTGAAAGAAGTAAACTTCTACAGTGGCA
TACTGTTGCTTGCCTGTATATCTGTCGATCGGTACCTCGC
TATAGTACACGCCACACGGACGCTCACACAAAAACGCTAT
TTGGTGAAATTCATATGCTTGAGCATATGGGGCCTTTCCC
TTTTGCTTGCTTTGCCGGTCCTCCTTTTCCGACGGACTGT
CTACTCCTCTAATGTGTCCCCGGCTTGTTATGAGGACATG
GGGAACAACACCGCTAACTGGCGCATGCTCCTGAGGATAC
TCCCACAAAGTTTTGGCTTCATCGTCCCGCTCCTTATAAT
GCTCTTTTGCTACGGTTTCACCTTGCGAACTCTTTTTAAG
GCTCATATGGGCCAGAAGCACAGAGCTATGCGCGTGATTT
TCGCCGTGGTCCTTATCTTCTTGTTGTGCTGGCTCCCTTA
CAATTTGGTTCTGCTGGCGGATACTCTGATGCGAACGCAA
GTTATTCAGGAGACTTGCGAGCGGCGAAATCATATAGATA
GGGCACTTGACGCCACTGAGATCTTGGGGATACTCCATTC
CTGTCTCAACCCGCTCATATATGCGTTCATTGGCCAAAAG
TTCCGACATGGTCTGCTCAAGATTCTCGCGATTCACGGAC
TTATCAGTAAAGACAGTCTGCCAAAAGATTCACGCCCCTC
ATTTGTTGGATCTTCCTCCGGACATACATCAACTACGCTT
TAA
Optimized transgene sequence for CX3CR1
(SEQ ID NO. 23):
ATGGATCAATTCCCCGAAAGCGTCACCGAAAATTTTGAGT
ATGACGATTTGGCTGAAGCGTGTTATATAGGCGACATAGT
TGTGTTCGGCACTGTTTTCTTGTCCATCTTTTACTCCGTT
ATTTTCGCCATTGGACTTGTGGGCAATCTGCTTGTGGTGT
TCGCACTTACCAATAGCAAGAAGCCAAAGAGCGTCACGGA
TATTTATTTGCTGAATCTGGCTTTGTCTGATCTGCTTTTC
GTGGCTACTCTCCCCTTTTGGACGCATTACCTGATCAATG
AAAAGGGGCTGCATAACGCTATGTGCAAATTTACCACAGC
GTTTTTTTTCATCGGTTTTTTTGGCTCAATCTTTTTCATA
ACCGTTATTAGCATTGATCGCTACCTTGCGATTGTACTTG
CCGCTAATAGTATGAACAATCGCACGGTGCAACACGGAGT
TACAATATCATTGGGAGTATGGGCGGCTGCAATTCTCGTC
GCCGCGCCACAATTTATGTTCACCAAACAAAAAGAGAACG
AGTGCTTGGGGGATTATCCCGAGGTCCTCCAGGAGATCTG
GCCTGTTCTGAGGAACGTGGAAACAAATTTCCTCGGATTT
CTGCTCCCCCTCCTTATCATGTCATACTGCTATTTTCGCA
TTATCCAAACATTGTTTTCTTGTAAAAATCACAAGAAAGC
CAAGGCGATCAAGCTGATTCTGCTTGTTGTGATCGTCTTT
TTCCTCTTTTGGACCCCTTATAATGTAATGATTTTTTTGG
AAACACTCAAACTCTACGACTTCTTTCCATCCTGCGATAT
GCGGAAGGATCTGCGGCTGGCGCTCTCCGTAACCGAAACT
GTAGCTTTTAGTCATTGTTGTTTGAATCCTTTGATCTATG
CGTTTGCCGGGGAAAAGTTTAGAAGATACCTGTATCACCT
CTATGGAAAGTGTTTGGCCGTTTTGTGTGGTCGATCTGTC
CACGTTGATTTCTCCTCCTCAGAGTCCCAACGCAGCAGAC
ACGGGTCTGTACTCTCCTCAAACTTCACATATCATACGAG
CGACGGAGATGCATTGCTGCTTTTGTGA
Optimized transgene sequence for KLRC2
(SEQ ID NO. 24):
ATGAATAAACAAAGAGGAACATTCTCCGAGGTATCTCTGG
CTCAGGACCCTAAAAGACAGCAACGCAAACCTAAAGGAAA
TAAATCAAGCATCAGTGGCACAGAACAGGAAATATTTCAA
GTGGAATTGAATCTGCAGAATCCCTCCTTGAACCACCAGG
GCATCGACAAAATTTATGATTGTCAGGGGCTCCTGCCGCC
GCCCGAAAAGTTGACAGCCGAGGTGCTGGGCATCATCTGT
ATAGTCTTGATGGCGACGGTTCTTAAGACAATTGTCCTTA
TACCCTTTCTGGAGCAAAACAATTTTTCACCCAATACTCG
AACCCAAAAAGCTCGGCACTGCGGCCACTGTCCCGAGGAG
TGGATAACATACTCAAACTCTTGCTATTATATCGGCAAAG
AAAGAAGAACTTGGGAGGAGAGTTTGCTCGCTTGTACGAG
CAAAAATAGCAGTCTCCTTTCCATCGATAACGAAGAGGAG
ATGAAATTCCTTGCGTCAATACTGCCTAGTTCTTGGATTG
GCGTCTTTCGGAACTCCTCACACCACCCTTGGGTAACTAT
AAACGGCCTCGCATTCAAGCATAAGATCAAGGACTCTGAC
AACGCGGAGCTTAATTGTGCGGTTCTGCAAGTGAACCGGT
TGAAGAGTGCCCAGTGCGGATCATCCATGATATACCACTG
TAAACACAAGCTGTAG
Optimized transgene sequence for TGFBR2
(SEQ ID NO. 25):
ATGGGTAGAGGGCTGCTGCGCGGTTTGTGGCCGCTCCATA
TCGTTCTTTGGACGAGGATTGCATCCACTATCCCTCCGCA
TGTGCAAAAGTCCGTAAACAACGACATGATCGTCACAGAC
AATAATGGCGCGGTAAAGTTTCCTCAACTCTGTAAATTTT
GTGACGTGAGGTTTAGCACCTGCGATAACCAGAAGAGCTG
CATGTCAAATTGTTCTATTACCTCCATATGCGAAAAACCC
CAGGAGGTCTGCGTCGCAGTGTGGAGAAAGAACGACGAGA
ACATAACCTTGGAAACTGTTTGCCATGATCCGAAATTGCC
GTATCATGACTTTATTCTCGAGGACGCGGCCAGTCCAAAA
TGCATTGTAATGATAATATAATTTTCAGCGAGGAATACAA
CACGAGCAACCCTGACCTTCTCTTGGTCATCTTCCAGGTC
ACTGGCATATCCCTCCTGCCGCCGTTGGGGGTTGCGATTA
GTGTAATTATTATATTTTATTGCTACAGAGTGTAG
Optimized transgene sequence for KIR2DS1
(SEQ ID NO. 26):
ATGTCCCTCACGGTTGTTTCAATGGCCTGTGTAGGCTTCT
TCCTCCTCCAAGGCGCCTGGCCCCATGAGGGAGTGCATAG
AAAGCCTTCCCTCTTGGCACATCCCGGTCGCCTTGTTAAA
TCCGAGGAGACTGTTATTCTTCAATGTTGGTCTGATGTCA
TGTTCGAGCACTTCCTTTTGCACCGGGAAGGCATGTTCAA
CGATACGTTGCGCTTGATCGGAGAGCATCATGATGGTGTT
AGTAAAGCCAACTTTTCTATTTCACGGATGAAACAAGACC
TGGCTGGGACATACAGATGCTACGGATCCGTCACACATTC
TCCGTATCAATTGTCTGCTCCTTCTGACCCTTTGGACATA
GTAATAATCGGCCTTTATGAAAAGCCGTCCTTGTCTGCTC
AACCTGGGCCAACTGTACTGGCCGGAGAAAACGTGACCCT
CTCATGCTCCTCCCGCTCCTCTTACGACATGTACCATCTC
TCCCGGGAGGGGGAAGCCCACGAACGACGGCTTCCGGCGG
GCACTAAAGTAAATGGCACGTTTCAAGCGAATTTCCCGCT
CGGTCCCGCCACCCACGGCGGAACCTATCGGTGCTTTGGC
TCCTTCCGAGACAGCCCTTATGAGTGGAGCAAGTCAAGCG
ATCCGCTGCTCGTGTCTGTTACTGGCAATCCAAGCAACTC
ATGGCCGAGCCCCACGGAACCGAGTAGCGAAACAGGCAAT
CCCAGACACCTCCATGTCCTCATTGGGACGTCCGTCGTCA
AGATTCCATTTACAATTTTGTTGTTCTTCTTGTTGCACCG
ATGGTGTTCAGACAAAAAGAACGCTGCTGTAATGGATCAA
GAACCTGCGGGCAATCGCACTGTTAACTCCGAGGATAGCG
ACGAACAAGATCATCAAGAGGTGTCTTATGCGTGA
Optimized transgene sequence for KIR2DS2
(SEQ ID NO. 27):
ATGAGCTTGATGGTAGTGTCTATGGCGTGTGTAGGATTTT
TCCTGCTTCAAGGCGCTTGGCCGCATGAAGGCGTGCATAG
AAAGCCCTCCCTGCTCGCCCATCCTGGGCCGCTGGTAAAA
AGCGAGGAGACAGTTATTCTTCAATGTTGGAGTGATGTTA
GATTCGAACATTTTTTGCTGCACCGGGAGGGGAAATACAA
GGATACGCTCCACCTTATCGGAGAGCATCATGACGGAGTC
AGTAAAGCAAACTTCTCTATCGGCCCTATGATGCAGGACT
TGGCGGGCACGTACCGATGCTACGGTTCTGTGACCCATTC
CCCCTATCAACTGTCCGCCCCCTCCGACCCTCTGGACATC
GTCATCACTGGACTCTACGAAAAACCATCCTTGTCTGCTC
AACCCGGCCCCACTGTGCTCGCCGGGGAGAGTGTCACGCT
TAGCTGTTCCTCTCGATCCTCCTATGACATGTACCATCTT
TCTCGCGAAGGAGAAGCACATGAAAGACGATTCAGTGCCG
GTCCGAAAGTGAATGGCACATTCCAGGCCGACTTTCCGCT
GGGACCTGCCACGCACGGGGGCACGTACCGATGTTTTGGG
AGTTTTCGGGATAGCCCATATGAGTGGAGTAATTCAAGCG
ACCCGCTCTTGGTGTCTGTGACTGGGAACCCCAGCAACTC
CTGGCCGAGCCCCACCGAACCTTCTAGCAAGACTGGCAAC
CCCCGACACTTGCACGTGCTCATCGGAACGAGTGTCGTCA
AAATCCCTTTTACCATTCTTCTCTTTTTCTTGCTTCATCG
ATGGTGTTCAAATAAAAAGAACGCCGCAGTGATGGATCAA
GAACCGGCAGGCAATCGGACCGTAAACTCCGAGGACTCCG
ACGAACAGGACCATCAAGAGGTGAGTTACGCATGA
Optimized transgene sequence for KIR2DS3
(SEQ ID NO. 28):
ATGTCTCTGATGGTTATCAGCATGGCCTGTGTGGGATTCT
TCTGGCTCCAAGGCGCGTGGCCGCACGAGGGATTCCGAAG
AAAACCCTCTCTCCTGGCCCACCCCGGCAGATTGGTAAAG
AGCGAGGAAACTGTAATACTTCAGTGCTGGAGTGATGTCA
TGTTCGAACACTTTCTTTTGCATCGGGAAGGGACATTTAA
CGATACTTTGAGACTGATCGGAGAGCATATTGACGGAGTG
AGCAAGGCGAATTTCTCAATCGGAAGGATGCGCCAGGATC
TGGCAGGCACATACAGATGCTATGGTTCTGTTCCTCATTC
ACCGTACCAATTCAGCGCTCCATCCGATCCTCTCGACATT
GTAATAACTGGTCTTTATGAGAAACCGTCACTCTCCGCCC
AACCTGGCCCAACCGTGTTGGCGGGCGAGAGTGTCACTCT
GTCCTGCTCATCTTGGAGTTCATACGACATGTATCATTTG
TCTACTGAAGGGGAAGCCCACGAGAGGCGGTTCTCCGCCG
GGCCAAAAGTTAATGGAACCTTTCAGGCTGACTTCCCTCT
CGGACCTGCTACCCAAGGAGGAACCTACAGATGTTTCGGA
TCTTTTCACGACTCTCCCTATGAGTGGAGCAAGAGCTCCG
ATCCCTTGCTCGTATCCGTTACGGGCAATCCGTCCAACTC
CTGGCCTTCTCCGACGGAACCCTCATCAAAAACCGGCAAC
CCTCGACACCTGCATGTCTTGATCGGAACTAGTGTCGTCA
AGCTCCCATTCACCATACTTCTCTTCTTTTTGCTGCATCG
GTGGTGTTCCGATAAAAAAAACGCTAGTGTTATGGATCAA
GGCCCCGCCGGTAACCGGACTGTGAATCGAGAAGATTCAG
ATGAACAGGACCATCAAGAGGTCAGCTATGCCTGA
Optimized transgene sequence for KIR2DS4
(SEQ ID NO. 29):
ATGAGTCTCATGGTAATAATCATGGCTTGTGTCGGGTTTT
TCCTGCTTCAGGGGGCTTGGCCGCAGGAGGGAGTTCACAG
AAAGCCCTCTTTCCTGGCGCTCCCCGGTCATCTTGTAAAA
TCTGAAGAAACTGTAATACTGCAGTGTTGGAGCGACGTGA
TGTTTGAACATTTTCTCCTTCATCGGGAAGGGAAGTTCAA
TAATACCCTGCACCTGATCGGCGAACACCATGATGGAGTT
AGTAAAGCAAACTTCAGCATAGGCCCGATGATGCCGGTTC
TTGCGGGAACCTATAGGTGTTACGGGAGTGTTCCGCACTC
TCCTTACCAACTCTCCGCACCAAGCGATCCTCTCGATATG
GTGATTATCGGTCTTTACGAAAAACCGAGCCTGTCAGCTC
AACCTGGACCGACGGTCCAAGCAGGGGAAAATGTAACTCT
CAGTTGCTCCAGCCGCTCCTCCTATGATATGTACCATTTG
AGTCGGGAGGGAGAAGCACACGAACGGCGCCTGCCTGCCG
TACGGTCTATAAACGGCACCTTTCAAGCGGACTTCCCTCT
CGGCCCCGCAACTCATGGCGGGACGTACAGATGCTTTGGA
TCCTTCCGGGACGCGCCATATGAGTGGAGCAATTCCAGCG
ATCCACTGCTCGTGTCCGTTACCGGAAATCCAAGCAACTC
CTGGCCGAGCCCTACTGAACCAAGTTCTAAGACTGGCAAC
CCGAGACACCTGCATGTGCTTATTGGCACCTCAGTAGTGA
AGATCCCGTTTACTATCTTGCTCTTCTTTCTTCTTCATCG
CTGGTGTAGCGATAAAAAGAATGCGGCCGTGATGGATCAA
GAGCCAGCAGGTAACCGGACAGTAAACAGCGAAGATTCCG
ACGAGCAAGATCACCAGGAAGTTTCTTATGCATGA
Optimized transgene sequence for KIR2DS5
(SEQ ID NO. 30):
ATGAGTCTTATGGTGATTTCCATGGCCTGTGTTGCTTTTT
TCCTCCTGCAGGGTGCTTGGCCTCACGAAGGGTTCCGCAG
GAAGCCATCTCTCCTTGCCCACCCTGGTCCTCTTGTAAAG
TCAGAGGAAACGGTTATCTTGCAATGTTGGAGCGATGTGA
TGTTTGAGCATTTCCTGCTCCACCGGGAGGGAACATTTAA
CCATACCCTCCGCCTCATCGGAGAACATATCGACGGCGTG
AGCAAGGGAAACTTTTCAATAGGGAGGATGACCCAGGATT
TGGCCGGGACATATCGCTGTTATGGCTCAGTCACTCACTC
ACCATATCAACTCAGTGCTCCGAGCGATCCACTTGACATT
GTGATTACCGGCCTCTATGAGAAACCTAGTCTCTCCGCTC
AACCTGGTCCGACCGTTTTGGCCGGCGAATCCGTCACCCT
TTCCTGCTCTTCCCGCTCCTCCTATGACATGTACCATCTG
AGTAGAGAGGGCGAGGCGCATGAGCGGAGACTTCCAGCCG
GTCCAAAAGTTAATCGCACCTTTCAGGCCGATTTCCCGCT
GGACCCTGCGACCCACGGCGGGACGTACCGCTGTTTCGGA
TCATTTCGGGACTCACCTTATGAGTGGAGTAAAAGTTCAG
ATCCTTTGCTTGTCTCCGTTACGGGCAATTCAAGCAACTC
CTGGCCCTCTCCCACGGAACCCTCTAGTGAAACCGGAAAT
CCCCGCCATCTCCATGTGTTGATTGGAACCTCCGTCGTAA
AGTTGCCTTTCACGATCCTGCTGTTCTTCCTTTTGCATCG
GTGGTGCTCTAATAAAAAGAACGCTTCCGTGATGGATCAA
GGCCCGGCCGGTAACCGCACGGTCAATAGAGAAGATAGTG
ACGAACAAGACCACCAAGAAGTGAGCTACGCGTGA
Optimized transgene sequence for KIR2DL1
(SEQ ID NO. 31):
ATGAGCTTGCTCGTCGTGTCAATGGCCTGCGTCGGTTTCT
TCCTTCTCCAGGGTGCATGGCCTCACGAGGGAGTGCACCG
AAAACCAAGTTTGCTGGCTCACCCTGGCCCGTTGGTGAAG
TCAGAGGAAACGGTCATATTGCAATGTTGGAGCGATGTAA
TGTTTGAACATTTCCTCCTGCACCGAGAGGGCATGTTCAA
TGACACGCTGCGGCTTATTGGGGAGCACCACGACGGCGTG
TCTAAGGCGAACTTCTCAATCTCCCGGATGACGCAAGACC
TTGCCGGGACTTATAGATGCTACGGGAGTGTCACGCATTC
CCCTTATCAAGTGTCTGCCCCTTCCGACCCCCTGGACATA
GTCATCATAGGCCTGTATGAAAAACCGTCCTTGAGTGCTC
AACCGGGTCCCACAGTCCTCGCTGGCGAGAATGTTACTTT
GAGTTGCTCCTCACGCTCTTCCTATGACATGTATCACCTG
AGCCGGGAGGGGGAGGCTCACGAGCGGCGACTGCCAGCAG
GGCCAAAGGTAAACGGAACTTTTCAAGCAGATTTTCCGCT
CGGCCCCGCAACCCACGGCGGCACATATCGGTGTTTTGGA
TCCTTCCACGACTCCCCCTACGAATGGTCCAAGTCATCTG
ATCCTCTGCTTGTCTCCGTCACCGGCAACCCGTCAAATTC
CTGGCCTTCACCCACCGAACCCTCCTCAAAAACCGGCAAC
CCGAGGCATTTGCATATCCTTATTGGCACAAGCGTAGTTA
TCATCCTCTTCATTTTGCTGTTTTTTTTGCTTCACCGCTG
GTGCTCCAATAAGAAAAATGCAGCAGTTATGGATCAGGAG
AGTGCAGGGAACCGCACCGCAAACAGCGAGGATTCCGATG
AACAAGACCCGCAAGAGGTGACCTACACACAGCTTAACCA
CTGCGTTTTCACGCAAAGGAAGATCACCCGACCATCCCAA
CGACCCAAGACCCCGCCCACCGATATAATTGTATATACCG
AGCTCCCAAACGCTGAGTCTCGATCAAAAGTTGTCTCCTG
CCCATGA
Optimized transgene sequence for KIR2DL2
(SEQ ID NO. 32):
ATGTCATTGATGGTGGTTTCTATGGCCTGCGTGGGTTTTT
TTCTTCTGCAAGGAGCTTGGCCCCACGAAGGAGTTCATAG
AAAGCCTAGCCTCCTTGCGCATCCTGGCCGCTTGGTGAAA
AGCGAAGAAACCGTGATACTTCAGTGTTGGTCTGATGTTC
GGTTTGAACACTTCTTGTTGCATCGGGAGGGCAAATTCAA
GGACACACTTCACCTGATTGGGGAACATCACGATGGAGTA
TCAAAAGCTAACTTCTCTATCGGACCGATGATGCAAGATC
TCGCCGGCACCTATCGGTGTTATGGGAGTGTGACTCACTC
TCCGTATCAATTGTCTGCACCAAGCGACCCACTGGATATT
GTGATAACCGGCTTGTATGAGAAGCCAAGTTTGTCCGCTC
AACCCGGTCCAACCGTTCTTGCCGGCGAGAGCGTGACTCT
TTCCTGCTCCTCCCGAAGCAGTTATGATATGTACCACCTT
TCCCGGGAGGGCGAGGCTCACGAGTGTAGATTCTCTGCCG
GCCCTAAAGTCAACGGGACCTTCCAAGCAGACTTTCCGCT
GGGACCGGCTACTCACGGTGGCACTTATCGCTGCTTTGGT
TCTTTTCGCGACTCCCCATACGAGTGGAGCAACAGCTCTG
ACCCGCTCCTTGTGTCCGTCATCGGGAACCCTTCTAATTC
ATGGCCCTCACCCACCGAGCCGTCATCTAAGACCGGGAAC
CCACGCCATCTTCATATTCTTATTGGCACATCCGTTGTGA
TTATACTTTTCATTCTTCTTTTTTTCCTCCTTCATCGGTG
GTGTTCAAACAAGAAAAACGCCGCGGTCATGGACCAGGAA
AGTGCTGGCAATCGAACAGCCAATAGCGAGGACTCAGACG
AACAGGACCCACAGGAGGTTACGTACACTCAACTGAACCA
CTGTGTTTTCACCCAACGGAAGATCACTCGCCCCTCCCAA
AGACCGAAAACGCCCCCTACCGATATAATTGTATATGCTG
AACTGCCAAATGCCGAAAGTAGATCCAAGGTAGTTTCTTG
TCCTTGA
Optimized transgene sequence for KIR2DL3
(SEQ ID NO. 33):
ATGTCTCTCATGGTAGTCTCTATGGTTTGCGTGGGCTTCT
TTCTCCTTCAAGGCGCTTGGCCACACGAGGGAGTCCACCG
AAAACCGTCACTCTTGGCGCATCCGGGCCCCTTGGTAAAA
AGCGAAGAAACGGTTATCCTTCAATGCTGGTCCGATGTAA
GATTTCAGCACTTCCTCCTCCATCGGGAAGGCAAATTTAA
GGATACCCTCCATCTGATCGGAGAGCATCATGACGGGGTA
TCTAAGGCGAACTTCTCTATCGGGCCCATGATGCAGGACC
TCGCTGGAACGTATCGATGCTACGGATCTGTAACGCACTC
ACCTTACCAACTCAGTGCTCCTTCCGACCCATTGGACATT
GTTATAACCGGACTCTATGAAAAACCCTCACTGAGCGCAC
AACCGGGCCCCACCGTGCTCGCGGGGGAGTCCGTGACGTT
GAGTTGCTCTTCACGGTCCTCTTACGACATGTACCACCTG
TCTCGAGAGGGGGAAGCTCACGAACGGAGATTTTCAGCAG
GCCCAAAGGTCAATGGCACATTCCAAGCAGATTTCCCGCT
TGGCCCCGCTACCCATGGGGGCACGTATAGGTGTTTTGGT
TCATTTCGGGACTCTCCGTATGAATGGTCCAATAGCAGTG
ATCCGTTGCTCGTTTCTGTAACCGGAAATCCCAGTAATAG
CTGGCCTAGTCCGACTGAGCCTAGTTCTGAAACCGGCAAC
CCTCGACACCTTCATGTACTGATCGGCACCTCTGTAGTGA
TAATTCTCTTCATTCTCCTCCTTTTTTTTCTGCTCCACCG
CTGGTGCTGCAACAAGAAGAACGCTGTCGTGATGGATCAG
GAACCCGCTGGCAACAGAACAGTTAATCGGGAGGATAGTG
ATGAGCAAGACCCCCAAGAGGTGACATATGCACAACTCAA
CCATTGCGTGTTCACTCAACGGAAAATAACTCGGCCATCT
CAACGGCCGAAAACCCCTCCCACTGACATAATCGTATACA
CCGAATTGCCAAATGCTGAGCCATGA
Optimized transgene sequence for KIR2DL4
(SEQ ID NO. 34):
ATGTCAATGTCCCCGACGGTAATTATCCTTGCATGTCTTG
GATTCTTTCTCGATCAAAGCGTATGGGCACATGTCGGTGG
ACAAGACAAGCCTTTCTGCTCCGCTTGGCCATCCGCCGTC
GTCCCGCAGGGGGGCCACGTCACACTTCGCTGCCACTATC
GGAGAGGTTTCAATATATTCACTCTCTACAAAAAGGATGG
GGTCCCCGTGCCGGAACTGTATAATAGAATATTCTGGAAC
TCTTTTTTGATTTCACCCGTGACGCCCGCTCACGCGGGCA
CATATAGATGCAGGGGATTTCATCCGCACTCACCAACGGA
GTGGAGCGCCCCGAGCAACCCGTTGGTTATTATGGTGACC
GGACTTTACGAAAAACCCTCATTGACGGCTAGGCCAGGGC
CCACAGTTCGGGCGGGCGAAAACGTAACGCTGTCTTGCTC
TTCCCAATCCTCTTTCGACATCTACCATTTGTCACGAGAG
GGCGAGGCTCATGAACTCAGGCTCCCAGCCGTGCCATCAA
TTAACGGGACCTTCCAGGCAGATTTTCCGTTGGGTCCTGC
AACTCATGGAGAAACCTACCGGTGTTTTGGAAGCTTCCAC
GGTTCCCCCTATGAATGGTCCGATCCGTCTGATCCATTGC
CCGTATCCGTTACTGGGAACCCGTCATCCAGCTGGCCTTC
TCCTACGGAACCTAGTTTTAAGACGGGAATAGCCCGCCAC
CTGCATGCCGTTATTAGATACAGTGTTGCTATCATTTTGT
TTACCATCCTTCCCTTCTTTCTGCTTCATCGGTGGTGTTC
AAAAAAGAAAGATGCTGCGGTTATGAATCAGGAACCAGCG
GGACATCGCACAGTTAATAGGGAAGATTCAGACGAACAAG
ACCCGCAGGAGGTCACATATGCACAGTTGGATCATTGTAT
TTTTACACAAAGAAAGATTACAGGACCATCCCAGCGCTCC
AAACGACCGTCAACCGATACATCAGTTTGCATTGAACTGC
CGAACGCGGAGCCGCGGGCGCTGTCCCCTGCACATGAGCA
CCATAGTCAGGCGCTGATGGGCAGCTCACGCGAAACGACG
GCCCTTTCTCAAACTCAACTGGCGAGTTCTAATGTCCCAG
CGGCTGGCATTTGA
Optimized transgene sequence for KIR2DL5A
(SEQ ID NO. 35):
ATGTCTCTCATGGTCATTTCAATGGCTTGTGTGGGTTTTT
TTCTTCTGCAGGGCGCTTGGACTCACGAAGGGGGGCAAGA
TAAGCCCCTTCTCTCCGCATGGCCGAGTGCGGTCGTCCCG
CGAGGCGGGCATGTGACGCTTCTTTGTCGCTCCCGGTTGG
GGTTTACCATTTTTTCCTTGTATAAGGAGGACGGAGTCCC
TGTTCCGGAGCTGTATAATAAGATATTCTGGAAGTCCATA
CTTATGGGGCCAGTAACGCCCGCCCACGCCGGCACCTACC
GCTGCCGGGGCTCCCACCCGAGATCCCCGATAGAGTGGAG
CGCCCCGTCTAATCCTTTGGTTATCGTTGTTACTGGGTTG
TTCGGAAAGCCTAGTCTGTCTGCCCAACCCGGCCCTACGG
TCCGGACTGGCGAAAACGTCACCCTTAGCTGTTCCAGTCG
GTCAAGTTTCGATATGTATCATTTGAGTAGAGAAGGCCGG
GCGCACGAACCAAGACTGCCTGCCGTGCCCTCTGTTAATG
GAACATTCCAGGCTGACTTCCCTCTTGGACCGGCTACTCA
TGGAGGCACTTACACTTGCTTTGGATCACTGCATGATTCA
CCTTACGAATGGAGTGACCCGTCCGACCCTTTGCTTGTGT
CTGTAACCGGCAACTCCTCTTCTTCCTCCTCTTCACCAAC
TGAACCGAGTTCCAAAACTGGGATAAGAAGGCACCTTCAC
ATACTCATCGGGACCTCAGTTGCAATTATCCTCTTTATCA
TCCTCTTCTTCTTCCTGCTGCATTGCTGCTGCTCCAATAA
GAAAAATGCGGCGGTAATGGACCAAGAGCCCGCAGGGGAT
AGGACCGTCAATAGGGAGGACTCTGACGATCAAGACCCCC
AAGAAGTTACCTATGCTCAATTGGATCATTGTGTCTTTAC
TCAAACAAAGATCACTTCTCCTTCCCAACGCCCAAAGACT
CCTCCAACCGACACCACTATGTATATGGAGCTGCCTAACG
CTAAGCCTCGGAGCCTCTCTCCGGCTCACAAGCACCACTC
ACAAGCACTGCGAGGATCATCACGAGAAACTACCGCATTG
AGCCAAAATAGAGTGGCGTCCAGCCATGTTCCCGCCGCCG
GAATATGA
Optimized transgene sequence for KIR2DL5B
(SEQ ID NO. 36):
ATGTCACTGATGGTGGTGTCCATGGCGTGCGTTGGCTTCT
TTCTCCTGCAGGGGGCATGGACGCATGAGGGCGGTCAGGA
CAAGCCTCTGTTGTCCGCGTGGCCTAGTGCGGTTGTTCCC
CGGGGCGGTCACGTCACCCTTTTGTGTCGGAGCAGACTGG
GATTCACAATCTTCTCCCTTTATAAAGAAGATGGGGTCCC
CGTACCCGAGCTCTACAACAAGATCTTTTGGAAGTCAATA
CTCATGGGGCCCGTAACACCCGCACATGCCGGGACGTACC
GCTGCCGGGGAAGTCACCCCCGAAGCCCTATTGAATGGAG
TGCACCCTCCAACCCGCTCGTCATTGTGGTGACTGGCCTG
TTCGGGAAGCCCTCACTCAGCGCTCAACCCGGACCCACCG
TCCGAACCGGGGAGAACGTGACTCTCTCTTGCTCCTCCCG
GAGTAGCTTTGACATGTATCACCTTTCCCGAGAGGGGCGG
GCACATGAACCTAGACTGCCAGCGGTTCCTTCCGTGGATG
GCACTTTTCAGGCCGACTTTCCCCTGGGACCTGCTACTCA
CGGGGGGACTTACACATGTTTTTCATCCTTGCACGACTCA
CCCTACGAGTGGAGCGACCCCTCCGATCCCCTTTTGGTAT
CCGTCACCGGAAACAGTTCCTCAAGCTCCTCCAGTCCGAC
AGAACCCTCTTCCAAGACCGGGATACGCCGCCACCTGCAC
ATTCTCATCGGAACTTCAGTTGCGATTATCTTGTTCATCA
TACTGTTCTTCTTCCTGTTGCATTGCTGTTGCTCCAATAA
AAAGAATGCAGCAGTAATGGATCAAGAGCCCGCGGGAGAC
AGGACAGTCAACCGAGAGGACTCCGACGATCAAGACCCTC
AAGAGGTCACATATGCACAACTGGACCACTGTGTCTTTAC
GCAAACGAAAATAACCAGCCCGTCCCAACGACCAAAGACT
CCACCCACGGACACGACAATGTATATGGAGCTTCCAAACG
CTAAGCCGAGATCATTGAGTCCTGCTCACAAACATCATTC
CCAGGCCCTGCGGGGGTCCAGTAGGGAAACAACGGCGCTG
TCCCAAAACCGCGTTGCTAGTTCACATGTCCCCGCCGCCG
GAATTTGA
Optimized transgene sequence for MIR21
(SEQ ID NO. 37):
CCAGTTTTCTTGCCGTTCTGTAAGTGTTTTATTCTTAGTG
TGATTTTTTTCCATTGGGATGTTTTTGATTGAACTTGTTC
ATTTTGTTTTGCTTGGGAGGAAAATAAACAATTTTACTTT
TTTCCTTTAGGAGCATTATGAGCATTATGTCAGAATAGAA
TAGAATTGGGGTTCGATCTTAACAGGCCAGAAATGCCTGG
GTTTTTTTGGTTTGTTTTTGTTTTTGTTTTTTTATCAAAT
CCTGCCTGACTGTCTGCTTGTTTTGCCTACCATCGTGACA
TCTCCATGGCTGTACCACCTTGTCGGGTAGCTTATCAGAC
TGATGTTGACTGTTGAATCTCATGGCAACACCAGTCGATG
GGCTGTCTGACATTTTGGTATCTTTCATCTGACCATCCAT
ATCCAATGTTCTCATTTAAACATTACCCAGCATCATTGTT
TATAATCAGAAACTCTGGTCCTTCTGTCTGGTGGCACTTA
GAGTCTTTTGTGCCATAATGCAGCAGTATGGAGGGAGGAT
TTTATGGAGAAATGGGGATAGTCTTCATGACCACAAATAA
ATAAAGGAAAACTAAGCTGCATTGTGGGTTTTGAAAAGGT
TATTATACTTCTTAACAATTCTTTTTTTCAGGGACTTTTC
TAGCTGTATGACTGTTACTTGACCTTCTTTGA
Optimized transgene sequence for MIR181B1
(SEQ ID NO. 38):
CTTATTTGTCTTCTTTTGTAGTCTTTTGAAATGGCATAAA
AATGCATAAAATATATGACTAAAGGTACTGTTGTTTCTGT
CTCCCATCCCCTTCAGATACTTACAGATACTGTAAAGTGA
GTAGAATTCTGAGTTTTGAGGTTGCTTCAGTGAACATTCA
ACGCTGTCGGTGAGTTTGGAATTAAAATCAAAACCATCGA
CCGTTGATTGTACCCTATGGCTAACCATCATCTACTCCAT
GGTGCTCAGAATTCGCTGAAGACAGGAAACCAAAGGTGGA
CACACCAGGACTTTCTCTTCCCTGTGCAGAGATTATTTTT
TAAAAGGTCACAATCAACATTCATTGCTGTCGGTGGGTTG
AACTGTGTGGACAAGCTCACTGAACAATGAATGCAACTGT
GGCCCCGCTTTTTGCTGTCACAATCAACAGATATTCCATC
TTTGAAAGATGTGTTCAAAATAGTACTATTGTTCTTTAAG
TTTTCCAATTCGTCAGCATCTTCTGGTTTATTTATTCAAT
TTATATCATGTGATGAGATTTCAATGAAAAAGACTCTGGT
TTAAACAAGAATAATGCTTCTCTATCTTTTGTATGCTATA
AACAGAGCATGTTTTAGAAAAGGCAAAACTATTTTGTCCA
TCAAACGTACAGTATCATCATCTAGGTTAGCTCACCTTCC
TACTGTTGTAGTGTGAGTGAAAAAAAGACA
Optimized transgene sequence for MIR181A1
(SEQ ID NO. 39):
TACCACAAAGTACTTTGAATAACAAATTGTCCCTGTCTTT
AACAGTGAGATAATTCCATCTCTGGAACTAGCCCAATATC
GGCCATGTTTTTGCTTAATGAAACCGATCCTTTTCTCTCA
TACAATGTGATGTGGAGGTTTGCCAAACTCTTTGTTGGAA
GAATCATGCTTCTTATTTGTCTTCTTTTGTAGTCTTTTGA
AATGGCATAAAAATGCATAAAATATATGACTAAAGGTACT
GTTGTTTCTGTCTCCCATCCCCTTCAGATACTTACAGATA
CTGTAAAGTGAGTAGAATTCTGAGTTTTGAGGTTGCTTCA
GTGAACATTCAACGCTGTCGGTGAGTTTGGAATTAAAATC
AAAACCATCGACCGTTGATTGTACCCTATGGCTAACCATC
ATCTACTCCATGGTGCTCAGAATTCGCTGAAGACAGGAAA
CCAAAGGTGGACACACCAGGACTTTCTCTTCCCTGTGCAG
AGATTATTTTTTAAAAGGTCACAATCAACATTCATTGCTG
TCGGTGGGTTGAACTGTGTGGACAAGCTCACTGAACAATG
AATGCAACTGTGGCCCCGCTTTTTGCTGTCACAATCAACA
GATATTCCATCTTTGAAAGATGTGTTCAAAATAGTACTAT
TGTTCTTTAAGTTTTCCAATTCGTCAGCATCTTCTGGTTT
ATTTATTCAATTTATATCATGTGATGAGAT
Optimized transgene sequence for MIR144
(SEQ ID NO. 40):
CTGGGTCTGTCTGCCTGTCTGCTTGTTAGTGTGACCAAAG
GGAACCTTCTGCATCATTACGCCATCTCTGGCTTGTTTAA
CACTGGCCCTGGGTCCCTATGAGATCTTAACAGACCCTAG
CTCCAGTCCCCTTCCCATAACCCACCTGGGCTGTGCCTGA
CCACAGAATCAAGGAGACGCTGGCCTGCGAGGGAGCTGTA
GAGCAGGGAGCAGGAAGCTGTGTGTGTCCAGCCCTGACCT
GTCCTGTTCTGCCCCCAGCCCCTCACAGTGCTTTTCAAGC
CATGCTTCCTGTGCCCCCAGTGGGGCCCTGGCTGGGATAT
CATCATATACTGTAAGTTTGCGATGAGACACTACAGTATA
GATGATGTACTAGTCCGGGCACCCCCAGCTCTGGAGCCTG
ACAAGGAGGACAGGAGAGATGCTGCAAGCCCAAGAAGCTC
TCTGCTCAGCCTGTCACAACCTACTGACTGCCAGGGCACT
TGGGAATGGCAAGGAAACCGTTACCATTACTGAGTTTAGT
AATGGTAATGGTTCTCTTGCTATACCCAGAAAACGTGCCA
GGAAGAGAACTCAGGACCCTGAAGCAGACTACTGGAAGGG
AGACTCCAGCTCAAACAAGGCAGGGGTGGGGGCGTGGGAT
TGGGGGTAGGGGAGGGAATAGATACATTTTCTCTTTCCTG
TTGTAA
Optimized transgene sequence for MIR150
(SEQ ID NO. 41):
GGGAGGGGGCCGCAACCTCCTATTCCCCTCTGGGTCTCCG
TCCCCTCCCTCTGGAGTCCACACTCCCTCTTTGCCCCTTG
CTGGTTCTCTACTGCCCCCAGCATAGGGTGGAGTGGGTGT
GCAGTTTCTGCGACTCAGGGTGGCGTCCCCCCAACCTGTC
CCTGCCCCTTCCTGCCCTCTTTGATGCGGCCCCACTTCCT
CTGGCAGGAACCCCCGCCCTCCCTGGACCTGGGTATAAGG
CAGGGACTGGGCCCACGGGGAGGCAGCGTCCCCGAGGCAG
CAGCGGCAGCGGCGGCTCCTCTCCCCATGGCCCTGTCTCC
CAACCCTTGTACCAGTGCTGGGCTCAGACCCTGGTACAGG
CCTGGGGGACAGGGACCTGGGGACCCCGGCACCGGCAGGC
CCCAAGGGGTGAGGTGAGCGGGCATTGGGACCTCCCCTCC
CTGTACTCCCATCTCTGCTGCGGCTTTTATGCGTCTCTCC
CCTTCGGGTCCCACATATCCTCTGGTGCGCTCCTGCCTCA
CCGCCCCCACCCCATGCCTGTCGTCCCCACCTCTGTGTGA
TGCGCAAAGTACACCTGTTTCTATTGTACCTGCCTCTCGC
GGTGGTCTGTGCTCTCCCCAGCTCTGCAAAACCCCTCCTC
CCCATGTGCCACAACCCTGGGCCACCGTGTGTCCTGTCCT
GTTC

### Example 3: Enhanced CD16 Signaling

For improved cell therapy (e.g., stem cell therapy, adaptive immunotherapy, etc.), cells of interest can be engineered to exhibit enhanced CD16 signaling (e.g., enhanced endogenous CD16 signaling, introduced transgene comprising CD16, etc.). The cells of interest can be stem cells, such as isolated stem cells (e.g., embryonic stem cells) or induced stem cells (e.g., iPSCs). The cells of interest can be immune cells (e.g., NK cells). Such immune cells can be derived from the stem cells as disclosed herein. Alternatively, such immune cells can be immune cell lines (e.g., NK cell lines).

For example, for improved adaptive immunotherapy, NK cells can be engineered to exhibit enhanced CD16 signaling

hnCD16 amino acid sequence (SEQ ID NO. 1):
MWFLTTLLLWVPVDGQVDTTKAVITLQPPWVSVFQEETVT
LHCEVLHLPGSSSTQWFLNGTATQTSTPSYRITSASVNDS
GEYRCQRGLSGRSDPIQLEIHRGWLLLQVSSRVFTEGEPL
ALRCHAWKDKLVYNVLYYRNGKAFKFFHWNSNLTILKTNI
SHNGTYHCSGMGKHRYTSAGISVTVKELFPAPVLNASVTS
PLLEGNLVTLSCETKLLLQRPGLQLYFSFYMGSKTLRGRN
TSSEYQILTARREDSGLYWCEAATEDGNVLKRSPELELQV
LGLQLPTPVWFHVSFCLVMVLLFAVDTGLYFSVKTNIRSS
TRDWKDHKFKWRKDPQDK

Engineered NK Cells:

NK-92 cells were engineered to exhibit enhanced CD16 signaling. The engineered NK-92 cells were modified to express CD64/CD16A fusion protein (i.e., hnCD16) (SEQ ID NO. 1).

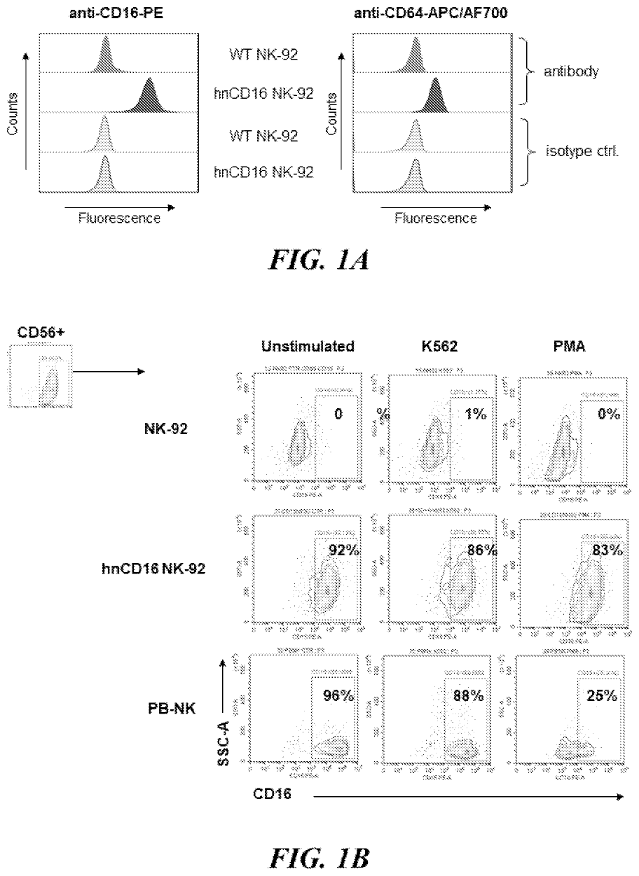

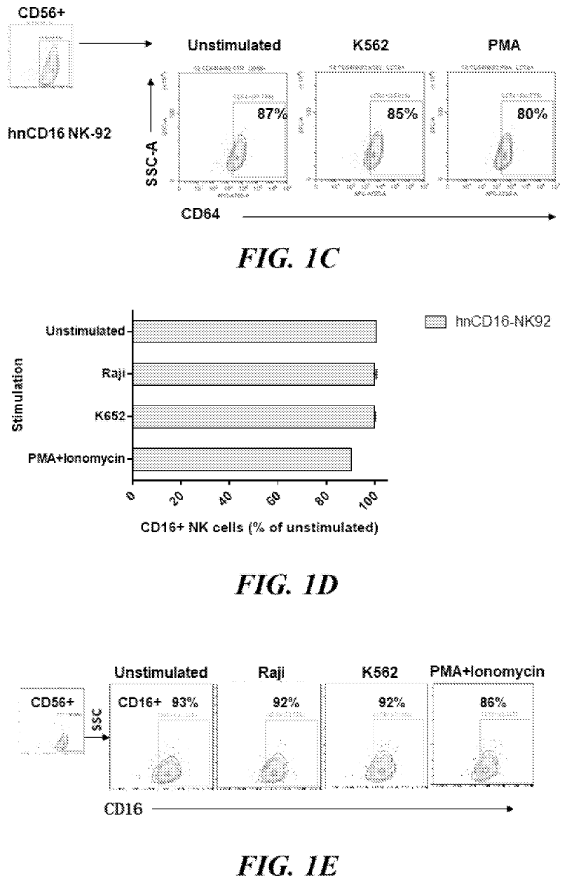

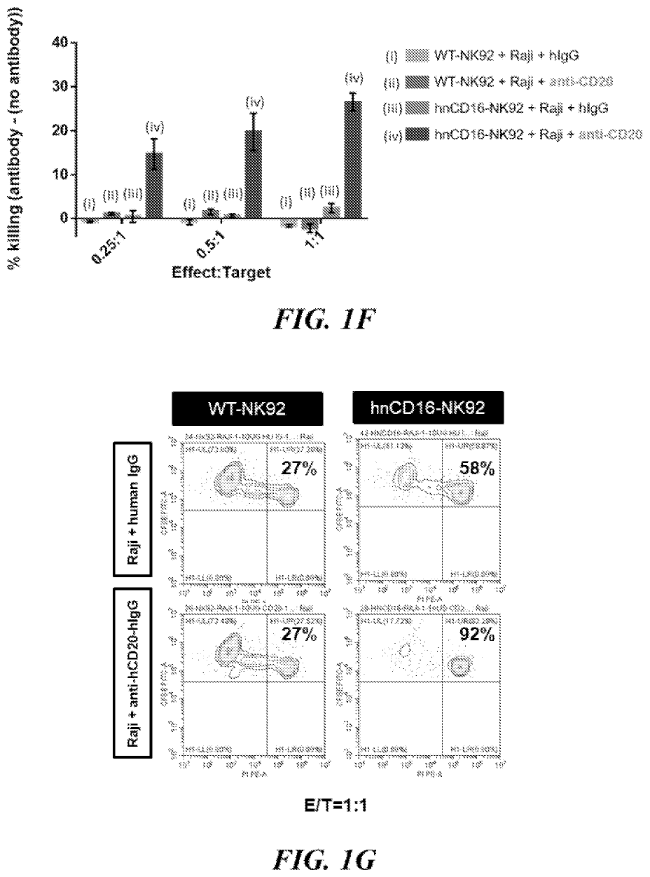

The resulting hnCD16 NK-92 cells were validated by identifying enhanced expression of both CD16 (e.g., via anti-CD16-PE antibody) and CD64 (e.g., via anti-CD64-APC/AF700 antibody) using fluorescence-activated cell sorting (FACS), as shown in FIG. 1A. Wild-type (WT) NK-92 cells were used as control.

The hnCD16 construct sequence can comprise “FHVS” (SEQ ID NO. 2). The hnCD16 construct sequence can comprise “WFHVS” (SEQ ID NO. 3). The hnCD16 construct sequence can comprise “FHVSF” (SEQ ID NO. 4). The hnCD16 construct sequence can comprise “WFHVSF” (SEQ ID NO. 5). The hnCD16 construct sequence can comprise “VWFHVSFC” (SEQ ID NO. 6). The hnCD16 construct sequence can comprise “PVWFHVSFCL” (SEQ ID NO. 7). The hnCD16 construct sequence can comprise “TPVWFHVSFCLV” (SEQ ID NO. 8).

Enhanced Targeting:

The hnCD16 NK-92 cells were cultured alone (unstimulated, control) or in the presence of K562 cells capable of activating NK cells (K562) or phorbol 12-myristate 13-acetate (PMA) to activate CD16 and induce cleavage thereof. Data revealed that the hnCD16 NK-92 cells were highly resistant to the activation-induced cleavage of CD16a, as compared to peripheral blood (PB) NK cells as a control (FIG. 1B). For example, treatment with PMA marginally reduced the percentage of CD16+ cells from 92% to 85% for the hnCD16 NK-92 cells, whereas the same treatment reduced the percentage of CD16+ cells from 96% to 25% (FIG. 1B). Persistency of hnCD16 in the hnCD16 NK-92 cells was also confirmed by using anait-CD64 antibody (FIG. 1C). Also, it was observed that hnCD16 NK-92 cells did not downregulate endogenous CD16 expression upon stimulation (e.g., K652 or PMA) (FIGS. 1D and 1E).

Enhanced Co-Antibody Therapy

The target cells (Raji cells) were treated with (i) the hnCD16 NK-92 cells and (ii) either anti-CD20 antibody or hIgG as a control. Data revealed that the hnCD16 NK-92 cells in combination with anti-CD20 antibody induced enhanced death (e.g., at least partially ADCC-mediated death) of the target cells as compared to controls (e.g., wildtype NK92 cells with anti-CD20 antibody, or the hnCD16 NK-92 cells with hIgG) (FIGS. 1F and 1G).

iPSC Expressing hnCD16 Differentiated into iNK

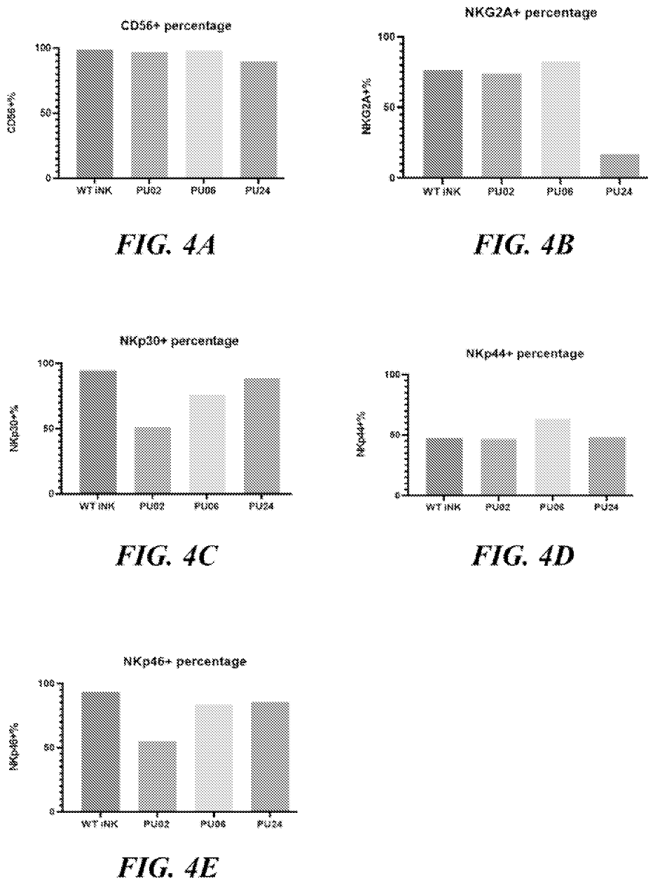

Engineered iPSC as disclosed herein (e.g., iPSCs engineered with enhanced or introduced CD16 signaling (e.g., hnCD16, a CD16 variant)) were subjected for differentiation into NK cells (e.g., iNK cells). In some cases, the iNK cells (e.g., about 5×10⁵ culture containing cells) were collected and used for staining (e.g., staining for NK specific markers), e.g., with NKG2A-PE, NKp30-PE, NKp44-PE, NKp46-PE, and/or CD56-APC antibody. As shown in FIGS. 4A-4E respectively, CD56⁺ cells were detected in the differentiated cells, and NKG2A⁺, NKp30⁺, NKp44⁺, and/or NKp46⁺ cells were detected in the CD56⁺ population among three exemplified clones, PU02, PU06, and PU24, which expressed enhanced CD16 signaling via hnCD16. Therefore, iPSC expressing enhanced or introduced CD17 signaling were proven to be able to differentiate into iNK cells.

Enhanced CD16 Signaling in NK Cells

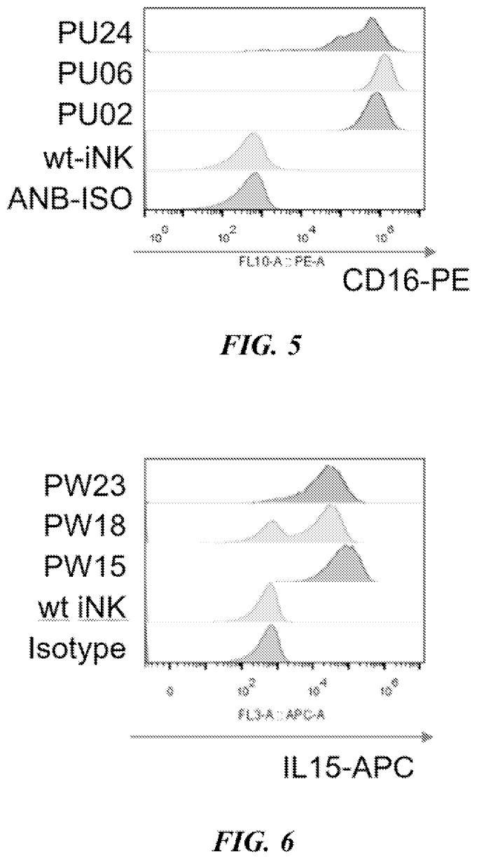

As depicted in FIG. 5, enhanced CD16 signaling (e.g., overexpression of CD16, such as hnCD16) in NK cells (e.g., wild-type iNK cells or iNK clones with enhanced CD16 signaling, such as PU02, PU06, and PU24 clones), were detected by FACS with PE conjugated anti-CD16 antibody to be more abundant when compared to controls wild-type (wt) NK-92 cells and ANB-ISO. As such, hnCD16 were indeed overexpressed in hnCD16 NK-92 cells.

Combination of NK Cells with an Antibody

NK cells (e.g., NK92 cell line, NK cell derived from stem cells such as iPSCs) as disclosed herein can be used with a separate dosage of antibody for an enhancement of cytotoxicity against target cells (e.g., cancer cells). For example, NK cells engineered to express enhanced CD16 signaling (e.g., hnCD16, CD16-CD64 fusion protein, etc.) can induce cell lysis in certain target cells (e.g., Raji cells) in vitro with a combination of a certain antibody (e.g., anti-CD20 mAb), and percentage of lysed target cells is higher in the combination treatment than the NK cells or the antibody alone.

### Example 4: Enhanced Survival and Persistency of NK Cells

For improved cell therapy (e.g., stem cell therapy, adaptive immunotherapy, etc.), cells of interest can be engineered to exhibit (i) reduced expression of at least one endogenous gene (e.g., one or more immune regulating polypeptides) and/or (ii) enhanced or introduced expression of at least one additional gene (e.g., one or more additional immune regulating polypeptides). Cells comprising (i) and/or (ii) as disclosed herein can exhibit enhanced function, such as a persistence level (or survival level). In some cases, having the combination of (i) and (ii) can synergistically improve function of the cells, as compared to having either one of (i) and (ii) alone, or a combination of individual effects of (i) and (ii), or none. The cells of interest can be stem cells, such as isolated stem cells (e.g., embryonic stem cells) or induced stem cells (e.g., iPSCs). The cells of interest can be immune cells (e.g., NK cells). Such immune cells can be derived from the stem cells as disclosed herein. Alternatively, such immune cells can be immune cell lines (e.g., NK cell lines).

Generation of Engineered NK Cells:

For example, for enhanced persistence of adaptive immunotherapy, NK cells can be engineered to comprise at least (i) a heterologous transcription factor (e.g., STAT) and (ii) reduced expression or activity of an endogenous cytokine receptor (e.g., endogenous IL receptor, such as IL-17R). Having the combination of (i) and (ii) in the engineered NK cell can synergistically improve persistence of the engineered NK cells as compared to having either one of (i) and (ii) alone, or none.

NK cells are generated from isolated ESCs or iPSCs. The NK cells are engineered to express a heterologous STAT (e.g., STAT3 and/or STAT5B). A gene encoding the heterologous STAT is incorporated into the NK cell's genome via either viral transduction or via action of a gene editing moiety as disclosed herein. The NK cells are also engineered to exhibit reduced expression or activity of endogenous IL-17R (i.e., STAT3⁺IL-17R⁻ NK cells). NK cells with either one of (i) the heterologous STAT and (ii) reduced expression or activity of IL-17R, or non-engineered NK cells are used as a control.

In Vitro Survival and Persistency:

The engineered STAT3⁺IL-17R⁻ NK cells can be cultured in vitro to assess viability and growth (or proliferative capacity) of the engineered STAT3⁺IL-17R⁻ NK cells in absence of an exogenous cytokine. The NK cells are cultured in culture medium without the addition of exogenous cytokines for 3-6 weeks. The engineered STAT3⁺IL-17R⁻ NK cells exhibit a significantly higher number of NK cells as compared to the control cells, indicating the enhanced survival and persistency of the engineered STAT3⁺IL-17R⁻ NK cells in vitro.

In Vivo Pharmacokinetics (PK):

The engineered STAT3⁺IL-17R⁻ NK cells can be administered in NCG mice having a Raji xenograft model. NCG mice are triple immunodeficient and lack functional/mature T, B, and NK cells, and have reduced macrophage and dendritic cell function to host the xenograft model. The engineered STAT3⁺IL-17R⁻ NK cells and the control cells are each administered into the respective Raji xenograft model mice via intravenous (IV) tail vein injection, at a dose of about 1×10⁶ cells per animal. Mice injected with the engineered STAT3⁺IL-17R⁻ NK cells exhibit higher NK cell concentrations in the peripheral blood from about 7 days to about 28 days post-infusion, demonstrating the enhanced survival and persistency of the engineered STAT3⁺IL-17R⁻ NK cells in vivo.

Generation of Other Engineered NK Cells:

Cells of interest (e.g., stem cells, immune cells, such as NK cells, etc.) can be engineered with (i) reduced expression of at least one endogenous gene (e.g., loss-of-function of one or more immune regulating polypeptides) and/or (ii) enhanced or introduced expression of at least one additional gene (e.g., at least one transgene encoding one or more additional immune regulating polypeptides). For example, NK cells (e.g., cord blood NK (CBNK) cells, NK cells derived from iPSCs, etc.) may have edits with loss-of-function of BCL3 transcription coactivator (BCL3); loss-of-function of Cbl proto-oncogene B (CBLB); loss-of-function of cyclin dependent kinase 8 (CDK8); loss-of-function of Fc fragment of IgE receptor Ig (FCER1G); loss-of-function of interleukin 17A (IL17A); loss-of-function of interleukin 17F (IL17F); loss-of-function of inositol polyphosphate-5-phosphatase D (INPP5D/SHIP1); loss-of-function of suppressor of cytokine signaling 1 (SOCS1); loss-of-function of suppressor of cytokine signaling 2 (SOCS2); loss-of-function of suppressor of cytokine signaling 3 (SOCS3); loss-of-function of signal transducer and activator of transcription 3 (STAT3); loss-of-function of tet methylcytosine dioxygenase 2 (TET2); loss-of-function of protein tyrosine phosphatase non-receptor type 2 (PTPN2); loss-of-function of protein tyrosine phosphatase non-receptor type 6 (PTPN6); loss-of-function of CD70 molecule (CD70); loss-of-function of CD38 molecule (CD38); loss-of-function of cytokine inducible SH2 containing protein (CISH); and/or a transgene for killer cell lectin like receptor C2 (KLRC2/NKG2C).

In some cases, the loss-of function gene editing can be fulfilled by one or more gene editing moieties as disclosed herein, such as CRIPSR/Cas9 system. Briefly, NK cells (e.g., CBNK cells) are recovered from cryopreservation and cultured using the medium (e.g., Lymphocyte Serum-Free Medium KBM 581 (Corning), 10% Human male AB serum, 1% MEM Non-Essential Amino Acids Solution (100×, Gibco), 1% L-Glutamine (200 mM) (Gibco), 0.02% Vitamin C, 200 U/mL IL-2). For each editing, cells (e.g., about 1×10⁷ cells) are isolated and transfected with guide RNA/Cas9 protein complex (RNP) (e.g., using Lonza Nucleofector 4D and P3 Primary Cell 4D-Nucleofector™ X Kit L). The guide RNA and Cas9 are transfected, e.g., with the ratio of 2:1 (guide RNA 75 picomole (pmol), Cas9 protein 150 pmol).

The transfected cells are recovered (e.g., using the medium with doubled Human male AB serum (20%)) before downstream assays. The editing efficiency is analyzed by fragment analysis or NGS.

The sequences of guide RNAs and primers for editing efficiency evaluation are listed in TABLE 4 and TABLE 5 below.

TABLE 4
Sequences of guide RNAs for NK cells with
enhanced survival and persistency

| SEQ | | | | |
|---|---|---|---|---|
| ID | Target | | gRNA | |
| NO | name | gRNA sequence | position | Strand |
| 190 | BCL3 | TTGACCAGCC | chr19: | − |
| | | GGTGCACAGC | 44756266- | |
| | | | 44756288 | |
| 59 | CBLB | AAATTCTCGG | chr3: | + |
| | | AGGATGCTCC | 105659009- | |
| | | | 105659031 | |
| 93 | CDK8 | CTTGCTCCAA | chr13: | − |
| | | GAAGTAGTTC | 26385300- | |
| | | | 26385322 | |
| 56 | CISH | AGTCCACGTC | chr3: | + |
| | | GGCCACCAGA | 50607666- | |
| | | | 50607688 | |
| 48 | FCER1G | CTTCAGTCGA | chr1: | − |
| | | CAGTAGAGGA | 161218055- | |
| | | | 161218077 | |
| 75 | IL17A | TGGTATTGGT | chr6: | − |
| | | ATTCCGGTTA | 52187728- | |
| | | | 52187750 | |
| 112 | INPP5D | CAACATCACC | chr2: | + |
| | | CGCTCCAAGG | 233060502- | |
| | | | 233060524 | |
| 97 | SOCS1 | TCCGTTCGCA | chr16: | + |
| | | CGCCGATTAC | 11255284- | |
| | | | 11255306 | |
| 90 | SOCS2 | CTTGAGCCCT | chr12: | + |
| | | CCGGGAATGG | 93572913- | |
| | | | 93572935 | |
| 103 | SOCS3 | CAGCAGGTTC | chr17: | + |
| | | GCCTCGCCGC | 78358919- | |
| | | | 78358941 | |
| 100 | STAT3 | TAAGACCCAG | chr17: | − |
| | | ATCCAGTCCG | 42322471- | |
| | | | 42322493 | |
| 68 | TET2 | CAGGACTCAC | chr4: | − |
| | | ACGACTATTC | 105234149- | |
| | | | 105234171 | |
| 191 | PTPN2 | TAGAGGAAAG | chr18: | + |
| | | TCCTGTACAT | 12802004- | |
| | | | 12802026 | |
| 192 | PTPN6 | TCGGCCCAGT | chr12: | + |
| | | CGCAAGAACC | 6951687- | |
| | | | 6951709 | |
| 193 | IL17F | GTACTTGCTG | chr6: | + |
| | | CTGTCGATAT | 52238923- | |
| | | | 52238945 | |
| 194 | CD38 | TATCAGCCAC | chr4: | − |
| | | TAATGAAGTT | 15816593- | |
| | | | 15816612 | |
| 195 | CD70 | GCCCGCAGGA | chr19: | + |
| | | CGCACCCATA | 6590941- | |
| | | | 6590960 | |

TABLE 5
Primer sequences for editing efficiency evaluation

| Gene | Forward primer | Reverse primer |
|---|---|---|
| | sequence (5′->3′) | sequence (5'->3') |
| BCL3 | TGGGGACATTTCCAAAGTATTCAAC | CTGCCGTAGGTTGTTGTAGATGTC |
| | (SEQ ID NO: 196) | (SEQ ID NO: 213) |
| CBLB | GCTACACGAGGAGGAGACACT (SEQ ID | ATGGGAGAGGGTTATGCCTTTG |
| | NO: 197) | (SEQ ID NO: 214) |
| CDK8 | TATTTTACAGCTGACATGGGCTTTG | TCATGGAATTTTAATTAGTTCTACT |
| | (SEQ ID NO: 198) | CACCA (SEQ ID NO: 215) |
| CISH | TACTGTCGGAGGTAGTCGGC (SEQ ID | TAAGGAGGATGCGCCTAGTGA |
| | NO: 199) | (SEQ ID NO: 216) |
| FCER1G | GATGCCATCCTGTTTCTGTATGGA (SEQ | GGAATGGGGGAAAGGTTATCAGT |
| | ID NO: 200) | AA (SEQ ID NO: 217) |
| IL17A | CCCGGACTGTGATGGTCAAC (SEQ ID | CACAGTGGTCCTTCCAGGTTT (SEQ |
| | NO: 201) | ID NO: 218) |
| INPP5D | GTTTCTGTAATGAGGAAGTTCTCCG | GTCCTTGCCTGTCCTGGAA (SEQ ID |
| | (SEQ ID NO: 202) | NO: 219) |
| SOCS1 | CAGGGGCCCCCAGTAGAATC (SEQ ID | TGTAGGATGGTAGCACACAACCA |
| | NO: 203) | G (SEQ ID NO: 220) |
| SOCS2 | TGACCCTTCTCTCATGACCCTG (SEQ ID | AGCAAGGGACGTATTTCCATCTTA |
| | NO: 204) | T (SEQ ID NO: 221) |
| SOCS3 | AGCTTGAGCACGCAGTCG (SEQ ID NO: | GAGAGCGGCTTCTACTGGAG (SEQ |
| | 205) | ID NO: 222) |
| STAT3 | ACCAGTGGAGACACCAGGATA (SEQ ID | AGTCTTTTCCCCTTCGAGGA (SEQ |
| | NO: 206) | ID NO: 223) |
| TET2 | ATCCAGAAGTAAATGGAGACACCAA | GAAGGTTCACTAACTGTGCGTTTT |
| | (SEQ ID NO: 207) | A (SEQ ID NO: 224) |
| PTPN2 | CCTGAAATCCTATGGCACCTGG (SEQ ID | CTGCCTTTGATCATTCACCAAACA |
| | NO: 208) | (SEQ ID NO: 225) |
| PTPN6 | GCTGCATCTGAGGCTTAGTCC (SEQ ID | AACCAGGAATGAGTGGTTCAGGG |
| | NO: 209) | (SEQ ID NO: 226) |
| IL17F | TTCATTGATGATGCCAATGTCAAGC | CTCTAACTGATTGCAGCGTCTTCT |
| | (SEQ ID NO: 210) | A (SEQ ID NO: 227) |
| CD38 | TAGACTGCATGTTAGACGAGATAA | GTTGCAAGGTACGGTCTGAG (SEQ |
| | (SEQ ID NO: 211) | ID NO: 228) |
| CD70 | CGCTAGCGGAGGTGATCG (SEQ ID NO: | AAGTGACTCGAGCGGCAG (SEQ ID |
| | 212) | NO: 229) |

Optimized sequence of transgene for killer cell lectin like receptor C2 (KLRC2/ NKG2C)
(SEQ ID No. 42):
ATGAATAAACAAAGAGGAACATTCTCCGAGGTATCTCTGG
CTCAGGACCCTAAAAGACAGCAACGCAAACCTAAAGGAAA
TAAATCAAGCATCAGTGGCACAGAACAGGAAATATTTCAA
GTGGAATTGAATCTGCAGAATCCCTCCTTGAACCACCAGG
GCATCGACAAAATTTATGATTGTCAGGGGCTCCTGCCGCC
GCCCGAAAAGTTGACAGCCGAGGTGCTGGGCATCATCTGT
ATAGTCTTGATGGCGACGGTTCTTAAGACAATTGTCCTTA
TACCCTTTCTGGAGCAAAACAATTTTTCACCCAATACTCG
AACCCAAAAAGCTCGGCACTGCGGCCACTGTCCCGAGGAG
TGGATAACATACTCAAACTCTTGCTATTATATCGGCAAAG
AAAGAAGAACTTGGGAGGAGAGTTTGCTCGCTTGTACGAG
CAAAAATAGCAGTCTCCTTTCCATCGATAACGAAGAGGAG
ATGAAATTCCTTGCGTCAATACTGCCTAGTTCTTGGATTG
GCGTCTTTCGGAACTCCTCACACCACCCTTGGGTAACTAT
AAACGGCCTCGCATTCAAGCATAAGATCAAGGACTCTGAC
AACGCGGAGCTTAATTGTGCGGTTCTGCAAGTGAACCGGT
TGAAGAGTGCCCAGTGCGGATCATCCATGATATACCACTG
TAAACACAAGCTGTAG

In Vitro Screening for Cell Persistence (e.g., NK Cell Persistence) Related Genes

NK cells can be utilized as a model population of cells to examine persistence enhancing gene edits. A population of NK cells can be engineered comprise (i) reduced expression of at least one endogenous immune regulating polypeptide comprising one or more members selected from the group consisting of BCL3, CBLB, CDK8, FCER1G, IL17A, IL17F, SHIP1, SOCS1, SOCS2, SOCS3, STAT3, TET3, PTPN6, and CD70, and/or (ii) the enhanced or introduced expression of NKG2C. In a mixture of cells comprising such population of engineered NK cells and other cells (e.g., other NK cells) that do not comprise (i) and/or (ii), the persistence level of the population of engineered NK cells in such mixture can be characterized by (i) an enrichment level of the population of engineered NK cells within the mixture in a sub-optimal environment that is greater than (ii) an enrichment level of the population of engineered NK cells within the mixture in an optimal environment. The sub-optimal environment can comprise a lower amount (or concentration) of exogenous cytokine (e.g., exogenous IL, such as IL-2). For example, the sub-optimal environment can comprise an amount of the exogenous cytokine that is at least about 1%, at least about 5%, at least about 10%, at least about 20%, at least about 30%, at least about 40%, at least about 50%, at least about 60%, at least about 70%, at least about 80%, at least about 90%, or more lower than that in the optimal environment. The sub-optimal environment can be an in vitro medium, and the optimal environment can be an in vitro medium. The sub-optimal environment can be an in vivo environment (e.g., blood stream of a subject), and the optimal environment can be an in vitro medium. The enrichment level of the population of engineered NK cells within such mixture can be ascertained by identifying an amount (or proportion) of cells exhibiting (i) the reduced expression of at least one endogenous immune regulating polypeptide comprising one or more members selected from the group consisting of BCL3, CBLB, CDK8, FCER1G, IL17A, IL17F, SHIP1, SOCS1, SOCS2, SOCS3, STAT3, TET3, PTPN6, and CD70, and/or (ii) the enhanced or introduced expression of NKG2C.

Without wishing to be bound by theory, NK cells with reduced expression or activity level of a gene of interest (e.g., loss-of-function of FCER1G) may survive or persist longer in a sub-optimal environment (e.g., low cytokine). In a more optimal environment (e.g., high cytokine), NKs cells may exhibit high survival rate or persistence, regardless of whether the NK cells comprise the reduced expression or activity level of such gene of interest (e.g., FCER1G). Thus, exhibiting a higher enrichment (e.g., INDEL %) in a mixture with different types of cells in the sub-optimal (or more challenging) environment can suggest that such loss-of-function gene may induce enhanced survival or persistence to the cell.

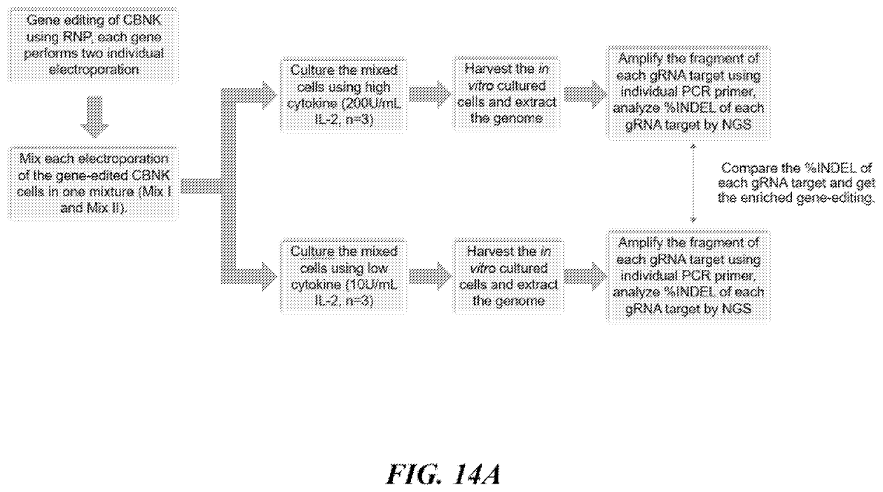

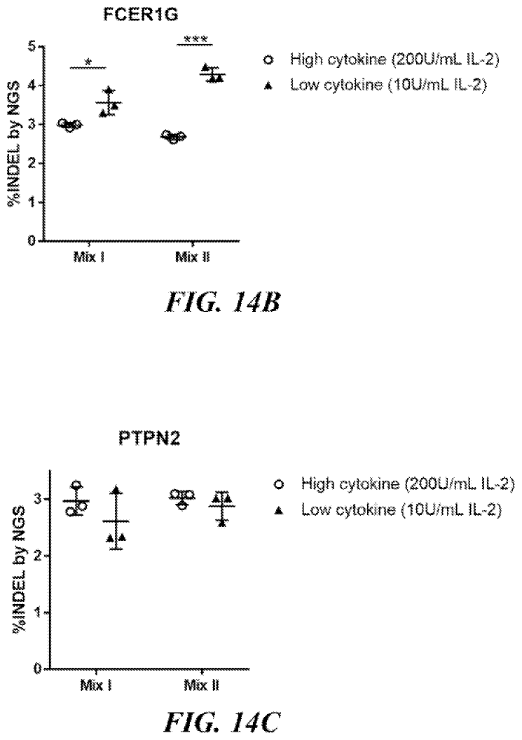

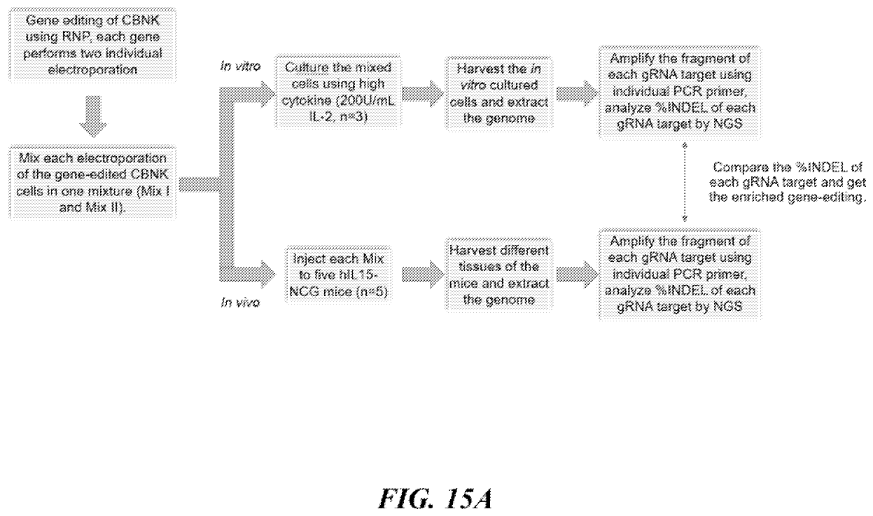

For example, to find the gene related to NK persistency, one or more of such population of engineered NK cells (e.g., eighteen kinds of gene-edited NK cells, such as gene-edited CBNK cells) were mixed in a mixture as disclosed herein and cultured using high cytokine (200 U/mL IL-2) or low cytokine (10 U/mL IL-2). Editing percentage (% INDEL) of each gene was analyzed by NGS after 8 days culturing. Two mixtures were prepared using two individual electroporation of each gene, three replicates were set for each culturing condition. A flowchart showing the method design is depicted in FIG. 14A.

Persistency with Low or High Cytokines (or in Sub-Optimal Environment or a More Optimal Environment)

By comparing the editing percentage of low-cytokine-cultured NK cells with high-cytokine-cultured NK cells, FCER1G deficient editing showed increased percentage when cultured with low cytokines, which indicated that FCER1G deficient NK cells had better persistency in low cytokine conditions, while PTPN2 had no significant difference in the same assay, as illustrated in FIG. 14B and FIG. 14C, respectively.

Thus, having reduced expression or activity level of an immune regulator polypeptide that is not PTPN2 (e.g., FCER1G) in a cell (e.g., stem cell, immune cell, such as NK cell, etc.) can induce enhanced persistence or survival rate in the cell.

In Vivo Screening of the NK Persistency Related Genes

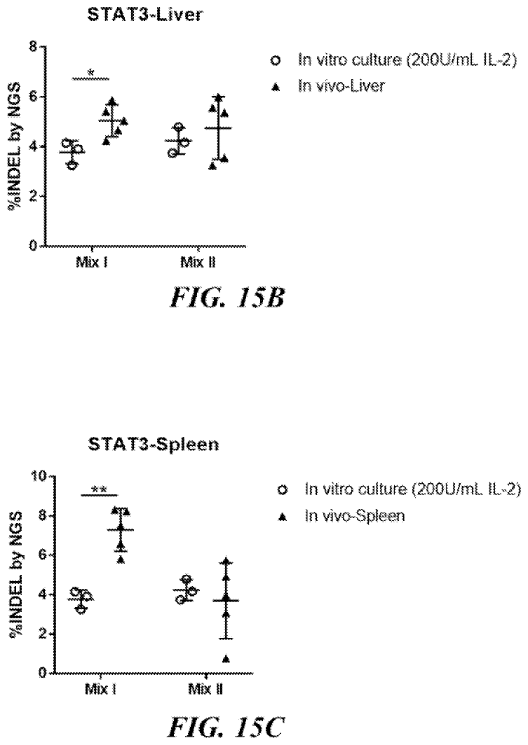

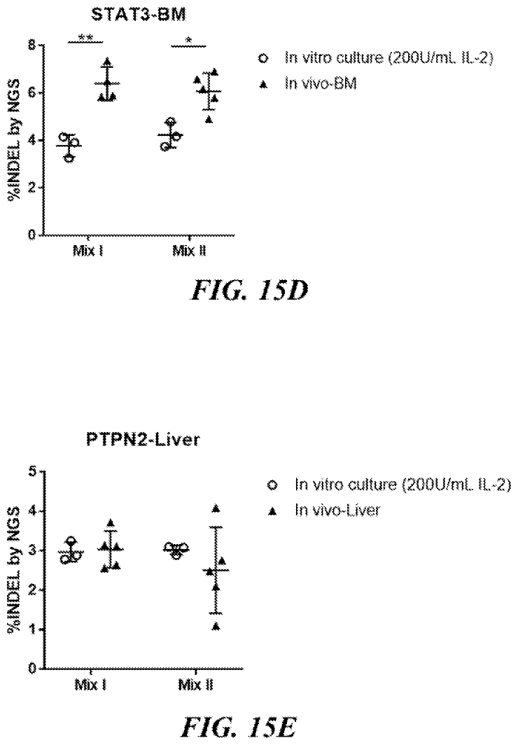

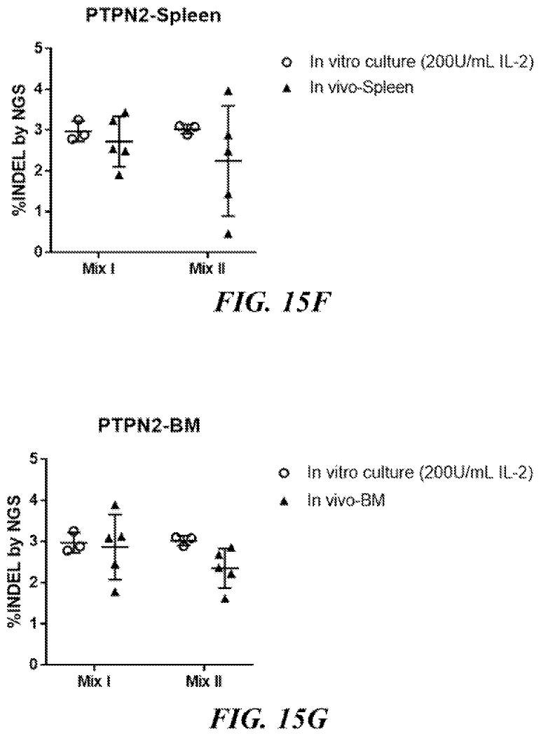

Similar to the in vitro screening for cell persistence, one or more of such population of engineered NK cells (e.g., eighteen kinds of gene-edited NK cells, such as gene-edited CBNK cells) were mixed in a mixture as disclosed herein and cultured using high cytokine (200 U/mL IL-2) (more optimal environment) or injected to hIL-15-NCG mice (sub-optimal environment), each mixed NK cells were injected to 5 mice with an amount (e.g., an amount of 1×10⁷ NK cells/mouse). Over time (e.g., after 8 days), in vitro cultured NK cells or mouse tissue are harvested to extract the genome. Editing percentage (% INDEL) of each gene is analyzed by NGS. Two mixtures were prepared using two individual electroporation of each gene, three replicates were set for in vitro culturing, five mice were used for each mixed NK cells injection. A flowchart showing the method design is depicted in FIG. 15A.

Persistency In Vivo

As shown in FIG. 15B-FIG. 15G, by comparing the editing percentage of NK cells in mouse tissues with high cytokine cultured NK cells, STAT3 deficient editing shown increased percentage in mouse tissue compared to cultured using high cytokine, which indicated that STAT3 deficient NK cells have better survival and persistency in vivo, while PTPN2 had no significant difference in the same assay.

Thus, having reduced expression or activity level of an immune regulator polypeptide that is not PTPN2 (e.g., STAT3) in a cell (e.g., stem cell, immune cell, such as NK cell, etc.) can induce enhanced persistence or survival rate in the cell.

### Example 5: Engineered CAR NK Cells

For improved cell therapy (e.g., adaptive immunotherapy, etc.), cells of interest can be engineered to comprise a heterologous chimeric polypeptide (e.g., a chimeric antigen receptor) comprising an antigen binding moiety against a specific antibody of a target cell (e.g., a cancer or tumor cell), to exhibit enhanced cytotoxicity against such target cell. The cells of interest can be immune cells (e.g., NK cells). Such immune cells can be derived from the stem cells as disclosed herein. For example, for NK cells derived from stem cells, one or more genetic modifications as disclosed herein can be introduced at (A) the stem cell state (e.g., iPSC state), (B) the hematopoietic stem cell state, and/or (C) the NK cell state. Alternatively, such immune cells can be immune cell lines (e.g., NK cell lines).

Engineered Anti-CD19 NK Cells

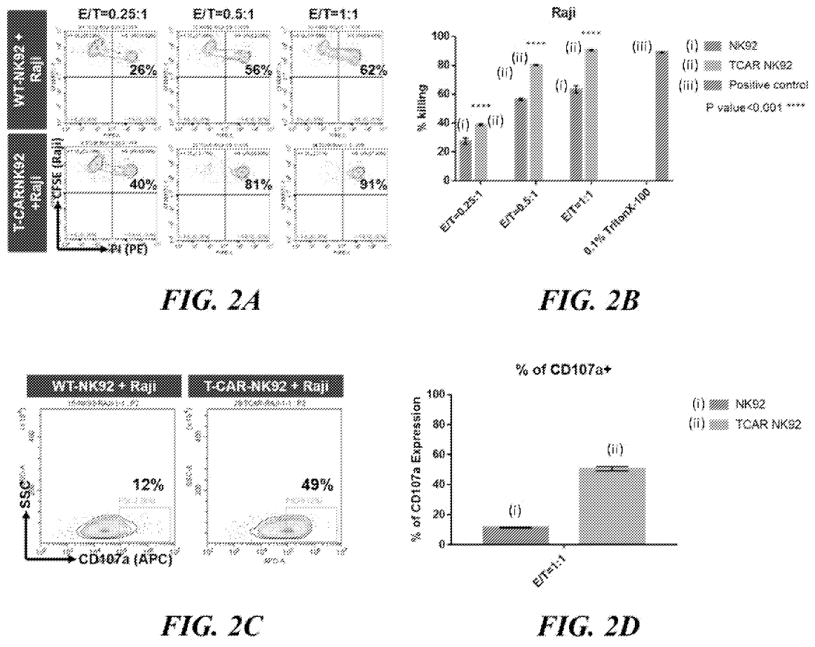

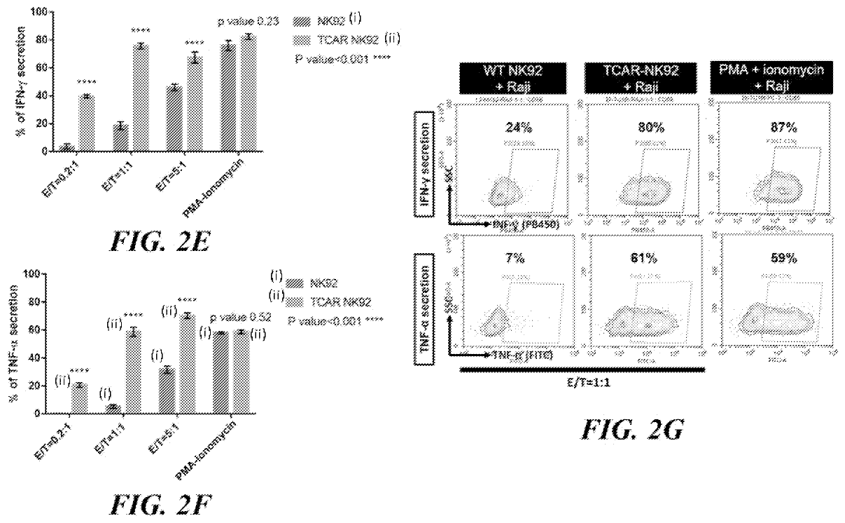

NK cells (e.g., NK-92 cells) were engineered to express anti-CD19 CAR, then cultured in the presence of CD19+ Raji cells to assess targeting of the Raji cells by the engineered anti-CD19 NK cells. Wild type (WT) NK-92 cells were used as control. The anti-CD19 CAR NK cells exhibited enhanced cytotoxicity against the Raji cells (as ascertained by a reduced number of alive Raji cells) as compared to the control (FIGS. 2A and 2B). In addition, when cultured in the presence of the Raji cells, the anti-CD19 CAR NK cells exhibited enhanced expression of endogenous CD107a (indicative of cytotoxic granule release) as compared to the control (FIGS. 2C and 2D). Furthermore, when cultured in the presence of the Raji cells, the anti-CD19 CAR NK cells exhibited enhanced cytokine production (e.g., IFN-gamma and/or TNF-alpha production) as compared to the control (FIGS. 2E-2G).

NK Cells Expressing CD33-CAR

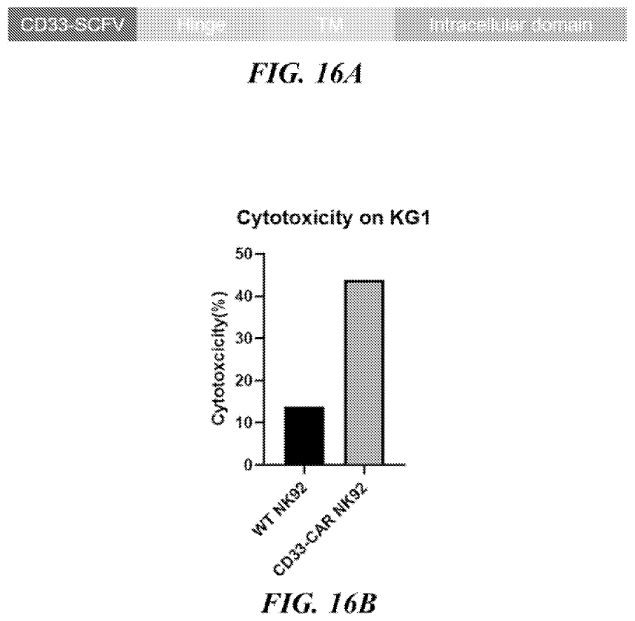

NK cells expressing a chimeric polypeptide receptor comprising an antigen binding moiety capable of specifically binding to CD33 were generated. Schematic of CD33 CAR structure design is shown in FIG. 16A. Targeted cytotoxicity of NK92 cells with CD33-CAR integration on KG1 cells, a tumor cell line with high expression of CD33, with an E/T (Effector/Target) ratio of 1:1 was tested. WT-NK92 cell were used as unmodified control. As shown in FIG. 16B, the targeted cytotoxicity of CD33-CAR on KG1 cells were greatly improved compared to control.

NK Cells Expressing BCMA-CAR

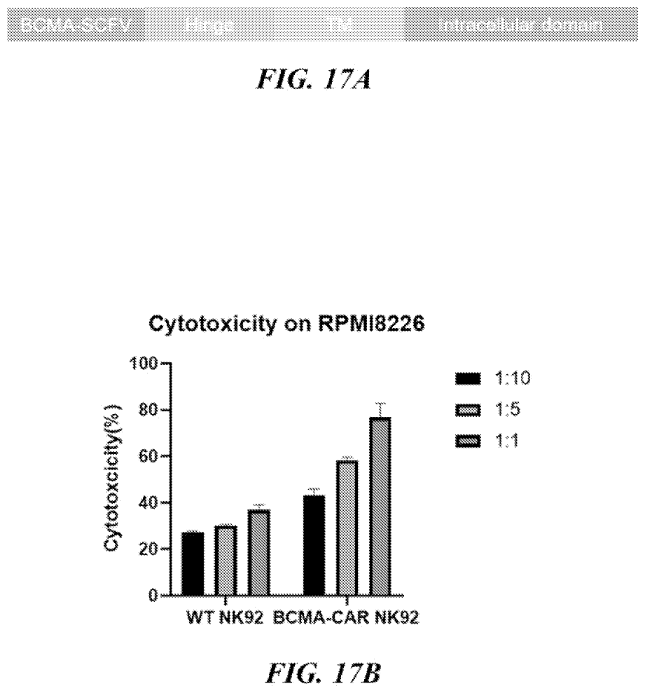

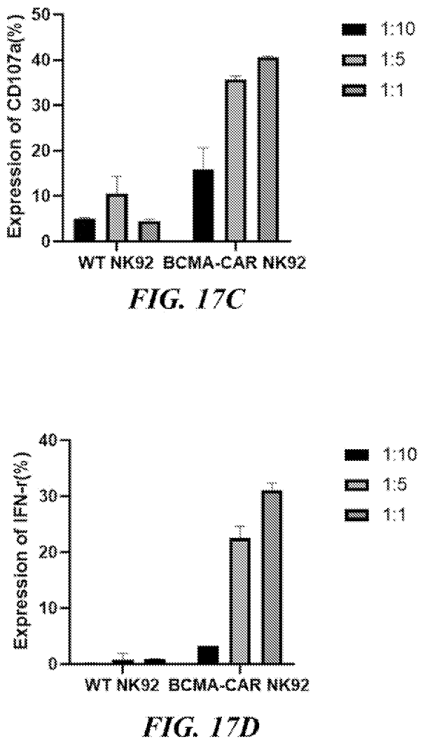

NK cells expressing a chimeric polypeptide receptor comprising an antigen binding moiety capable of specifically binding to BCMA was generated. Schematic of BCMA CAR structure design is shown in FIG. 17A. Targeted cytotoxicity, CD107a and INF-r expression of NK92 cells with BCMA-CAR integration on RPMI8826 cell, a tumor cell line with high expression of BCMA, are shown in FIGS. 17B-17D. E/T (Effector/Target) ratios used were 1:1; 1:5 and 1:10. WT-NK92 cell were used as unmodified control.

Different Chimeric Polypeptide Receptor Design Constructs

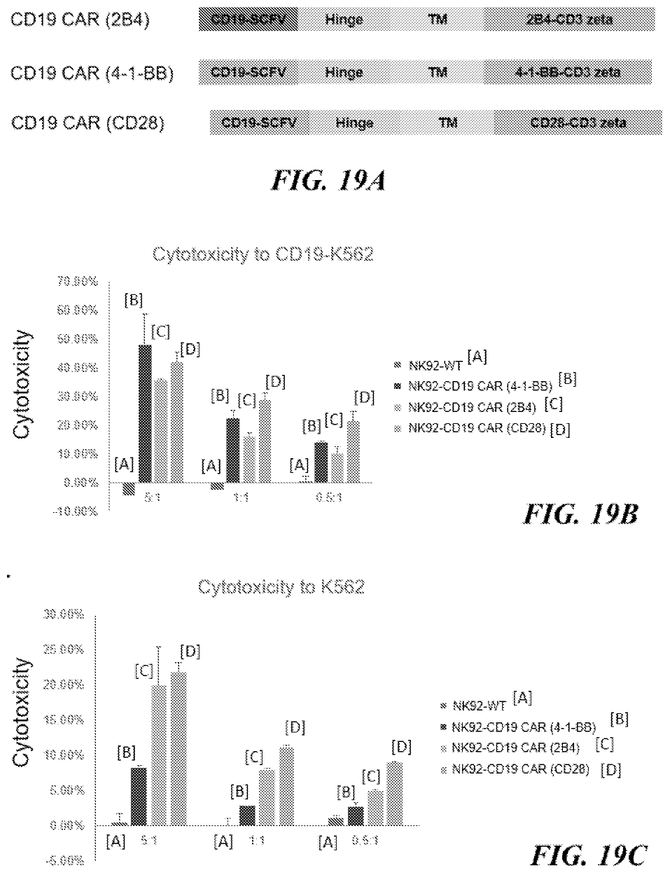

FIG. 19A illustrates different chimeric polypeptide receptor (e.g., CAR) constructs. FIG. 19A, Top schematically illustrates CD19 CAR (2B4) structure design. TM short for Transmembrane domain; SCFV short for single chain variable fragment. FIG. 19A, Middle schematically illustrates CD19 CAR (4-1-BB) structure design. TM short for Transmembrane domain; SCFV short for single chain variable fragment. FIG. 19A, bottom schematically illustrates CD19 CAR (CD28) structure design. TM short for Transmembrane domain; SCFV short for single chain variable fragment.

FIGS. 19B and 19C shows targeted cytotoxicity against target cells by NK cells expressing one of the chimeric polypeptide receptor design shown in FIG. 19A. Referring to FIG. 19B, targeted cytotoxicity of various CD19-CAR NK92 on CD19-K562 cells (E/T (Effector/Target) equals 5:1; 1:1 and 0.5:1) demonstrates that NK cells expressing CAR constructs with 4-1-BB signaling domain, 2B4 signaling domain, and/or CD28 signaling domain exhibited targeted cytotoxicity against CD19-presenting K562 target cells. WT-NK92 cell were used as unmodified control, CD19-K562 is K562 engineered with CD19 highly expressed. Referring to FIG. 19C, non-specific cytotoxicity of CD19-CAR NK92 on K562 cells that are not engineered to express CD19 at a high level (E/T (Effector/Target) equals 5:1; 1:1 and 0.5:1) demonstrated lower degree of cytotoxicity, indicating that 4-1-BB, 2B4, and/or CD28 intracellular signaling domains are useful in designing various CAR constructs, e.g., for immunotherapies such as NK cell therapies.

### Example 6: Engineered hIL15 NK Cells

For improved cell therapy (e.g., stem cell therapy, adaptive immunotherapy, etc.), cells of interest can be engineered to exhibit enhanced cytokine signaling (e.g., enhanced IL-15 signaling by enhanced or introduced IL-15, such as heterologous secretory IL-15, heterologous membrane-bound IL-15, heterologous IL-15 cytokine-IL15 receptor fusion, etc.). The cells of interest can be stem cells, such as isolated stem cells (e.g., embryonic stem cells) or induced stem cells (e.g., iPSCs). The cells of interest can be immune cells (e.g., NK cells). Such immune cells can be derived from the stem cells as disclosed herein. Alternatively, such immune cells can be immune cell lines (e.g., NK cell lines).

Engineered NK Cells:

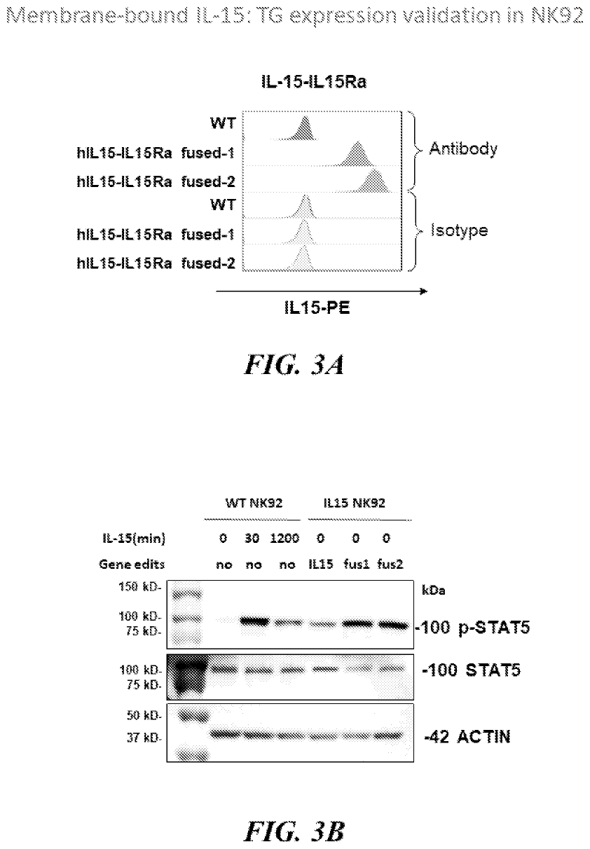

NK-92 cells were engineered with (i) hIL-15 knock in or (ii) hIL-15-hIL15R fusion polypeptide knock in. Two variants of the hIL-15-hIL15R fusion polypeptide were tested. The first variant (i.e., hIL15-IL15Ra fused-1 or “fus1”) was designed with a linker between hIL-15 and hIL15R, which linker comprising one or more (e.g., at least 1, 2, 3, 4, 5, 6, 7, 8, or more repeats) of “GGGGS” (SEQ ID NO. 9), e.g., “GGGGSGGGGSGGGGSGGGGSGGGGSGGGGS” (SEQ ID NO. 10). The second variant (i.e., hIL15-IL15Ra fused-2 or “fus2”) was designed with a linker between hIL-15 and hIL15R, which linker comprising one or more (e.g., at least 1, 2, 3, 4, 5, 6, 7, 8, or more repeats) of “GGGGS” (SEQ ID NO. 9) and one or more (e.g., at least 1, 2, 3, 4, 5, 6, 7, 8, or more repeats) of “EGKSSGSGSESKST” (SEQ ID NO. 11), e.g., “EGKSSGSGSESKSTEGKSSGSGSESKSTGGGGS” (SEQ ID NO. 12). NK-92 cells with either of the hIL-15-hIL15R fusion polypeptide variant knocked-in were positive for hIL-15 (FIG. 3A).

In addition, the engineered NK-92 cells expressing either variant of the hIL-15-hIL15R fusion polypeptide for enhanced IL-15 signaling exhibited longer persistency as compared to control NK-92 cells engineered express secretory form of IL-15. Western blotting analysis revealed increased phosphorylation of IL-15-stimulated STATS in the NK-92 cells expressing either hIL15-IL15Ra fused-1 (fus1) or hIL15-IL15Ra fused-2 (fus2), as compared to the secretory IL-15 (IL15) (FIG. 3B).

hIL15-IL15Ra fused-1 sequence (SEQ ID NO. 13):
MRISKPHLRSISIQCYLCLLLNSHFLTEAGIHVFILGCFS
AGLPKTEANWVNVISDLKKIEDLIQSMHIDATLYTESDVH
PSCKVTAMKCFLLELQVISLESGDASIHDTVENLIILANN
SLSSNGNVTESGCKECEELEEKNIKEFLQSFVHIVQMFIN
TSGGGGSGGGGSGGGGSGGGGSGGGGSGGGGSGITCPPPM
SVEHADIWVKSYSLYSRERYICNSGFKRKAGTSSLTECVL
NKATNVAHWTTPSLKCIRDPALVHQRPAPPSTVTTAGVTP
QPESLSPSGKEPAASSPSSNNTAATTAAIVPGSQLMPSKS
PSCYLKSRQTPPLASVEMEAMEALPVTWGTSSRDEDLENC
SHHL
hIL15-IL15Ra fused-2 sequence (SEQ ID NO. 14):
MRISKPHLRSISIQCYLCLLLNSHFLTEAGIHVFILGCFS
AGLPKTEANWVNVISDLKKIEDLIQSMHIDATLYTESDVH
PSCKVTAMKCFLLELQVISLESGDASIHDTVENLIILANN
SLSSNGNVTESGCKECEELEEKNIKEFLQSFVHIVQMFIN
TSEGKSSGSGSESKSTEGKSSGSGSESKSTGGGGSGITCP
PPMSVEHADIWVKSYSLYSRERYICNSGFKRKAGTSSLTE
CVLNKATNVAHWTTPSLKCIRDPALVHQRPAPPSTVTTAG
VTPQPESLSPSGKEPAASSPSSNNTAATTAAIVPGSQLMP
SKSPSTGTTEISSHESSHGTPSQTTAKNWELTASASHQPP
GVYPQGHSDTTVAISTSTVLLCGLSAVSLLACYLKSRQTP
PLASVEMEAMEALPVTWGTSSRDEDLENCSHHL

Membrane-Bound IL-15 Expression in iNK Cells Differentiated from hIL15-IL15Ra Fused-1 iPSC Clones

The expression of IL15 was confirmed in several iNK cells differentiated from hIL15-IL15Ra fused-1 iPSC clones, PW15, PW18, and PW23. As shown in FIG. 6, Fluorescence-activated Cell Sorting (FACS) was performed to quantify the surface expression of IL-15 in clones expressing membrane-bound IL-15 in comparison with controls, wild-type (wt) iNK cells and isotype. Thus, the clones were validated for expected overexpression level of membrane-bound IL-15.

In-Vitro Growth of eNK Cells Differentiated from Engineered iPSC

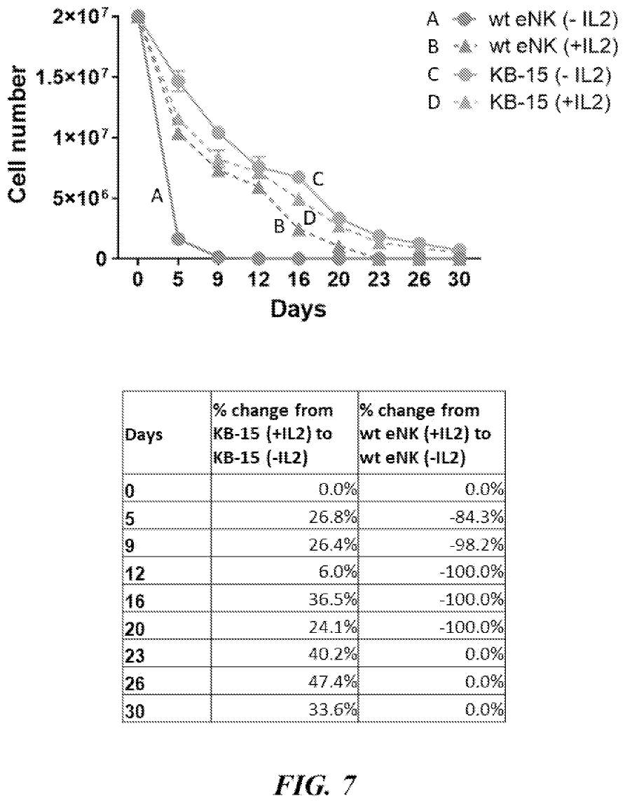

KB-15 cells were eNK cells (e.g., NK cells differentiated from iPSCs and subsequently expanded) differentiated from iPSC clones expressing hIL15-IL15Ra, aCD19 CAR, and a CD16 variant for enhanced CD16 signaling. The in-vitro growth of 2×10⁷ KB-15 cultured with or without IL-2 (100 U/mL) was monitored for 30 days. The cultured cells was collected and counted every 3 to 4 days, and the medium was renewed with corresponding medium meanwhile. As shown in FIG. 7, KB-15 cells were able to grow in the absence of exogenous cytokines as vigorously as the ones in the presence of exogenous cytokines.

For the engineered NK cells comprising enhanced IL-15 signaling (e.g., KB-15 NK cells comprising hIL15-IL15Ra), the engineered NK cells cultured in a medium substantially free of exogenous IL-2 exhibited enhanced persistence (e.g., on day 5, day 9, day 12, day 16, day 23, day 26, etc.) than the engineered NK cells cultured in a medium comprising exogenous IL-2.

Without wishing to be bound by theory, adding any exogenous IL-2 to the engineered NK cells comprising enhanced IL-15 signaling can interfere with the auto-activation of IL-15 or auto-activated IL-15 signaling pathway, thereby reducing the persistence or survival level of the engineered NK cells. For example, membrane-bound IL-15 can have a capability to induce downstream IL signaling (e.g., STAT pathway) to a higher degree than secreted IL (e.g., IL-2, IL-15), but the secreted IL at sufficient amount can act as a competitor for the same membrane receptor to reduce the chance of binding between the membrane-bound IL-15 and the respective receptor (e.g., IL-15R) and the signaling thereof. In contrast, for wild type NK cells (wt eNK in FIG. 7), absence of exogenous IL-2 in the medium resulted in reduced persistence level because the wild type NK cells did not have any membrane-bound IL-15 (e.g., IL15-IL15R fusion) to promote self-activation of the IL signaling pathway for persistence. Thus, when the engineered NK cells comprising enhanced IL-15 signaling is administered in vivo, where an amount of cytokines such as IL-2 in the blood stream or in a tissue of interest may be low (or sub-optimal), the engineered NK cells can exhibit optimal persistence or survival via self-induced activation of IL-15.

IPSC Expressing Membrane-Bound IL-15 Differentiated into iNK

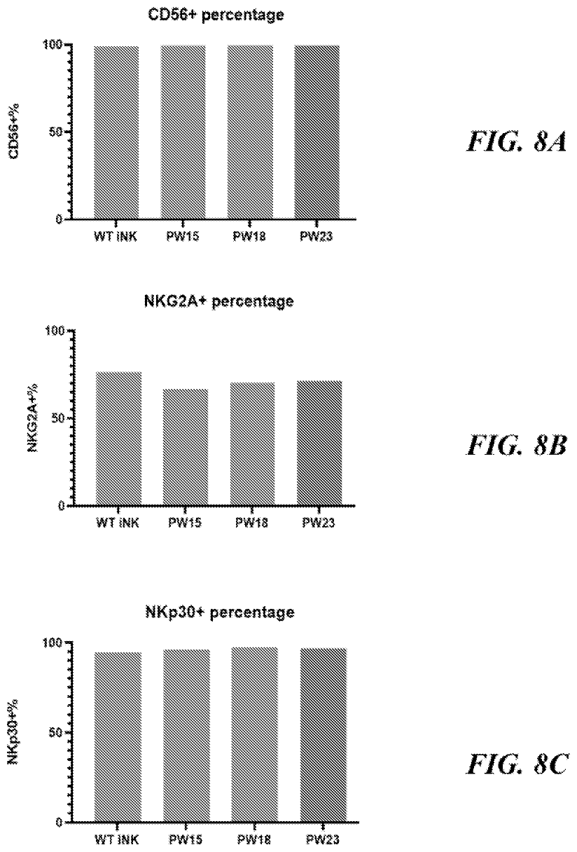

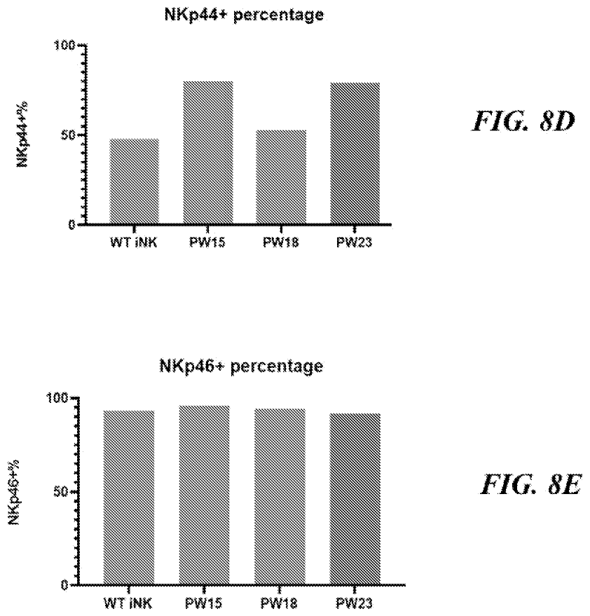

The engineered iPSC were subjected for iNK differentiation. Specifically, 5×10⁵ culture containing cells was collected and used for staining with NKG2A-PE, NKp30-PE, NKp44-PE, NKp46-PE and CD56-APC antibody. As shown in FIGS. 8A-8E respectively, CD56⁺ cells could be detected in the total differentiated cells, and NKG2A⁺, NKp30⁺, NKp44⁺, and NKp46⁺ cells could be detected in the CD56⁺ population among three exemplified clones, PW15, PW18 and PW23, which expressed membrane-bound IL-15. Therefore, iPSC expressing membrane-bound IL-15 were proven to be able to differentiate into iNK cells.

In-Vivo Pharmacokinetic Measurements of eNK Cells Expressing Membrane-Bound IL-15

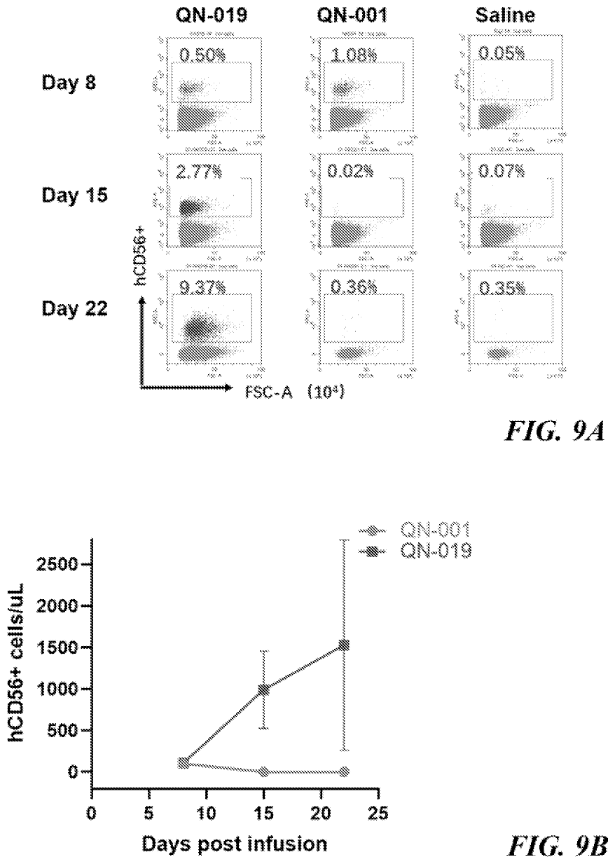

Triple immunodeficient NCG mice having a Nalm-6 xenograft model were used in an in-vivo pharmacokinetic assay. The mice lacked functional or mature T, B, and NK cells, and had reduced macrophage and dendritic cell function to host the xenograft model. eNK cells differentiated from iPSC edited with aCD19 CAR, a CD16 variant for enhanced CD16 signaling, and membrane-bound IL-15 (QN-019) were administered in the NCG mice, via intravenous (IV) tail vein injection, at a dose of about 1×10⁷ cells per animal. Similarly, eNK cells differentiated from WT iPSC (QN-001) were administered as a control. As shown in FIGS. 9A-9B, NK cell concentrations in the peripheral blood at 8, 15 and 22 days post-infusion were detected by FACS with CD56-APC antibody. CD56+ QN-019 cells proliferated significantly faster than QN-001 cells after infusion into the xenograft model, which proved a preferred pharmacokinetics profile in QN-019 cells.

iPSC Expressing Secretory IL-15 Differentiated into iNK

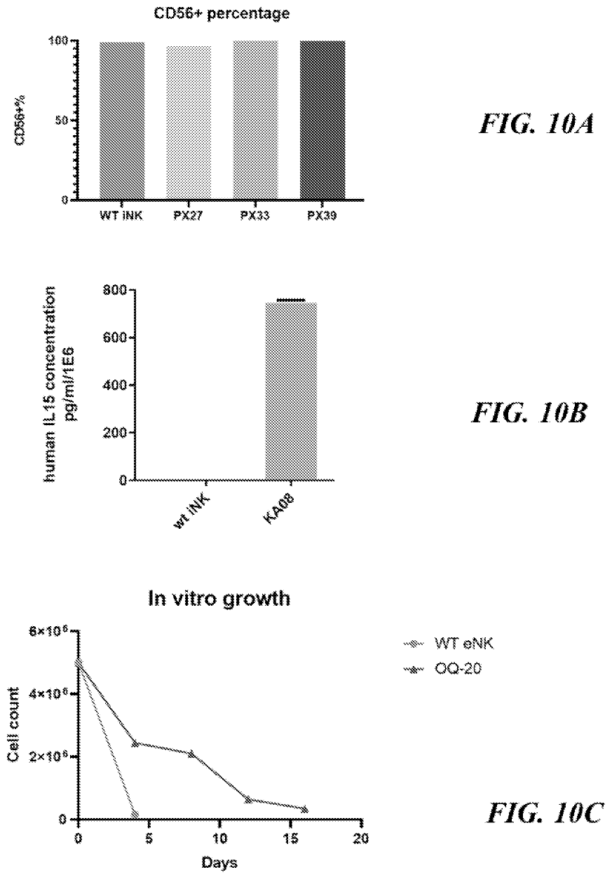

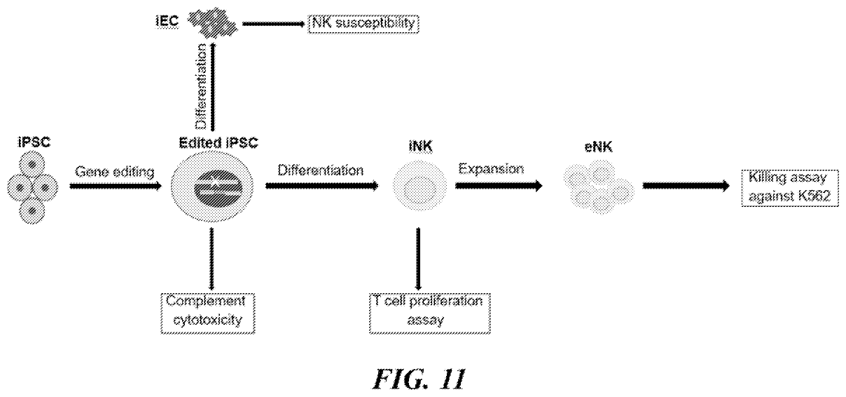

The engineered iPSC were subjected for iNK differentiation. Three clones expressing secretory IL-15, PX27, PX33, and PX39, were tested. Specifically, 1×10⁵ culture containing cells was collected and used for staining with CD56-APC antibody. FIG. 10A with the percentage of CD56⁺ cells in the total differentiated cells illustrates that iPSC expressing secretory IL-15 could differentiate into iNK.

Validation of Secretory IL-15 in Culture Medium

1×10⁶ iNK cells differentiated from iPSC clones expressing secreted IL-15, aCD19 CAR, and a variant for enhanced CD16 signaling (KA08) were cultured for 2 days without renewing the medium, and the supernatant was collected and diluted 10-fold to measure human IL-15 the Human IL-15 ELISA kit. FIG. 10B showing the concentration of IL-15 in iNK culture medium proves that the iNK cells were validated for secreting human IL-15 into culture medium.

In-Vitro Growth of eNK Cells Expressing Secretory IL-15

FIG. 10C shows the in-vitro growth curve of 5×10⁶ eNK cells differentiated from iPSC clones expressing secretory IL-15, aCD19 CAR, and a variant for enhanced CD16 signaling (OQ-20). The cultured cells were collected and counted every 4 days, and the medium in absence of exogenous cytokines was renewed with corresponding medium meanwhile. The growth of the cells within 16 days was recorded and plotted as curves. Therefore, it has been proven that eNK cells expressing secretory IL-15 facilitated in-vitro growth without exogenous cytokines.

### Example 7: Immune Regulator Polypeptide Gene Edits

For improved cell therapy (e.g., stem cell therapy, adaptive immunotherapy, etc.), cells of interest can be engineered to exhibit (i) reduced expression of one or more immune regulating polypeptides (e.g., one or more endogenous immune regulating polypeptides) and/or (ii) enhanced or introduced expression of one or more additional immune regulating polypeptides (e.g., one or more heterologous immune regulating polypeptides). Cells comprising (i) and/or (ii) as disclosed herein can exhibit enhanced function, such as a persistence level (or survival level), hypo-immunity (e.g., resistance against immune rejection or cytotoxicity), growth rate, cytotoxicity against a target cell (e.g., tumor cell), etc.

In some cases, having the combination of (i) and (ii) can synergistically improve function of the cells, as compared to having either one of (i) and (ii) alone, or a combination of individual effects of (i) and (ii), or none. In some cases, having reduced expression of two or more immune regulating polypeptides can synergistically improve function of the cells, as compared to having an individual member of the reduced expression of the two or more immune regulating polypeptides, or a combination of individual effects of such individual members. In some cases, having enhanced/introduced expression of two or more additional immune regulating polypeptides can synergistically improve function of the cells, as compared to having an individual member of the enhanced/introduced expression of the two or more additional immune regulating polypeptides, or a combination of individual effects of such individual members.

The cells of interest can be stem cells, such as isolated stem cells (e.g., embryonic stem cells) or induced stem cells (e.g., iPSCs). The cells of interest can be immune cells (e.g., NK cells). Such immune cells can be derived from the stem cells as disclosed herein. Alternatively, such immune cells can be immune cell lines (e.g., NK cell lines).

Engineered Cells:

Table 2 illustrates example combinations of modified expression or activity of the plurality of immune regulator polypeptides. A combination of modified expression or activity of the plurality of immune regulator polypeptides from Table 2 may be introduced in cells (e.g., engineered NK cells) to, for example, reduce or avoid immune response (e.g., immune attack, such as adaptive immune rejection) from a host's body upon administration of the cells to the host's body. A combination of modified expression or activity of the plurality of immune regulator polypeptides from Table 2 may comprise (i) reduced expression or activity of one or more first immune regulator polypeptides (column 2) and (ii) enhanced expression or activity of one or more second immune regulator polypeptides (column 3). In some cases, a combination of modified expression or activity of the plurality of immune regulator polypeptides from Table 2 may comprise (i) knock-out of one or more endogenous immune regulator polypeptide genes (column 2) and (ii) knock-in of one or more heterologous immune regulator polypeptide genes (column 3).

TABLE 2

| Modified | Reduced expression or activity of | Enhanced expression or activity of |
|---|---|---|
| expressions | immune regulator polypeptide genes | immune regulator polypeptide genes |
| or activities | (e.g., endogenous genes) | (e.g., heterologous genes) |
| 1 | B2M, CIITA, CD80, CD86, ICAM1, | IL2Ri, PD-L1, PD-L2, TGF-b, B2M-HLA- |
| | ICOSL, CD40L, MIC-A/B, ULBP-1 | E, HLA-G, CD47, IL-10, CCL-21, CD46, |
| | | CD55, CD59 |
| 2 | B2M, CIITA | PD-L1, B2M-HLA-E, CD47 |
| 3 | B2M, CIITA, CD80, CD86, ICAM1, | IL2Ri, PD-L1, PD-L2, TGF-b, B2M-HLA- |
| | CD40L, MIC-A/B | E, HLA-G, CD47 |
| 4 | B2M, CIITA, CD80, CD86, ICAM1, | IL2Ri, PD-L1, PD-L2, TGF-b, B2M-HLA- |
| | ICOSL, CD40L | E, CD47 |
| 5 | B2M, CIITA | IL2Ri, PD-L1, B2M-HLA-E, CD47 |
| 6 | B2M, CIITA, CD80, CD86 | PD-L1, B2M-HLA-E, CD47 |
| 7 | B2M, CIITA, ICAM1 | PD-L1, B2M-HLA-E, CD47 |
| 8 | B2M, CIITA, ICOSL | PD-L1, B2M-HLA-E, CD47 |
| 9 | B2M, CIITA, CD40L | PD-L1, B2M-HLA-E, CD47 |
| 10 | B2M, CIITA, CD80, CD86, ICAM1, | PD-L1, B2M-HLA-E, CD47 |
| | ICOSL, CD40L | |
| 11 | B2M, CIITA | IL2Ri, PD-L1, PD-L2, TGF-b, B2M-HLA- |
| | | E, CD47 |
| 12 | B2M, CIITA, MIC-A/B, ULBP-1 | PD-L1, B2M-HLA-E, HLA-G, CD47 |
| 13 | B2M, CIITA, MIC-A/B, ULBP-1 | PD-L1, B2M-HLA-E, CD47 |
| 14 | B2M, CIITA | PD-L1, B2M-HLA-E, HLA-G, CD47 |
| 15 | B2M, CIITA | PD-L1, B2M-HLA-E, CD47, IL-10, CCL- |
| | | 21 |
| 16 | B2M, CIITA | PD-L1, B2M-HLA-E, CD47, CD46, |
| | | CD55, CD59 |
| 17 | B2M, CIITA | PD-L1, B2M-HLA-E, CD47, CD55 |
| 18 | B2M | PD-L1, B2M-HLA-E, CD47 |
| 19 | B2M, CIITA | PD-L1, B2M-HLA-E |
| 20 | CIITA, CD80, CD86, ICAM1, ICOSL, | PD-L1, B2M-HLA-E, CD47 |
| | CD40L | |
| 21 | CIITA | IL2Ri, PD-L1, PD-L2, TGF-b, B2M-HLA- |
| | | E, CD47 |
| 22 | CIITA, CD80, CD86, ICAM1, ICOSL, | IL2Ri, PD-L1, PD-L2, TGF-b, B2M-HLA- |
| | CD40L | E, CD47 |
| 23 | B2M, CIITA, CD80, CD40L | PD-L1, B2M-HLA-E, CD47, CCL-21, |
| | | CD55 |
| 24 | CD80, CD86, ICAM1, CD40L | PD-L1, PD-L2, TGF-b, CD47 |
| 25 | B2M, CIITA | CD47 |

Generation of Modified NK Cells

For achieving hypo-immunity, NK cells can be engineered to carry certain transgenes and/or loss-of-function of genes of interest, such as the non-limiting exemplary guide RNA sequences are show in Table 6 below.

TABLE 6
Guide RNA sequences for generating
engineered NK cells with hypo-immunity

| SEQ ID | Target | Guide RNA | Guide RNA |
|---|---|---|---|
| NO | Name | sequence | position |
| 230 | NLRC5 | TTGCCGAGCT | chr16: |
| | | GGAGGCCAAC | 57020719-57020741: |
| | | | − |
| 231 | NLRC5 | ATCTTGGCGT | chr16: |
| | | TCAGCCATTC | 57020785-57020807: |
| | | | − |
| 232 | NLRC5 | ATGTCCAGGG | chr16: |
| | | TTCGGACACC | 57020897-57020919: |
| | | | + |
| 233 | RFXANK | GATGAGGTCT | chr19: |
| | | TCTGCAGGCT | 19193957-19193979: |
| | | | − |
| 234 | RFXANK | GTTCAGGATT | chr19: |
| | | CACAGGCTCA | 19194084-19194106: |
| | | | − |
| 235 | RFXANK | GAAACACTGG | chr19: |
| | | CATCCGGTTC | 19194100-19194122: |
| | | | − |
| 236 | RFXAP | CCGTTAGGTA | chr13: |
| | | CCTGTGCGAA | 36819566-36819588: |
| | | | + |
| 237 | RFXAP | GGCGGACCTG | chr13: |
| | | TTAGACACTT | 36819624-36819646: |
| | | | + |
| 238 | RFXAP | TGGCCAAGCA | chr13: |
| | | GCGCAAACCG | 36819815-36819837: |
| | | | + |
| 239 | CD80 | ATGGTCCGGT | chr3: |
| | | TCTTGTACTC | 119544693-119544715: |
| | | | + |
| 240 | CD80 | GAAATTGAGG | chr3: |
| | | TATGGACACT | 119557672-119557694: |
| | | | + |
| 241 | CD80 | TTGAGGTATG | chr3: |
| | | GACACTTGGA | 119557676-119557698: |
| | | | + |
| 242 | CD7 | CGGAACCGTC | chr17: |
| | | TGTCCGTAGT | 82316810-82316832: |
| | | | + |
| 243 | CD7 | CGCTGGCTCG | chr17: |
| | | CGGCCTGCCT | 82317430-82317452: |
| | | | − |
| 244 | CD7 | CGCGAGCCAG | chr17: |
| | | CGCCAGAAGC | 82317441-82317463: |
| | | | + |
| 245 | TAP2 | TCAAGACTTT | chr6: |
| | | GTTGTTCACC | 32837822-32837844: |
| | | | + |
| 246 | TAP2 | CATAGTCCTG | chr6: |
| | | GCAGCCCTTG | 32838118-32838140: |
| | | | + |
| 247 | TAP2 | GACTGTCTCC | chr6: |
| | | CTGAGAGCCC | 32837987-32838009: |
| | | | − |
| 248 | TAP1 | CTTGTAGAAT | chr6: |
| | | CCAGTCAGTG | 32852449-32852471: |
| | | | + |
| 249 | TAP1 | GTAGAATCCA | chr6: |
| | | GTCAGTGAGG | 32852452-32852474: |
| | | | + |
| 250 | TAP1 | TTACGGGCCG | chr6: |
| | | CCTCACTGAC | 32852459−32852481: |
| | | | − |
| 251 | TAPBP | GAACCAACAC | chr6: |
| | | TCGATCACCG | 33313815-33313837: |
| | | | + |
| 252 | TAPBP | GGTGATCGAG | chr6: |
| | | TGTTGGTTCG | 33313811-33313833: |
| | | | − |
| 253 | TAPBP | GATCGAGTGT | chr6: |
| | | TGGTTCGTGG | 33313808-33313830: |
| | | | − |

Optimized transgene sequence for NECTIN3/CD113
(SEQ ID NO. 262)
ATGGCGAGAACTCTGCGGCCAAGTCCACTTTGCCCTGGGG
GAGGAAAGGCTCAACTGTCTTCCGCCAGCCTCCTTGGGGC
CGGGCTGTTGCTCCAGCCACCCACACCGCCGCCCCTGCTG
CTCTTGCTTTTTCCTCTGTTGCTCTTTTCAAGGCTGTGCG
GTGCTCTCGCGGGGCCGATTATCGTAGAACCCCATGTAAC
TGCAGTATGGGGAAAGAACGTGTCCTTGAAGTGCCTTATT
GAGGTAAATGAGACAATCACGCAAATTAGTTGGGAGAAGA
TTCATGGAAAAAGTAGCCAAACAGTAGCTGTCCACCATCC
ACAGTATGGGTTCTCAGTACAGGGGGAGTACCAGGGGCGG
GTACTTTTTAAAAATTACTCCCTTAACGATGCGACTATCA
CCCTGCACAATATTGGTTTCTCAGATAGCGGCAAATACAT
TTGTAAGGCTGTGACATTCCCACTTGGAAACGCTCAATCC
TCAACGACGGTTACTGTTCTCGTTGAGCCTACTGTATCCC
TCATAAAGGGTCCGGACTCTTTGATTGATGGTGGTAACGA
GACTGTCGCAGCCATCTGTATAGCAGCGACTGGGAAGCCT
GTAGCGCATATTGATTGGGAGGGGGATTTGGGGGAGATGG
AGTCTACAACCACCAGTTTCCCCAATGAGACAGCTACAAT
CATTTCACAATACAAGTTGTTTCCAACGCGGTTCGCCCGG
GGACGCCGAATAACCTGTGTAGTTAAGCACCCGGCATTGG
AGAAGGACATTCGGTACTCCTTCATTCTGGATATTCAGTA
TGCTCCTGAAGTAAGTGTGACAGGTTACGATGGGAACTGG
TTTGTCGGACGGAAGGGTGTTAATTTGAAGTGCAACGCGG
ATGCAAACCCACCCCCTTTTAAGTCTGTCTGGTCTCGATT
GGACGGACAATGGCCCGACGGGTTGTTGGCGTCAGATAAT
ACTCTTCATTTTGTTCATCCCCTGACTTTTAATTATAGCG
GTGTCTACATCTGCAAAGTCACCAACTCCCTTGGTCAGCG
ATCCGACCAGAAGGTGATTTATATATCCGATCCTCCAACT
ACGACAACTTTGCAACCCACGATTCAGTGGCACCCGTCAA
CCGCTGACATAGAGGACTTGGCGACTGAACCAAAAAAACT
TCCTTTTCCGTTGAGTACACTCGCAACGATAAAAGATGAT
ACCATTGCCACTATAATTGCTTCCGTCGTTGGGGGGGCTC
TGTTTATCGTTCTTGTGTCCGTGTTGGCTGGAATATTCTG
CTACCGGCGCCGGCGAACCTTCCGCGGCGATTACTTCGCC
AAGAATTACATTCCTCCCTCTGACATGCAAAAGGAATCAC
AAATAGACGTGTTGCAACAGGACGAGCTTGATAGTTATCC
TGACTCTGTTAAGAAGGAAAATAAGAATCCAGTGAATAAT
CTTATACGAAAGGACTATCTGGAAGAGCCCGAAAAAACAC
AATGGAACAATGTGGAGAATTTGAACCGATTTGAGCGACC
CATGGATTACTATGAGGATCTTAAGATGGGAATGAAGTTC
GTTTCAGATGAGCATTATGATGAGAATGAAGATGACCTTG
TCAGCCACGTGGACGGTTCTGTCATCAGCAGAAGGGAATG
GTATGTCTAG
Optimized transgene sequence for ADORA2A/A2AR
(SEQ ID NO. 43)
ATGCCCATTATGGGCTCATCCGTCTATATAACAGTGGAGC
TCGCTATTGCCGTGCTTGCAATTCTCGGCAACGTATTGGT
CTGTTGGGCTGTTTGGCTTAACTCCAACCTTCAGAACGTG
ACTAATTATTTTGTAGTATCACTTGCTGCAGCCGATATAG
CTGTGGGGGTGCTCGCTATTCCTTTTGCGATTACAATATC
CACGGGCTTTTGTGCAGCATGTCACGGGTGTCTCTTCATA
GCCTGCTTCGTACTGGTGTTGACACAGTCATCCATTTTTA
GCCTTCTTGCCATCGCTATAGATCGCTATATTGCGATTCG
AATCCCCTTGCGCTATAACGGGCTGGTAACTGGCACAAGG
GCCAAAGGTATTATTGCTATATGTTGGGTCCTCTCATTTG
CTATTGGATTGACTCCGATGCTGGGGTGGAATAATTGTGG
ACAGCCGAAGGAGGGCAAAAACCATTCACAAGGCTGTGGG
GAAGGACAGGTAGCCTGCCTTTTTGAGGATGTGGTACCCA
TGAACTATATGGTCTATTTCAATTTCTTTGCATGCGTCCT
TGTACCGCTTTTGCTCATGCTGGGGGTTTACCTCAGGATA
TTTCTTGCCGCCCGAAGGCAACTTAAACAGATGGAATCTC
AACCGCTCCCAGGGGAGCGCGCCAGAAGTACACTGCAAAA
GGAAGTGCATGCCGCCAAAAGCCTCGCGATCATAGTTGGC
TTGTTTGCCTTGTGTTGGCTTCCCCTCCACATCATAAATT
GTTTTACTTTTTTTTGTCCCGACTGTTCACACGCGCCATT
GTGGCTCATGTACCTTGCGATTGTGCTCAGCCACACAAAC
TCAGTAGTTAATCCTTTCATCTACGCTTATAGAATTCGAG
AATTTCGCCAGACGTTTAGAAAGATTATACGGTCTCACGT
ATTGCGGCAGCAGGAACCTTTTAAGGCTGCTGGCACTTCT
GCTCGGGTGCTCGCGGCACATGGATCAGACGGCGAGCAGG
TCTCACTCAGACTGAACGGCCATCCACCAGGAGTTTGGGC
GAACGGCTCTGCTCCCCACCCTGAACGACGGCCTAATGGT
TACGCTCTTGGCCTGGTGTCTGGCGGTAGCGCCCAGGAAT
CACAAGGGAATACGGGTTTGCCAGATGTCGAGCTGCTTAG
CCACGAACTTAAAGGGGTTTGTCCAGAGCCGCCAGGCCTC
GATGATCCTCTTGCTCAGGACGGAGCTGGCGTGTCATGA

Generating Engineered NK Cells with Gene Knock-In

Human iPSC cells can be engineered by knocking in gene edits such as HLA-E, CD47, PDL2, HLA-G, TGF-beta, CCL21, IL10, CD46, CD55, and/or CD59. Such engineered iPSC cells can be differentiated into NK cells. Alternatively, human peripheral blood (PB)-NK cells can be engineered with AAV system. Possible functional readouts to test the engineered NK cells for hypo-immunity include mixed lymphocyte reaction (MLR), T cell activation assay, in vitro NK-cell-induced killing assay, and complement-dependent cytotoxicity.

Hypoimmunity Via Editing and Differentiating iPSC

A testing scheme for generating NK cells or endothelial cells (EC) derived from iPSC cells for hypo-immunity is shown in FIG. 11. Briefly, iPSC can be edited with different knock-ins and knock-outs. Subsequently, these edited iPSC can be subjected for differentiation into iNK which can be further expanded into eNK. iNK cells can be used for T cell proliferation assay, and eNK can be used for NK cytotoxicity test and hypoimmunity test. Alternatively, edited iPSC cells can be differentiated into iEC cells (e.g., endothelial cells derived from iPSCs) which can be used for NK susceptibility assay.

Methods of Generating Engineered iPSC

Several methods can be used to engineer iPSCs for NK cells with hypo-immunity. Exemplary methods are shown in Table 7.

TABLE 7
Methods for generating engineered iPSC

| KO genes | KO method | sgRNA sequence |
|---|---|---|
| B2M | RNP | GAGTAGCGCGAGCACAGCTA |
| | | (SEQ ID NO: 254) |
| CIITA | RNP | AGGCCCGGATGGCATCCTAG |
| | | (SEQ ID NO: 255) |
| ICAM1 | RNP | GGCAGGCTTGCATTCCCTCC |
| MICA | ABE | (SEQ ID NO: 257) |
| MICB | ABE | AGGGGCCATGGGGCTGGGCC |
| | | (SEQ ID NO: 258) |
| ULBP1 | ABE | ACTCACCGACCCATCCTGCC |
| | | (SEQ ID NO: 259) |

Briefly, RNP method is a method of electroporating target cells with pre-mixed ribonucleoprotein (RNP) containing Cas9 and sgRNA. After delivery to the cells, the RNP edits the genome region paired to the sgRNA. Adenine base editor (ABE) method is a method where Cas proteins can be fused to an enzyme that can deaminate a DNA nucleoside.

Confirmation of Edit-1 Clones

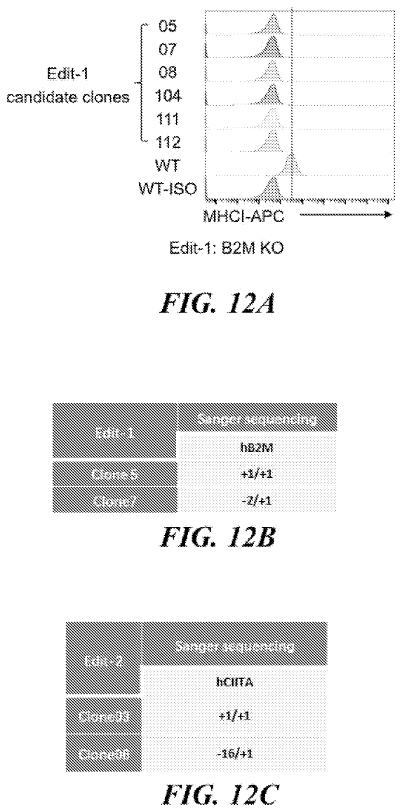

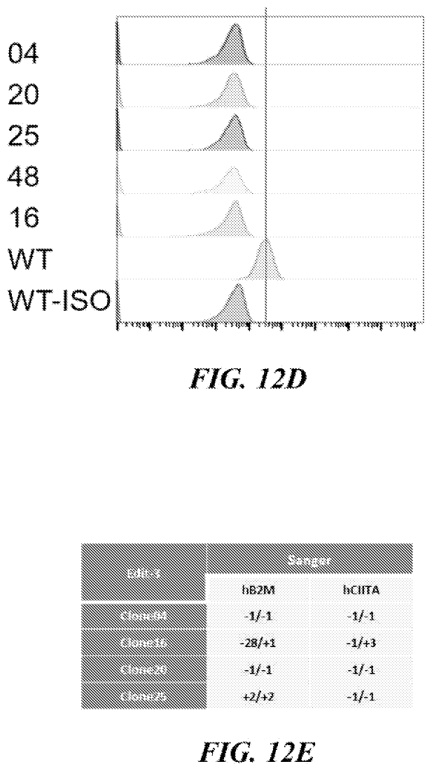

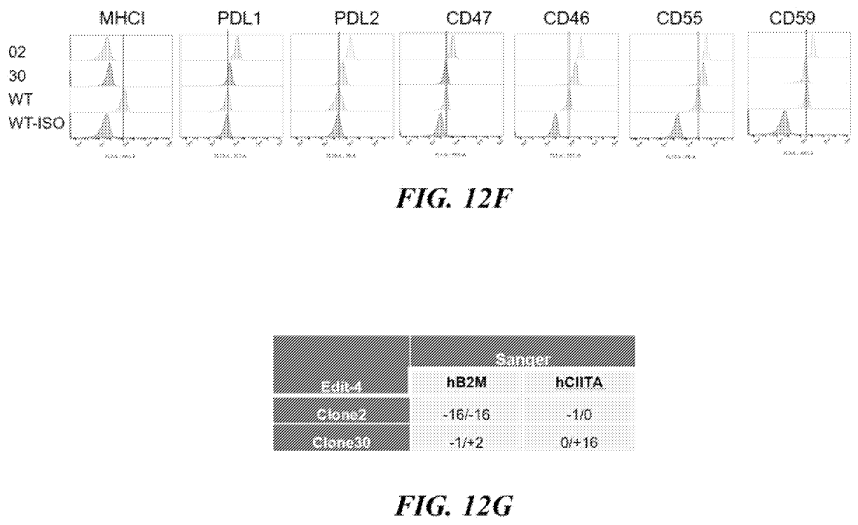

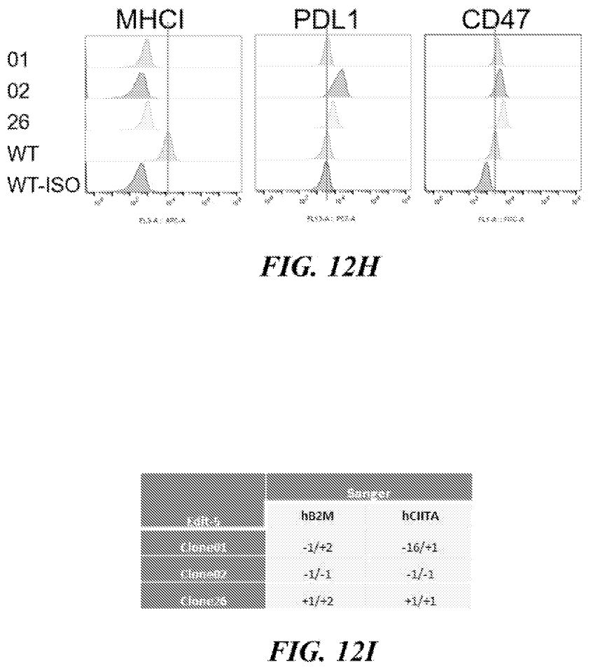

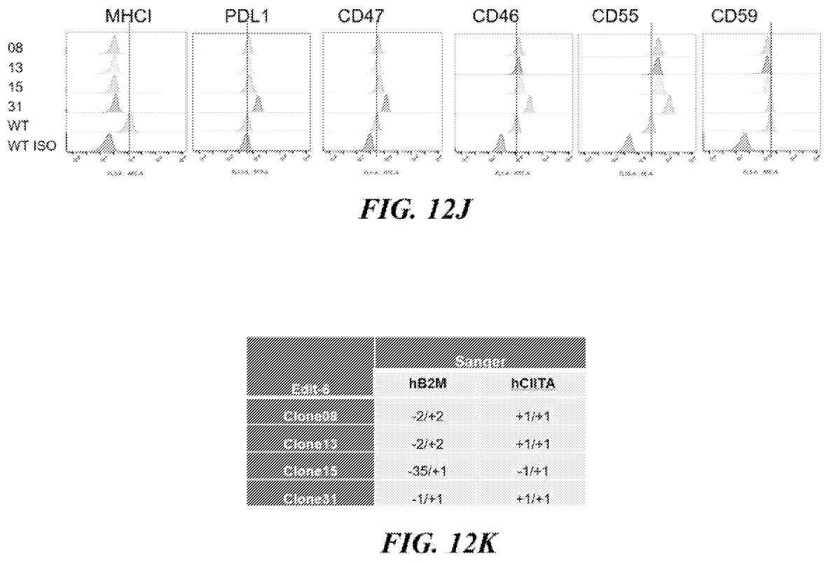

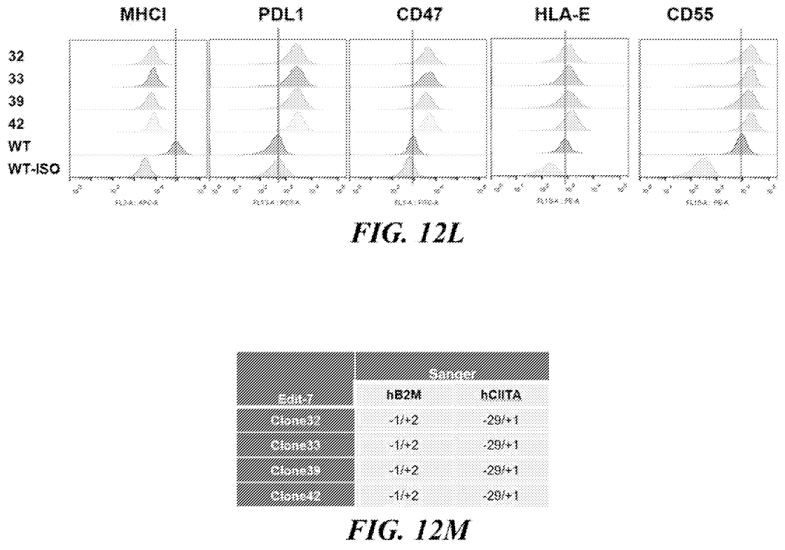

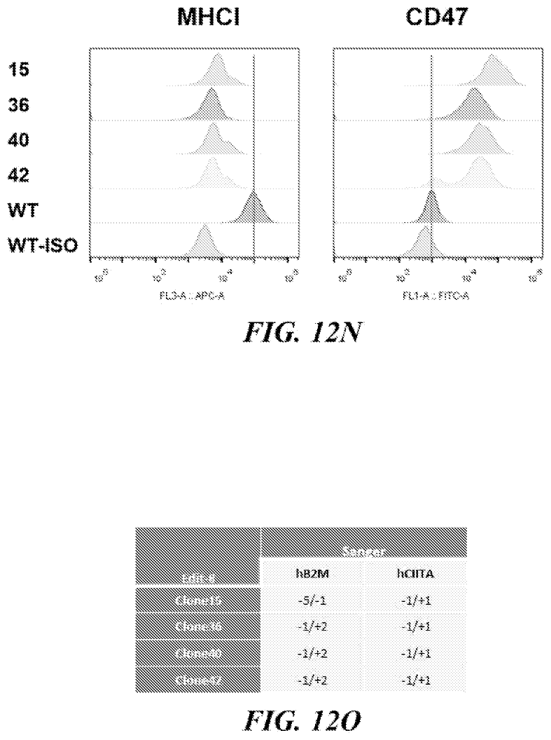

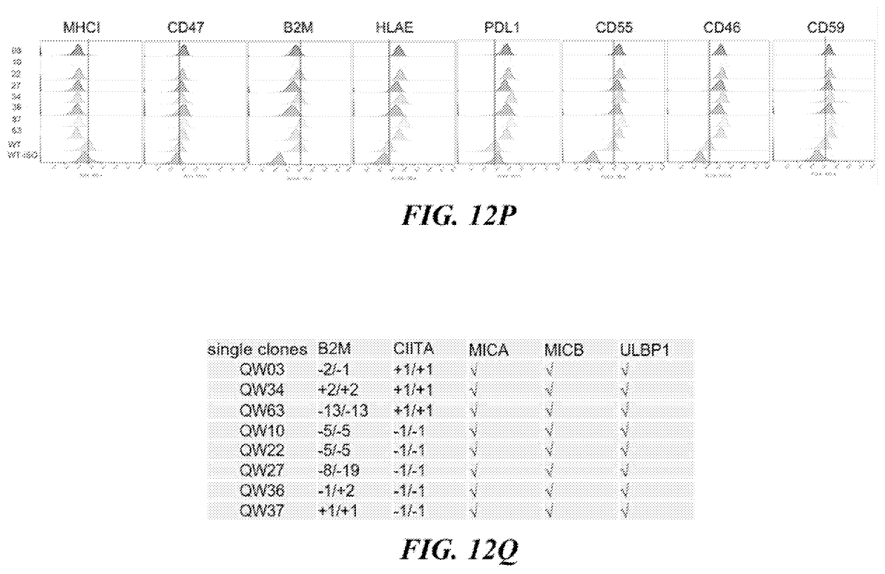

Several edit-1 clones, clones 05, 07, 08, 104, 111, 112, were derived by electroporating human iPSC with RNP targeting B2M. The Edit-1 clones were confirmed to be B2M knock-out by FACS analysis for MHC-I (see FIG. 12A) and by sanger sequencing (FIG. 12B). Briefly, clone 05 was sequenced to have an insertion of one nucleotide in both B2M alleles, and clone 07 was sequenced to have an insertion of one nucleotide in one B2M allele and a deletion of two nucleotides in the other allele.

Confirmation of Edit-2 Clones

Similarly, several edit-2 clones, clones 03 and 06, were derived by electroporating human iPSC with RNP targeting CIITA, followed by FACS sorting for single cells. The edit-2 clones were confirmed to be CIITA KO by sanger sequencing. As shown in FIG. 12C, clone 03 was sequenced to have an insertion of one nucleotide in both CIITA alleles, and clone 06 was sequenced to have an insertion of one nucleotide in one CIITA allele and a deletion of sixteen nucleotides in the other allele.

Confirmation of Edit-3 Clones

Several edit-3 clones, clones 04, 20, 25, 48, and 16, were derived by electroporating hiPSC with two RNPs, targeting B2M and CIITA, respectively. The Edit-3 clones were confirmed to be B2M knock-out by FACS analysis for MHC-I (see FIG. 12D) and sanger sequencing was used to confirm the knock-out of B2M and CIITA at the genomic level (see FIG. 12E).

Confirmation of Edit-4 Clones

Several Edit-4 clones, clones 02 and 30, were derived by electroporating hiPSC with a construct overexpressing PD-L1, PD-L2, TGF-β, HLA-E, HLA-G, CD47, IL-10, CCL-21, CD46, CD55, CD59 and two RNPs, targeting B2M and CIITA, respectively, followed by FACS sorting for B2M⁻; PDL1⁺; CD47⁺ single cells. The Edit-4 clones were confirmed to be B2M− by FACS analysis for MHC-I, and PDL1+, PD2+, CD47+, CD46+, CD55+ and CD59+ (see FIG. 12F) and sanger sequencing was used to confirm the knock-out of B2M and CIITA at the genomic level. For CIITA, only 1 allele was confirmed to be knock-out (see FIG. 12G).

Confirmation of Edit-5 Clones

Several Edit-5 clones, clones 01, 02, and 26, were derived by electroporating hiPSC with a construct overexpressing of PD-L1, HLA-E, CD47, IL-10, CCL-21 and two RNPs, targeting B2M and CIITA, respectively, followed by FACS sorting for B2M−; PDL1+; CD47+ single cells. The Edit-5 clones were confirmed to be B2M− by FACS analysis for MHC-I, and PDL1+, CD47+(see FIG. 12H) and sanger sequencing was used to confirm the KO of B2M and CIITA at the genomic level (see FIG. 12I).

Confirmation of Edit-6 Clones

Several Edit-6 clones, clones 08, 13, 15, and 31, were derived by electroporating hiPSC with a construct overexpressing PD-L1, HLA-E, CD47, CD46, CD55, CD59 and two RNPs, targeting B2M and CIITA, respectively, followed by FACS sorting for B2M−; PDL1+; CD47+ single cells. The Edit-6 clones were confirmed to be B2M⁻ by FACS analysis for MHC-I, and PDL1+, CD47+, CD46+, CD55+, CD59+ (see FIG. 12J) and sanger sequencing was used to confirm the KO of B2M and CIITA at the genomic level (see FIG. 12K).

Confirmation of Edit-7 Clones

Several Edit-7 clones, clones 32, 33, 39, and 42, were derived by electroporating hiPSC with a construct overexpressing PD-L1, HLA-E, CD47, CCL-21, CD55 and two RNPs, targeting B2M and CIITA, respectively, followed by FACS sorting for B2M⁻; PDL1+; CD47+ single cells. The Edit-7 clones were confirmed to be B2M⁻ by FACS analysis for MHC-I, and PDL1+, CD47+, HLA-E+, CD55+ (see FIG. 12L) and sanger sequencing was used to confirm the KO of B2M and CIITA at the genomic level (see FIG. 12M).

Confirmation of Edit-8 Clones

Several Edit-8 clones, clones 15, 36, 40, and 42, were derived by electroporating hiPSC with a construct overexpressing of CD47 and two RNPs, targeting B2M and CIITA, respectively, followed by FACS sorting for B2M⁻; CD47+ single cells. The Edit-8 clones were confirmed to be B2M− by FACS analysis for MHC-I, and CD47+(see FIG. 12N) and sanger sequencing was used to confirm the KO of B2M and CIITA at the genomic level (see FIG. 12O).

Confirmation of Edit-9 Clones

Several Edit-9 clones, clones 03, 10, 22, 27, 34, 36, 37, and 63, were derived by electroporating hiPSC with a construct overexpressing PD-L1, PD-L2, TGF-β, HLA-E, HLA-G, CD47, IL-10, CCL-21, CD46, CD55, CD59, two RNPs, targeting B2M and CIITA, and 1 Base Editor plasmid overexpressing sgRNA targeting MICA, MICB and ULBP1, followed by FACS sorting for B2M⁻; PDL1+; CD47+ single cells. The Edit-9 clones were confirmed to be B2M⁻ by FACS analysis for MHC-I, and CD47+, B2M+, HLA-E+, PDL1+, CD55+, CD46+ and CD59+(see FIG. 12P) and sanger sequencing was used to confirm the KO of B2M and CIITA at the genomic level. The KO of MICA/MICB/ULBP1 were confirmed using next generation sequencing. The symbol ✓ represents the gene was knocked out successfully (see FIG. 12Q).

Resistance to Antibody-Mediated Complement Cytotoxicity

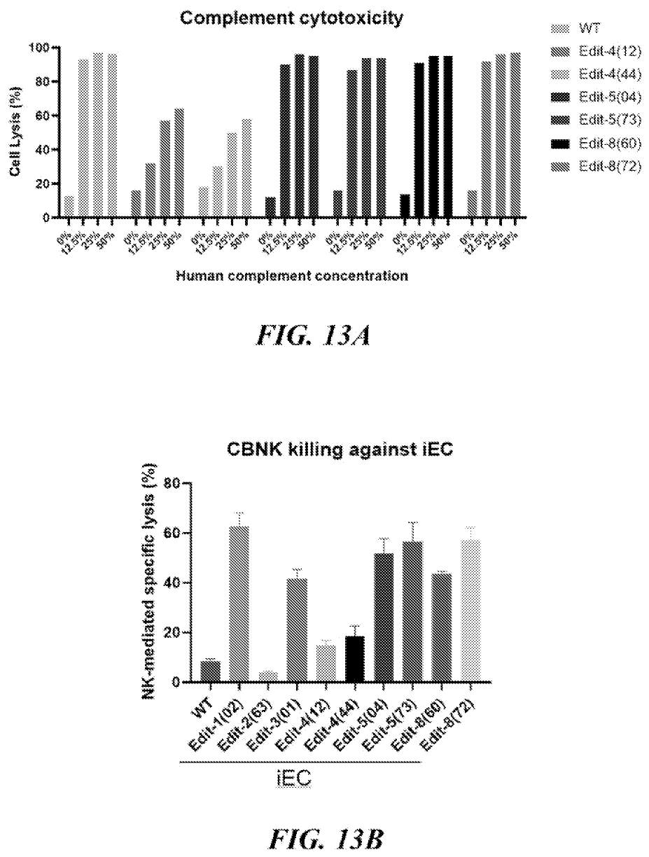

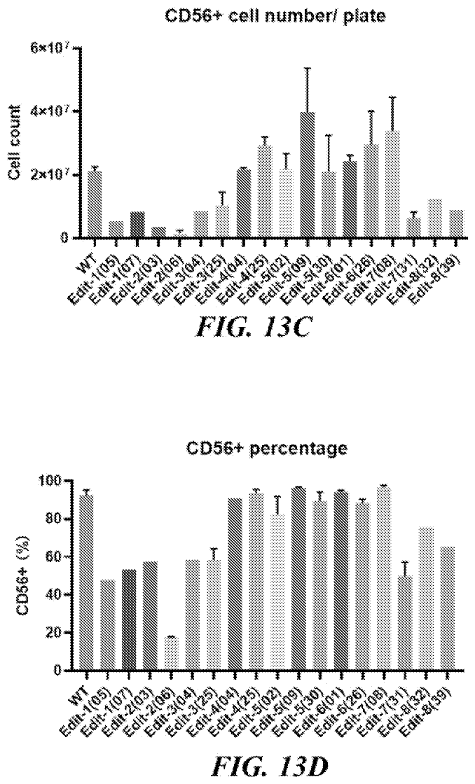

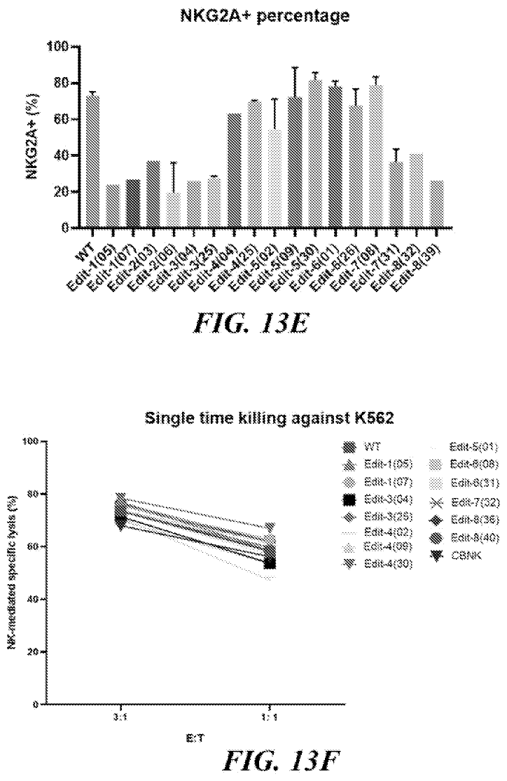

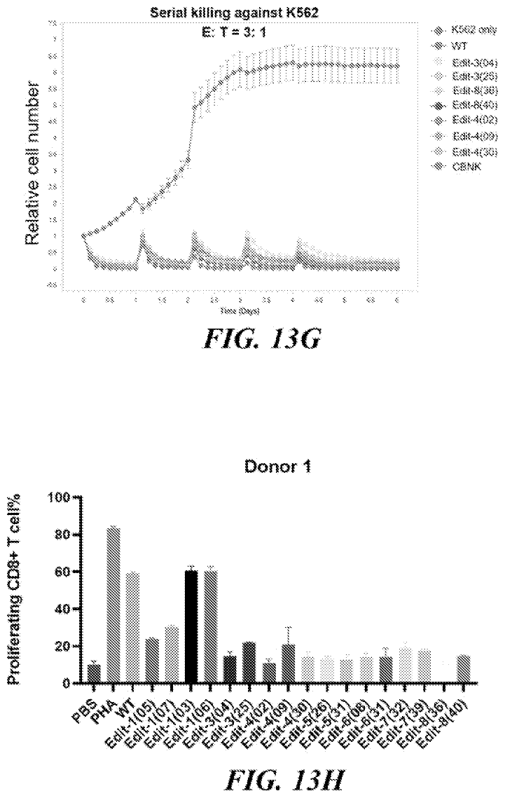

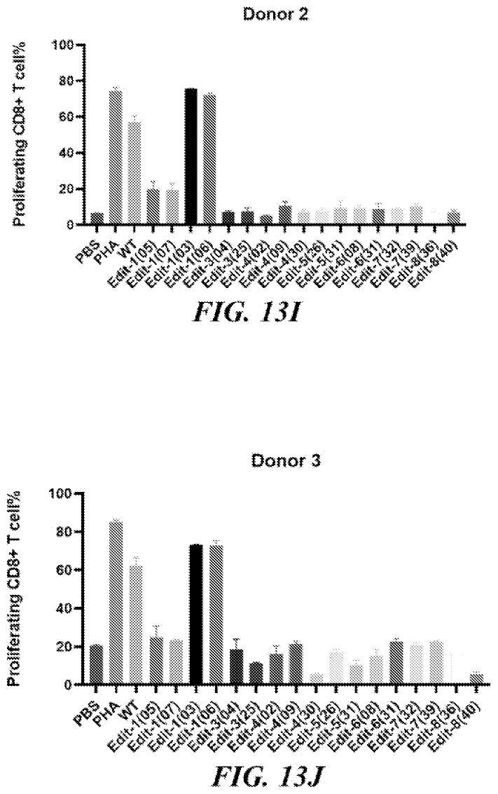

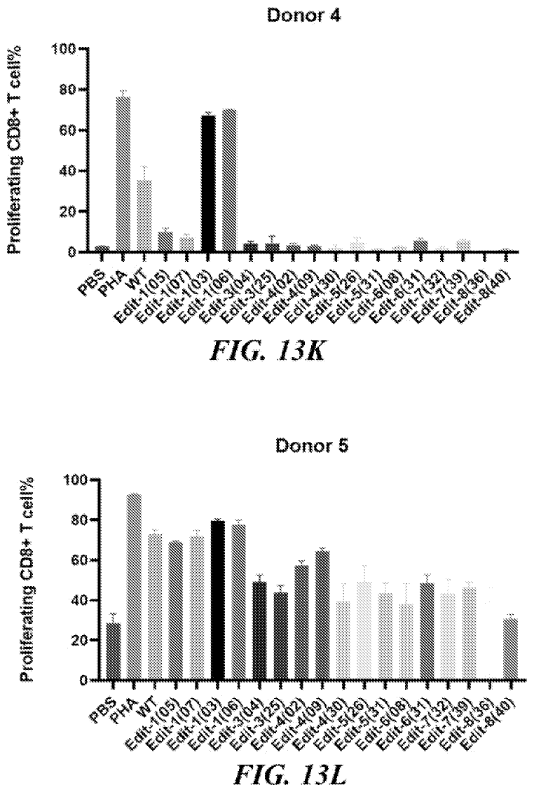

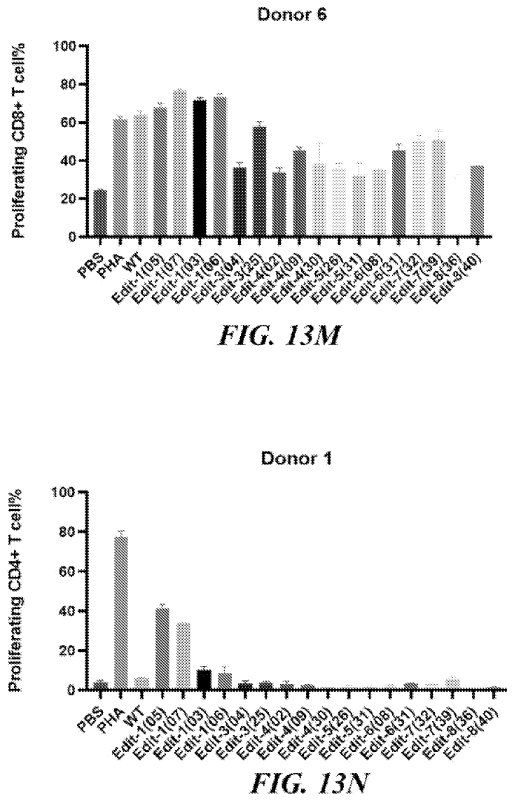

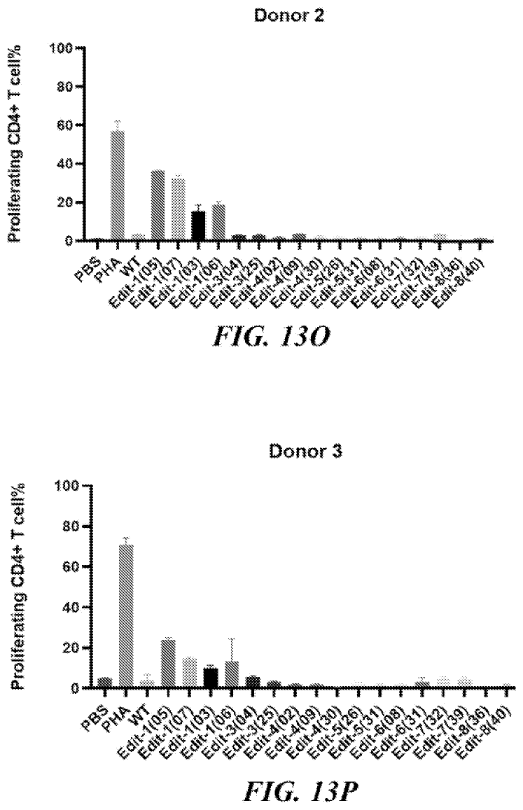

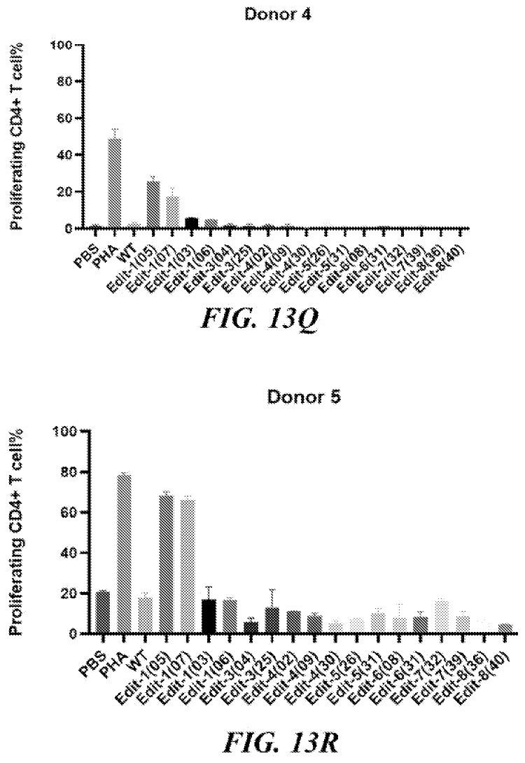

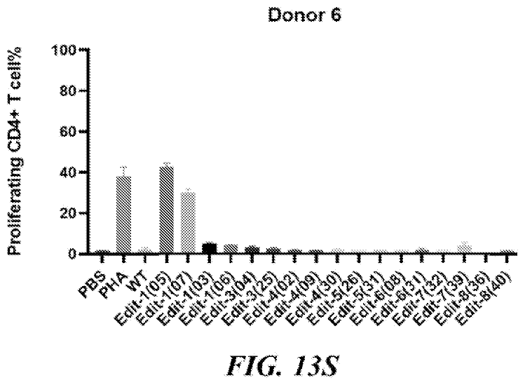

As shown in FIG. 13A, different edited iPSC clones were prelabeled with SSEA-4 antibody, followed by co-incubating for 45 min with human complement at different concentration ranging from 0%˜50%. The results demonstrated that iPSC with Edit-4 but not Edit-5 or Edit-8 were resistant to antibody-mediated complement cytotoxicity.

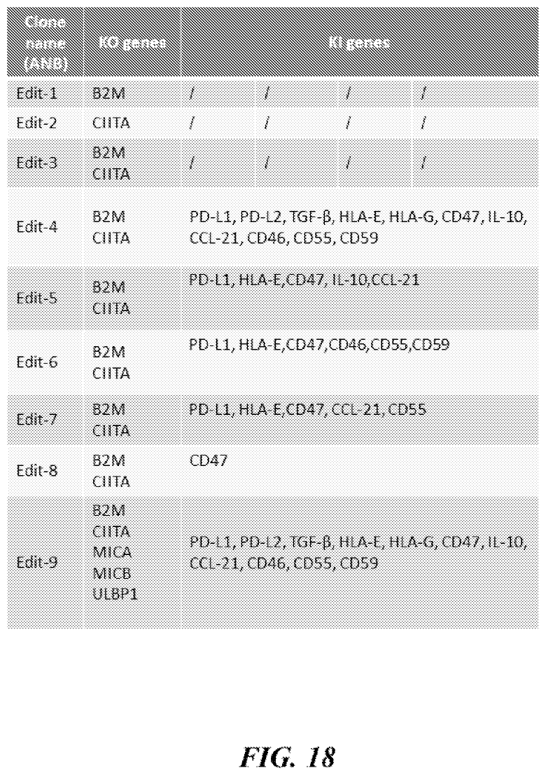

Without wishing to be bound by theory, the enhanced resistance to antibody-mediated complement cytotoxicity may be attributed to having enhanced or introduced expression of one or more of the following immune regulator polypeptides: PD-L2, TGF-beta, CD46, CD55, CD59, and HLA-G (e.g., at least one or more of PD-L2, TGF-beta, CD46, CD55, and CD59). Thus, without wishing to be bound by theory, cells comprising Edit-9 (see FIG. 18) may also exhibit enhanced resistance to antibody-mediated complement cytotoxicity, as compared to cells with either Edit-5 or Edit-8.

Without withing to be bound by theory, iPSCs with Edit-4 or Edit-9 can exhibit enhance resistance to antibody-mediated complement cytotoxicity in vitro or in vivo, as compared to that of iPSCs with either Edit-5 or Edit-9. Without withing to be bound by theory, differentiated cells (e.g., endothelial cells, immune cells, etc.) derived from iPSCs comprising Edit-4 or Edit-9 can exhibit enhance resistance to antibody-mediated complement cytotoxicity in vitro or in vivo, as compared to that of comparably differentiated cells derived from iPSCs with either Edit-5 or Edit-9. Without withing to be bound by theory, immune cells derived from iPSCs comprising Edit-4 or Edit-9 can exhibit enhance resistance to antibody-mediated complement cytotoxicity in vitro or in vivo, as compared to that of immune cells derived from iPSCs with either Edit-5 or Edit-9. Without withing to be bound by theory, NK cells derived from iPSCs comprising Edit-4 or Edit-9 can exhibit enhance resistance to antibody-mediated complement cytotoxicity in vitro or in vivo, as compared to that of NK cells derived from iPSCs with either Edit-5 or Edit-9.

CBNK Cytotoxicity

Targeted cells iEC differentiated from corresponding iPSC were pre-labeled with CFSE, and then co-cultured for 4 h with CBNK at an Effector: Target ratio of 5:1. After 4 h, PI was added for staining dead cells. FACS was used to detect the percentage of dead cell. iEC without co-cultured with CBNK was regarded as spontaneous target cell death. To calculate the percentage of specific NK cell-mediated target cell lysis, the following formula was used: NK-mediated specific lysis (%)=(% PI positive target cells in coculture−% PI positive target cells in spontaneous cell death)/(100−% PI positive target cells in spontaneous cell death). The quantification is shown in FIG. 13B, which indicates iEC differentiated from corresponding iPSC with Edit-4 but not Edit-5 or Edit-8 were resistant to CBNK cytotoxicity

Efficiency of Differentiation from iPSC

The engineered iPSC were subjected for iNK differentiation. 200 ul of the culture containing cells was collected and used for CD56 and NKG2A staining After staining, the cells were resuspended in 100 ul FACS buffer (PBS+1% BSA). 80 ul of the cell suspension were collected for analysis. The yield of CD56+ cell number was calculated as followed: (Absolute CD56+ cells collected by FACS)*18 ml/(200 ul*0.8)=(Absolute CD56+ cells collected by FACS)*112.5 (see FIG. 13C); the CD56+ and NKG2A+ percentage were shown in FIGS. 13D and 13E, respectively. Suggested from the above figures, iPSC with different edits could all differentiate into iNK, with varied yields and efficiencies.

Functional Property of NK Cells with Hypo-Immunity

FIG. 13F shows single time killing against K562. Corresponding eNK was co-cultured with CFSE-prelabeled K562 cells at a ratio of E:T=3:1 or 1:1. After 4 h, PI was added for staining dead cells. FACS was used to detect percentage of dead cell. K562 without co-cultured with eNK was regarded as spontaneous target cell death. To calculate the percentage of specific NK cell-mediated target cell lysis, the following formula was used: NK-mediated specific lysis (%)=(% PI positive target cells in coculture−% PI positive target cells in spontaneous cell death)/(100−% PI positive target cells in spontaneous cell death).

FIG. 13G shows serial killing against K562. Corresponding eNK was co-cultured with K562-EGFP cells at a ratio of E:T=3:1, the co-culture was performed in Incucyte. K562-EGFP cells were monitored by the imaging with a frequency of every 3 h. K562 was added daily for 6 days. K562-GFP only sample was set as a control.

FIGS. 13F and 13G indicate that B2M/CIITA double Knock-Out eNK did not show any hyporesponsive phenotype against K562 cells, and most eNK with transgenes had comparable cytotoxicity to WT eNK, when tested against K562 cells. Without wishing to be bound by theory, the hyporesonsivitiy or cytotoxicity against other target cells can be different.

To test whether the iNK with different edits would stimulate the proliferation of CD8+ T cells in PBMC, corresponding eNK was co-cultured with CFSE-labeled PBMC. PBS was used a negative control while PHA as a positive control. After 5 days, the co-cultured cells were stained for CD3, CD4 and CD8. CD3⁺CD8⁺CFSE^{low} were regarded as proliferating CD8⁺ T cells. PBMC from different donors were tested in FIGS. 13H-13M. The data show that B2M/CIITA double Knock-Out NK with or without transgenes did not stimulate CD8+ T cell proliferation.

Similarly, corresponding eNK was co-cultured with CFSE-labeled PBMC. PBS was used a negative control while PHA as a positive control. After 5 days, the co-cultured cells were stained for CD3, CD4 and CD8. CD3⁺CD4⁺CFSE^{low} were regarded as proliferating CD4⁺ T cells. PBMC from different donors were tested in FIGS. 13N-13S. The data show that B2M/CIITA double Knock-Out NK with or without transgenes did not stimulate CD4+ T cell proliferation.

### EMBODIMENTS

The following non-limiting embodiments provide illustrative examples of the invention, but do not limit the scope of the invention.

Embodiment 1. An engineered NK cell, comprising:
a secretory IL-15 that is heterologous to the engineered NK cell; and
one or more of:
   (i) a CD16 variant for enhanced CD16 signaling as compared to a control cell, wherein the CD16 variant is heterologous to the cell, or
   (ii) a chimeric polypeptide receptor comprising an antigen binding moiety capable of binding to an antigen, wherein the antigen is not CD19,
wherein the engineered cell lacks a heterologous IL-15R,
optionally wherein:
(1) the engineered NK cell further comprises the CD16 variant; and/or
(2) the engineered NK cell further comprises the chimeric polypeptide receptor; and/or
(3) the engineered NK cell comprises (i); and/or
(4) the engineered NK cell comprises (ii); and/or
(5) the engineered NK cell comprises both (i) and (ii); and/or
(6) the engineered NK cell further comprises a heterologous polynucleotide encoding the secretory IL-15; and/or
(7) the antigen comprises one or more members selected from the group consisting of: BCMA, CD20, CD22, CD30, CD33, CD38, CD70, Kappa, Lewis Y, NKG2D ligand, ROR1, NY-ESO-1, NY-ESO-2, MART-1, and gp100; and/or
(8) the antigen comprises a NKG2D ligand selected from the group consisting of MICA, MICB, ULBP1, ULBP2, ULBP3, ULBP4, ULBP5, AND ULBP6; and/or
(9) the engineered NK cell is derived from an isolated stem cell or an induced stem cell.

Embodiment 2. An engineered NK cell derived from an isolated stem cell or an induced stem cell, comprising a secretory IL-15 that is heterologous to the engineered NK cell, wherein the engineered cell lacks a heterologous IL-15R,
optionally wherein the engineered NK cell further comprises a heterologous polynucleotide encoding the secretory IL-15.

Embodiment 3. An engineered NK cell, comprising:
a membrane-bound IL-15 that is heterologous to the engineered NK cell,
wherein the engineered NK cell further comprise one or more of:
   (a) a heterologous cytokine that is not IL-15,
   (b) reduced expression or activity of endogenous immune regulator polypeptide, wherein the endogenous immune regulator polypeptide is not B2M, or
   (c) a safety switch,
optionally wherein:
(1) the engineered NK cell comprises the heterologous cytokine that is not IL-15; and/or
(2) the engineered NK cell comprises the reduced expression or activity of the endogenous immune regulator polypeptide, wherein the endogenous immune regulator polypeptide is not B2M; and/or
(3) the engineered NK cell comprises the safety switch, optionally wherein the safety switch comprises an inducible cell death moiety; and/or
(4) the engineered NK cell comprises two or more of (a)-(c); and/or
(5) the engineered NK cell comprises (a), (b), and (c); and/or
(6) the engineered NK cell is derived from an isolated stem cell or an induced stem cell; and/or
(7) the membrane bound IL-15 is a part of a chimeric membrane receptor comprising the IL-15 and at least a portion of IL-15R; and/or
(8) the chimeric membrane receptor comprises a sequence selected from the group consisting of (i) four or more adjacent repeats of SEQ ID NO. 9, (ii) SEQ ID NO. 10, (iii) SEQ ID NO. 11, (iv) a combination of SEQ ID NO. 9 and SEQ ID NO. 11, (v) SEQ ID NO. 12, (vi) SEQ ID NO. 13, and (vii) SEQ ID NO. 14).

Embodiment 4. An engineered NK cell, comprising:
a CD16 variant for enhanced CD16 signaling in the engineered NK cell, the CD16 variant comprising at least a portion of CD16 and at least a portion of CD64,
wherein the engineered NK cell exhibits reduced expression or activity of an endogenous immune regulator polypeptide,
optionally wherein:
(1) the engineered NK cell further comprises a heterologous IL-15 or a fragment thereof; and/or
(2) the engineered NK cell further comprises a receptor comprising a heterologous IL-15R or a fragment thereof; and/or
(3) the engineered NK cell is derived from an isolated stem cell or an induced stem cell.

Embodiment 5. An engineered NK cell, comprising reduced activity of endogenous IL-17 signaling, wherein the engineered NK cell is derived from an isolated stem cell or an induced stem cell,
optionally wherein:
(1) the engineered NK cell comprises reduced expression of endogenous IL-17 or endogenous IL-17R; and/or
(2) the engineered NK cell comprises reduced expression of endogenous IL-17 and endogenous IL-17R; and/or
(3) the endogenous IL-17 is IL-17A; and/or
(4) the endogenous IL-17 is IL-17F; and/or
(5) the endogenous IL-17R comprises IL-17RA, IL-17RB, IL-17RC, IL-17RD, or IL-17RE; and/or
(6) the endogenous IL-17R comprises IL-17RA; and/or
(7) the engineered NK cell further comprises a heterologous IL-15 or a fragment thereof; and/or
(8) the engineered NK cell further comprises a receptor comprising a heterologous IL-15R or a fragment thereof; and/or
(9) the engineered NK cell is derived from an isolated stem cell or an induced stem cell; and/or
(10) the engineered NK cell exhibits enhanced expression profile of a NK cell marker as compared to a control cell without reduced expression or activity of endogenous IL-17; and/or
(11) the NK cell marker comprises human CD57 or killer immunoglobulin-like receptors (KIR); and/or
(12) the engineered NK cell exhibits enhanced survival in the presence of tumor cells as compared to a control cell without reduced activity of endogenous IL-17 signaling.

Embodiment 6. An engineered NK cell, comprising a heterologous STAT,
wherein the engineered NK cell is derived from an isolated stem cell or an induced stem cell,
optionally wherein:
(1) the engineered NK cell exhibits enhanced survival in the presence of tumor cells as compared to a control cell without the heterologous STAT; and/or
(2) the heterologous STAT comprises STAT1, STAT2, STAT3, STAT4, STAT3, STAT4, STAT5A, STAT5B, or STAT6; and/or
(3) the heterologous STAT comprises STAT3; and/or
(4) the heterologous STAT comprises STAT5B; and/or
(5) the engineered NK cell further comprises a heterologous IL-15 or a fragment thereof; and/or
(6) the engineered NK cell further comprises a receptor comprising a heterologous IL-15R or a fragment thereof.

Embodiment 7. An engineered NK cell, comprising:
reduced expression or activity of endogenous Killer-cell immunoglobulin-like receptor (KIR),
wherein the engineered NK cell further comprises one or more of:
   (a) a chimeric polypeptide receptor comprising an antigen binding moiety capable of binding to an antigen,
   (b) a heterologous cytokine,
   (c) a CD16 variant for enhanced CD16 signaling as compared to a control cell, wherein the CD16 variant is heterologous to the engineered NK cell,
   (d) a heterologous immune regulator polypeptide, and/or
   (e) reduced expression or activity of endogenous immune regulator polypeptide,
optionally wherein:
(1) the engineered NK cell comprises the chimeric polypeptide receptor; and/or
(2) the engineered NK cell comprises the heterologous cytokine; and/or
(3) the engineered NK cell comprises the CD16 variant; and/or
(4) the engineered NK cell comprises the heterologous immune regulator polypeptide; and/or
(5) the engineered NK cell comprises the reduced expression or activity of endogenous immune regulator polypeptide; and/or
(6) the engineered NK cell comprises two or more of (a)-(e); and/or
(7) the engineered NK cell comprises three or more of (a)-(e); and/or
(8) the engineered NK cell comprises four or more of (a)-(e); and/or
(9) the engineered NK cell comprises all of (a)-(e); and/or
(10) the KIR comprises KIR2D; and/or
(11) the KIR2D comprises KIR2DL1, KIR2DL2, KIR2DL3, KIR2DL4, KIR2DL5A, KIR2DL5B, KIR2DS1, KIR2DS2, KIR2DS3, KIR2DS4, or KIR2DS5; and/or
(12) the KIR comprises KIR3D; and/or
(13) the KIR3D comprises KIR3DL1, KIR3DL2, KIR3DL3, or KIR3DS1; and/or
(14) the engineered NK cell further comprises a heterologous IL-15 or a fragment thereof; and/or
(15) the engineered NK cell further comprises a receptor comprising a heterologous IL-15R or a fragment thereof; and/or
(16) the engineered NK cell is derived from an isolated stem cell or an induced stem cell.

Embodiment 8. An engineered NK cell, comprising:
reduced expression or activity of one or more of:
   (i) endogenous CD94,
   (ii) endogenous CD96,
   (iii) endogenous TGF beta receptor, and/or
   (iv) endogenous SHIP2,
wherein the engineered cell is derived from an isolated stem cell or an induced stem cell,
optionally wherein:
(1) the engineered NK cell comprises reduced expression or activity of the endogenous CD94; and/or
(2) the engineered NK cell comprises reduced expression or activity of the endogenous CD96;
and/or
(3) the engineered NK cell comprises reduced expression or activity of the endogenous TGF beta receptor; and/or
(4) the engineered NK cell comprises reduced expression or activity of the endogenous SHIP2;
and/or
(5) the engineered NK cell comprises reduced expression or activity of two or more of (i)-(iv);
and/or
(6) the engineered NK cell comprises reduced expression or activity of three or more of (i)-(iv); and/or
(7) the engineered NK cell comprises reduced expression or activity of (i)-(iv); and/or
(8) the engineered NK cell further comprises a heterologous IL-15 or a fragment thereof; and/or
(9) the engineered NK cell further comprises a receptor comprising a heterologous IL-15R or a fragment thereof; and/or
(10) the engineered NK cell is derived from an isolated stem cell or an induced stem cell.

Embodiment 9. An engineered NK cell, comprising:
reduced expression or activity of one or more of:
   (i) endogenous CD80,
   (ii) endogenous CD86,
   (iii) endogenous ICOSL,
   (iv) endogenous CD40L,
   (v) endogenous MICA or MICB, and/or
   (vi) endogenous NKG2DL,
wherein the engineered cell is derived from an isolated stem cell or an induced stem cell,
optionally wherein:
(1) the engineered NK cell comprises reduced expression or activity of endogenous CD80; and/or
(2) the engineered NK cell comprises reduced expression or activity of endogenous CD86; and/or
(3) the engineered NK cell comprises reduced expression or activity of endogenous ICOSL; and/or
(4) the engineered NK cell comprises reduced expression or activity of endogenous CD40L; and/or
(5) the engineered NK cell comprises reduced expression or activity of endogenous MICA or MICB; and/or
(6) the engineered NK cell comprises reduced expression or activity of endogenous NKG2D; and/or
(7) the engineered NK cell comprises reduced expression or activity of two or more of (i)-(vi); and/or
(8) the engineered NK cell comprises reduced expression or activity of three or more of (i)-(vi); and/or
(9) the engineered NK cell comprises reduced expression or activity of four or more of (i)-(vi); and/or
(10) the engineered NK cell comprises reduced expression or activity of five or more of (i)-(vi); and/or
(11) the engineered NK cell comprises reduced expression or activity of (i)-(vi); and/or
(12) the engineered NK cell further comprises a heterologous IL-15 or a fragment thereof; and/or
(13) the engineered NK cell further comprises a receptor comprising a heterologous IL-15R or a fragment thereof.

Embodiment 10. An engineered NK cell, comprising:
reduced expression or activity of endogenous ICAM1,
wherein the engineered cell further comprises one or more of:
   (a) a chimeric polypeptide receptor comprising an antigen binding moiety capable of binding to an antigen,
   (b) a heterologous cytokine, and/or
   (c) a CD16 variant for enhanced CD16 signaling as compared to a control cell, wherein the CD16 variant is heterologous to the engineered NK cell,
optionally wherein:
(1) the engineered NK cell comprises the chimeric polypeptide receptor; and/or
(2) the engineered NK cell comprises the heterologous cytokine; and/or
(3) the engineered NK cell comprises the CD16 variant; and/or
(4) the engineered NK cell comprises two or more of (a)-(c); and/or
(5) the engineered NK cell comprises all of (a)-(c); and/or
(6) the engineered NK cell further comprises a heterologous IL-15 or a fragment thereof; and/or
(7) the engineered NK cell further comprises a receptor comprising a heterologous IL-15R or a fragment thereof; and/or
(8) the engineered NK cell is derived from an isolated stem cell or an induced stem cell.

Embodiment 11. An engineered NK cell, comprising reduced expression or activity of endogenous ICAM1, wherein the engineered cell is derived from an induced stem cell.
optionally wherein:
(1) the engineered NK cell further comprises a heterologous IL-15 or a fragment thereof; and/or
(2) the engineered NK cell further comprises a receptor comprising a heterologous IL-15R or a fragment thereof; and/or
(3) the engineered NK cell is derived from an isolated stem cell or an induced stem cell.

Embodiment 12. An engineered NK cell, comprising one or more of: (i) a heterologous PD-L2 or (ii) a heterologous TGF-beta,
optionally wherein:
(1) the engineered NK cell comprises the heterologous PD-L2; and/or
(2) the engineered NK cell comprises the heterologous TGF-beta; and/or
(3) the engineered NK cell comprises (i) the heterologous PD-L2 and (ii) the heterologous TGF-beta; and/or
(4) the engineered NK cell further comprises a heterologous IL-15 or a fragment thereof; and/or
(5) the engineered NK cell further comprises a receptor comprising a heterologous IL-15R or a fragment thereof; and/or
(6) the engineered NK cell is derived from an isolated stem cell or an induced stem cell.

Embodiment 13. An engineered NK cell comprising one or more of:
(i) a heterologous CCL21,
(ii) a heterologous IL-10,
(iii) a heterologous CD46,
(iv) a heterologous CD55, and/or
(v) a heterologous CD59,
wherein the engineered cell is derived from an isolated stem cell or an induced stem cell,
optionally wherein:
(1) the engineered NK cell comprises the heterologous CCL21; and/or
(2) the engineered NK cell comprises the heterologous IL-10; and/or
(3) the engineered NK cell comprises the heterologous CD46; and/or
(4) the engineered NK cell comprises the heterologous CD55; and/or
(5) the engineered NK cell comprises the heterologous CD59; and/or
(6) the engineered NK cell comprises two or more of (i)-(v); and/or
(7) the engineered NK cell comprises three or more of (i)-(v); and/or
(8) the engineered NK cell comprises four or more of (i)-(v); and/or
(9) the engineered NK cell comprises all of (i)-(v); and/or
(10) the engineered NK cell further comprises a heterologous IL-15 or a fragment thereof; and/or
(11) the engineered NK cell further comprises a receptor comprising a heterologous IL-15R or a fragment thereof.

Embodiment 14. An engineered NK cell derived from an induced stem cell, comprising a heterologous cytokine that is not IL-15,
optionally wherein the heterologous cytokine that is not IL-15 comprises IL-21.

Embodiment 15. An engineered NK cell derived from an induced stem cell, comprising a heterologous IL-21,
optionally wherein:
(1) the engineered NK cell further comprises a heterologous IL-15 or a fragment thereof; and/or
(2) the engineered NK cell further comprises a receptor comprising a heterologous IL-15R or a fragment thereof.

Embodiment 16. An engineered NK cell comprising:
a chimeric polypeptide receptor comprising an antigen binding moiety capable of specifically binding to CD38,
wherein the engineered NK cell is derived from an isolated embryonic stem cell or an induced stem cell,
optionally wherein:
(1) the engineered NK cell further comprises a heterologous IL-15 or a fragment thereof; and/or
(2) the engineered NK cell further comprises a receptor comprising a heterologous IL-15R or a fragment thereof.

Embodiment 17. An engineered NK cell comprising:
a chimeric polypeptide receptor comprising CD8 transmembrane domain and one or more of (i) 2B4 signaling domain and (ii) DAP10 signaling domain,
optionally wherein:
(1) the chimeric polypeptide receptor comprises 2B4 signaling domain; and/or
(2) the chimeric polypeptide receptor comprises DAP10 signaling domain; and/or
(3) the chimeric polypeptide receptor comprises (i) 2B4 signaling domain and (ii) DAP10 signaling domain; and/or
(4) the engineered NK cell further comprises a heterologous IL-15 or a fragment thereof; and/or
(5) the engineered NK cell further comprises a receptor comprising a heterologous IL-15R or a fragment thereof; and/or
(6) the engineered NK cell is derived from an isolated stem cell or an induced stem cell.

Embodiment 18. An engineered NK cell derived from an isolated stem cell or an induced stem cell, the engineered NK cell comprising:
a chimeric polypeptide receptor comprising one or more of (i) CD8 transmembrane domain, (ii) 2B4 signaling domain, or (iii) DAP10 signaling domain,
optionally wherein:
(1) the chimeric polypeptide receptor comprises CD8 transmembrane domain; and/or
(2) the chimeric polypeptide receptor comprises 2B4 signaling domain; and/or
(3) the chimeric polypeptide receptor comprises DAP10 signaling domain; and/or
(4) the chimeric polypeptide receptor comprises two or more of (i)-(iii); and/or
(5) the chimeric polypeptide receptor comprises all of (i)-(iii); and/or
(6) the engineered NK cell further comprises a heterologous IL-15 or a fragment thereof; and/or
(7) the engineered NK cell further comprises a receptor comprising a heterologous IL-15R or a fragment thereof.

Embodiment 19. An engineered NK cell, comprising:
a chimeric polypeptide receptor comprising an antigen binding moiety capable of specifically binding to CD23,
optionally wherein:
(1) the engineered NK cell further comprises a heterologous IL-15 or a fragment thereof; and/or
(2) the engineered NK cell further comprises a receptor comprising a heterologous IL-15R or a fragment thereof; and/or
(3) the engineered NK cell is derived from an isolated stem cell or an induced stem cell.

Embodiment 20. An engineered NK cell, comprising:
a chimeric polypeptide receptor comprising an antigen binding moiety capable of specifically binding to CD123,
wherein the engineered NK cell is derived from an isolated stem cell or an induced stem cell,
optionally wherein:
(1) the engineered NK cell further comprises a heterologous IL-15 or a fragment thereof; and/or
(2) the engineered NK cell further comprises a receptor comprising a heterologous IL-15R or a fragment thereof.

Embodiment 21. An engineered NK cell, comprising:
a chimeric polypeptide receptor comprising an antigen binding moiety capable of specifically binding to CD7,
wherein the engineered NK cell exhibits reduced expression or activity of endogenous CD7,
optionally wherein:
(1) expression or activity of TCR is not modified in the engineered NK cell; and/or
(2) the engineered NK cell further comprises a heterologous IL-15 or a fragment thereof; and/or
(3) the engineered NK cell further comprises a receptor comprising a heterologous IL-15R or a fragment thereof; and/or
(4) the engineered NK cell is derived from an isolated stem cell or an induced stem cell.

Embodiment 22. An engineered NK cell, comprising a combination of modified expression or activity of a plurality of immune regulator polypeptides identified in Table 2,
wherein the combination of modified expression or activity of the plurality of immune regulator polypeptides comprises (i) reduced expression or activity of one or more first immune regulator polypeptides and (ii) enhanced expression or activity of one or more second immune regulator polypeptides,
optionally wherein:
(1) the one or more first immune regulator polypeptides are endogenous to the engineered NK cell; and/or
(2) the one or more second immune regulator polypeptides are heterologous to the engineered NK cell; and/or
(3) the engineered NK cell further comprises a chimeric polypeptide receptor comprising an antigen binding moiety capable of binding to an antigen; and/or
(4) the engineered NK cell further comprises a heterologous cytokine; and/or
(5) the engineered NK cell further comprises reduced expression or activity of an endogenous cytokine;
(6) the engineered NK cell further comprises a CD16 variant for enhanced CD16 signaling in the engineered NK cell; and/or
(7) the engineered NK cell further comprises a safety switch; and/or
(8) the engineered NK cell exhibits enhanced cytotoxicity against a target cell as compared to a control cell; and/or
(9) the engineered NK cell induces reduced immune response in a host cell as compared to a control cell,
   optionally wherein the host cell is an immune cell; and/or
(10) the engineered NK cell is derived from an isolated stem cell,
   optionally wherein:
   (a) the isolated stem cell comprises an embryonic stem cell; and/or
   (b) the induced stem cell comprises an induced pluripotent stem cell.

Embodiment 23. The engineered NK cell of any one of the preceding embodiments 1-22,
optionally wherein:
(1) the engineered NK cell exhibits reduced expression or activity of endogenous CD38; and/or
(2) expression or activity of endogenous CD38 of the engineered NK cell is not modified; and/or
(3) the heterologous IL-15 or the fragment thereof is secreted by the engineered NK cell; and/or
(4) the heterologous IL-15 or the fragment thereof is membrane-bound; and/or
(5) the engineered NK cell further comprises a chimeric polypeptide receptor comprising an antigen binding moiety capable of binding to an antigen; and/or
(6) the engineered NK cell further comprises an additional chimeric polypeptide receptor comprising an antigen binding moiety capable of binding to an antigen that is different; and/or
(7) the antigen binding moiety of the chimeric polypeptide receptor is a multispecific binding moiety capable of specifically binding to two or more antigens that are different; and/or
(8) the antigen comprises one or more members selected from the group consisting of: BCMA, CD19, CD20, CD22, CD30, CD33, CD38, CD70, Kappa, Lewis Y, NKG2D ligand, ROR1, NY-ESO-1, NY-ESO-2, MART-1, and gp100; and/or
(9) the antigen comprises a NKG2D ligand selected from the group consisting of MICA, MICB, ULBP1, ULBP2, ULBP3, ULBP4, ULBP5, AND ULBP6; and/or
(10) the engineered NK cell further comprises a safety switch capable of effecting death of the engineered NK cell; and/or
(11) the safety switch comprises one or more members selected from the group consisting of caspase (e.g., caspase 3, 7, or 9), thymidine kinase, cytosine deaminase, modified EGFR, and B-cell CD20; and/or
(12) the engineered NK cell further comprises a heterologous cytokine; and/or
(13) the heterologous cytokine comprises one or more members selected from the group consisting of IL6, IL7, IL9, IL10, IL11, IL12, IL15, IL18, and IL21; and/or
(14) the engineered NK cell further comprises a heterologous immune regulator polypeptide; and/or
(15) the heterologous immune regulator polypeptide comprises one or more members selected from the group consisting of HLA-E, CD47, CD113, PDL1, PDL2, A2AR, HLA-G, TGF-beta, CCL21, IL10, CD46, CD55, and CD59; and/or
(16) the engineered NK cell exhibits reduced expression or activity of an endogenous immune regulator polypeptide; and/or
(17) the endogenous immune regulator polypeptide comprises an immune checkpoint inhibitor or a hypo-immunity regulator; and/or
(18) the immune checkpoint inhibitor comprises one or more members selected from the group consisting of PD1, CTLA-4, TIM-3, KIR2D, CD94, NKG2A, NKG2D, IT, CD96, LAG3, TIGIT, TGF beta receptor, and 2B4; and/or
(19) the immune checkpoint inhibitor comprises SHIP2;
(20) the hypo-immunity regulator comprises one or more members selected from the group consisting of B2M, CIITA, TAP1, TAP2, tapasin, NLRC5, RFXANK, RFX5, RFXAP, CD80, CD86, ICOSL, CD40L, ICAM1, MICA, MICB, ULBP1, HLA-E, CD47, CD113, PDL1, PDL2, A2AR, HLA-G, TGF-beta, CCL21, IL10, CD46, CD55, and CD59; and/or
(21) comprises a CD16 variant for enhanced CD16 signaling as compared to a control cell, wherein the CD16 variant is heterologous to the engineered NK cell; and/or
(22) the CD16 variant comprises a sequence selected from the group consisting of: SEQ ID NOs. 1-8;

(23) the engineered NK cell exhibits enhanced cytotoxicity against a target cell as compared to a control cell; and/or
(24) the engineered NK cell induces reduced immune response in a host cell as compared to a control cell; and/or
(25) the host cell is an immune cell; and/or
(26) the isolated stem cell comprises an embryonic stem cell; and/or
(27) the induced stem cell comprises an induced pluripotent stem cell.

Embodiment 24. The engineered NK cell of any one of the preceding embodiments 1-23,
optionally wherein:
(1) the engineered NK cell is for use in a method for inducing death of a target cell, optionally wherein the target cell is a cancer cell or a tumor cell; and/or
(2) the engineered NK cell is for use in a method for treating a subject in need thereof, wherein the subject has or is suspected of having a condition, optionally wherein the condition is cancer or tumor,
   optionally wherein the engineered NK cell is either autologous or allogeneic to the subject;
(3) the engineered NK cell is for the manufacture of medicament for inducing death of a target cell, optionally wherein the target cell is a cancer cell or a tumor cell; and/or
(4) the engineered NK cell is for the manufacture of medicament for treating a subject in need thereof, wherein the subject has or is suspected of having a condition, optionally wherein the condition is cancer or tumor,
   optionally wherein the engineered NK cell is either autologous or allogeneic to the subject.

Embodiment 25. A method comprising:
obtaining a cell from a subject; and
generating, from the cell, the engineered NK cell of any one of the preceding embodiments 1-24.

Embodiment 26. A method comprising:
administering to a subject in need thereof a population of NK cells comprising the engineered NK cell of any one of the preceding embodiments 1-24,
optionally wherein the method further comprises administering to the subject a separate therapeutic agent,
   further optionally wherein the separate therapeutic agent is a chemotherapeutic agent.

Embodiment 27. A composition comprising:
the engineered NK cell of any one of the preceding embodiments 1-24, and
a separate therapeutic agent capable of binding to CD20.

Embodiment 28. A composition comprising:
an engineered immune cell comprising one or more members comprising:
   (i) a heterologous IL-15 or a fragment thereof,
   (ii) a CD16 variant for enhanced CD16 signaling as compared to a control cell, wherein the CD16 variant is heterologous to the engineered immune cell, and/or
   (iii) a chimeric polypeptide receptor comprising an antigen binding moiety capable of binding to an antigen; and
a separate therapeutic agent capable of binding to CD20;
optionally wherein:
(1) the engineered immune cell comprises the heterologous IL-15; and/or
(2) the heterologous IL-15 or the fragment thereof is secreted by the engineered immune cell; and/or
(3) the heterologous IL-15 or the fragment thereof is membrane-bound; and/or
(4) the engineered immune cell comprises the CD16 variant; and/or
(5) the CD16 variant comprises a sequence selected from the group consisting of: SEQ ID NOs. 1-8; and/or
(6) the engineered immune cell comprises the chimeric polypeptide receptor; and/or
(7) the engineered immune cell comprises two or more of (i)-(iii); and/or
(8) the engineered immune cell comprises (i)-(iii); and/or
(9) the engineered immune cell comprises an engineered NK cell; and/or
(10) the engineered immune cell comprises an engineered T cell; and/or
(11) the engineered cell exhibits reduced expression or activity of endogenous CD38; and/or
(12) expression or activity of endogenous CD38 of the engineered cell is not modified;
(13) the antigen binding moiety of the chimeric polypeptide receptor is a multispecific binding moiety capable of specifically binding to two or more antigens that are different; and/or
(14) the antigen comprises one or more members selected from the group consisting of: BCMA, CD19, CD20, CD22, CD30, CD33, CD38, CD70, Kappa, Lewis Y, NKG2D ligand, ROR1, NY-ESO-1, NY-ESO-2, MART-1, and gp100; and/or
(15) the antigen comprises a NKG2D ligand selected from the group consisting of MICA, MICB, ULBP1, ULBP2, ULBP3, ULBP4, ULBP5, AND ULBP6; and/or
(16) the engineered immune cell further comprises a safety switch capable of effecting death of the engineered immune cell; and/or
(17) the safety switch comprises one or more members selected from the group consisting of caspase (e.g., caspase 3, 7, or 9), thymidine kinase, cytosine deaminase, modified EGFR, and B-cell CD20; and/or
(18) the engineered immune cell further comprises a heterologous cytokine; and/or
(19) the heterologous cytokine comprises one or more members selected from the group consisting of IL6, IL7, IL9, IL10, IL11, IL12, IL15, IL18, and IL21; and/or
(20) the engineered immune cell further comprises a heterologous immune regulator polypeptide; and/or
(21) the heterologous immune regulator polypeptide comprises one or more members selected from the group consisting of HLA-E, CD47, CD113, PDL1, PDL2, A2AR, HLA-G, TGF-beta, CCL21, IL10, CD46, CD55, and CD59; and/or
(22) the engineered immune cell exhibits reduced expression or activity of an endogenous immune regulator polypeptide; and/or
(23) the endogenous immune regulator polypeptide comprises an immune checkpoint inhibitor or a hypo-immunity regulator; and/or
(24) the immune checkpoint inhibitor comprises one or more members selected from the group consisting of PD1, CTLA-4, TIM-3, KIR2D, CD94, NKG2A, NKG2D, IT, CD96, LAG3, TIGIT, TGF beta receptor, and 2B4; and/or
(25) the immune checkpoint inhibitor comprises SHIP2; and/or
(26) the hypo-immunity regulator comprises one or more members selected from the group consisting of B2M, CIITA, TAP1, TAP2, tapasin, NLRC5, RFXANK, RFX5, RFXAP, CD80, CD86, ICOSL, CD40L, ICAM1, MICA, MICB, ULBP1, HLA-E, CD47, CD113, PDL1, PDL2, A2AR, HLA-G, TGF-beta, CCL21, IL10, CD46, CD55, and CD59; and/or
(27) the engineered NK cell exhibits enhanced cytotoxicity against a target cell as compared to a control cell; and/or
(28) the engineered NK cell induces reduced immune response in a host cell as compared to a control cell; and/or
(29) the host cell is an immune cell; and/or
(30) the engineered NK cell further comprises a receptor comprising a heterologous IL-15R or a fragment thereof; and/or
(31) the engineered NK cell is derived from an isolated stem cell or an induced stem cell; and/or
(32) the isolated stem cell comprises an embryonic stem cell; and/or
(33) the induced stem cell comprises an induced pluripotent stem cell.

Embodiment 29. A method comprising administering to a subject in need thereof the composition of Embodiment 27 or Embodiment 28,
optionally wherein the separate therapeutic agent is a chemotherapeutic agent.

Embodiment 30. A population of engineered NK cells, wherein:
an engineered NK cell of the population of engineered NK cells comprises a heterologous polypeptide, wherein the heterologous polypeptide comprises a heterologous IL-15, and
a persistence level of the population of engineered NK cells in an environment that is substantially free of an exogenous interleukin-2 (IL-2) is at least about 5% greater than a control persistence level of a comparable population of NK cells in a control environment comprising the exogenous IL-2,
optionally wherein:
(1) the heterologous IL-15 is a membrane-bound IL-15,
   optionally wherein the heterologous polypeptide is a fusion polypeptide comprising the heterologous IL-15 and at least a portion of IL-15 receptor; and/or
(2) the heterologous IL-15 is a secretory IL-15; and/or
(3) the persistence level is at least about 10% greater than the control persistence level; and/or
(4) the persistence level is at least about 20% greater than the control persistence level; and/or
(5) the persistence level is at least about 30% greater than the control persistence level; and/or
(6) the persistence level and the control persistence level are observed after at least about 5 days in the environment and the control environment, respectively; and/or
(7) the persistence level and the control persistence level are observed after at least about 15 days in the environment and the control environment, respectively; and/or
(8) the persistence level and the control persistence level are observed after at least about 21 days in the environment and the control environment, respectively; and/or
(9) an amount of the exogeneous interleukin in the environment is between about 10 units per milliliter (U/ml) and about 500 U/mL; and/or
(10) the environment is in vitro; and/or
(11) the persistence level is ascertained subsequent to administration of the population of engineered NK cells into a body of a subject in need thereof; and/or
(12) the engineered NK cell comprises a heterologous polynucleotide sequence encoding the heterologous polypeptide; and/or
(13) the engineered NK cell further comprises a heterologous CD16 variant for enhanced CD16 signaling; and/or
(14) the engineered NK cell further comprises a chimeric polypeptide receptor comprising an antigen binding moiety capable of binding to an antigen; and/or
(15) the engineered NK cell comprises reduced expression or activity level of an endogenous gene encoding the antigen; and/or
(16) the engineered NK cell comprises reduced expression or activity level of endogenous CD38; and/or
(17) the engineered NK cell is derived from an isolated stem cell or an induced stem cell.

Embodiment 31. A population of engineered NK cells, wherein:
an engineered NK cell of the population comprises a heterologous secretory IL-15, and
the population of engineered NK cells exhibits a signaling level of an endogenous downstream signaling protein of IL-15 that is at least about 0; and/or 1-fold greater than a control signaling level of the endogenous downstream signaling protein of a control population of NK cells lacking the heterologous secretory IL-15,
optionally wherein:
(1) the signaling level is a phosphorylation level of the endogenous downstream signaling protein; and/or
(2) the signaling level is at least 0.5-fold greater than the control signaling level; and/or
(3) the signaling level is at least 1-fold greater than the control signaling level; and/or
(4) the signaling level is at least 5-fold greater than the control signaling level; and/or
(5) the endogenous downstream signaling protein comprises STAT; and/or
(6) the endogenous downstream signaling protein comprises STATS; and/or
(7) the signaling level is observed in an environment that is substantially free of an exogenous interleukin; and/or
(8) the exogenous interleukin comprises IL-2 and/or IL-15; and/or
(9) the engineered NK cell does not comprise a heterologous IL-15 receptor; and/or
(10 the engineered NK cell further comprises a heterologous polynucleotide sequence encoding the heterologous secretory IL-15; and/or
(11) the engineered NK cell further comprises a heterologous CD16 variant for enhanced CD16 signaling; and/or
(12) the engineered NK cell further comprises a chimeric polypeptide receptor comprising an antigen binding moiety capable of binding to an antigen; and/or
(13) the engineered NK cell comprises reduced expression or activity level of an endogenous gene encoding the antigen; and/or
(14) the engineered NK cell comprises reduced expression or activity level of endogenous CD38;
and/or
(15) the engineered NK cell is derived from an isolated stem cell or an induced stem cell.

Embodiment 32. A population of engineered NK cells, wherein:
an engineered NK cell of the population of engineered NK cells comprises at least one heterologous hypo-immunity regulator polypeptide comprising one or more members selected from the group consisting of PD-L2, TGF-beta, CD46, CD55, and CD59, and
the population of engineered NK cells exhibits enhanced resistance against immune rejection, as compared to that of a control population of NK cells lacking the at least one heterologous hypo-immunity regulator polypeptide,
optionally wherein:
(1) the enhanced resistance against immune rejection is at least about 5%; and/or
(2) the enhanced resistance against immune rejection is at least about 10%; and/or
(3) the enhanced resistance against immune rejection is at least about 20%; and/or
(4) the enhanced resistance against immune rejection is at least about 50%; and/or
(5) the enhanced resistance against immune rejection is ascertained in vitro in a medium comprising at least about 10% human complement; and/or
(6) the enhanced resistance against immune rejection is ascertained in vitro in a medium comprising at least about 20% human complement; and/or
(7) the enhanced resistance against immune rejection is ascertained in vitro in a medium comprising at least about 40% human complement; and/or
(8) the enhanced resistance against immune rejection is ascertained subsequent to administration of the population of engineered NK cells into a body of a subject in need thereof; and/or
(9) the engineered NK cell comprises a heterologous polynucleotide sequence encoding the at least one heterologous hypo-immunity regulator polypeptide; and/or
(10) the at least one heterologous hypo-immunity regulator polypeptide comprises PD-L2; and/or
(11) the at least one heterologous hypo-immunity regulator polypeptide comprises TGF-beta; and/or
(12) the at least one heterologous hypo-immunity regulator polypeptide comprises CD46; and/or
(13) the at least one heterologous hypo-immunity regulator polypeptide comprises CD55; and/or
(14) the at least one heterologous hypo-immunity regulator polypeptide comprises CD59; and/or
(15) the at least one heterologous hypo-immunity regulator polypeptide comprises two or more members selected from the group consisting of PD-L2, TGF-beta, CD46, CD55, and CD59; and/or
(16) the engineered NK cell further comprises at least an additional heterologous hypo-immunity regulator polypeptide comprising one or more members selected from the group consisting of HLA-G, PD-L1, HLA-E, CD47, IL-10, CCL-21, and CD59; and/or
(17) the engineered NK cell further comprises at least an additional heterologous hypo-immunity regulator polypeptide comprising HLA-G; and/or
(18) the engineered NK cell further comprises at least an additional heterologous hypo-immunity regulator polypeptide comprising PD-L1; and/or
(19) the engineered NK cell further comprises at least an additional heterologous hypo-immunity regulator polypeptide comprising HLA-E; and/or
(20) the engineered NK cell further comprises at least an additional heterologous hypo-immunity regulator polypeptide comprising CD47; and/or
(21) the engineered NK cell further comprises at least an additional heterologous hypo-immunity regulator polypeptide comprising IL-10; and/or
(22) the engineered NK cell further comprises at least an additional heterologous hypo-immunity regulator polypeptide comprising CCL-21; and/or
(23) the engineered NK cell further comprises at least an additional heterologous hypo-immunity regulator polypeptide comprising CD59; and/or
(24) the engineered NK cell further comprises reduced expression or activity of at least one endogenous immune regulator polypeptide comprising one or more members selected from the group consisting of MICA, MICB, ULBP1, B2M, CIITA, and ICAM-1; and/or
(25) the engineered NK cell further comprises reduced expression or activity of at least one endogenous immune regulator polypeptide comprising one or more members selected from the group consisting of MICA, MICB, and ULBP1; and/or
(26) the engineered NK cell further comprises reduced expression or activity of at least one endogenous immune regulator polypeptide comprising MICA; and/or
(27) the engineered NK cell further comprises reduced expression or activity of at least one endogenous immune regulator polypeptide comprising MICB; and/or
(28) the engineered NK cell further comprises reduced expression or activity of at least one endogenous immune regulator polypeptide comprising ULBP1; and/or
(29) the engineered NK cell further comprises reduced expression or activity of at least one endogenous immune regulator polypeptide comprising one or more members selected from the group consisting of B2M and CIITA; and/or
(30) the engineered NK cell further comprises reduced expression or activity of at least one endogenous immune regulator polypeptide comprising ICAM-1; and/or
(31) the engineered NK cell further comprises a heterologous IL-15; and/or
(32) the engineered NK cell further comprises a heterologous CD16 variant for enhanced CD16 signaling; and/or
(33) the engineered NK cell further comprises a chimeric polypeptide receptor comprising an antigen binding moiety capable of binding to an antigen; and/or
(34) the engineered NK cell comprises reduced expression or activity level of an endogenous gene encoding the antigen; and/or
(35) the engineered NK cell comprises reduced expression or activity level of endogenous CD38; and/or
(36) the engineered NK cell is derived from an isolated stem cell or an induced stem cell.

Embodiment 33. A population of engineered NK cells, wherein:
an engineered NK cell of the population of engineered NK cells comprises a plurality of heterologous hypo-immunity regulator polypeptides, and
the population of engineered NK cells exhibits enhanced resistance against immune rejection by at least about 5%, as compared to that of a control population of NK cells lacking one or more of the plurality of heterologous hypo-immunity regulator polypeptides,
optionally wherein:
(1) the enhanced resistance against immune rejection is at least about 5%; and/or
(2) the enhanced resistance against immune rejection is at least about 10%; and/or
(3) the enhanced resistance against immune rejection is at least about 20%; and/or
(4) the enhanced resistance against immune rejection is at least about 50%; and/or
(5) the enhanced resistance against immune rejection is ascertained in vitro in a medium comprising at least about 10% human complement; and/or
(6) the enhanced resistance against immune rejection is ascertained in vitro in a medium comprising at least about 20% human complement; and/or
(7) the enhanced resistance against immune rejection is ascertained in vitro in a medium comprising at least about 40% human complement; and/or
(8) the engineered NK cell comprises at least one heterologous polynucleotide sequence encoding the plurality of heterologous hypo-immunity regulator polypeptides; and/or
(9) the plurality of heterologous hypo-immunity regulator polypeptides comprises two or more members selected from the group consisting of PD-L2, TGF-beta, CD46, CD55, CD59, HLA-G, PD-L1, HLA-E, CD47, IL-10, CCL-21, and CD59; and/or
(10) the plurality of heterologous hypo-immunity regulator polypeptides comprises three or more members selected from the group consisting of PD-L2, TGF-beta, CD46, CD55, CD59, HLA-G, PD-L1, HLA-E, CD47, IL-10, CCL-21, and CD59; and/or
(11) the plurality of heterologous hypo-immunity regulator polypeptides comprises four or more members selected from the group consisting of PD-L2, TGF-beta, CD46, CD55, CD59, HLA-G, PD-L1, HLA-E, CD47, IL-10, CCL-21, and CD59; and/or
(12) the plurality of heterologous hypo-immunity regulator polypeptides comprises five or more members selected from the group consisting of PD-L2, TGF-beta, CD46, CD55, CD59, HLA-G, PD-L1, HLA-E, CD47, IL-10, CCL-21, and CD59; and/or
(13) the plurality of heterologous hypo-immunity regulator polypeptides comprises two or more members selected from the group consisting of PD-L2, TGF-beta, CD46, CD55, CD59, and HLA-G; and/or
(14) the plurality of heterologous hypo-immunity regulator polypeptides comprises PD-L2; and/or
(15) the plurality of heterologous hypo-immunity regulator polypeptides comprises TGF-beta; and/or
(16) the plurality of heterologous hypo-immunity regulator polypeptides comprises CD46; and/or
(17) the plurality of heterologous hypo-immunity regulator polypeptides comprises CD55; and/or
(18) the plurality of heterologous hypo-immunity regulator polypeptides comprises CD59; and/or
(19) the plurality of heterologous hypo-immunity regulator polypeptides comprises HLA-G; and/or
(20) the plurality of heterologous hypo-immunity regulator polypeptides comprises PD-L1; and/or
(21) the plurality of heterologous hypo-immunity regulator polypeptides comprises HLA-E; and/or
(22) the plurality of heterologous hypo-immunity regulator polypeptides comprises CD47; and/or
(23) the plurality of heterologous hypo-immunity regulator polypeptides comprises IL-10; and/or
(24) the plurality of heterologous hypo-immunity regulator polypeptides comprises CCL-21; and/or
(25) the plurality of heterologous hypo-immunity regulator polypeptides comprises CD59; and/or
(26) the engineered NK cell further comprises reduced expression or activity of an endogenous immune regulator polypeptide comprising one or more members selected from the group consisting of MICA, MICB, ULBP1, B2M, CIITA, and ICAM-1; and/or
(27) the engineered NK cell further comprises reduced expression or activity of the endogenous immune regulator polypeptide comprising one or more members selected from the group consisting of MICA, MICB, and ULBP1; and/or
(28) the engineered NK cell further comprises reduced expression or activity of the endogenous immune regulator polypeptide comprising MICA; and/or
(29) the engineered NK cell further comprises reduced expression or activity of the endogenous immune regulator polypeptide comprising MICB; and/or
(30) the engineered NK cell further comprises reduced expression or activity of the endogenous immune regulator polypeptide comprising ULBP1; and/or
(31) the engineered NK cell further comprises reduced expression or activity of the endogenous immune regulator polypeptide comprising one or more members selected from the group consisting of B2M and CIITA1; and/or
(32) the engineered NK cell further comprises reduced expression or activity of the endogenous immune regulator polypeptide comprising ICAM-1; and/or
(33) the engineered NK cell further comprises a heterologous IL-15; and/or
(34) the engineered NK cell further comprises a heterologous CD16 variant for enhanced CD16 signaling; and/or
(35) the engineered NK cell further comprises a chimeric polypeptide receptor comprising an antigen binding moiety capable of binding to an antigen; and/or
(36) the engineered NK cell comprises reduced expression or activity level of an endogenous gene encoding the antigen; and/or
(37) the engineered NK cell comprises reduced expression or activity level of endogenous CD38; and/or
(38) the engineered NK cell is derived from an isolated stem cell or an induced stem cell.

Embodiment 34. A population of engineered NK cells, wherein:
an engineered NK cell of the population of engineered NK cells comprises (i) reduced expression of at least one endogenous immune regulating polypeptide that is not PTPN2, and/or (ii) enhanced or introduced expression of NKG2C, and
a persistence level of the population of engineered NK cells that is greater than a control persistence level of a control population of NK cells lacking (i) and/or (ii),
optionally wherein:
(1) the control population of NK cells comprises reduced expression of endogenous PTPN2; and/or
(2) the persistence level is at least about 10% greater than the control persistence level; and/or
(3) the persistence level is at least about 20% greater than the control persistence level; and/or
(4) the persistence level is at least about 50% greater than the control persistence level; and/or
(5) the persistence level is at least about 100% greater than the control persistence level; and/or
(6) the engineered NK cell comprises the reduced expression of the at least one endogenous immune regulating polypeptide comprising one or more members selected from the group consisting of BCL3, CBLB, CDK8, FCER1G, IL17A, IL17F, SHIP1, SOCS1, SOCS2, SOCS3, STAT3, TET3, PTPN6, and CD70; and/or
(7) the engineered NK cell comprises the reduced expression of the at least one endogenous immune regulating polypeptide comprising two or more members selected from the group consisting of BCL3, CBLB, CDK8, FCER1G, IL17A, IL17F, SHIP1, SOCS1, SOCS2, SOCS3, STAT3, TET3, PTPN6, and CD70; and/or
(8) the at least one endogenous immune regulating polypeptide comprises BCL3; and/or
(9) the at least one endogenous immune regulating polypeptide comprises CBLB; and/or
(10) the at least one endogenous immune regulating polypeptide comprises CDK8; and/or
(11) the at least one endogenous immune regulating polypeptide comprises FCER1G; and/or
(12) the at least one endogenous immune regulating polypeptide comprises IL17A; and/or
(13) the at least one endogenous immune regulating polypeptide comprises IL17F; and/or
(14) the at least one endogenous immune regulating polypeptide comprises SHIP1; and/or
(15) the at least one endogenous immune regulating polypeptide comprises SOCS1; and/or
(16) the at least one endogenous immune regulating polypeptide comprises SOCS2; and/or
(17) the at least one endogenous immune regulating polypeptide comprises SOCS3; and/or
(18) the at least one endogenous immune regulating polypeptide comprises STAT3; and/or
(19) the at least one endogenous immune regulating polypeptide comprises TET3; and/or
(20) the at least one endogenous immune regulating polypeptide comprises PTPN6; and/or
(21) the at least one endogenous immune regulating polypeptide comprises CD70; and/or
(22) the engineered NK cell comprises the enhanced or introduced expression of NKG2C; and/or
(23) the engineered NK cell comprises enhanced expression of endogenous NKG2C; and/or
(24) the engineered NK cell comprises a heterologous NKG2C; and/or
(25) the engineered NK cell comprises the reduced expression of the at least one endogenous immune regulating polypeptide comprising one or more members selected from the group consisting of NLRC5, RFXANK, RFXAP, CD80, CD7, TAP2, TAP1, TAPBP; and/or
(26) the engineered NK cell comprises the reduced expression of the at least one endogenous immune regulating polypeptide comprising two or more members selected from the group consisting of NLRC5, RFXANK, RFXAP, CD80, CD7, TAP2, TAP1, TAPBP; and/or
(27) the engineered NK cell comprises the reduced expression of the at least one endogenous immune regulating polypeptide comprising three or more members selected from the group consisting of NLRC5, RFXANK, RFXAP, CD80, CD7, TAP2, TAP1, TAPBP; and/or
(28) the engineered NK cell comprises the reduced expression of the at least one endogenous immune regulating polypeptide comprising four or more members selected from the group consisting of NLRC5, RFXANK, RFXAP, CD80, CD7, TAP2, TAP1, TAPBP; and/or
(29) the engineered NK cell comprises the reduced expression of the at least one endogenous immune regulating polypeptide comprising NLRC5; and/or
(30) the engineered NK cell comprises the reduced expression of the at least one endogenous immune regulating polypeptide comprising RFXANK; and/or
(31) the engineered NK cell comprises the reduced expression of the at least one endogenous immune regulating polypeptide comprising RFXAP; and/or
(32) the engineered NK cell comprises the reduced expression of the at least one endogenous immune regulating polypeptide comprising CD80; and/or
(33) the engineered NK cell comprises the reduced expression of the at least one endogenous immune regulating polypeptide comprising CD7; and/or
(34) the engineered NK cell comprises the reduced expression of the at least one endogenous immune regulating polypeptide comprising TAP2; and/or
(35) the engineered NK cell comprises the reduced expression of the at least one endogenous immune regulating polypeptide comprising TAP1; and/or
(36) the engineered NK cell comprises the reduced expression of the at least one endogenous immune regulating polypeptide comprising TAPBP; and/or
(37) the engineered NK cell comprises (i), and the control population of NK cells lack (i); and/or
(38) the engineered NK cell comprises (ii), and the control population of NK cells lack (ii); and/or
(39) the engineered NK cell further comprises a heterologous IL-15; and/or
(40) the engineered NK cell further comprises a heterologous CD16 variant for enhanced CD16 signaling; and/or
(41) the engineered NK cell further comprises a chimeric polypeptide receptor comprising an antigen binding moiety capable of binding to an antigen; and/or
(42) the engineered NK cell comprises reduced expression or activity level of an endogenous gene encoding the antigen; and/or
(43) the engineered NK cell comprises reduced expression or activity level of endogenous CD38; and/or
(44) the engineered NK cell is derived from an isolated stem cell or an induced stem cell; and/or
(45) the at least one endogenous immune regulating polypeptide does not comprise SOCS1, SOCS2, and/or SOCS3; and/or
(46) the engineered NK cell does not comprise enhanced or introduced expression of NKG2C.

Embodiment 35. The population of engineered NK cells of any one of the preceding Embodiments 30-34,
optionally wherein:
(1) the chimeric polypeptide receptor is a multispecific binding moiety capable of specifically binding to two or more antigens that are different; and/or
(2) the antigen comprises one or more members selected from the group consisting of: BCMA, CD19, CD20, CD22, CD30, CD33, CD38, CD70, Kappa, Lewis Y, NKG2D ligand, ROR1, NY-ESO-1, NY-ESO-2, MART-1, and gp100; and/or
(3) the antigen comprises a NKG2D ligand selected from the group consisting of MICA, MICB, ULBP1, ULBP2, ULBP3, ULBP4, ULBP5, AND ULBP6; and/or
(4) the engineered immune cell further comprises a safety switch capable of effecting death of the engineered immune cell; and/or
(5) the safety switch comprises one or more members selected from the group consisting of caspase (e; and/org; and/or, caspase 3, 7, or 9), thymidine kinase, cytosine deaminase, modified EGFR, and B-cell CD20.

Embodiment 36. The population of engineered NK cells of any one of the preceding Embodiments 30-35,
optionally wherein:
(1) the population of engineered NK cells is for use in a method for inducing death of a target cell, optionally wherein the target cell is a cancer cell or a tumor cell; and/or
(2) the population of engineered NK cells is for treating a subject in need thereof, wherein the subject has or is suspected of having a condition, optionally wherein the condition is cancer or tumor,
   further optionally wherein the engineered NK cell is either autologous or allogeneic to the subject; and/or
(3) the population of engineered NK cells is for the manufacture of medicament for inducing death of a target cell, optionally wherein the target cell is a cancer cell or a tumor cell; and/or
(4) the population of engineered NK cells is for the manufacture of medicament for treating a subject in need thereof, wherein the subject has or is suspected of having a condition, optionally wherein the condition is cancer or tumor,
   further optionally wherein the engineered NK cell is either autologous or allogeneic to the subject.

Embodiment 37. A composition comprising the population of engineered NK cells of any one of the preceding Embodiments 30-35,
optionally wherein the composition further comprises a separate therapeutic agent,
   further optionally wherein the therapeutic agent is a chemotherapeutic agent.

Embodiment 38. A method comprising:
obtaining one or more cells from a subject; and
generating, from the one or more cells, the population of engineered NK cells of any one of the preceding Embodiments 30-35,
optionally wherein the one or more cells are NK cells, hematopoietic stem cells, or fibroblasts.

Embodiment 39. A method comprising engineering one or more induced pluripotent stem cells to generate the population of engineered NK cells of any one of the preceding Embodiments 30-35.

Embodiment 40. A method comprising administering to a subject in need thereof the population of engineered NK cells of any one of the preceding Embodiments 30-35,
optionally wherein the method further comprises administering to the subject a separate therapeutic agent,
   further optionally wherein the separate therapeutic agent is a chemotherapeutic agent.

It shall be understood that different aspects of the invention can be appreciated individually, collectively, or in combination with each other. Various aspects of the invention described herein may be applied to any of the particular applications disclosed herein. The compositions of matter including the engineered immune cells of the present disclosure may be utilized in the method section including methods of use and production disclosed herein, or vice versa.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-106. (canceled)

107. A population of engineered NK cells, wherein
a) the engineered NK cell is derived from an isolated stem cell or an induced stem cell,
b) an engineered NK cell of the population of engineered NK cells comprises a heterologous polypeptide or the nucleotide coding sequence thereof, a reduced expression of an endogenous immune regulating polypeptide or modification of the nucleotide coding sequence of the endogenous immune regulating polypeptide, or an enhanced or introduced expression of NKG2C; and
wherein the cell optionally comprises at least one feature selected from the group consisting of:
(i) a CD16 variant for enhanced CD16 signaling, or the nucleotide coding sequence thereof;
(ii) a chimeric polypeptide receptor (CAR) comprising an antigen binding moiety capable of binding to an antigen, or the nucleotide coding sequence thereof;
(iii) a heterologous hypo-immunity regulator polypeptide or an endogenous immune regulator polypeptide for enhanced resistance against immune rejection, or the nucleotide coding sequence thereof;
(iv) a safety switch capable of effecting death of the engineered immune cell, or the nucleotide coding sequence thereof; and
(v) one or more enhanced or introduced genes and/or one or more reduced expression level of endogenous genes for improved function in tumor microenvironment.

108. The population of claim 107, wherein the heterologous polypeptide comprises a heterologous IL-15 or a fragment thereof, preferably the heterologous polypeptide comprises a membrane-bound IL-15, a fusion polypeptide comprising the heterologous IL-15, a portion of IL-15 receptor, or a secretory IL-15, preferably an IL15-IL15R fusion; and/or the reduced expression of the endogenous immune regulating polypeptide for enhancing persistence comprises a reduced expression of one or more members selected from the group consisting of BCL3, CBLB, CDK8, FCER1G, IL17A, IL17F, SHIP1, SOCS1, SOCS2, SOCS3, STAT3, TET3, PTPN6, and CD70.

109. The population of claim 107, wherein the CD16 variant for enhanced CD16 signaling is selected from the group consisting of:
(a) a heterologous CD16 variant, preferably hnCD16;
(b) at least a portion of CD16;
(c) at least a portion of CD64; and
(d) CD16-CD64 fusion protein.

110. The population of claim 107, wherein the CAR specifically recognizes an antigen selected from the group consisting of BCMA, CD19, CD20, CD22, CD30, CD33, CD38, CD70, κ, Lewis Y, NKG2D ligand, ROR1, NY-ESO-1, NY-ESO-2, MART-1, and gp100, preferably the CAR specifically recognizes a tumor antigen selected from the group consisting of CD19, CD33, and BCMA.

111. The population of claim 107, wherein
a) the engineered NK cell is derived from an isolated stem cell or an induced stem cell,
b) an engineered NK cell of the population of engineered NK cells comprises the following:
(i) a CAR specifically recognizes an antigen selected from the group consisting of CD33, CD19, and BCMA, preferably a CAR specifically recognizes an antigen of CD19, or the nucleotide coding sequences of the CAR;
(ii) a heterologous CD16 variant for enhanced CD16 signaling, preferably a hnCD16, or the nucleotide coding sequences thereof; and
(iii) a heterologous IL-15, preferably an IL15-IL15R fusion, or the nucleotide coding sequences thereof.

112. The population of claim 111, wherein an engineered NK cell of the population of engineered NK cells further comprises:
(iv) a hypo-immunity regulator polypeptide or the nucleotide coding sequence thereof, or modified expression of an endogenous hypo-immunity regulator polypeptide or modification of the nucleotide coding sequence of the endogenous hypo-immunity regulator polypeptide for enhanced resistance against immune rejection.

113. The population of claim 112, wherein:
(i) the hypo-immunity regulator polypeptide for enhanced resistance against immune rejection comprises one or more members selected from the group consisting of: HLA-E, CD47, CD113, PDL1, PDL2, A2AR, HLA-G, TGF-beta, CCL21, IL10, CD46, CD55, CD59; and/or
(ii) the modified expression of the endogenous hypo-immunity regulator polypeptide for enhanced resistance against immune rejection comprises a reduced expression or activity of one or more members selected from the group consisting of MICA, MICB, ULBP1, B2M, CIITA, ICAM-1, TAP1, TAP2, tapasin, NLRC5, RFXANK, RFX5, RFXAP, CD80, CD7, TAPBP, CD86, ICOSL, CD40L, and NKG2DL.

114. The population of claim 113, wherein the engineered NK cell of the population of engineered NK cells comprises:
(i) an enhanced or introduced expression of HLA-E, CD47, PDL1, PDL2, HLA-G, TGF-beta, CCL21, IL10, CD46, CD55, and/or CD59; and
(ii) a reduced expression or activity of B2M, MICA, MICB, ULBP1 and/or CIITA.

115. The population of claim 114, wherein the engineered NK cell of the population of engineered NK cells comprises:
(i) an enhanced or introduced expression of HLA-E and HLA-G; and
(ii) a reduced expression of B2M and CIITA.

116. The population of claim 115, wherein:
the engineered NK cell of the population of engineered NK cells further comprises:
an enhanced or introduced expression of PDL1 and/or CD47.

117. The population of claim 107, wherein:
(i) a persistence level of the population of engineered NK cells in an environment that is substantially free of an exogenous interleukin-2 (IL-2) is at least about 5%, at least about 10%, at least about 20%, or at least about 30% greater than a control persistence level of a comparable population of NK cells in a control environment comprising the exogenous IL-2, preferably the persistence level and the control persistence level are observed after at least about 5 days, at least about 15 days, or at least about 21 days in the environment and the control environment, respectively; and/or
(ii) the population of engineered NK cells exhibits a signaling level of an endogenous downstream signaling protein of IL-15 that is at least about 0.1-fold, at least 0.5-fold, at least 1-fold, or at least 5-fold greater than a control signaling level of the endogenous downstream signaling protein of a control population of NK cells lacking the heterologous secretory IL-15, preferably the endogenous downstream signaling protein comprises STAT, more preferably the endogenous downstream signaling protein comprises STAT5; and/or
(iii) the population of engineered NK cells exhibits enhanced resistance against immune rejection by at least about 5%, at least about 10%, at least about 20%; or at least about 50%, as compared to that of a control population of NK cells lacking the at least one heterologous hypo-immunity regulator polypeptide; preferably the enhanced resistance against immune rejection is ascertained in vitro in a medium comprising at least about 10%, at least about 20%, or at least about 40% human complement.

118. A composition comprising the population of claim 107 and optionally one or more co-therapeutic agents, preferably the co-therapeutic agents are selected from the group consisting of cytotoxic agents, chemotherapeutic agents, growth inhibitory agents, agents used in radiation therapy, anti-angiogenesis agents, apoptotic agents, anti-tubulin agents, and other agents to treat cancer, more preferably the co-therapeutic agent is a chemotherapeutic agent.

119. A method for treating a disease in a subject, comprising administering the population of claim 107 to the subject, preferably the disease is cancer or tumor.

120. A method for treating a disease in a subject, comprising administering the composition of claim 118 to the subject, preferably the disease is cancer or tumor.


SEQUENCE LISTING (262 sequences)

SEQ ID NO: 1 - protein, 338 aa, synthetic construct
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVSFCLVMVLLFAVDTGLYFSVKTNIRSSTRDWKDHKFKWRKDPQDK

SEQ ID NO: 2 - protein, 4 aa, synthetic construct
FHVS

SEQ ID NO: 3 - protein, 5 aa, synthetic construct
WFHVS

SEQ ID NO: 4 - protein, 5 aa, synthetic construct
FHVSF

SEQ ID NO: 5 - protein, 6 aa, synthetic construct
WFHVSF

SEQ ID NO: 6 - protein, 8 aa, synthetic construct
VWFHVSFC

SEQ ID NO: 7 - protein, 10 aa, synthetic construct
PVWFHVSFCL

SEQ ID NO: 8 - protein, 12 aa, synthetic construct
TPVWFHVSFCLV

SEQ ID NO: 9 - protein, 5 aa, synthetic construct
GGGGS

SEQ ID NO: 10 - protein, 30 aa, synthetic construct
GGGGSGGGGSGGGGSGGGGSGGGGSGGGGS

SEQ ID NO: 11 - protein, 14 aa, synthetic construct
EGKSSGSGSESKST

SEQ ID NO: 12 - protein, 33 aa, synthetic construct
EGKSSGSGSESKSTEGKSSGSGSESKSTGGGGS

SEQ ID NO: 13 - protein, 430 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGGGGSGGGGSGGGGSGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 14 - protein, 433 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEGKSSGSGSESKSTEGKSSGSGSESKSTGGGGSGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 15 - DNA, 540 bp, synthetic construct
  source 1..540: mol_type = other DNA
atggcggtgttcaaaacaacgctgtggcggctcatctctggcaccttgggcataatatgcttgtcccttatgtctactctcggcattctgctcaaaaatagcttcacgaagctgtctatagaacctgcgtttacccccggacccaacatcgagttgcagaaagactccgactgctgttcctgtcaagaaaaatgggtcggataccgatgcaactgttactttatttcctctgagcagaaaacctggaatgagagccgacatctgtgtgcaagtcagaagtctagccttttgcaattgcaaaacaccgacgagcttgacttcatgtcctcttcccaacaattctattggatcgggcttagctattccgaagagcacaccgcatggctttgggagaatgggagcgcactgagccaatacctgtttccttcattcgaaacctttaacactaagaattgtatcgcttacaatcctaatggtaatgctctggatgaaagttgcgaggataaaaatcgctatatttgtaagcagcaattgatttaa

SEQ ID NO: 16 - DNA, 1710 bp, synthetic construct
  source 1..1710: mol_type = other DNA
atggaaaaaaagtggaaatactgcgccgtgtactacataatacagattcactttgtcaaaggagtatgggagaaaacagtgaacacagaagagaacgtatatgcaacattgggatctgatgtgaatctcacgtgccagacgcagaccgtaggctttttcgttcaaatgcaatggtccaaagtaacgaataaaatagacctcatcgccgtatatcatccgcaatatggtttctattgcgcgtacggcagaccatgcgagagcctcgtcaccttcaccgagactccggagaatggcagcaagtggacgcttcacctgcggaatatgagctgtagcgtgtccggaagatatgaatgcatgttggtgctgtatcccgagggtatacagactaagatctacaatctgctcattcaaacgcacgtaactgctgacgaatggaactcaaatcacaccattgaaatagagatcaatcagactctggagataccttgttttcaaaattcctccagcaaaataagttcagagtttacttatgcctggagtgtggaggacaatggcacacaagaaaccttgatttctcaaaaccacctcatttcaaactccaccttgttgaaggaccgggtaaagttgggaacggactacagactgcatctgtcccccgtgcaaatatttgacgacggcaggaagttctcctgtcacataagagtaggacctaacaagattctcagatcttcaactaccgtaaaagtgtttgctaagcccgagatccccgtaatcgtggagaacaattccacagacgtactcgtcgagcgcagattcacctgtttgttgaaaaacgtttttcccaaagccaatatcacgtggtttatcgatgggtccttccttcacgatgagaaggagggcatctatattacaaacgaggaaaggaaggggaaagatggatttcttgaactcaaatccgttctgactcgcgtccactccaataagccagcacaaagcgataatttgactatctggtgcatggcgttgtcccccgtgccgggcaacaaggtttggaacatctcttccgaaaaaataaccttcctcttgggctccgagatttcatctaccgaccctccattgtccgtaaccgagtctacactcgacactcagccgtcccctgcctcttccgtgagtcctgcacgctaccccgcgactagctctgtgacactggttgacgtatcagcattgaggcctaacactactcctcaaccatcaaattcatctatgacaacccggggtttcaattatccatggacttcctccggcacggacacaaaaaaatcagtctcacgaattccttccgaaacatactcatccagcccaagcggcgcgggttccacattgcatgataacgtatttaccagtactgcacgggccttcagcgaagtacctacaactgcgaacggatctacaaaaaccaatcatgttcacataactggcatcgttgtgaataaacccaaagacggcatgtcatggccagtgatagttgccgcattgctcttctgctgcatgatcctgtttggcctgggagtccgcaaatggtgtcagtatcagaaagagataatggagcgaccgcctcctttcaaaccccctccacctcccataaagtacacgtgtattcaggaacctaatgaaagcgaccttccctaccatgagatggagactctttag

SEQ ID NO: 17 - DNA, 1098 bp, synthetic construct
  source 1..1098: mol_type = other DNA
atgcttggtcaagttgtaactcttatcctgttgttgctgctcaaagtgtaccaaggcaaaggctgtcaaggctctgctgaccatgtcgtttcaataagcggtgttcccttgcaactccaacccaattccatacaaaccaaggtcgatagtatcgcttggaagaagctcctgccgtcccaaaatgggtttcatcacatattgaaatgggaaaacggttcccttccatcaaacacgtccaacgacagattctcctttatcgtaaaaaacctcagtctgctcatcaaagctgctcaacagcaagattcagggctttattgcctggaagttactagcatttctggcaaggtccagaccgccacctttcaagtgttcgtgtttgataaggtcgagaagccgcggctccaaggacagggaaaaattctggaccgagggagatgccaggtcgcgctgagttgcctcgtgagccgcgatggaaatgtatcttacgcttggtaccggggcagcaagttgatccagaccgcgggcaatctgacataccttgacgaggaggtcgacatcaacggcacacatacttacacttgcaatgtgagtaaccctgtatcctgggagtcccatacgctgaatctgacacaagactgccaaaacgcccatcaagagttccgattttggccctttctggttatcatcgtaatattgagtgctttgttccttggtacattggcctgtttctgtgtctggcgacggaaacgcaaggaaaagcagagcgagacaagccctaaagagtttttgaccatatacgaggatgtcaaggacctgaaaacaagaaggaatcatgaacaagagcagactttccccggcggaggttctaccatatattctatgattcaaagtcaaagcagcgcgcccacctcacaagagcctgcgtatactttgtattcccttattcaaccttcacggaaaagcgggtccaggaagaggaatcatagcccgtcctttaattcaactatctatgaagtcattggaaagagtcagcctaaggcacaaaatcctgcgaggctctcccggaaggaattggaaaactttgacgtatactcctag

SEQ ID NO: 18 - DNA, 1083 bp, synthetic construct
  source 1..1083: mol_type = other DNA
atgaaccctacggacattgcggatactaccctcgatgagtcaatttatagcaattattacctttatgaatcaatccccaagccgtgcaccaaggaaggcattaaagcctttggtgaactctttcttccacctctgtattcacttgtgtttgtttttggactgctcggcaactcagttgtggtgctggtactctttaaatacaaaagactccgcagcatgaccgatgtgtacctcttgaatctggcaatatctgacctcctgtttgtattctccctccccttttggggatattacgctgctgatcaatgggtttttgggttgggcctctgcaaaatgatcagctggatgtacctcgtgggattttactccgggattttctttgtcatgctcatgtccatcgaccgataccttgccatagttcatgcggtcttttctttgcgggcccgaaccctgacatacggcgttatcacgagcctggctacgtggagcgtggctgttttcgcgagcctccctggctttctcttttcaacctgctacacggagcgcaaccatacctattgcaaaactaaatactcacttaactccactacttggaaggtgctctccagtttggaaatcaatattttgggcctggttatccctctgggcataatgttgttttgctattccatgatcataagaacattgcaacactgcaagaacgagaaaaaaaataaggccgtaaaaatgatatttgcagtagttgtcctttttcttggtttttggacgccctataacatagtactgttcttggaaactttggtagaactggaggtgctccaagattgtacattcgaaagatatttggactatgccattcaagctaccgaaactctcgctttcgtgcattgctgtctgaaccccataatatactttttcctcggcgaaaaattccgaaaatacatactccagctcttcaaaacctgccggggcctctttgtactgtgccaatactgtggcttgctccaaatatatagcgcggacacaccatcttcctcctacacacaaagtactatggaccatgaccttcacgatgcgttgtag

SEQ ID NO: 19 - DNA, 1110 bp, synthetic construct
  source 1..1110: mol_type = other DNA
atgacccccacggactttacttccccgattccaaatatggcggacgactatggcagcgaatccacgagctccatggaggactatgtgaatttcaatttcaccgatttctattgcgagaagaacaatgttcgacagttcgcctcccacttcctgccaccactgtactggctggtattcatcgtgggggcgctgggaaattccctggtcatacttgtatattggtactgtacccgcgtaaaaactatgacggatatgtttctcctcaaccttgcgatagctgatctgctttttttggtcacgctgcccttctgggcaatcgcagcggcagatcagtggaaattccaaacctttatgtgcaaagtcgtgaattccatgtataagatgaatttttattcatgcgtgcttctcataatgtgcattagtgttgatcggtatatagccatcgcccaggcgatgagggctcatacctggcgcgagaaacgactcctgtactccaaaatggtatgtttcaccatctgggtacttgccgctgcactttgcatacctgaaattttgtactcccaaattaaagaggagtccgggattgcaatttgcacaatggtctaccctagcgatgaatcaactaaacttaaatctgcggtgcttactctcaaagttatactcgggttctttcttccctttgtcgtcatggcctgctgctatactattataatacatacgttgatacaggcaaagaaatccagcaaacataaggcgttgaaagtcactatcacagtgttgactgtgttcgtgttgtctcaatttccatacaattgtatattgctggttcaaacaatagacgcctatgcaatgttcatctccaattgtgcggtttctactaacatcgacatttgcttccaagtcacccaaactatcgcattcttccattcatgcttgaatccggtactttatgtatttgtcggcgagaggtttcgcagagatttggtcaaaaccctcaagaaccttgggtgcatcagccaagcgcaatgggtatcatttacgcgccgggaggggtcactgaaactcagtagtatgcttctggaaactactagtggggcgctctccctgtga

SEQ ID NO: 20 - DNA, 1029 bp, synthetic construct
  source 1..1029: mol_type = other DNA
atggcggaacacgattaccacgaggattatggctttagcagctttaacgattccagccaggaggaacatcaggactttctgcaattctccaaagtatttttgccatgcatgtatctggtggtgtttgtgtgtggcctcgttggaaatagtctcgttctcgtaatatccatattttaccacaaactgcaatctctcaccgatgtgttccttgtaaacctgccccttgccgatctcgtctttgtatgtacactgccgttttgggcatatgctggcatacacgaatgggtatttgggcaggttatgtgcaaatcacttcttggaatctatacgataaacttctatacaagcatgcttattctcacttgcattaccgtggacagattcatagtggtggtgaaggcgaccaaagcgtataaccaacaagctaaacgaatgacttgggggaaggtcaccagccttttgatctgggttatctcacttcttgtgtctcttccacaaataatctatgggaacgtctttaatcttgacaaattgatttgcggataccatgacgaagcgatcagtactgtagtattggcgacacagatgacgcttggattctttctcccgcttctgacgatgatcgtttgttattccgttatcattaaaaccctgctgcatgctggcgggtttcaaaaacacagatctctcaagataattttccttgtgatggccgtgttcctccttacccaaatgccgttcaatcttatgaagtttatacgctcaactcattgggagtattacgctatgacctcttttcattacacaatcatggtgactgaagccattgcgtatcttcgcgcttgtctcaacccggttctctatgcattcgtgtccctgaaatttagaaagaacttctggaagttggtcaaagacattggctgtttgccttatctgggagtcagccatcaatggaagagtagtgaggataacagcaaaacgttttccgcctcccataacgttgaggcaacatcaatgtttcaactctag

SEQ ID NO: 21 - DNA, 1083 bp, synthetic construct
  source 1..1083: mol_type = other DNA
atgcttagtaccagccgaagtagatttataagaaacacaaacgagtctggggaggaggtgacgactttttttgattatgattatggcgcgccgtgccataaatttgacgttaaacaaataggagcccaactgctccctccattgtattccctggtgttcatattcggcttcgtgggaaacatgctggtggtgcttatattgatcaactgtaaaaagctcaaatgtcttaccgatatttatctccttaatctcgcaataagcgatttgcttttcttgatcactctccccctttgggcccattccgctgctaacgaatgggttttcggcaacgcgatgtgtaaacttttcaccggcttgtaccatatcggctacttcggcggcatattctttataatacttttgactatcgaccgctacctcgctatcgtccacgcggtctttgctcttaaggcaaggacggtaacctttggagtcgttacctccgttatcacttggctggtggccgtgtttgcctccgttcccggcataatttttacaaaatgccagaaagaggattcagtatatgtctgtgggccctatttcccccggggatggaataatttccatacaatcatgcgaaatatactgggactcgtacttccacttcttataatggttatatgctactccggaatcttgaaaacacttctcagatgtcggaatgagaagaagcgccatcgagccgtgcgagtgatttttaccatcatgatcgtatacttcttgttctggacaccgtacaacatcgtcatattgctgaatactttccaagaattttttggcctttccaactgcgaaagcacctctcaactggatcaagcgactcaggtgacagaaaccttggggatgactcactgctgcattaacccgataatttacgcgttcgtgggcgagaagtttcgacgatatttgagcgtgttcttccggaagcacatcaccaaaagattttgcaagcaatgcccagtcttttatcgagaaaccgtagatggagttacgagtacgaacactccatccactggcgagcaagaagtgtcagcaggactttaa

SEQ ID NO: 22 - DNA, 1083 bp, synthetic construct
  source 1..1083: mol_type = other DNA
atggaggattttaacatggagagtgactcctttgaagatttctggaaaggggaggacctcagtaattactcatactcttctaccctcccgccattcttgctcgacgcggcaccctgcgagcccgagagtcttgagataaacaaatattttgtagtgataatttacgcccttgtgttcttgctttccttgttgggcaatagcttggtgatgctggtcatcctgtactcacgggtgggacggtccgttaccgacgtgtacctcctgaatctggccctcgcggacttgctttttgccctgactttgccaatatgggcagctagcaaagtcaatggctggatatttggaacctttctgtgtaaggtcgtgtcactgttgaaagaagtaaacttctacagtggcatactgttgcttgcctgtatatctgtcgatcggtacctcgctatagtacacgccacacggacgctcacacaaaaacgctatttggtgaaattcatatgcttgagcatatggggcctttcccttttgcttgctttgccggtcctccttttccgacggactgtctactcctctaatgtgtccccggcttgttatgaggacatggggaacaacaccgctaactggcgcatgctcctgaggatactcccacaaagttttggcttcatcgtcccgctccttataatgctcttttgctacggtttcaccttgcgaactctttttaaggctcatatgggccagaagcacagagctatgcgcgtgattttcgccgtggtccttatcttcttgttgtgctggctcccttacaatttggttctgctggcggatactctgatgcgaacgcaagttattcaggagacttgcgagcggcgaaatcatatagatagggcacttgacgccactgagatcttggggatactccattcctgtctcaacccgctcatatatgcgttcattggccaaaagttccgacatggtctgctcaagattctcgcgattcacggacttatcagtaaagacagtctgccaaaagattcacgcccctcatttgttggatcttcctccggacatacatcaactacgctttaa

SEQ ID NO: 23 - DNA, 1068 bp, synthetic construct
  source 1..1068: mol_type = other DNA
atggatcaattccccgaaagcgtcaccgaaaattttgagtatgacgatttggctgaagcgtgttatataggcgacatagttgtgttcggcactgttttcttgtccatcttttactccgttattttcgccattggacttgtgggcaatctgcttgtggtgttcgcacttaccaatagcaagaagccaaagagcgtcacggatatttatttgctgaatctggctttgtctgatctgcttttcgtggctactctccccttttggacgcattacctgatcaatgaaaaggggctgcataacgctatgtgcaaatttaccacagcgttttttttcatcggtttttttggctcaatctttttcataaccgttattagcattgatcgctaccttgcgattgtacttgccgctaatagtatgaacaatcgcacggtgcaacacggagttacaatatcattgggagtatgggcggctgcaattctcgtcgccgcgccacaatttatgttcaccaaacaaaaagagaacgagtgcttgggggattatcccgaggtcctccaggagatctggcctgttctgaggaacgtggaaacaaatttcctcggatttctgctccccctccttatcatgtcatactgctattttcgcattatccaaacattgttttcttgtaaaaatcacaagaaagccaaggcgatcaagctgattctgcttgttgtgatcgtctttttcctcttttggaccccttataatgtaatgatttttttggaaacactcaaactctacgacttctttccatcctgcgatatgcggaaggatctgcggctggcgctctccgtaaccgaaactgtagcttttagtcattgttgtttgaatcctttgatctatgcgtttgccggggaaaagtttagaagatacctgtatcacctctatggaaagtgtttggccgttttgtgtggtcgatctgtccacgttgatttctcctcctcagagtcccaacgcagcagacacgggtctgtactctcctcaaacttcacatatcatacgagcgacggagatgcattgctgcttttgtga

SEQ ID NO: 24 - DNA, 696 bp, synthetic construct
  source 1..696: mol_type = other DNA
atgaataaacaaagaggaacattctccgaggtatctctggctcaggaccctaaaagacagcaacgcaaacctaaaggaaataaatcaagcatcagtggcacagaacaggaaatatttcaagtggaattgaatctgcagaatccctccttgaaccaccagggcatcgacaaaatttatgattgtcaggggctcctgccgccgcccgaaaagttgacagccgaggtgctgggcatcatctgtatagtcttgatggcgacggttcttaagacaattgtccttataccctttctggagcaaaacaatttttcacccaatactcgaacccaaaaagctcggcactgcggccactgtcccgaggagtggataacatactcaaactcttgctattatatcggcaaagaaagaagaacttgggaggagagtttgctcgcttgtacgagcaaaaatagcagtctcctttccatcgataacgaagaggagatgaaattccttgcgtcaatactgcctagttcttggattggcgtctttcggaactcctcacaccacccttgggtaactataaacggcctcgcattcaagcataagatcaaggactctgacaacgcggagcttaattgtgcggttctgcaagtgaaccggttgaagagtgcccagtgcggatcatccatgatataccactgtaaacacaagctgtag

SEQ ID NO: 25 - DNA, 576 bp, synthetic construct
  source 1..576: mol_type = other DNA
atgggtagagggctgctgcgcggtttgtggccgctccatatcgttctttggacgaggattgcatccactatccctccgcatgtgcaaaagtccgtaaacaacgacatgatcgtcacagacaataatggcgcggtaaagtttcctcaactctgtaaattttgtgacgtgaggtttagcacctgcgataaccagaagagctgcatgtcaaattgttctattacctccatatgcgaaaaaccccaggaggtctgcgtcgcagtgtggagaaagaacgacgagaacataaccttggaaactgtttgccatgatccgaaattgccgtatcatgactttattctcgaggacgcggccagtccaaaatgcattatgaaggaaaaaaagaagcctggcgagactttctttatgtgctcatgttcaagcgatgagtgtaatgataatataattttcagcgaggaatacaacacgagcaaccctgaccttctcttggtcatcttccaggtcactggcatatccctcctgccgccgttgggggttgcgattagtgtaattattatattttattgctacagagtgtag

SEQ ID NO: 26 - DNA, 915 bp, synthetic construct
  source 1..915: mol_type = other DNA
atgtccctcacggttgtttcaatggcctgtgtaggcttcttcctcctccaaggcgcctggccccatgagggagtgcatagaaagccttccctcttggcacatcccggtcgccttgttaaatccgaggagactgttattcttcaatgttggtctgatgtcatgttcgagcacttccttttgcaccgggaaggcatgttcaacgatacgttgcgcttgatcggagagcatcatgatggtgttagtaaagccaacttttctatttcacggatgaaacaagacctggctgggacatacagatgctacggatccgtcacacattctccgtatcaattgtctgctccttctgaccctttggacatagtaataatcggcctttatgaaaagccgtccttgtctgctcaacctgggccaactgtactggccggagaaaacgtgaccctctcatgctcctcccgctcctcttacgacatgtaccatctctcccgggagggggaagcccacgaacgacggcttccggcgggcactaaagtaaatggcacgtttcaagcgaatttcccgctcggtcccgccacccacggcggaacctatcggtgctttggctccttccgagacagcccttatgagtggagcaagtcaagcgatccgctgctcgtgtctgttactggcaatccaagcaactcatggccgagccccacggaaccgagtagcgaaacaggcaatcccagacacctccatgtcctcattgggacgtccgtcgtcaagattccatttacaattttgttgttcttcttgttgcaccgatggtgttcagacaaaaagaacgctgctgtaatggatcaagaacctgcgggcaatcgcactgttaactccgaggatagcgacgaacaagatcatcaagaggtgtcttatgcgtga

SEQ ID NO: 27 - DNA, 915 bp, synthetic construct
  source 1..915: mol_type = other DNA
atgagcttgatggtagtgtctatggcgtgtgtaggatttttcctgcttcaaggcgcttggccgcatgaaggcgtgcatagaaagccctccctgctcgcccatcctgggccgctggtaaaaagcgaggagacagttattcttcaatgttggagtgatgttagattcgaacattttttgctgcaccgggaggggaaatacaaggatacgctccaccttatcggagagcatcatgacggagtcagtaaagcaaacttctctatcggccctatgatgcaggacttggcgggcacgtaccgatgctacggttctgtgacccattccccctatcaactgtccgccccctccgaccctctggacatcgtcatcactggactctacgaaaaaccatccttgtctgctcaacccggccccactgtgctcgccggggagagtgtcacgcttagctgttcctctcgatcctcctatgacatgtaccatctttctcgcgaaggagaagcacatgaaagacgattcagtgccggtccgaaagtgaatggcacattccaggccgactttccgctgggacctgccacgcacgggggcacgtaccgatgttttgggagttttcgggatagcccatatgagtggagtaattcaagcgacccgctcttggtgtctgtgactgggaaccccagcaactcctggccgagccccaccgaaccttctagcaagactggcaacccccgacacttgcacgtgctcatcggaacgagtgtcgtcaaaatcccttttaccattcttctctttttcttgcttcatcgatggtgttcaaataaaaagaacgccgcagtgatggatcaagaaccggcaggcaatcggaccgtaaactccgaggactccgacgaacaggaccatcaagaggtgagttacgcatga

SEQ ID NO: 28 - DNA, 915 bp, synthetic construct
  source 1..915: mol_type = other DNA
atgtctctgatggttatcagcatggcctgtgtgggattcttctggctccaaggcgcgtggccgcacgagggattccgaagaaaaccctctctcctggcccaccccggcagattggtaaagagcgaggaaactgtaatacttcagtgctggagtgatgtcatgttcgaacactttcttttgcatcgggaagggacatttaacgatactttgagactgatcggagagcatattgacggagtgagcaaggcgaatttctcaatcggaaggatgcgccaggatctggcaggcacatacagatgctatggttctgttcctcattcaccgtaccaattcagcgctccatccgatcctctcgacattgtaataactggtctttatgagaaaccgtcactctccgcccaacctggcccaaccgtgttggcgggcgagagtgtcactctgtcctgctcatcttggagttcatacgacatgtatcatttgtctactgaaggggaagcccacgagaggcggttctccgccgggccaaaagttaatggaacctttcaggctgacttccctctcggacctgctacccaaggaggaacctacagatgtttcggatcttttcacgactctccctatgagtggagcaagagctccgatcccttgctcgtatccgttacgggcaatccgtccaactcctggccttctccgacggaaccctcatcaaaaaccggcaaccctcgacacctgcatgtcttgatcggaactagtgtcgtcaagctcccattcaccatacttctcttctttttgctgcatcggtggtgttccgataaaaaaaacgctagtgttatggatcaaggccccgccggtaaccggactgtgaatcgagaagattcagatgaacaggaccatcaagaggtcagctatgcctga

SEQ ID NO: 29 - DNA, 915 bp, synthetic construct
  source 1..915: mol_type = other DNA
atgagtctcatggtaataatcatggcttgtgtcgggtttttcctgcttcagggggcttggccgcaggagggagttcacagaaagccctctttcctggcgctccccggtcatcttgtaaaatctgaagaaactgtaatactgcagtgttggagcgacgtgatgtttgaacattttctccttcatcgggaagggaagttcaataataccctgcacctgatcggcgaacaccatgatggagttagtaaagcaaacttcagcataggcccgatgatgccggttcttgcgggaacctataggtgttacgggagtgttccgcactctccttaccaactctccgcaccaagcgatcctctcgatatggtgattatcggtctttacgaaaaaccgagcctgtcagctcaacctggaccgacggtccaagcaggggaaaatgtaactctcagttgctccagccgctcctcctatgatatgtaccatttgagtcgggagggagaagcacacgaacggcgcctgcctgccgtacggtctataaacggcacctttcaagcggacttccctctcggccccgcaactcatggcgggacgtacagatgctttggatccttccgggacgcgccatatgagtggagcaattccagcgatccactgctcgtgtccgttaccggaaatccaagcaactcctggccgagccctactgaaccaagttctaagactggcaacccgagacacctgcatgtgcttattggcacctcagtagtgaagatcccgtttactatcttgctcttctttcttcttcatcgctggtgtagcgataaaaagaatgcggccgtgatggatcaagagccagcaggtaaccggacagtaaacagcgaagattccgacgagcaagatcaccaggaagtttcttatgcatga

SEQ ID NO: 30 - DNA, 915 bp, synthetic construct
  source 1..915: mol_type = other DNA
atgagtcttatggtgatttccatggcctgtgttgcttttttcctcctgcagggtgcttggcctcacgaagggttccgcaggaagccatctctccttgcccaccctggtcctcttgtaaagtcagaggaaacggttatcttgcaatgttggagcgatgtgatgtttgagcatttcctgctccaccgggagggaacatttaaccataccctccgcctcatcggagaacatatcgacggcgtgagcaagggaaacttttcaatagggaggatgacccaggatttggccgggacatatcgctgttatggctcagtcactcactcaccatatcaactcagtgctccgagcgatccacttgacattgtgattaccggcctctatgagaaacctagtctctccgctcaacctggtccgaccgttttggccggcgaatccgtcaccctttcctgctcttcccgctcctcctatgacatgtaccatctgagtagagagggcgaggcgcatgagcggagacttccagccggtccaaaagttaatcgcacctttcaggccgatttcccgctggaccctgcgacccacggcgggacgtaccgctgtttcggatcatttcgggactcaccttatgagtggagtaaaagttcagatcctttgcttgtctccgttacgggcaattcaagcaactcctggccctctcccacggaaccctctagtgaaaccggaaatccccgccatctccatgtgttgattggaacctccgtcgtaaagttgcctttcacgatcctgctgttcttccttttgcatcggtggtgctctaataaaaagaacgcttccgtgatggatcaaggcccggccggtaaccgcacggtcaatagagaagatagtgacgaacaagaccaccaagaagtgagctacgcgtga

SEQ ID NO: 31 - DNA, 1047 bp, synthetic construct
  source 1..1047: mol_type = other DNA
atgagcttgctcgtcgtgtcaatggcctgcgtcggtttcttccttctccagggtgcatggcctcacgagggagtgcaccgaaaaccaagtttgctggctcaccctggcccgttggtgaagtcagaggaaacggtcatattgcaatgttggagcgatgtaatgtttgaacatttcctcctgcaccgagagggcatgttcaatgacacgctgcggcttattggggagcaccacgacggcgtgtctaaggcgaacttctcaatctcccggatgacgcaagaccttgccgggacttatagatgctacgggagtgtcacgcattccccttatcaagtgtctgccccttccgaccccctggacatagtcatcataggcctgtatgaaaaaccgtccttgagtgctcaaccgggtcccacagtcctcgctggcgagaatgttactttgagttgctcctcacgctcttcctatgacatgtatcacctgagccgggagggggaggctcacgagcggcgactgccagcagggccaaaggtaaacggaacttttcaagcagattttccgctcggccccgcaacccacggcggcacatatcggtgttttggatccttccacgactccccctacgaatggtccaagtcatctgatcctctgcttgtctccgtcaccggcaacccgtcaaattcctggccttcacccaccgaaccctcctcaaaaaccggcaacccgaggcatttgcatatccttattggcacaagcgtagttatcatcctcttcattttgctgttttttttgcttcaccgctggtgctccaataagaaaaatgcagcagttatggatcaggagagtgcagggaaccgcaccgcaaacagcgaggattccgatgaacaagacccgcaagaggtgacctacacacagcttaaccactgcgttttcacgcaaaggaagatcacccgaccatcccaacgacccaagaccccgcccaccgatataattgtatataccgagctcccaaacgctgagtctcgatcaaaagttgtctcctgcccatga

SEQ ID NO: 32 - DNA, 1047 bp, synthetic construct
  source 1..1047: mol_type = other DNA
atgtcattgatggtggtttctatggcctgcgtgggtttttttcttctgcaaggagcttggccccacgaaggagttcatagaaagcctagcctccttgcgcatcctggccgcttggtgaaaagcgaagaaaccgtgatacttcagtgttggtctgatgttcggtttgaacacttcttgttgcatcgggagggcaaattcaaggacacacttcacctgattggggaacatcacgatggagtatcaaaagctaacttctctatcggaccgatgatgcaagatctcgccggcacctatcggtgttatgggagtgtgactcactctccgtatcaattgtctgcaccaagcgacccactggatattgtgataaccggcttgtatgagaagccaagtttgtccgctcaacccggtccaaccgttcttgccggcgagagcgtgactctttcctgctcctcccgaagcagttatgatatgtaccacctttcccgggagggcgaggctcacgagtgtagattctctgccggccctaaagtcaacgggaccttccaagcagactttccgctgggaccggctactcacggtggcacttatcgctgctttggttcttttcgcgactccccatacgagtggagcaacagctctgacccgctccttgtgtccgtcatcgggaacccttctaattcatggccctcacccaccgagccgtcatctaagaccgggaacccacgccatcttcatattcttattggcacatccgttgtgattatacttttcattcttctttttttcctccttcatcggtggtgttcaaacaagaaaaacgccgcggtcatggaccaggaaagtgctggcaatcgaacagccaatagcgaggactcagacgaacaggacccacaggaggttacgtacactcaactgaaccactgtgttttcacccaacggaagatcactcgcccctcccaaagaccgaaaacgccccctaccgatataattgtatatgctgaactgccaaatgccgaaagtagatccaaggtagtttcttgtccttga

SEQ ID NO: 33 - DNA, 1026 bp, synthetic construct
  source 1..1026: mol_type = other DNA
atgtctctcatggtagtctctatggtttgcgtgggcttctttctccttcaaggcgcttggccacacgagggagtccaccgaaaaccgtcactcttggcgcatccgggccccttggtaaaaagcgaagaaacggttatccttcaatgctggtccgatgtaagatttcagcacttcctcctccatcgggaaggcaaatttaaggataccctccatctgatcggagagcatcatgacggggtatctaaggcgaacttctctatcgggcccatgatgcaggacctcgctggaacgtatcgatgctacggatctgtaacgcactcaccttaccaactcagtgctccttccgacccattggacattgttataaccggactctatgaaaaaccctcactgagcgcacaaccgggccccaccgtgctcgcgggggagtccgtgacgttgagttgctcttcacggtcctcttacgacatgtaccacctgtctcgagagggggaagctcacgaacggagattttcagcaggcccaaaggtcaatggcacattccaagcagatttcccgcttggccccgctacccatgggggcacgtataggtgttttggttcatttcgggactctccgtatgaatggtccaatagcagtgatccgttgctcgtttctgtaaccggaaatcccagtaatagctggcctagtccgactgagcctagttctgaaaccggcaaccctcgacaccttcatgtactgatcggcacctctgtagtgataattctcttcattctcctccttttttttctgctccaccgctggtgctgcaacaagaagaacgctgtcgtgatggatcaggaacccgctggcaacagaacagttaatcgggaggatagtgatgagcaagacccccaagaggtgacatatgcacaactcaaccattgcgtgttcactcaacggaaaataactcggccatctcaacggccgaaaacccctcccactgacataatcgtatacaccgaattgccaaatgctgagccatga

SEQ ID NO: 34 - DNA, 1134 bp, synthetic construct
  source 1..1134: mol_type = other DNA
atgtcaatgtccccgacggtaattatccttgcatgtcttggattctttctcgatcaaagcgtatgggcacatgtcggtggacaagacaagcctttctgctccgcttggccatccgccgtcgtcccgcaggggggccacgtcacacttcgctgccactatcggagaggtttcaatatattcactctctacaaaaaggatggggtccccgtgccggaactgtataatagaatattctggaactcttttttgatttcacccgtgacgcccgctcacgcgggcacatatagatgcaggggatttcatccgcactcaccaacggagtggagcgccccgagcaacccgttggttattatggtgaccggactttacgaaaaaccctcattgacggctaggccagggcccacagttcgggcgggcgaaaacgtaacgctgtcttgctcttcccaatcctctttcgacatctaccatttgtcacgagagggcgaggctcatgaactcaggctcccagccgtgccatcaattaacgggaccttccaggcagattttccgttgggtcctgcaactcatggagaaacctaccggtgttttggaagcttccacggttccccctatgaatggtccgatccgtctgatccattgcccgtatccgttactgggaacccgtcatccagctggccttctcctacggaacctagttttaagacgggaatagcccgccacctgcatgccgttattagatacagtgttgctatcattttgtttaccatccttcccttctttctgcttcatcggtggtgttcaaaaaagaaagatgctgcggttatgaatcaggaaccagcgggacatcgcacagttaatagggaagattcagacgaacaagacccgcaggaggtcacatatgcacagttggatcattgtatttttacacaaagaaagattacaggaccatcccagcgctccaaacgaccgtcaaccgatacatcagtttgcattgaactgccgaacgcggagccgcgggcgctgtcccctgcacatgagcaccatagtcaggcgctgatgggcagctcacgcgaaacgacggccctttctcaaactcaactggcgagttctaatgtcccagcggctggcatttga

SEQ ID NO: 35 - DNA, 1128 bp, synthetic construct
  source 1..1128: mol_type = other DNA
atgtctctcatggtcatttcaatggcttgtgtgggtttttttcttctgcagggcgcttggactcacgaaggggggcaagataagccccttctctccgcatggccgagtgcggtcgtcccgcgaggcgggcatgtgacgcttctttgtcgctcccggttggggtttaccattttttccttgtataaggaggacggagtccctgttccggagctgtataataagatattctggaagtccatacttatggggccagtaacgcccgcccacgccggcacctaccgctgccggggctcccacccgagatccccgatagagtggagcgccccgtctaatcctttggttatcgttgttactgggttgttcggaaagcctagtctgtctgcccaacccggccctacggtccggactggcgaaaacgtcacccttagctgttccagtcggtcaagtttcgatatgtatcatttgagtagagaaggccgggcgcacgaaccaagactgcctgccgtgccctctgttaatggaacattccaggctgacttccctcttggaccggctactcatggaggcacttacacttgctttggatcactgcatgattcaccttacgaatggagtgacccgtccgaccctttgcttgtgtctgtaaccggcaactcctcttcttcctcctcttcaccaactgaaccgagttccaaaactgggataagaaggcaccttcacatactcatcgggacctcagttgcaattatcctctttatcatcctcttcttcttcctgctgcattgctgctgctccaataagaaaaatgcggcggtaatggaccaagagcccgcaggggataggaccgtcaatagggaggactctgacgatcaagacccccaagaagttacctatgctcaattggatcattgtgtctttactcaaacaaagatcacttctccttcccaacgcccaaagactcctccaaccgacaccactatgtatatggagctgcctaacgctaagcctcggagcctctctccggctcacaagcaccactcacaagcactgcgaggatcatcacgagaaactaccgcattgagccaaaatagagtggcgtccagccatgttcccgccgccggaatatga

SEQ ID NO: 36 - DNA, 1128 bp, synthetic construct
  source 1..1128: mol_type = other DNA
atgtcactgatggtggtgtccatggcgtgcgttggcttctttctcctgcagggggcatggacgcatgagggcggtcaggacaagcctctgttgtccgcgtggcctagtgcggttgttccccggggcggtcacgtcacccttttgtgtcggagcagactgggattcacaatcttctccctttataaagaagatggggtccccgtacccgagctctacaacaagatcttttggaagtcaatactcatggggcccgtaacacccgcacatgccgggacgtaccgctgccggggaagtcacccccgaagccctattgaatggagtgcaccctccaacccgctcgtcattgtggtgactggcctgttcgggaagccctcactcagcgctcaacccggacccaccgtccgaaccggggagaacgtgactctctcttgctcctcccggagtagctttgacatgtatcacctttcccgagaggggcgggcacatgaacctagactgccagcggttccttccgtggatggcacttttcaggccgactttcccctgggacctgctactcacggggggacttacacatgtttttcatccttgcacgactcaccctacgagtggagcgacccctccgatccccttttggtatccgtcaccggaaacagttcctcaagctcctccagtccgacagaaccctcttccaagaccgggatacgccgccacctgcacattctcatcggaacttcagttgcgattatcttgttcatcatactgttcttcttcctgttgcattgctgttgctccaataaaaagaatgcagcagtaatggatcaagagcccgcgggagacaggacagtcaaccgagaggactccgacgatcaagaccctcaagaggtcacatatgcacaactggaccactgtgtctttacgcaaacgaaaataaccagcccgtcccaacgaccaaagactccacccacggacacgacaatgtatatggagcttccaaacgctaagccgagatcattgagtcctgctcacaaacatcattcccaggccctgcgggggtccagtagggaaacaacggcgctgtcccaaaaccgcgttgctagttcacatgtccccgccgccggaatttga

SEQ ID NO: 37 - DNA, 672 bp, synthetic construct
  source 1..672: mol_type = other DNA
ccagttttcttgccgttctgtaagtgttttattcttagtgtgatttttttccattgggatgtttttgattgaacttgttcattttgttttgcttgggaggaaaataaacaattttacttttttcctttaggagcattatgagcattatgtcagaatagaatagaattggggttcgatcttaacaggccagaaatgcctgggtttttttggtttgtttttgtttttgtttttttatcaaatcctgcctgactgtctgcttgttttgcctaccatcgtgacatctccatggctgtaccaccttgtcgggtagcttatcagactgatgttgactgttgaatctcatggcaacaccagtcgatgggctgtctgacattttggtatctttcatctgaccatccatatccaatgttctcatttaaacattacccagcatcattgtttataatcagaaactctggtccttctgtctggtggcacttagagtcttttgtgccataatgcagcagtatggagggaggattttatggagaaatggggatagtcttcatgaccacaaataaataaaggaaaactaagctgcattgtgggttttgaaaaggttattatacttcttaacaattctttttttcagggacttttctagctgtatgactgttacttgaccttctttga

SEQ ID NO: 38 - DNA, 710 bp, synthetic construct
  source 1..710: mol_type = other DNA
cttatttgtcttcttttgtagtcttttgaaatggcataaaaatgcataaaatatatgactaaaggtactgttgtttctgtctcccatccccttcagatacttacagatactgtaaagtgagtagaattctgagttttgaggttgcttcagtgaacattcaacgctgtcggtgagtttggaattaaaatcaaaaccatcgaccgttgattgtaccctatggctaaccatcatctactccatggtgctcagaattcgctgaagacaggaaaccaaaggtggacacaccaggactttctcttccctgtgcagagattattttttaaaaggtcacaatcaacattcattgctgtcggtgggttgaactgtgtggacaagctcactgaacaatgaatgcaactgtggccccgctttttgctgtcacaatcaacagatattccatctttgaaagatgtgttcaaaatagtactattgttctttaagttttccaattcgtcagcatcttctggtttatttattcaatttatatcatgtgatgagatttcaatgaaaaagactctggtttaaacaagaataatgcttctctatcttttgtatgctataaacagagcatgttttagaaaaggcaaaactattttgtccatcaaacgtacagtatcatcatctaggttagctcaccttcctactgttgtagtgtgagtgaaaaaaagaca

SEQ ID NO: 39 - DNA, 710 bp, synthetic construct
  source 1..710: mol_type = other DNA
taccacaaagtactttgaataacaaattgtccctgtctttaacagtgagataattccatctctggaactagcccaatatcggccatgtttttgcttaatgaaaccgatccttttctctcatacaatgtgatgtggaggtttgccaaactctttgttggaagaatcatgcttcttatttgtcttcttttgtagtcttttgaaatggcataaaaatgcataaaatatatgactaaaggtactgttgtttctgtctcccatccccttcagatacttacagatactgtaaagtgagtagaattctgagttttgaggttgcttcagtgaacattcaacgctgtcggtgagtttggaattaaaatcaaaaccatcgaccgttgattgtaccctatggctaaccatcatctactccatggtgctcagaattcgctgaagacaggaaaccaaaggtggacacaccaggactttctcttccctgtgcagagattattttttaaaaggtcacaatcaacattcattgctgtcggtgggttgaactgtgtggacaagctcactgaacaatgaatgcaactgtggccccgctttttgctgtcacaatcaacagatattccatctttgaaagatgtgttcaaaatagtactattgttctttaagttttccaattcgtcagcatcttctggtttatttattcaatttatatcatgtgatgagat

SEQ ID NO: 40 - DNA, 686 bp, synthetic construct
  source 1..686: mol_type = other DNA
ctgggtctgtctgcctgtctgcttgttagtgtgaccaaagggaaccttctgcatcattacgccatctctggcttgtttaacactggccctgggtccctatgagatcttaacagaccctagctccagtccccttcccataacccacctgggctgtgcctgaccacagaatcaaggagacgctggcctgcgagggagctgtagagcagggagcaggaagctgtgtgtgtccagccctgacctgtcctgttctgcccccagcccctcacagtgcttttcaagccatgcttcctgtgcccccagtggggccctggctgggatatcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggaaaccgttaccattactgagtttagtaatggtaatggttctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaa

SEQ ID NO: 41 - DNA, 684 bp, synthetic construct
  source 1..684: mol_type = other DNA
gggagggggccgcaacctcctattcccctctgggtctccgtcccctccctctggagtccacactccctctttgccccttgctggttctctactgcccccagcatagggtggagtgggtgtgcagtttctgcgactcagggtggcgtccccccaacctgtccctgccccttcctgccctctttgatgcggccccacttcctctggcaggaacccccgccctccctggacctgggtataaggcagggactgggcccacggggaggcagcgtccccgaggcagcagcggcagcggcggctcctctccccatggccctgtctcccaacccttgtaccagtgctgggctcagaccctggtacaggcctgggggacagggacctggggaccccggcaccggcaggccccaaggggtgaggtgagcgggcattgggacctcccctccctgtactcccatctctgctgcggcttttatgcgtctctccccttcgggtcccacatatcctctggtgcgctcctgcctcaccgcccccaccccatgcctgtcgtccccacctctgtgtgatgcgcaaagtacacctgtttctattgtacctgcctctcgcggtggtctgtgctctccccagctctgcaaaacccctcctccccatgtgccacaaccctgggccaccgtgtgtcctgtcctgttc

SEQ ID NO: 42 - DNA, 696 bp, synthetic construct; identical to SEQ ID NO: 24
  source 1..696: mol_type = other DNA

SEQ ID NO: 43 - DNA, 1239 bp, synthetic construct
  source 1..1239: mol_type = other DNA
atgcccattatgggctcatccgtctatataacagtggagctcgctattgccgtgcttgcaattctcggcaacgtattggtctgttgggctgtttggcttaactccaaccttcagaacgtgactaattattttgtagtatcacttgctgcagccgatatagctgtgggggtgctcgctattccttttgcgattacaatatccacgggcttttgtgcagcatgtcacgggtgtctcttcatagcctgcttcgtactggtgttgacacagtcatccatttttagccttcttgccatcgctatagatcgctatattgcgattcgaatccccttgcgctataacgggctggtaactggcacaagggccaaaggtattattgctatatgttgggtcctctcatttgctattggattgactccgatgctggggtggaataattgtggacagccgaaggagggcaaaaaccattcacaaggctgtggggaaggacaggtagcctgcctttttgaggatgtggtacccatgaactatatggtctatttcaatttctttgcatgcgtccttgtaccgcttttgctcatgctgggggtttacctcaggatatttcttgccgcccgaaggcaacttaaacagatggaatctcaaccgctcccaggggagcgcgccagaagtacactgcaaaaggaagtgcatgccgccaaaagcctcgcgatcatagttggcttgtttgccttgtgttggcttcccctccacatcataaattgttttacttttttttgtcccgactgttcacacgcgccattgtggctcatgtaccttgcgattgtgctcagccacacaaactcagtagttaatcctttcatctacgcttatagaattcgagaatttcgccagacgtttagaaagattatacggtctcacgtattgcggcagcaggaaccttttaaggctgctggcacttctgctcgggtgctcgcggcacatggatcagacggcgagcaggtctcactcagactgaacggccatccaccaggagtttgggcgaacggctctgctccccaccctgaacgacggcctaatggttacgctcttggcctggtgtctggcggtagcgcccaggaatcacaagggaatacgggtttgccagatgtcgagctgcttagccacgaacttaaaggggtttgtccagagccgccaggcctcgatgatcctcttgctcaggacggagctggcgtgtcatga

SEQ ID NO: 44 - DNA, 20 bp, synthetic construct
  source 1..20: mol_type = other DNA
agcagtgctgcccacagcat

SEQ ID NO: 45 - DNA, 20 bp, synthetic construct
  source 1..20: mol_type = other DNA
gtgatcggagcactcctgac

SEQ ID NO: 46 - DNA, 20 bp, synthetic construct
  source 1..20: mol_type = other DNA
tctggacaccctaaccgtga

SEQ ID NO: 47 - DNA, 20 bp, synthetic construct
  source 1..20: mol_type = other DNA
ggacaattccatacagaaac

SEQ ID NO: 48 - DNA, 20 bp, synthetic construct
  source 1..20: mol_type = other DNA
cttcagtcgacagtagagga

SEQ ID NO: 49 - DNA, 20 bp, synthetic construct
  source 1..20: mol_type = other DNA
tcagagtctcgtaagtctcc

SEQ ID NO: 50 - DNA, 20 bp, synthetic construct
  source 1..20: mol_type = other DNA
ccttgtgccgctgaaatcca

SEQ ID NO: 51 - DNA, 20 bp, synthetic construct
  source 1..20: mol_type = other DNA
tgaacctggctaccaggacc

SEQ ID NO: 52 - DNA, 20 bp, synthetic construct
  source 1..20: mol_type = other DNA
gtacccaccgccatactacc

SEQ ID NO: 53 - DNA, 20 bp, synthetic construct
  source 1..20: mol_type = other DNA
ggacgatgtgcagcggccac

SEQ ID NO: 54 - DNA, 20 bp, synthetic construct
  source 1..20: mol_type = other DNA
tgctggcgatacgcgtccac

SEQ ID NO: 55 - DNA, 20 bp, synthetic construct
  source 1..20: mol_type = other DNA
ccgacttctgaacgtgcggt

SEQ ID NO: 56 - DNA, 20 bp, synthetic construct
  source 1..20: mol_type = other DNA
agtccacgtcggccaccaga

SEQ ID NO: 57 - DNA, 20 bp, synthetic construct
  source 1..20: mol_type = other DNA
tcgggcctcgctggccgtaa

SEQ ID NO: 58 - DNA, 20 bp, synthetic construct
  source 1..20: mol_type = other DNA
gggttccattacggccagcg

SEQ ID NO: 59 - DNA, 20 bp, synthetic construct
  source 1..20: mol_type = other DNA
aaattctcggaggatgctcc

SEQ ID NO: 60 - DNA, 20 bp, synthetic construct
  source 1..20: mol_type = other DNA
agaataatgtcgaagttgcc

SEQ ID NO: 61 - DNA, 20 bp, synthetic construct
  source 1..20: mol_type = other DNA
gggcaacttcgacattattc

SEQ ID NO: 62 - DNA, 20 bp, synthetic construct
  source 1..20: mol_type = other DNA
ggcacagtagaagccgtatt

SEQ ID NO: 63 - DNA, 20 bp, synthetic construct
  source 1..20: mol_type = other DNA
aggcacagtagaagccgtat

SEQ ID NO: 64 - DNA, 20 bp, synthetic construct
  source 1..20: mol_type = other DNA
acggcttctactgtgcctat

SEQ ID NO: 65 - DNA, 20 bp, synthetic construct
  source 1..20: mol_type = other DNA
tatcgttcacggtcagcgac

SEQ ID NO: 66 - DNA, 20 bp, synthetic construct
  source 1..20: mol_type = other DNA
tcgctgaccgtgaacgatac

SEQ ID NO: 67 - DNA, 20 bp, synthetic construct
  source 1..20: mol_type = other DNA
cgctgaccgtgaacgataca

SEQ ID NO: 68 - DNA, 20 bp, synthetic construct
  source 1..20: mol_type = other DNA
caggactcacacgactattc

SEQ ID NO: 69 - DNA, 20 bp, synthetic construct
  source 1..20: mol_type = other DNA
gtgtagatggattaggactc

SEQ ID NO: 70 - DNA, 20 bp, synthetic construct
  source 1..20: mol_type = other DNA
cttatggtcaaataacgact

SEQ ID NO: 71 - DNA, 20 bp, synthetic construct
  source 1..20: mol_type = other DNA
attgcaaagcgacaacccaa

SEQ ID NO: 72 - DNA, 20 bp, synthetic construct
  source 1..20: mol_type = other DNA
gcgacaacccaaaggttgtg

SEQ ID NO: 73 - DNA, 20 bp, synthetic construct
  source 1..20: mol_type = other DNA
cgacaacccaaaggttgtga

SEQ ID NO: 74 - DNA, 20 bp, synthetic construct
  source 1..20: mol_type = other DNA
tattccggttatggatgttc

SEQ ID NO: 75 - DNA, 20 bp, synthetic construct
  source 1..20: mol_type = other DNA
tggtattggtattccggtta

SEQ ID NO: 76 - DNA, 20 bp, synthetic construct
  source 1..20: mol_type = other DNA
ggatcggttgtagtaatctg

SEQ ID NO: 77 - DNA, 20 bp, synthetic construct
  source 1..20: mol_type = other DNA
cctcctcgtgctggcggcgg

SEQ ID NO: 78 - DNA, 20 bp, synthetic construct
  source 1..20: mol_type = other DNA
ttgacttaattcctcgagat

SEQ ID NO: 79 - DNA, 20 bp, synthetic construct
  source 1..20: mol_type = other DNA
catacaaacggcctatctcg

SEQ ID NO: 80 - DNA, 20 bp, synthetic construct
  source 1..20: mol_type = other DNA
ttcgcagaaggctgagatcc

SEQ ID NO: 81 - DNA, 20 bp, synthetic construct
  source 1..20: mol_type = other DNA
cgctcagcaccgtgtagcgg

SEQ ID NO: 82 - DNA, 20 bp, synthetic construct
  source 1..20: mol_type = other DNA
cgctacacggtgctgagcgt

SEQ ID NO: 83 - DNA, 20 bp, synthetic construct
  source 1..20: mol_type = other DNA
gattggactctcttacagtg

SEQ ID NO: 84 - DNA, 20 bp, synthetic construct
  source 1..20: mol_type = other DNA
acagtgaggagcacaccgcc

SEQ ID NO: 85 - DNA, 20 bp, synthetic construct
  source 1..20: mol_type = other DNA
ggagcacaccgcctggttgt

SEQ ID NO: 86 - DNA, 20 bp, synthetic construct
  source 1..20: mol_type = other DNA
gaagctcattgttgggatcc

SEQ ID NO: 87 - DNA, 20 bp, synthetic construct
  source 1..20: mol_type = other DNA
ctccagagaagctcattgtt

SEQ ID NO: 88 - DNA, 20 bp, synthetic construct
  source 1..20: mol_type = other DNA
gctccagagaagctcattgt

SEQ ID NO: 89 - DNA, 20 bp, synthetic construct
  source 1..20: mol_type = other DNA
ctgcggtgccttgagccctc

SEQ ID NO: 90 - DNA, 20 bp, synthetic construct
  source 1..20: mol_type = other DNA
cttgagccctccgggaatgg

SEQ ID NO: 91 - DNA, 20 bp, synthetic construct
  source 1..20: mol_type = other DNA
ttcgccagacgcgccgcctg

SEQ ID NO: 92 - DNA, 20 bp, synthetic construct
  source 1..20: mol_type = other DNA
ttgctccaagaagtagttca

SEQ ID NO: 93 - DNA, 20 bp, synthetic construct
  source 1..20: mol_type = other DNA
cttgctccaagaagtagttc

SEQ ID NO: 94 - DNA, 20 bp, synthetic construct
  source 1..20: mol_type = other DNA
ctgaactacttcttggagca

SEQ ID NO: 95 - DNA, 20 bp, synthetic construct
  source 1..20: mol_type = other DNA
gcgcgtgatgcgccggtaat

SEQ ID NO: 96 - DNA, 20 bp, synthetic construct
  source 1..20: mol_type = other DNA
gccggtaatcggcgtgcgaa

SEQ ID NO: 97 - DNA, 20 bp, synthetic construct
  source 1..20: mol_type = other DNA
tccgttcgcacgccgattac

SEQ ID NO: 98 - DNA, 20 bp, synthetic construct
  source 1..20: mol_type = other DNA
atggttccacggactggatc

SEQ ID NO: 99 - DNA, 20 bp, synthetic construct
  source 1..20: mol_type = other DNA
tggttccacggactggatct

SEQ ID NO: 100 - DNA, 20 bp, synthetic construct
  source 1..20: mol_type = other DNA
taagacccagatccagtccg

SEQ ID NO: 101 - DNA, 20 bp, synthetic construct
  source 1..20: mol_type = other DNA
gtcgcggatcagaaaggtgc

SEQ ID NO: 102 - DNA, 20 bp, synthetic construct
  source 1..20: mol_type = other DNA
cggatcagaaaggtgccggc

SEQ ID NO: 103 - DNA, 20 bp, synthetic construct
  source 1..20: mol_type = other DNA
cagcaggttcgcctcgccgc

SEQ ID NO: 104 - DNA, 20 bp, synthetic construct
  source 1..20: mol_type = other DNA
cggcccacgtagtacacgat

SEQ ID NO: 105 - DNA, 20 bp, synthetic construct
  source 1..20: mol_type = other DNA
gcataacccgggcgcctcgg

SEQ ID NO: 106 - DNA, 20 bp, synthetic construct
  source 1..20: mol_type = other DNA
ccgaggcgcccgggttatgc

SEQ ID NO: 107 - DNA, 20 bp, synthetic construct
  source 1..20: mol_type = other DNA
aggtcgcagccgccgcgcag

SEQ ID NO: 108 - DNA, 20 bp, synthetic construct
  source 1..20: mol_type = other DNA
gtagtaaagcgcctccggcc

SEQ ID NO: 109 - DNA, 20 bp, synthetic construct
  source 1..20: mol_type = other DNA
ggtagtaaagcgcctccggc

SEQ ID NO: 110 - DNA, 20 bp, synthetic construct
  source 1..20: mol_type = other DNA
tggactcgctggcacgcacg

SEQ ID NO: 111 - DNA, 20 bp, synthetic construct
  source 1..20: mol_type = other DNA
tgttgccatggttccagcag

SEQ ID NO: 112 - DNA, 20 bp, synthetic construct
  source 1..20: mol_type = other DNA
caacatcacccgctccaagg

SEQ ID NO: 113 - DNA, 20 bp, synthetic construct
  source 1..20: mol_type = other DNA
cgactggccagggcgcctgt

SEQ ID NO: 114 - DNA, 20 bp, synthetic construct
  source 1..20: mol_type = other DNA
tgcagatcccacaggcgccc

SEQ ID NO: 115 - DNA, 20 bp, synthetic construct
  source 1..20: mol_type = other DNA
acgactggccagggcgcctg

SEQ ID NO: 116 - DNA, 20 bp, synthetic construct
  source 1..20: mol_type = other DNA
aaagaagaggaggatagcaa

SEQ ID NO: 117 - DNA, 20 bp, synthetic construct
  source 1..20: mol_type = other DNA
aagaagaggaggatagcaaa

SEQ ID NO: 118 - DNA, 20 bp, synthetic construct
  source 1..20: mol_type = other DNA
aatggaccaagagcctgcgg

SEQ ID NO: 119 - DNA, 20 bp, synthetic construct
  source 1..20: mol_type = other DNA
accacagtcaggtcttgagg

SEQ ID NO: 120 - DNA, 20 bp, synthetic construct
  source 1..20: mol_type = other DNA
agaagaggaggatagcaaag

SEQ ID NO: 121 - DNA, 20 bp, synthetic construct
  source 1..20: mol_type = other DNA
agatttggagcttggttctg

SEQ ID NO: 122 - DNA, 20 bp, synthetic construct
  source 1..20: mol_type = other DNA
agggaacagaacagtgaaca

SEQ ID NO: 123 - DNA, 20 bp, synthetic construct
  source 1..20: mol_type = other DNA
agtacctaatcacagcatgc

SEQ ID NO: 124 - DNA, 20 bp, synthetic construct
  source 1..20: mol_type = other DNA
agtcatcctgcaatgttggt

SEQ ID NO: 125 - DNA, 20 bp, synthetic construct
  source 1..20: mol_type = other DNA
atagatggagctgcaggaca

SEQ ID NO: 126 - DNA, 20 bp, synthetic construct
  source 1..20: mol_type = other DNA
caatcagaatgtgcaggtgt

SEQ ID NO: 127 - DNA, 20 bp, synthetic construct
  source 1..20: mol_type = other DNA
cagggaacagaacagtgaac

SEQ ID NO: 128 - DNA, 20 bp, synthetic construct
  source 1..20: mol_type = other DNA
cagtcgcatgacgcaagacc

SEQ ID NO: 129 - DNA, 20 bp, synthetic construct
  source 1..20: mol_type = other DNA
ccagagggccggtccacaca

SEQ ID NO: 130 - DNA, 20 bp, synthetic construct
  source 1..20: mol_type = other DNA
ccatgatgcttgcccttgca

SEQ ID NO: 131 - DNA, 20 bp, synthetic construct
  source 1..20: mol_type = other DNA
ccatgtgtggaccggccctc

SEQ ID NO: 132 - DNA, 20 bp, synthetic construct
  source 1..20: mol_type = other DNA
cccatgatgcaagaccttgc

SEQ ID NO: 133 - DNA, 20 bp, synthetic construct
  source 1..20: mol_type = other DNA
ccctgcaagggcaagcatca

SEQ ID NO: 134 - DNA, 20 bp, synthetic construct
  source 1..20: mol_type = other DNA
ccgttgaccttgggccctgc

SEQ ID NO: 135 - DNA, 20 bp, synthetic construct
  source 1..20: mol_type = other DNA
cctgcaaggtcttgcatcat

SEQ ID NO: 136 - DNA, 20 bp, synthetic construct
  source 1..20: mol_type = other DNA
cgggacacagaacagtgaac

SEQ ID NO: 137 - DNA, 20 bp, synthetic construct
  source 1..20: mol_type = other DNA
cgggggacagaacagtgaat

SEQ ID NO: 138 - DNA, 20 bp, synthetic construct
  source 1..20: mol_type = other DNA
ctcgttggacagatccatga

SEQ ID NO: 139 - DNA, 20 bp, synthetic construct
  source 1..20: mol_type = other DNA
gaaagagccgaagcatctgt

SEQ ID NO: 140 - DNA, 20 bp, synthetic construct
  source 1..20: mol_type = other DNA
gaaagggattttgaccactg

SEQ ID NO: 141 - DNA, 20 bp, synthetic construct
  source 1..20: mol_type = other DNA
gagagagaaggtttctcata

SEQ ID NO: 142 - DNA, 20 bp, synthetic construct
  source 1..20: mol_type = other DNA
gcacagagaagggaagttta

SEQ ID NO: 143 - DNA, 20 bp, synthetic construct
  source 1..20: mol_type = other DNA
gcaggagacaactttggatc

SEQ ID NO: 144 - DNA, 20 bp, synthetic construct
  source 1..20: mol_type = other DNA
gcatctgtaggtccctgcaa

SEQ ID NO: 145 - DNA, 20 bp, synthetic construct
  source 1..20: mol_type = other DNA
gctgcagggggcctggccac

SEQ ID NO: 146 - DNA, 20 bp, synthetic construct
  source 1..20: mol_type = other DNA
ggcaagcatcatgggaccga

SEQ ID NO: 147 - DNA, 20 bp, synthetic construct
  source 1..20: mol_type = other DNA
gggacacagaacagtgaaca

SEQ ID NO: 148 - DNA, 20 bp, synthetic construct
  source 1..20: mol_type = other DNA
gtacaagatggtatctgtag

SEQ ID NO: 149 - DNA, 20 bp, synthetic construct
  source 1..20: mol_type = other DNA
gtacacgatgatatctgttg

SEQ ID NO: 150 - DNA, 20 bp, synthetic construct
  source 1..20: mol_type = other DNA
gtacacgctggtatctgtta

SEQ ID NO: 151 - DNA, 20 bp, synthetic construct
  source 1..20: mol_type = other DNA
gtctcttgctcctctgagaa

SEQ ID NO: 152 - DNA, 20 bp, synthetic construct
  source 1..20: mol_type = other DNA
tacaagatggtatctgtagg

SEQ ID NO: 153 - DNA, 20 bp, synthetic construct
  source 1..20: mol_type = other DNA
tacacgatgatatctgttgg

SEQ ID NO: 154 - DNA, 20 bp, synthetic construct
  source 1..20: mol_type = other DNA
taccatctatccagggaggg

SEQ ID NO: 155 - DNA, 20 bp, synthetic construct
  source 1..20: mol_type = other DNA
tcccagtgaccctctggaca

SEQ ID NO: 156 - DNA, 20 bp, synthetic construct
  source 1..20: mol_type = other DNA
tcctgcaaggacaggcatca

SEQ ID NO: 157 - DNA, 20 bp, synthetic construct
  source 1..20: mol_type = other DNA
tcctgcagctccatctatcc

SEQ ID NO: 158 - DNA, 20 bp, synthetic construct
  source 1..20: mol_type = other DNA
tctgtccaacgaggcgtgag

SEQ ID NO: 159 - DNA, 20 bp, synthetic construct
  source 1..20: mol_type = other DNA
tgcaatgttggtcagatgtc

SEQ ID NO: 160 - DNA, 20 bp, synthetic construct
  source 1..20: mol_type = other DNA
tgctgtgattaggtactcag

SEQ ID NO: 161 - DNA, 20 bp, synthetic construct
  source 1..20: mol_type = other DNA
tgttactcactccccctatc

SEQ ID NO: 162 - DNA, 20 bp, synthetic construct
  source 1..20: mol_type = other DNA
ttcacacagagaaaaatcac

SEQ ID NO: 163 - DNA, 20 bp, synthetic construct
  source 1..20: mol_type = other DNA
ctactgtaaacatccttgac

SEQ ID NO: 164 - DNA, 20 bp, synthetic construct
  source 1..20: mol_type = other DNA
tgttcagaggagctttcagt

SEQ ID NO: 165 - DNA, 20 bp, synthetic construct
  source 1..20: mol_type = other DNA
acatcataaccgatttcaaa

SEQ ID NO: 166 - DNA, 20 bp, synthetic construct
  source 1..20: mol_type = other DNA
acaggctgaccgatttctcc

SEQ ID NO: 167 - DNA, 20 bp, synthetic construct
  source 1..20: mol_type = other DNA
cctaaaacactgatttcaaa

SEQ ID NO: 168 - DNA, 20 bp, synthetic construct
  source 1..20: mol_type = other DNA
ttctggaagctggtttcaca

SEQ ID NO: 169 - DNA, 20 bp, synthetic construct
  source 1..20: mol_type = other DNA
ttcagcagcacagttaatac

SEQ ID NO: 170 - DNA, 20 bp, synthetic construct
  source 1..20: mol_type = other DNA
ccttagcagcacgtaaatat

SEQ ID NO: 171 - DNA, 20 bp, synthetic construct
  source 1..20: mol_type = other DNA
atttttgaggcagcacaata

SEQ ID NO: 172 - DNA, 20 bp, synthetic construct
  source 1..20: mol_type = other DNA
agtaaagtagcagcacataa

SEQ ID NO: 173 - DNA, 20 bp, synthetic construct
  source 1..20: mol_type = other DNA
gggcttagctgcttgtgagc

SEQ ID NO: 174 - DNA, 20 bp, synthetic construct
  source 1..20: mol_type = other DNA
acaccaagtcgtgttcacag

SEQ ID NO: 175 - DNA, 20 bp, synthetic construct
  source 1..20: mol_type = other DNA
agtactgtagcagcacatca

SEQ ID NO: 176 - DNA, 20 bp, synthetic construct
  source 1..20: mol_type = other DNA
tttgctgctctagaaattta

SEQ ID NO: 177 - DNA, 20 bp, synthetic construct
  source 1..20: mol_type = other DNA
ctctagcagcacgtaaatat

SEQ ID NO: 178 - DNA, 20 bp, synthetic construct
  source 1..20: mol_type = other DNA
ctaaagcagcacagtaatat

SEQ ID NO: 179 - DNA, 20 bp, synthetic construct
  source 1..20: mol_type = other DNA
cctagaaatagcactggact

SEQ ID NO: 180 - DNA, 20 bp, synthetic construct
  source 1..20: mol_type = other DNA
gctatttctaggtaacacac

SEQ ID NO: 181 - DNA, 20 bp, synthetic construct
  source 1..20: mol_type = other DNA
gctttgagaactgaattcca

SEQ ID NO: 182 - DNA, 20 bp, synthetic construct
  source 1..20: mol_type = other DNA
ctttgagaactgaattccat

SEQ ID NO: 183 - DNA, 20 bp, synthetic construct
  source 1..20: mol_type = other DNA
gctgaagaactgaatttcag

SEQ ID NO: 184 - DNA, 20 bp, synthetic construct
  source 1..20: mol_type = other DNA
tttctagcaccatctgaaat

SEQ ID NO: 185 - DNA, 20 bp, synthetic construct
  source 1..20: mol_type = other DNA
tcattataaccgatttcaga

SEQ ID NO: 186 - DNA, 20 bp, synthetic construct
  source 1..20: mol_type = other DNA
ccaagaacactgatttcaaa

SEQ ID NO: 187 - DNA, 20 bp, synthetic construct
  source 1..20: mol_type = other DNA
ttcaggaagctggtttcata

SEQ ID NO: 188 - DNA, 20 bp, synthetic construct
  source 1..20: mol_type = other DNA
gtaagcatgtgccgcacttg

SEQ ID NO: 189 - DNA, 20 bp, synthetic construct
  source 1..20: mol_type = other DNA
cttgtcaaatacacggagcg

SEQ ID NO: 190 - DNA, 20 bp, synthetic construct
  source 1..20: mol_type = other DNA
ttgaccagccggtgcacagc

SEQ ID NO: 191 - DNA, 20 bp, synthetic construct
  source 1..20: mol_type = other DNA
tagaggaaagtcctgtacat

SEQ ID NO: 192 - DNA, 20 bp, synthetic construct
  source 1..20: mol_type = other DNA
tcggcccagtcgcaagaacc

SEQ ID NO: 193 - DNA, 20 bp, synthetic construct
  source 1..20: mol_type = other DNA
gtacttgctgctgtcgatat

SEQ ID NO: 194 - DNA, 20 bp, synthetic construct
  source 1..20: mol_type = other DNA
tatcagccactaatgaagtt

SEQ ID NO: 195 - DNA, 20 bp, synthetic construct
  source 1..20: mol_type = other DNA
gcccgcaggacgcacccata

SEQ ID NO: 196 - DNA, 25 bp, synthetic construct
  source 1..25: mol_type = other DNA
tggggacatttccaaagtattcaac

SEQ ID NO: 197 - DNA, 21 bp, synthetic construct
  source 1..21: mol_type = other DNA
gctacacgaggaggagacact

SEQ ID NO: 198 - DNA, 25 bp, synthetic construct
  source 1..25: mol_type = other DNA
tattttacagctgacatgggctttg

SEQ ID NO: 199 - DNA, 20 bp, synthetic construct
  source 1..20: mol_type = other DNA
tactgtcggaggtagtcggc

SEQ ID NO: 200 - DNA, 24 bp, synthetic construct
  source 1..24: mol_type = other DNA
gatgccatcctgtttctgtatgga

SEQ ID NO: 201 - DNA, 20 bp, synthetic construct
  source 1..20: mol_type = other DNA
cccggactgtgatggtcaac

SEQ ID NO: 202 - DNA, 25 bp, synthetic construct
  source 1..25: mol_type = other DNA
gtttctgtaatgaggaagttctccg

SEQ ID NO: 203 - DNA, 20 bp, synthetic construct
  source 1..20: mol_type = other DNA
caggggcccccagtagaatc

SEQ ID NO: 204 - DNA, 22 bp, synthetic construct
  source 1..22: mol_type = other DNA
tgacccttctctcatgaccctg

SEQ ID NO: 205 - DNA, 18 bp, synthetic construct
  source 1..18: mol_type = other DNA
agcttgagcacgcagtcg

SEQ ID NO: 206 - DNA, 21 bp, synthetic construct
  source 1..21: mol_type = other DNA
accagtggagacaccaggata

SEQ ID NO: 207 - DNA, 25 bp, synthetic construct
  source 1..25: mol_type = other DNA
atccagaagtaaatggagacaccaa

SEQ ID NO: 208 - DNA, 22 bp, synthetic construct
  source 1..22: mol_type = other DNA
cctgaaatcctatggcacctgg

SEQ ID NO: 209 - DNA, 21 bp, synthetic construct
  source 1..21: mol_type = other DNA
gctgcatctgaggcttagtcc

SEQ ID NO: 210 - DNA, 25 bp, synthetic construct
  source 1..25: mol_type = other DNA
ttcattgatgatgccaatgtcaagc

SEQ ID NO: 211 - DNA, 24 bp, synthetic construct
  source 1..24: mol_type = other DNA
tagactgcatgttagacgagataa

SEQ ID NO: 212 - DNA, 18 bp, synthetic construct
  source 1..18: mol_type = other DNA
cgctagcggaggtgatcg

SEQ ID NO: 213 - DNA, 24 bp, synthetic construct
  source 1..24: mol_type = other DNA
ctgccgtaggttgttgtagatgtc

SEQ ID NO: 214 - DNA, 22 bp, synthetic construct
  source 1..22: mol_type = other DNA
atgggagagggttatgcctttg

SEQ ID NO: 215 - DNA, 30 bp, synthetic construct
  source 1..30: mol_type = other DNA
tcatggaattttaattagttctactcacca

SEQ ID NO: 216 - DNA, 21 bp, synthetic construct
  source 1..21: mol_type = other DNA
taaggaggatgcgcctagtga

SEQ ID NO: 217 - DNA, 25 bp, synthetic construct
  source 1..25: mol_type = other DNA
ggaatgggggaaaggttatcagtaa

SEQ ID NO: 218 - DNA, 21 bp, synthetic construct
  source 1..21: mol_type = other DNA
cacagtggtccttccaggttt

SEQ ID NO: 219 - DNA, 19 bp, synthetic construct
  source 1..19: mol_type = other DNA
gtccttgcctgtcctggaa

SEQ ID NO: 220 - DNA, 24 bp, synthetic construct
  source 1..24: mol_type = other DNA
tgtaggatggtagcacacaaccag

SEQ ID NO: 221 - DNA, 25 bp, synthetic construct
  source 1..25: mol_type = other DNA
agcaagggacgtatttccatcttat

SEQ ID NO: 222 - DNA, 20 bp, synthetic construct
  source 1..20: mol_type = other DNA
gagagcggcttctactggag

SEQ ID NO: 223 - DNA, 20 bp, synthetic construct
  source 1..20: mol_type = other DNA
agtcttttccccttcgagga

SEQ ID NO: 224 - DNA, 25 bp, synthetic construct
  source 1..25: mol_type = other DNA
gaaggttcactaactgtgcgtttta

SEQ ID NO: 225 - DNA, 24 bp, synthetic construct
  source 1..24: mol_type = other DNA
ctgcctttgatcattcaccaaaca

SEQ ID NO: 226 - DNA, 23 bp, synthetic construct
  source 1..23: mol_type = other DNA
aaccaggaatgagtggttcaggg

SEQ ID NO: 227 - DNA, 25 bp, synthetic construct
  source 1..25: mol_type = other DNA
ctctaactgattgcagcgtcttcta

SEQ ID NO: 228 - DNA, 20 bp, synthetic construct
  source 1..20: mol_type = other DNA
gttgcaaggtacggtctgag

SEQ ID NO: 229 - DNA, 18 bp, synthetic construct
  source 1..18: mol_type = other DNA
aagtgactcgagcggcag

SEQ ID NO: 230 - DNA, 20 bp, synthetic construct
  source 1..20: mol_type = other DNA
ttgccgagctggaggccaac

SEQ ID NO: 231 - DNA, 20 bp, synthetic construct
  source 1..20: mol_type = other DNA
atcttggcgttcagccattc

SEQ ID NO: 232 - DNA, 20 bp, synthetic construct
  source 1..20: mol_type = other DNA
atgtccagggttcggacacc

SEQ ID NO: 233 - DNA, 20 bp, synthetic construct
  source 1..20: mol_type = other DNA
gatgaggtcttctgcaggct

SEQ ID NO: 234 - DNA, 20 bp, synthetic construct
  source 1..20: mol_type = other DNA
gttcaggattcacaggctca

SEQ ID NO: 235 - DNA, 20 bp, synthetic construct
  source 1..20: mol_type = other DNA
gaaacactggcatccggttc

SEQ ID NO: 236 - DNA, 20 bp, synthetic construct
  source 1..20: mol_type = other DNA
ccgttaggtacctgtgcgaa

SEQ ID NO: 237 - DNA, 20 bp, synthetic construct
  source 1..20: mol_type = other DNA
ggcggacctgttagacactt

SEQ ID NO: 238 - DNA, 20 bp, synthetic construct
  source 1..20: mol_type = other DNA
tggccaagcagcgcaaaccg

SEQ ID NO: 239 - DNA, 20 bp, synthetic construct
  source 1..20: mol_type = other DNA
atggtccggttcttgtactc

SEQ ID NO: 240 - DNA, 20 bp, synthetic construct
  source 1..20: mol_type = other DNA
gaaattgaggtatggacact

SEQ ID NO: 241 - DNA, 20 bp, synthetic construct
  source 1..20: mol_type = other DNA
ttgaggtatggacacttgga

SEQ ID NO: 242 - DNA, 20 bp, synthetic construct
  source 1..20: mol_type = other DNA
cggaaccgtctgtccgtagt

SEQ ID NO: 243 - DNA, 20 bp, synthetic construct
  source 1..20: mol_type = other DNA
cgctggctcgcggcctgcct

SEQ ID NO: 244 - DNA, 20 bp, synthetic construct
  source 1..20: mol_type = other DNA
cgcgagccagcgccagaagc

SEQ ID NO: 245 - DNA, 20 bp, synthetic construct
  source 1..20: mol_type = other DNA
tcaagactttgttgttcacc

SEQ ID NO: 246 - DNA, 20 bp, synthetic construct
  source 1..20: mol_type = other DNA
catagtcctggcagcccttg

SEQ ID NO: 247 - DNA, 20 bp, synthetic construct
  source 1..20: mol_type = other DNA
gactgtctccctgagagccc

SEQ ID NO: 248 - DNA, 20 bp, synthetic construct
  source 1..20: mol_type = other DNA
cttgtagaatccagtcagtg

SEQ ID NO: 249 - DNA, 20 bp, synthetic construct
  source 1..20: mol_type = other DNA
gtagaatccagtcagtgagg

SEQ ID NO: 250 - DNA, 20 bp, synthetic construct
  source 1..20: mol_type = other DNA
ttacgggccgcctcactgac

SEQ ID NO: 251 - DNA, 20 bp, synthetic construct
  source 1..20: mol_type = other DNA
gaaccaacactcgatcaccg

SEQ ID NO: 252 - DNA, 20 bp, synthetic construct
  source 1..20: mol_type = other DNA
ggtgatcgagtgttggttcg

SEQ ID NO: 253 - DNA, 20 bp, synthetic construct
  source 1..20: mol_type = other DNA
gatcgagtgttggttcgtgg

SEQ ID NO: 254 - DNA, 20 bp, synthetic construct
  source 1..20: mol_type = other DNA
gagtagcgcgagcacagcta

SEQ ID NO: 255 - DNA, 20 bp, synthetic construct
  source 1..20: mol_type = other DNA
aggcccggatggcatcctag

SEQ ID NO: 256 - skipped

SEQ ID NO: 257 - DNA, 20 bp, synthetic construct
  source 1..20: mol_type = other DNA
ggcaggcttgcattccctcc

SEQ ID NO: 258 - DNA, 20 bp, synthetic construct
  source 1..20: mol_type = other DNA
aggggccatggggctgggcc

SEQ ID NO: 259 - DNA, 20 bp, synthetic construct
  source 1..20: mol_type = other DNA
actcaccgacccatcctgcc

SEQ ID NO: 260 - protein, 33 aa, synthetic construct
  VARIANT 2: note = Any amino acid
  VARIANT 3: note = Any amino acid
  VARIANT 5: note = Any amino acid
  VARIANT 12: note = Any amino acid
  VARIANT 13: note = Any amino acid
  VARIANT 14: note = Any amino acid
  VARIANT 16: note = Any amino acid
  VARIANT 17: note = Any amino acid
  VARIANT 18: note = Any amino acid
  VARIANT 21: note = Any amino acid
  VARIANT 25: note = Any amino acid
  VARIANT 26: note = Any amino acid
  VARIANT 33: note = Any amino acid
DXXGXTPLHLAXXXGXXXVVXLLLXXGADVNAX

SEQ ID NO: 261 - protein, 33 aa, synthetic construct
  VARIANT 2: note = Any amino acid
  VARIANT 3: note = Any amino acid
  VARIANT 5: note = Any amino acid
  VARIANT 12: note = Any amino acid
  VARIANT 13: note = Any amino acid
  VARIANT 14: note = Any amino acid
  VARIANT 16: note = Any amino acid
  VARIANT 17: note = Any amino acid
  VARIANT 18: note = Any amino acid
  VARIANT 21: note = Any amino acid
  VARIANT 25: note = Any amino acid
  VARIANT 26: note = Any amino acid
  VARIANT 33: note = Any amino acid
DXXGXTPLHLAXXXGXXXLVXVLLXXGADVNAX

SEQ ID NO: 262 - DNA, 1650 bp, synthetic construct
  source 1..1650: mol_type = other DNA
atggcgagaactctgcggccaagtccactttgccctgggggaggaaaggctcaactgtcttccgccagcctccttggggccgggctgttgctccagccacccacaccgccgcccctgctgctcttgctttttcctctgttgctcttttcaaggctgtgcggtgctctcgcggggccgattatcgtagaaccccatgtaactgcagtatggggaaagaacgtgtccttgaagtgccttattgaggtaaatgagacaatcacgcaaattagttgggagaagattcatggaaaaagtagccaaacagtagctgtccaccatccacagtatgggttctcagtacagggggagtaccaggggcgggtactttttaaaaattactcccttaacgatgcgactatcaccctgcacaatattggtttctcagatagcggcaaatacatttgtaaggctgtgacattcccacttggaaacgctcaatcctcaacgacggttactgttctcgttgagcctactgtatccctcataaagggtccggactctttgattgatggtggtaacgagactgtcgcagccatctgtatagcagcgactgggaagcctgtagcgcatattgattgggagggggatttgggggagatggagtctacaaccaccagtttccccaatgagacagctacaatcatttcacaatacaagttgtttccaacgcggttcgcccggggacgccgaataacctgtgtagttaagcacccggcattggagaaggacattcggtactccttcattctggatattcagtatgctcctgaagtaagtgtgacaggttacgatgggaactggtttgtcggacggaagggtgttaatttgaagtgcaacgcggatgcaaacccacccccttttaagtctgtctggtctcgattggacggacaatggcccgacgggttgttggcgtcagataatactcttcattttgttcatcccctgacttttaattatagcggtgtctacatctgcaaagtcaccaactcccttggtcagcgatccgaccagaaggtgatttatatatccgatcctccaactacgacaactttgcaacccacgattcagtggcacccgtcaaccgctgacatagaggacttggcgactgaaccaaaaaaacttccttttccgttgagtacactcgcaacgataaaagatgataccattgccactataattgcttccgtcgttgggggggctctgtttatcgttcttgtgtccgtgttggctggaatattctgctaccggcgccggcgaaccttccgcggcgattacttcgccaagaattacattcctccctctgacatgcaaaaggaatcacaaatagacgtgttgcaacaggacgagcttgatagttatcctgactctgttaagaaggaaaataagaatccagtgaataatcttatacgaaaggactatctggaagagcccgaaaaaacacaatggaacaatgtggagaatttgaaccgatttgagcgacccatggattactatgaggatcttaagatgggaatgaagttcgtttcagatgagcattatgatgagaatgaagatgaccttgtcagccacgtggacggttctgtcatcagcagaagggaatggtatgtctag